(19) United States
(12) Patent Application Publication
(10) Pub. No.: US 2025/0376478 A1
(43) Pub. Date: Dec. 11, 2025

(54) CYCLIC UREA THIAZOLYL COMPOUNDS FOR TREATMENT OF HSV

(71) Applicant: Assembly Biosciences, Inc., South San Francisco, CA (US)

(72) Inventors: Hassan PAJOUHESH, South San Francisco, CA (US); Min ZHONG, South San Francisco, CA (US); Michael WALKER, South San Francisco, CA (US); Jiaxin YU, South San Francisco, CA (US); Jian ZHANG, South San Francisco, CA (US); Mark BURES, South San Francisco, CA (US)

(21) Appl. No.: 19/107,962

(22) PCT Filed: Aug. 28, 2023
(86) PCT No.: PCT/US2023/031285

§ 371 Date: Feb. 28, 2025

Related U.S. Application Data
(60) Provisional application No. 63/401,877, filed on Aug. 29, 2022.
(60) Provisional application No. 63/445,427, filed on Feb. 14, 2023.
(60) Provisional application No. 63/472,494, filed on Jun. 12, 2023.

(51) Int. Cl. C07D 491/107; A61K 31/427; A61K 31/513; A61K 31/527; A61K 31/5377; A61K 31/538; A61K 31/551; A61P 31/22; C07D 417/04; C07D 417/14; C07D 471/04; C07D 487/04; C07D 495/04; C07D 513/04

(52) U.S. Cl. CPC C07D 491/107; A61K 31/427; A61K 31/513; A61K 31/527; A61K 31/5377; A61K 31/538; A61K 31/551; A61P 31/22; C07D 417/04; C07D 417/14; C07D 471/04; C07D 487/04; C07D 495/04; C07D 513/04

(57) ABSTRACT

The present disclosure provides, in part, cyclic urea thiazolyl compounds, and pharmaceutical compositions thereof, and methods of the treatment and prophylaxis of HSV infections.

### CROSS-REFERENCE TO RELATED APPLICATION

This application claims benefit of U.S. Provisional Application No. 63/401,877, filed Aug. 29, 2022, U.S. Provisional Application No. 63/445,427, filed Feb. 14, 2023, and U.S. Provisional Application No. 63/472,494, filed Jun. 12, 2023, the contents of which are hereby incorporated by reference.

### BACKGROUND

Human herpes viruses are large-enveloped double-stranded DNA viruses that share the characteristic of establishing life-long infections in humans. This is accomplished by their ability to exist in the host either as a symptom free latent infection, where the virus lies dormant or, following activation, as a lytic infection with associated symptoms. These viral infections have widespread, worldwide prevalence and it is notable that over 90% of all humans are chronically infected with more than one human herpes virus.

Human herpes viruses are classified into three subfamilies (i.e., a, p and 7) based upon their biological characteristics and the family consists of eight members, i.e., Herpes Simplex Virus subtype type 1 and 2 (HSV1, HSV2), Varicella Zoster Virus (VZV), Epstein-Barr virus (EBV), Cytomegalovirus (CMV), and human herpes viruses 6-8 (HHV 6-8).

HSV1 and 2 infections can cause disease in immune competent individuals. Both subtypes cause cutaneous genital/anal and orolabial/nasal cavity (cold sore) lesions, although HSV2 is more commonly associated with the former and HSV1 the latter such that >80% of genital infections are believed to be caused by HSV2. Globally, over 500 million people have genital herpes infections. Symptoms vary but are typically most severe on first time of infection and can last for weeks to months. Approximately 50 to 80% of the world's population have orolabial HSV infection, which is the main cause of cold sores. HSV, and particularly HSV1, can also cause lesions on the fingers (Whitlows) and other areas of the skin.

The vast majority of HSV infected individuals will not experience any noticeable symptoms. However, some will experience recurrent outbreaks of infection. In the USA, 20 to 40% of the population will get recurrent labial HSV lesions. Significantly, orolabial cold sores and Whitlow's provide a very easy route for transmission of the virus to other individuals which can lead to rarer but much more serious HSV-related pathologies. For example, HSV-related ocular keratitis is a major cause of blindness. HSV can also cause encephalitis in neonates which is a life-threatening condition. Other disorders also believed to be caused by HSV include herpes gladiatorum, Mollaret's meningitis and possibly Bell's palsy.

Primary infection with, or reactivation of an existing herpes virus infection, can be a major cause of disease in immunocompromised individuals. Key at-risk immunocompromised populations include patients undergoing solid organ or stem cell transplantation, individuals with HIV/AIDS, and ICU patients.

Presently, there is no cure for HSV. Medicines have been developed that can to some degree prevent or shorten outbreaks, but there is a need for improved therapies for treating HSV infection and inhibiting viral replication.

Currently, nucleoside analogues, such as acyclovir and its prodrugs, e.g., valacyclovir and famciclovir, are used as agents against herpes viruses such as HSV. In order to exert their effects, these nucleoside analogues must first be phosphorylated by viral thymidine kinase (TK) and then subsequently converted by cellular kinases to the nucleoside triphosphate, which inhibits the activity of the viral DNA polymerase. If the virus has no functionally active TK, as is the case, for example, with resistant HHV1 mutants or with TK-negative viruses, the active substance is unable to exert its effects.

Nucleoside analogues are clinically administered at a dose as high as several hundred in mg to several grams per day and even in high doses, and over long treatment durations, these compounds do not completely prevent recurrent outbreaks of symptoms from HSV infection. High doses also lead to increased levels of adverse effects.

Viral shedding is also common in HSV patients and can asymptomatically facilitate the transmission of HSV to more individuals. Nucleoside analogues do little to address this and long-term suppressive treatment, e.g., with valacyclovir has been shown to reduce transmission risk only by 46%. Since the nucleoside analogues can incorporate into the genome DNA of a host via the host DNA polymerase, the mutagenicity of these agents is also a concern, as documented for the nucleoside analogue, ganciclovir.

Given the inadequacy of existing treatments, there is an urgent medical need to develop improved, well-tolerated anti-herpes treatments.

A class of compounds being investigated for HSV treatment are the helicase-primase inhibitors. Helicase-primase inhibitors are antiviral agents with a novel mechanism of action against HSV1 and 2. They inhibit the viral heterotrimeric complex consisting of helicase, primase, and cofactor subunits that have functions essential for viral DNA replication. They are not nucleoside analogues and do not require phosphorylation by TK to inhibit HSV replication and they are therefore potentially active against TK-deficient HSV, which as described above, is a major mechanism of resistance to nucleoside analogues, such as acyclovir.

Two examples of helicase-primase inhibitors are BILS-179 BS and amenamevir (Katsumata et al. (2018) Biochem Pharm 158 p 201-206). BILS-179 BS has been dosed orally but was suspended from early clinical trials due to adverse events.

One example of a helicase-primase inhibitor is pritelivir, a thiazolylamide derivative with the chemical name N-[5-(aminosulfonyl)-4-methyl-1,3-thiazol-2-yl]-N-methyl-2-[4-(2-pyridinyl)-phenyl]acetamide. This compound has been disclosed in WO200053591. WO2001047904 discloses thiazolyl amide derivatives and their use as antiviral medicaments. WO2000053591 discloses thiazolyl derivatives and their utilization as antiviral agents. WO2017174640 discloses aminothiazole derivatives useful as antiviral agents. WO2019068817 discloses enantiomers of substituted thiazoles as antiviral compounds.

There is still a need for additional antiviral compounds for the treatment and prophylaxis of HSV infections that have an improved profile with respect to safety, potency, selectivity and/or bioavailability.

### SUMMARY OF THE INVENTION

In one embodiment, the present disclosure provides a compound of Formula I

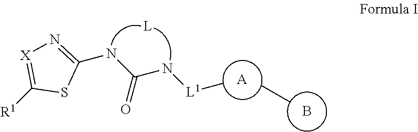

or a pharmaceutically acceptable salt thereof, wherein the variables are as described herein.

In another aspect, the disclosure provides pharmaceutical compositions comprising a compound of Formula I, or a pharmaceutically acceptable salt thereof, and a pharmaceutically acceptable excipient.

In another aspect, the disclosure provides a method of treating an HSV infection in a subject in need thereof, comprising administering to the subject a therapeutically effective amount of compound of Formula I, or a pharmaceutically acceptable salt thereof.

In another aspect, the disclosure provides a method of treating an HSV infection in a subject in need thereof, comprising administering to the subject a pharmaceutical composition comprising a therapeutically effective amount of a compound of Formula I, or a pharmaceutically acceptable salt thereof, and a pharmaceutically acceptable excipient.

In another aspect, the present invention provides a compound of Formula II

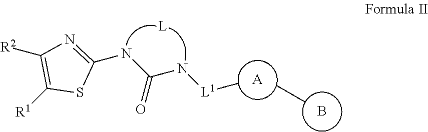

or a pharmaceutically acceptable salt thereof, wherein the variables are as described herein.

In another aspect, the disclosure provides pharmaceutical compositions comprising a compound of Formula II, or a pharmaceutically acceptable salt thereof, and a pharmaceutically acceptable excipient.

In another aspect, the disclosure provides a method of treating an HSV infection in a subject in need thereof, comprising administering to the subject a therapeutically effective amount of compound of Formula II, or a pharmaceutically acceptable salt thereof.

In another aspect, the disclosure provides a method of treating an HSV infection in a subject in need thereof, comprising administering to the subject a pharmaceutical composition comprising a therapeutically effective amount of a compound of Formula II, or a pharmaceutically acceptable salt thereof, and a pharmaceutically acceptable excipient.

### DETAILED DESCRIPTION OF THE INVENTION

The FDA-approved nucleoside drugs, acyclovir, and its prodrug valacyclovir, have been the mainstay of HSV treatment for many years. There have been no regulatory approvals for small-molecule drugs to treat HSV in over two decades, making it an area of significant unmet medical need.

Pritelivir has entered Phase III clinical development by AiCuris for treatment of HSV. Bayer filed patent applications for two scaffolds related to Pritelivir. The first application, WO200053591 (filed on 24 Feb. 2000), relates to compounds with an acyclic urea core. The second application, WO2001047904 (filed on 12 Dec. 2000), relates to compounds, including Pritelivir, with an acyclic amide core. The discovery of Pritelivir (BAY 57-1293) is outlined in G. Kleymann, “Discovery, SAR and Medicinal Chemistry of Herpesvirus Helicase Primase Inhibitors,” Curr. Med. Chem. —Anti-Infective Agents, 2004, 3, 69-83

Of the 227 exemplified acyclic urea compounds disclosed in WO200053591, in vitro HSV-1 and HSV-2 biological assay data are provided for only four Examples: 43, 123, 94 and 2. The structures of Examples 123 and 152 are shown below:

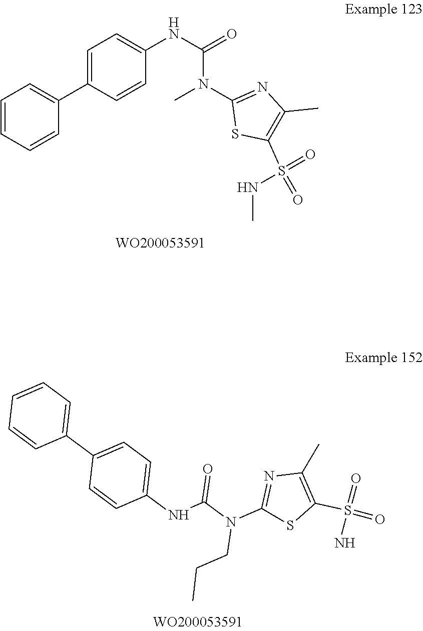 [SMILES: CNS(=O)(=O)c1sc(N(C)C(=O)Nc2ccc(-c3ccccc3)cc2)nc1C ; CCCN(C(=O)Nc1ccc(-c2ccccc2)cc1)c1nc(C)c(S(N)(=O)=O)s1]

Likewise, of the 132 exemplified acyclic amide compounds in WO2001047904, in vitro HSV-1 and HSV-2 biological assay data are provided for only seven Examples: 14, 57, 8, 23, 38, 87 and 126. The structures of Examples 87 and 38 are shown below:

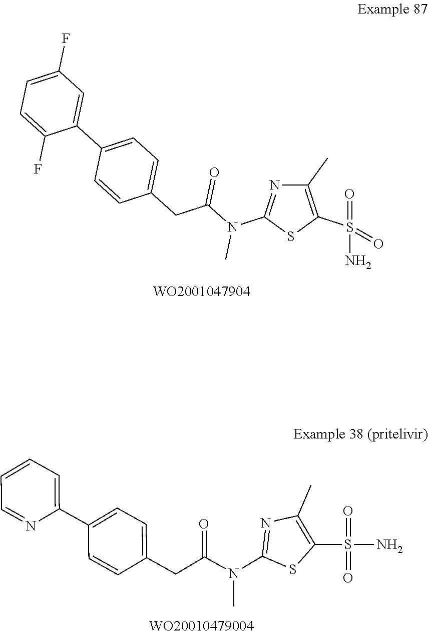 [SMILES: Cc1nc(N(C)C(=O)Cc2ccc(-c3cc(F)ccc3F)cc2)sc1S(N)(=O)=O ; Cc1nc(N(C)C(=O)Cc2ccc(-c3ccccn3)cc2)sc1S(N)(=O)=O ; "WO20010479004"]

Example 5, described herein, is a preferred compound of the present invention. It has a solubility in water at ˜pH 7.0 (measured at room temperature) of less than 5 g/ml. Animal studies were conducted to determine the half-life and clearance of Example 5. Intravenous dosing of a solution of the compound in rat, dog, monkey and mini-pig, at doses of 0.2 mg/kg, 0.15 mg/kg, 0.2 mg/kg and 0.25 mg/kg respectively, gave the terminal half-lives and clearances shown in the following table:

| Species | Half-life (days) | Clearance (L/hr/kg) |
|---|---|---|
| Rat | 0.8 | 0.02 |
| Dog | 2.3 | 0.002 |
| Monkey | 3.0 | 0.004 |
| Mini-pig | 5.6 | 0.0018 |

Based on the above pharmacokinetic data in multiple species, and using allometric scaling, the predicted human biological terminal half-life of the compound is 7.6 days (182 hours) with a clearance of 0.06 L/hr.

Table 1 shows comparison biological assay data for Example 5 of the present invention with the four prior art compounds shown above either lacking a substituent on the urea nitrogen atom between the carbonyl and phenyl moieties (acyclic ureas) or lacking a substituent on the carbon atom between the carbonyl and phenyl moieties (acyclic amides). The prior art compounds were prepared according to known procedures and all biological assay data presented in Table 1 was obtained using the biological assays described herein.

TABLE 1
Biological Activity Comparison of Example 5 with vs
Acyclic Urea and Amide Compounds in the Prior Art

| | | HSV-1 | HSV-2 |
|---|---|---|---|
| | | EC50 | EC50 |
| COMPOUND | Core Moiety | (nM) | (nM) |
| Example 5 | Cyclic Urea | 19 | 10 |
| Example 123 in WO200053591 | Acyclic Urea | 53 | 50 |
| Example152 in WO200053591 | Acyclic Urea | Not | 132 |
| | | tested | |
| Example 87 in WO2001047904 | Acyclic Amide | 17 | 17 |
| Example 38 in WO2001047904 | Acyclic Amide | 70 | 40 |
| (pritelivir) | | | |

The cyclic urea compounds of the present invention incorporate two novel structural changes absent in these prior art compounds. First, both nitrogen atoms have a covalent bond to a carbon atom making it a tetra-substituted urea. Second, the N-alkyl groups attached to the nitrogen atoms are linked to form a ring. These features are neither taught nor suggested in the Bayer patents referenced above.

Only one acyclic urea compound in WO200053591, Example 38, features a tetrasubstituted core ring (i.e., a core ring with substituents attached to both urea nitrogen atoms). More specifically, while all the exemplified compounds are substituted with a diverse group of substituents on the nitrogen which bears the thiazole heterocycle, only Example 38 is additionally substituted on the opposite urea nitrogen atom. Likewise, only one compound in WO2001047904, Example 45, has a substituent on the benzylic carbon atom next to the amide carbonyl, while there is a diverse set of substituents on the amide nitrogen.

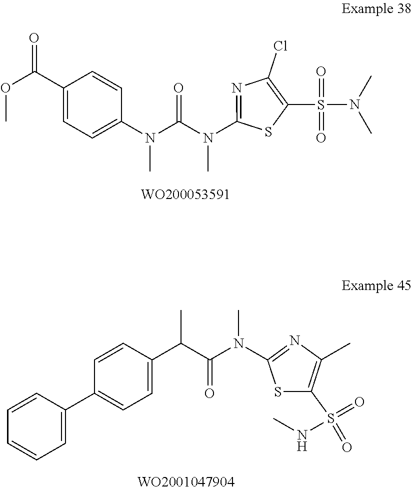 [SMILES: COC(=O)c1ccc(N(C)C(=O)N(C)c2nc(Cl)c(S(=O)(=O)N(C)C)s2)cc1 ; CNS(=O)(=O)c1sc(N(C)C(=O)C(C)c2ccc(-c3ccccc3)cc2)nc1C]

No biological data is provided for either of these compounds and there is no teaching, suggestion or motivation recited in WO200053591 or WO2001047904 to cyclize the urea moiety. Nevertheless, Applicants have discovered that the novel cyclic urea compounds described herein, particularly tetrahydropyrimidin-2(1H)-ones, are surprisingly active relative to their acyclic counterparts and have differing physical and biological properties.

Since no biological data was provided in the prior art for Examples 38 and 45 discussed above, Applicants prepared a novel acyclic tetra-substituted urea Reference Compound A for direct comparison of biological activity relative to Example 5. Applicants note that a direct comparison to Example 38 of WO200053591 might result in ambiguous results due to the presence of a methyl ester moiety on the terminal phenyl group.

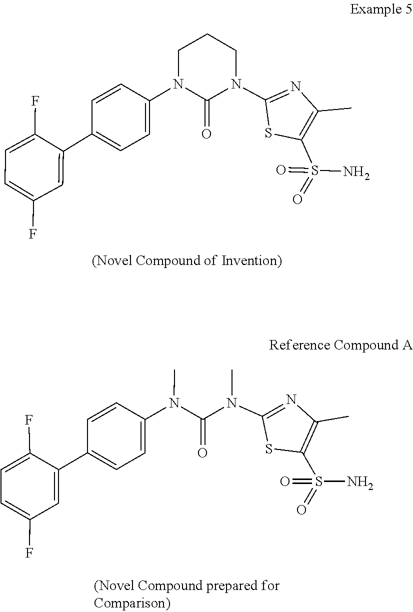 [SMILES: Cc1nc(N2CCCN(c3ccc(-c4cc(F)ccc4F)cc3)C2=O)sc1S(N)(=O)=O ; Cc1nc(N(C)C(=O)N(C)c2ccc(-c3cc(F)ccc3F)cc2)sc1S(N)(=O)=O]

As shown in Table 2, Reference Compound A is 44-fold less active than Example 5 in the HSV-1 assay and 92-fold less active than Example 5 in the HSV-2 assay. Applicants also note that compared to the acyclic amide analog Example 87, shown above, Reference Compound A is 50-fold less active in the HSV-1 assay and 54-fold less active in the HSV-2 assay.

TABLE 2
Biological Activity Comparison of Example 5
of present invention with Novel Acyclic Analog
Substituted on BOTH Urea Nitrogen Atoms

| | | HSV-1 EC50 | HSV-2 EC50 |
|---|---|---|---|
| COMPOUND | Core Moiety | (nM) | (nM) |
| Example 5 | Cyclic Urea | 19 | 10 |
| Reference Compound A | Acyclic Urea | 844 | 925 |

Without specific teachings in the prior art, it was not possible to predict a priori the effect that alkylation of the urea nitrogen atom between the urea carbonyl and phenyl group, as in Reference Compound A, would have on activity. The comparison data presented above demonstrates substantial diminishment of biological activity for an acylic tetrasubstituted urea. Without structural information showing how the compound binds to the target it is not possible to know why methylation reduces activity. There are multiple hypotheses that might explain this observation. Without being bound by theory, these include the following: 1) if the urea NH forms an H-bond to the target, methylation would remove the H-bond donating NH; 2) there might not be enough space in the target to accommodate the methyl group; 3) if the urea carbonyl forms an H-bond to the target, methylation could sterically interfere and weak the interaction; 4) methylation might alter the conformational dynamics of the urea limiting its ability to form a favored bond-conformation; 5) methylation will alter the physical properties the molecule which might reduce its propensity to partition into the inhibitor binding pocket; and 6) methylation will alter the physical properties the molecule which might reduce its ability to enter the cell or access the target once inside the cell. Therefore, the behavior observed with Example 5, where the biological activity is significantly increased by cyclizing the urea moiety, can be considered unexpected and surprising from a medicinal chemistry point of view.

Applicants further point out that generally in medicinal chemistry, cyclization of an acyclic moiety results in diminished activity because the rotational freedom is limited to one conformer, which is statistically unlikely to be the preferred confirmation at the binding site. Without being bound by theory, additional potential explanations for surprising results presented herein include but are not limited to:

1. Conformational Preference: The cyclization of Compound 5 may lead to a specific conformation that aligns more favorably with the target binding site, allowing for stronger interactions and increased biological activity. This preferred conformation could enhance binding affinity and efficacy.

2. Structural Rigidity: The cyclized form of Compound 5 might exhibit greater structural rigidity, leading to improved stability and a reduced entropic cost of binding. This could facilitate a more optimal binding geometry and enhance target engagement.

3. Spatial Constraints: The cyclization may enable the compound to adopt a three-dimensional shape that complements the target's binding pocket, leading to improved molecular recognition and enhanced biological activity.

Applicants have further discovered that among the novel cyclic ureas of the present invention. 6-member core rings (i.e., tetrahydro-2(1H)-pyrimidinones) are generally more active than the corresponding 5-membered core rings (i.e., 2-imidazolidinones). Consider the following two sets of compounds: Examples 5/104 and Examples 259/324 with the following structures:

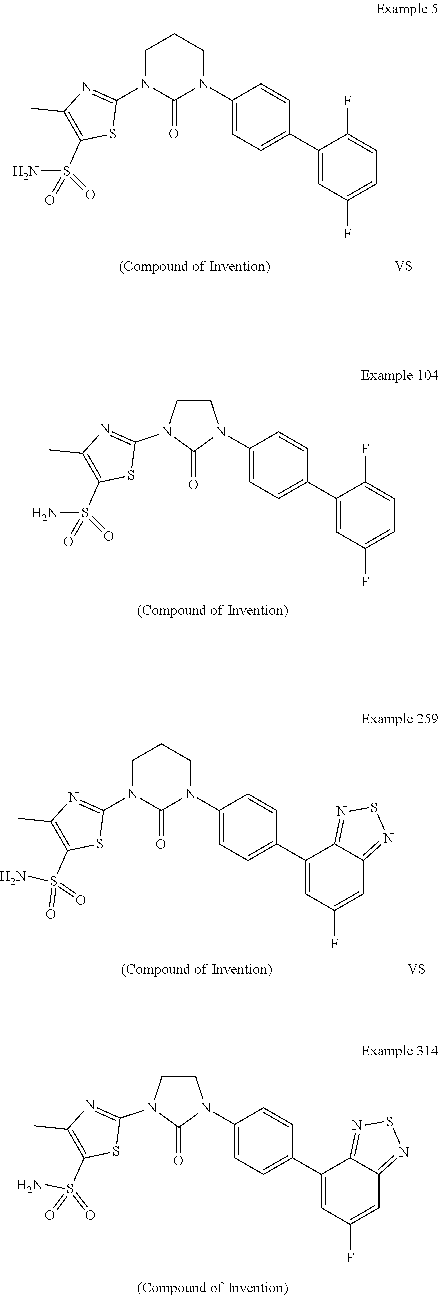 [SMILES: Cc1nc(N2CCCN(c3ccc(-c4cc(F)ccc4F)cc3)C2=O)sc1S(N)(=O)=O ; "VS" ; Cc1nc(N2CCN(c3ccc(-c4cc(F)ccc4F)cc3)C2=O)sc1S(N)(=O)=O ; Cc1nc(N2CCCN(c3ccc(-c4cc(F)cc5nsnc45)cc3)C2=O)sc1S(N)(=O)=O ; "VS" ; Cc1nc(N2CCN(c3ccc(-c4cc(F)cc5nsnc45)cc3)C2=O)sc1S(N)(=O)=O]

As shown in Table 3, Examples 5 is 91-fold more active than Example 104 (the 5-membered ring analog) in the HSV-1 assay and >200-fold more active in the HSV-2 assay. Similarly, Example 259 is 4-fold more active than Example 314 (the 5-membered ring analog).

TABLE 3
Biological Activity Comparison of Compounds
with 5- and 6-membered Cyclic Urea Cores

| | | HSV-1 EC50 | HSV-2 EC50 |
|---|---|---|---|
| COMPOUND | Core Ring Size | (nM) | (nM) |
| Example 5 | 6-membered Ring | 19 | 10 |
| Example 104 | 5-membered ring | 1740 | >2000 > 2 |
| Example 259 | 6-membered ring | 10 | 7 |
| Example 314 | 5-membered ring | Not tested | 28 |

The specific reasons behind this trend are unknown and would require a detailed analysis of the compounds' interactions with the target, including potential differences in conformational flexibility, binding affinity, and steric effects. Without being bound by theory, the larger size of the 6-membered core ring might contribute to improved binding interactions and enhanced potency compared to the 5-membered core ring, which could be a result of the increased conformational space accessible to the 6-membered ring.

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

The term “alkyl” as used herein refers to a saturated straight or branched hydrocarbon. Exemplary alkyl groups include, but are not limited to, straight or branched hydrocarbons of 1-6 or 1-4 carbon atoms, referred to herein as C₁₋₆ alkyl and C₁₋₄ alkyl, respectively. Examples include, but are not limited to, methyl, ethyl, n-propyl, isopropyl, 2-methyl-1-butyl, 3-methyl-2-butyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, t-butyl, n-pentyl, isopentyl, neopentyl, and n-hexyl.

The term “alkylene” as used herein refers to a biradical alkyl group.

The term “alkenyl” as used herein refers to an unsaturated straight or branched hydrocarbon having at least one carbon-carbon double bond. Exemplary alkenyl groups include, but are not limited to, a straight or branched group of 2-6 carbon atoms, referred to herein as C₂₋₆alkenyl. Examples include, but are not limited to, vinyl, allyl, butenyl, and pentenyl.

The term “alkynyl” as used herein refers to an unsaturated straight or branched hydrocarbon having at least one carbon-carbon triple bond. Exemplary alkynyl groups include, but are not limited to, straight or branched groups of 2-6 carbon atoms, referred to herein as C₂₋₆alkynyl. Examples include, but are not limited to, ethynyl, propynyl, butynyl, pentynyl, hexynyl, and methylpropynyl.

The term “alkoxy” as used herein refers to a straight or branched alkyl group attached to oxygen (i.e., alkyl-O—). Exemplary alkoxy groups include, but are not limited to, alkoxy groups of 1-6 or 1-4 carbon atoms, referred to herein as C₁₋₆alkoxy and C₁₋₄alkoxy, respectively. Examples include, but are not limited to, methoxy, ethoxy, and isopropoxy.

The term “alkoxyalkyl” as used herein refers to an alkyl group substituted with an alkoxy group. Exemplary alkoxyalkyl groups include, but are not limited to, a C₁₋₆alkyl group substituted with a C₁₋₃alkoxy or C₁₋₄alkoxy group, referred to herein as C₁₋₃alkoxyC₁₋₆alkyl and C₁₋₄alkoxyC₁₋₆alkyl, respectively. Examples include, but are not limited to, CH₃CH₂OCH₂—, CH₃OCH₂CH₂— and CH₃OCH₂—.

The term “cyano” as used herein refers to CN.

The term “monocycloalkyl” as used herein refers to a saturated monocyclic hydrocarbon group of, for example, 3-6 carbons, referred to herein as C₃₋₆monocycloalkyl. Examples include, btu are not limited to, cyclooctyl, cycloheptyl. cyclohexyl, cyclopentenyl, cyclobutyl and cyclopropyl.

The terms “halo” or “halogen” as used herein refer to F, Cl, Br or I.

The term “haloalkyl” as used herein refers to an alkyl group substituted with one or more halogen atoms. Exemplary haloalkyl groups include, but are not limited to, a C₁₋₆alkyl or C₁₋₄alkyl substituted with one or more halo groups, referred to herein as haloC₁₋₆alkyl and haloC₁₋₄alkyl, respectively. For example, haloC₁₋₆alkyl refers to a straight or branched alkyl group of 1-6 carbon atoms substituted with one or more halogen atoms. Examples include, but are not limited to, —CH₂F, —CHCl₂, —CHF₂, —CF₃, CF₃CH₂—, CH₃CF₂—, CF₃CCl₂— and CF₃CF₂—.

The term “haloalkoxy” as used herein refers to an alkoxy group substituted with one or more halogen atoms. Exemplary alkoxy groups include, but are not limited to, a C₁₋₆alkoxy or C₁₋₄alkoxy substituted with one or more halo groups, referred to herein as haloC₁₋₆alkoxy and haloC₁₋₄alkoxy, respectively. Examples include, but are not limited to, CCl₃O—, CF₃O—, CHF₂O—CF₃CH₂O—, and CF₃CF₂O—.

The terms “hydroxy” and “hydroxyl” as used herein refer to OH.

The term “hydroxyalkyl” as used herein refers to an alkyl group substituted with one or more hydroxy groups. Exemplary hydroxyalkyl groups include, but are not limited to, a C₁₋₆alkyl or C₁₋₄alkyl substituted with one or more hydroxy groups, referred to herein as hydroxyC₁₋₆alkyl and hydroxyC₁₋₄alkyl, respectively. Examples include, but are not limited to, HOCH₂—, HOCH₂CH₂—, CH₃CH(OH)CH₂—, (CH₃)₂C(OH)CH₂—, and HOCH₂CH(OH)CH₂—.

The term “hydroxyalkoxy” as used herein refers to an alkoxy group substituted with one or more hydroxy groups. Exemplary hydroxyalkoxy groups include, but are not limited to, a C₁₋₆alkoxy or C₁₋₄alkoxy substituted with one or more hydroxy groups, referred to herein as hydroxyC₁₋₆alkoxy and hydroxyC₁₋₄alkoxy, respectively. Examples include, but are not limited, to HOCH₂O—, HOCH₂CH₂O—, CH₃CH(OH)CH₂O—, (CH₃)₂C(OH)CH₂O—, and HOCH₂CH(OH)CH₂O—.

The term “RⁿR^{m}Nalkyl-,” as used herein refers to an alkyl group substituted with a RⁿR^{m}N— group, as defined herein. Exemplary RⁿR^{m}Nalkyl- groups include, but are not limited to, a C₁₋₆alkyl or C₁₋₄alkyl substituted with one or more RⁿR^{m}N— group groups, referred to herein as RⁿR^{m}NC₁₋₆alkyl and RⁿR^{m}NC₁₋₄alkyl, respectively. Examples include, but are not limited to NH₂CH₂—, NH(CH₃)CH₂—, N(CH₃)₂CH₂CH₂— and CH₃CH(NH₂)CH₂—.

The term “RⁿR^{m}Nalkoxy,” as used herein refers to an alkoxy group substituted with a RⁿR^{m}N— group, as defined herein. Exemplary RⁿR^{m}Nalkoxy groups include, but are not limited to, a C₁₋₆alkoxy or C₁₋₄alkoxy substituted with one or more RⁿR^{m}N— groups, referred to herein as RⁿR^{m}NC₁₋₆alkoxy and RⁿR^{m}NC₁₋₄alkoxy, respectively. Examples include, but are not limited to, NH₂CH₂—, NH(CH₃)CH₂O—, N(CH₃)₂CH₂CH₂O—, and CH₃CH(NH₂)CH₂O—.

As used herein, when a bicyclic ring is shown with a floating point of attachment and/or floating substituents, for example as in

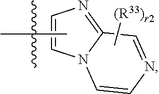

it signifies that the bicyclic ring can be attached via a carbon atom on either ring, and that the substituents (e.g., the R³³ group(s)) can be independently attached to either or both rings.

The terms “Individual,” “patient,” or “subject” are used interchangeably and include any animal, including mammals, preferably mice, rats, other rodents, rabbits, dogs, cats, swine, cattle, sheep, horses, or primates, and most preferably humans. The compounds or pharmaceutical compositions of the disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, dogs, primates, and the like). The mammal treated in the methods of the disclosure is desirably a mammal in which treatment of HSV infection is desired.

The term “modulation” includes antagonism (e.g., inhibition), agonism, partial antagonism and/or partial agonism.

The term “Pharmaceutically acceptable” includes molecular entities and compositions that do not produce an adverse, allergic, or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, fillers, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable excipients.

The term “pharmaceutically acceptable salt(s)” as used herein refers to salts of acidic or basic groups that may be present in compounds used in the compositions. Compounds included in the present compositions that are basic in nature can form a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

The term “therapeutically effective amount” or “effective amount” as used herein refers to the amount of the subject compound that will elicit the biological or medical response of a tissue, system or animal, (e.g., mammal or human) that is being sought by the researcher, veterinarian, medical doctor or other clinician. The compounds or pharmaceutical compositions of the disclosure are administered in therapeutically effective amounts to treat a disease. Alternatively, a therapeutically effective amount of a compound is the quantity required to achieve a desired therapeutic and/or prophylactic effect.

The term “treating” includes any effect, e.g., lessening, reducing, modulating, or eliminating, a viral infection, that results in the improvement of the disease.

The compounds of the disclosure may contain one or more chiral centers and, therefore, exist as stereoisomers. The term “stereoisomers” when used herein consist of all enantiomers or diastereomers. These compounds may be designated by the symbols “(+),” “(−),” “R” or “S,” depending on the configuration of substituents around the stereogenic carbon atom, but the skilled artisan will recognize that a structure may denote a chiral center implicitly. The present disclosure encompasses various stereoisomers of these compounds and mixtures thereof. Mixtures of enantiomers or diastereomers may be designated “(±)” in nomenclature, but the skilled artisan will recognize that a structure may denote a chiral center implicitly.

The compounds of the disclosure may contain one or more double bonds and, therefore, exist as geometric isomers resulting from the arrangement of substituents around a carbon-carbon double bond. The symbol [symbol] denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond.

Compounds of the disclosure may contain a carbocyclic or heterocyclic ring and therefore, exist as geometric isomers resulting from the arrangement of substituents around the ring. The arrangement of substituents around a carbocyclic or heterocyclic ring are designated as being in the “Z” or “E” configuration wherein the terms “Z” and “E” are used in accordance with IUPAC standards. Unless otherwise specified, structures depicting carbocyclic or heterocyclic rings encompass both “Z” and “E” isomers. Substituents around a carbocyclic or heterocyclic ring may also be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diasteromers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well-known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantiomeric and diastereoselective transformations and may involve the use of chiral auxiliaries. For examples, see Carreira and Kvaerno, Classics in Stereoselective Synthesis, Wiley-VCH: Weinheim, 2009.

The compounds disclosed herein can exist in solvated as well as unsolvated forms with pharmaceutically acceptable solvents such as water, ethanol, and the like, and it is intended that the disclosure embrace both solvated and unsolvated forms. In one embodiment, the compound is amorphous. In one embodiment, the compound is a single polymorph. In another embodiment, the compound is a mixture of polymorphs. In another embodiment, the compound is in a crystalline form.

The disclosure also embraces isotopically labeled compounds of the disclosure which are identical to those recited herein, except that one or more atoms are replaced by an atom having an atomic mass or mass number different from the atomic mass or mass number usually found in nature. Examples of isotopes that can be incorporated into compounds of the disclosure include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, P, ³²P, ³⁵S, ¹⁸F, and ³⁶Cl, respectively. For example, a compound of the disclosure may have one or more H atom replaced with deuterium.

Certain isotopically labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the disclosure can generally be prepared by following procedures analogous to those disclosed in the examples herein by substituting an isotopically labeled reagent for a non-isotopically labeled reagent.

The term “prodrug” refers to compounds that are transformed in vivo to yield a disclosed compound or a pharmaceutically acceptable salt, hydrate or solvate of the compound. The transformation may occur by various mechanisms (such as by esterase, amidase, phosphatase, oxidative and or reductive metabolism) in various locations (such as in the intestinal lumen or upon transit of the intestine, blood or liver). Prodrugs are well known in the art (for example, see Rautio, Kumpulainen, et al., Nature Reviews Drug Discovery 2008, 7, 255).

### Cyclic Urea Thiazolyl Compounds

In one aspect, the present disclosure provides a compound of Formula I

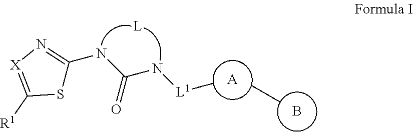

or a pharmaceutically acceptable salt thereof, wherein:

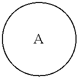

is selected from the group consisting of

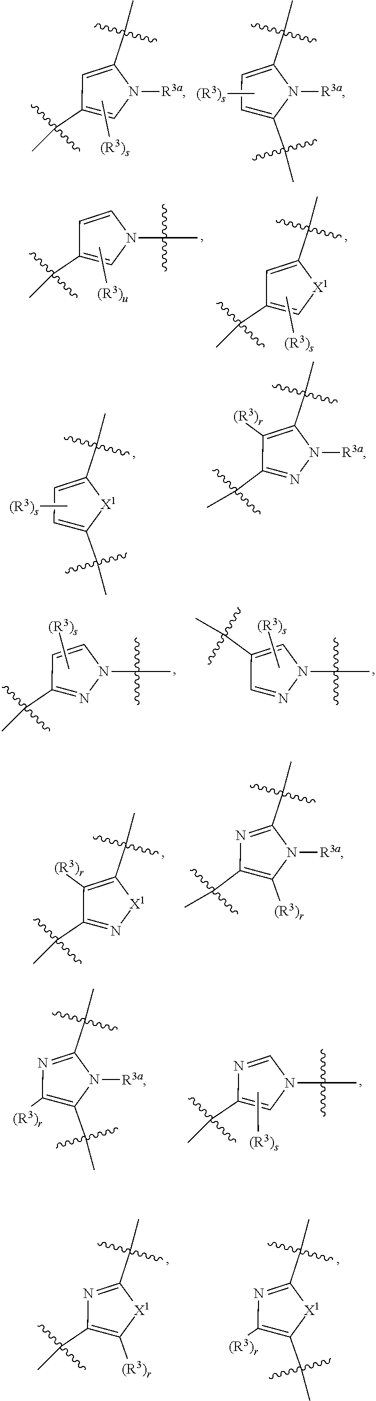

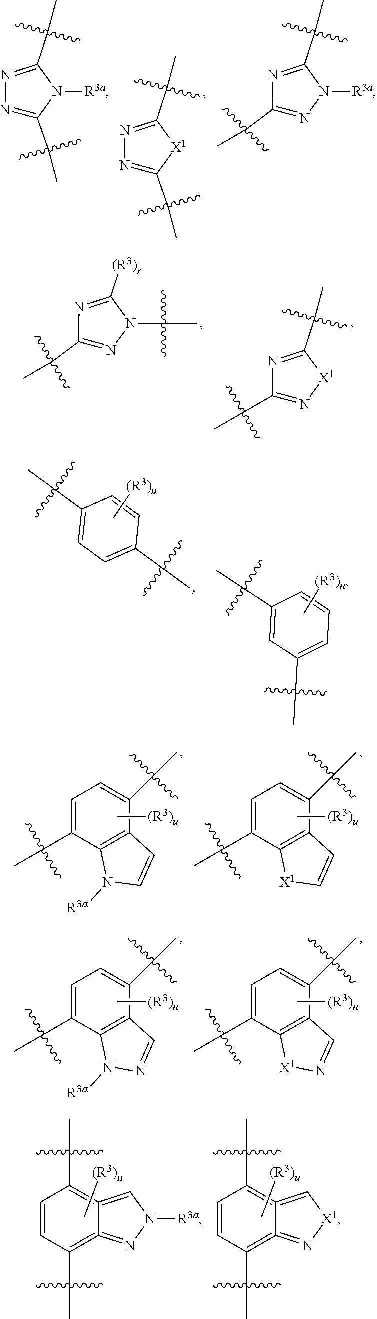

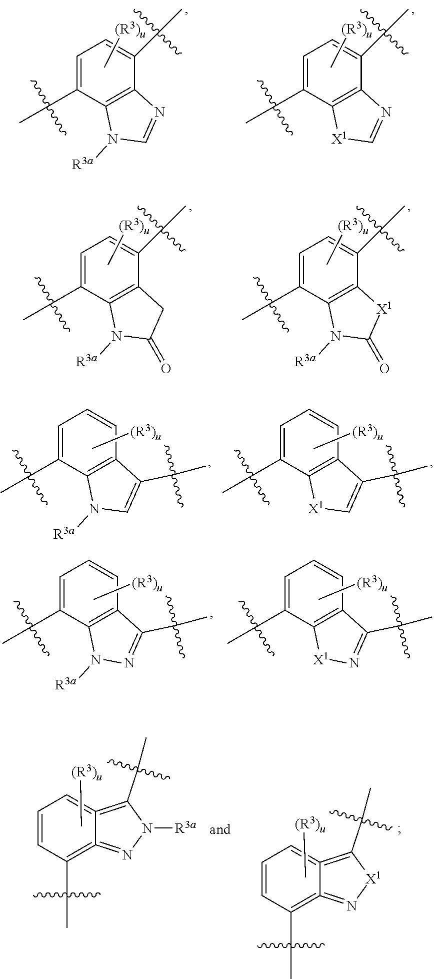

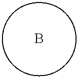

is selected from the group consisting of

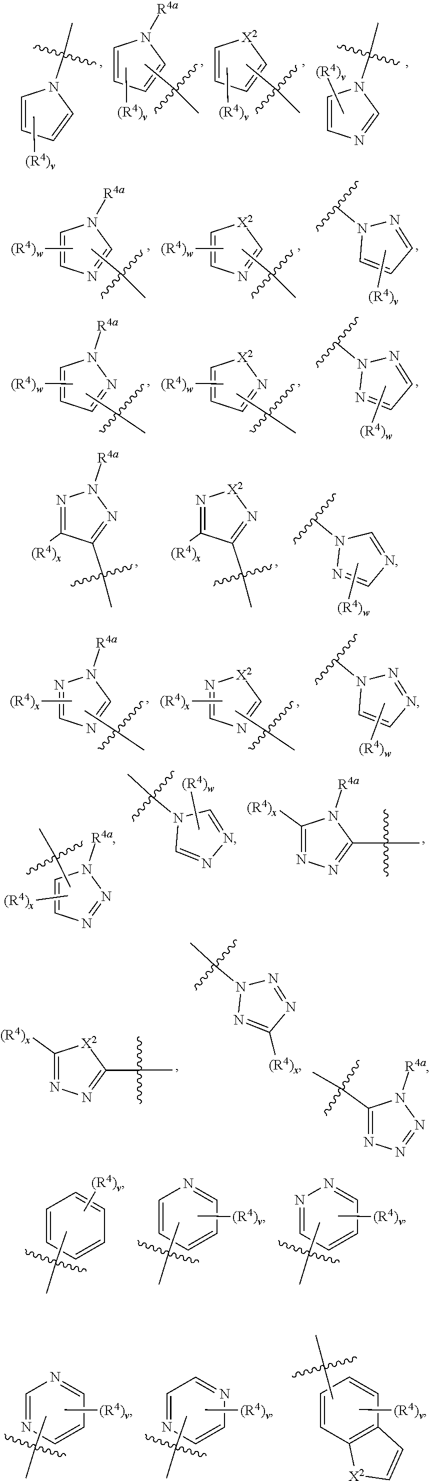

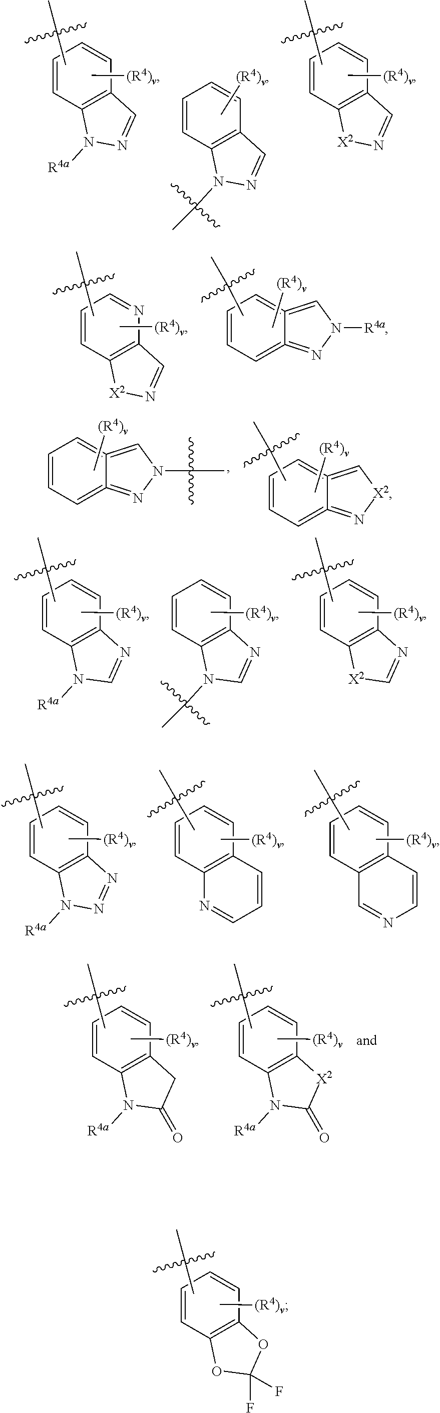

X is CR² or N;
X⁰ is O, S or NRX;
X¹, X², X³, X⁴ and X⁶ are independently selected from the group consisting of O and S;
X⁵ is CH₂, CF₂, O, S or NR^{y},
L is

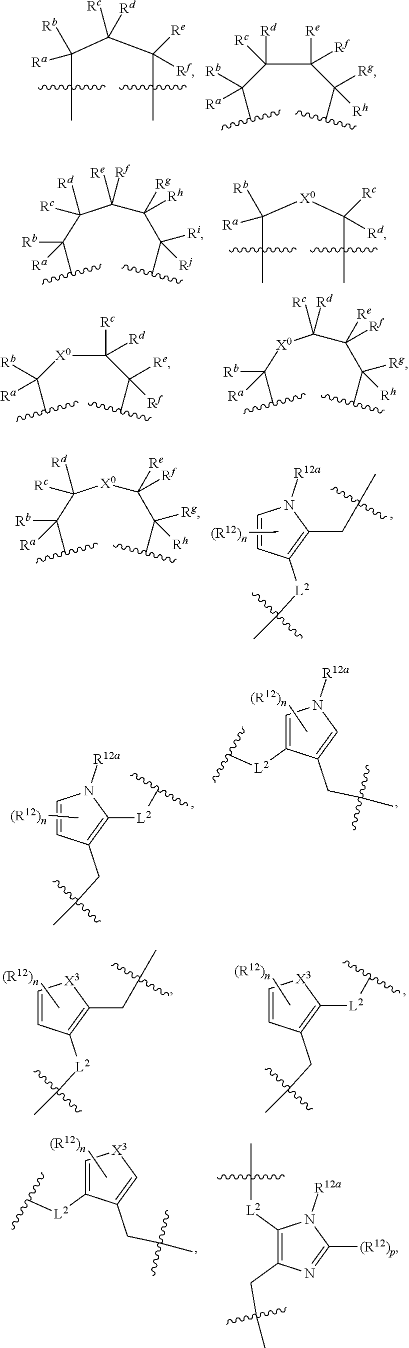

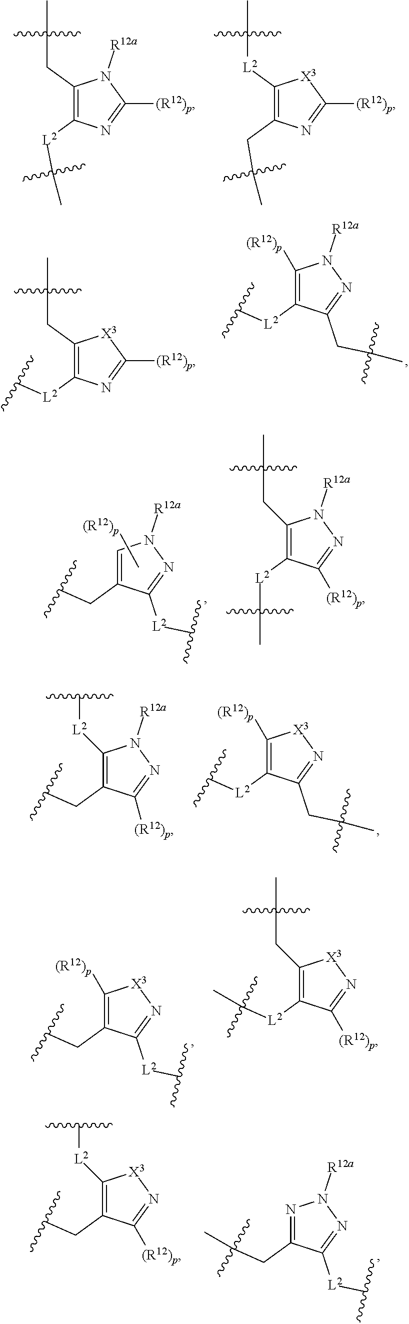

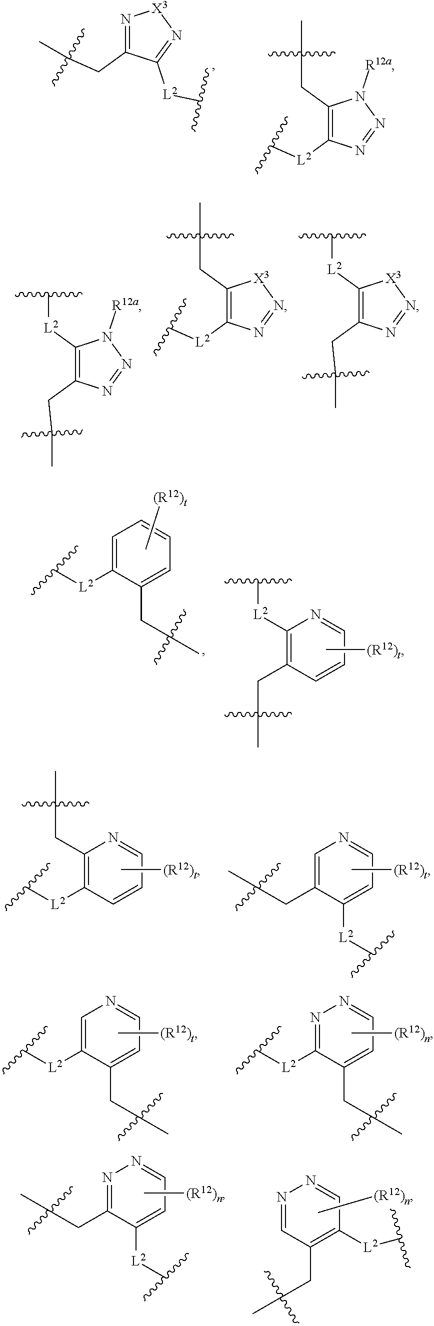

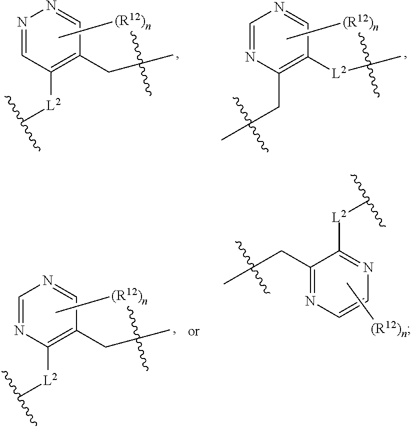

L¹ is a bond; or L¹ is —CH₂— or CH(CH₃)— when

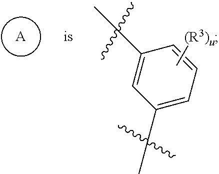

L² is —CH₂—, —CH₂CH₂— or —CH₂CH₂CH₂—;
R^{a}, R^{b}, R^{c}, R^{d}, R^{e}, R^{f}, R^{g}, R^{h}, Rⁱ and R^{j} are independently selected from the group consisting of hydrogen, halo, cyano, OH, NRⁿR^{m}, —C(O)OH, —C(O)OC₁₋₄alkyl, —C(O)NRⁿR^{m}, —SO₂NRⁿR^{m}, C₁₋₄alkyl, haloC₁₋₄alkyl, hydroxyC₁₋₄alkyl, C₁₋₄alkoxy, and R¹³, provided that only one of R^{a}, R^{b}, R^{c}, R^{d}, R^{e}, R^{f}, R^{h}, R^{h}, Rⁱ and R^{j} can be R¹³; or two R-groups together with the carbon atom to which they are attached form a cyclopropyl, cyclobutyl, cyclopentyl, cyclohexyl,

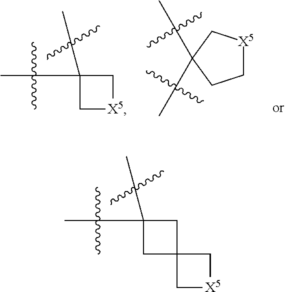

group;
R^{x} and R^{y} are independently selected from the group consisting of is hydrogen, C₁₋₄alkyl, and acetyl;
R¹ is

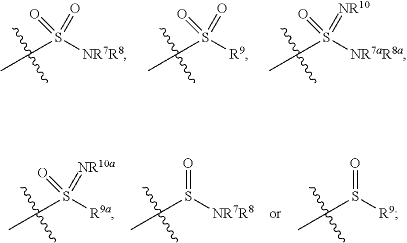

R² is hydrogen, halo, C₁₋₄alkyl, haloC₁₋₄alkyl, C₁₋₄alkoxy or haloC₁₋₄alkoxy;
R³ is independently selected for each occurrence from the group consisting of halo and cyano;
R^{3a}, R^{4a}, R^{11a}, R^{12a} and R^{14a} are independently selected from the group consisting of hydrogen, C₁₋₄alkyl, halo C₁₋₄alkyl and hydroxyC₁₋₄alkyl;
R⁴ is independently selected for each occurrence from the group consisting of halo, CN, OH, NRⁿR^{m}, C₁₋₄alkyl, haloC₁₋₄alkyl, C₂₋₄alkenyl, C₂₋₄alkynyl optionally substituted with hydroxyC₁₋₃alkyl, cyclopropyl optionally substituted with halo or cyano, and R^{4b}, provided that only one R⁴ group can be R^{4b};
R^{4b} is selected from the group consisting of

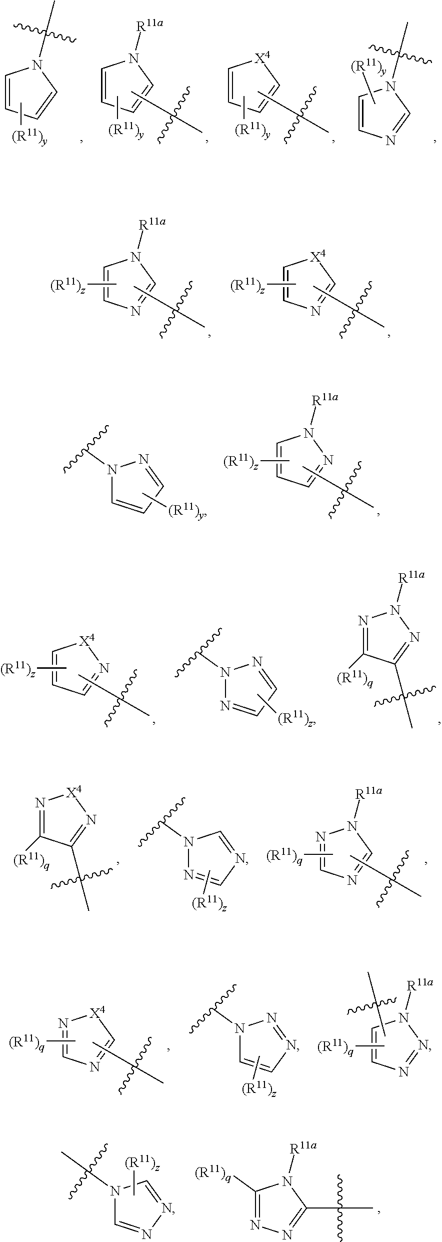

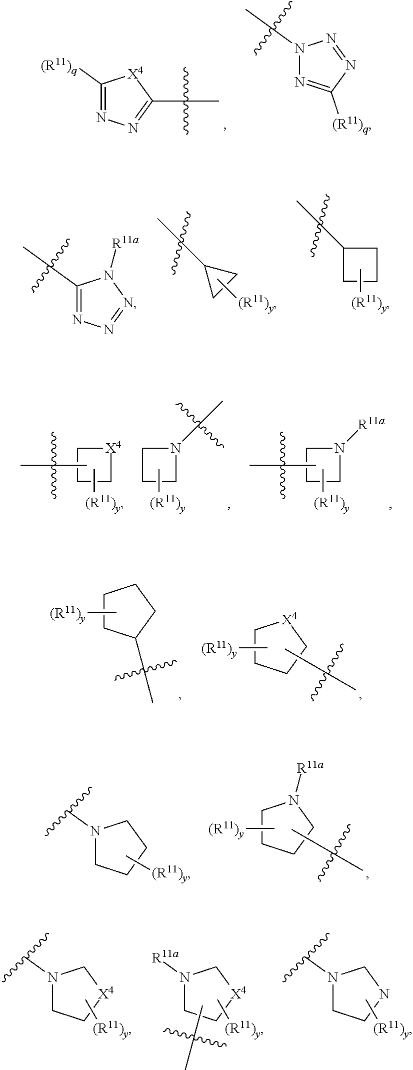

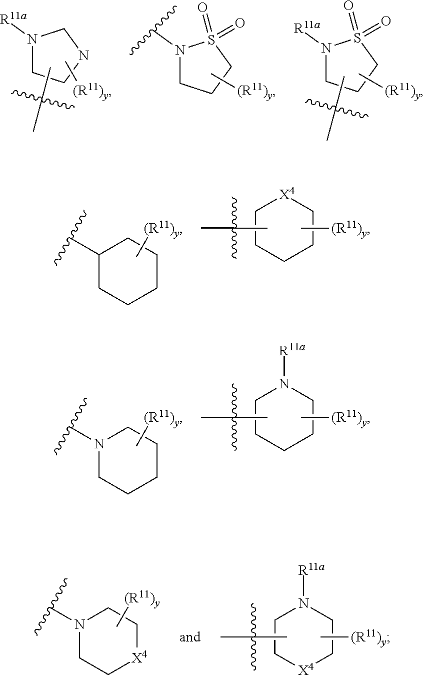

R⁷ and R⁸ are independently selected from the group consisting of hydrogen, C₁₋₄alkyl, acetyl, C₃₋₆monocycloalkyl, phenyl, and pyridyl; or R⁷ and R⁸ together with the N atom to which they are attached form an arizidinyl, azetidinyl, pyrrolidinly, piperidinyl, morpholinyl or thiomorpholinyl group;
R^{7a} and R^{8a} are independently selected from the group consisting of hydrogen, C₁₋₄alkyl and C₃₋₆monocycloalkyl, or R⁷ and R⁸ together with the N atom to which they are attached form an arizidinyl, azetidinyl, pyrrolidinyl, or piperidinyl morpholinyl or thiomorpholinyl group;
R⁹ and R^{9a} are independently selected from the group consisting or C₁₋₄alkyl and haloC₁₋₄alkyl;
R¹⁰ and R^{10a} are independently selected from the group consisting of hydrogen and C₁₋₄alkyl;
R¹¹, R¹² and R¹⁴ are independently selected for each occurrence from the group consisting of halo, CN, OH, NRⁿR^{m}, C₁₋₄alkyl, haloC₁₋₄alkyl, C₂₋₄alkenyl, C₂₋₄alkynyl, and cyclopropyl;
R¹³ is selected from the group consisting of

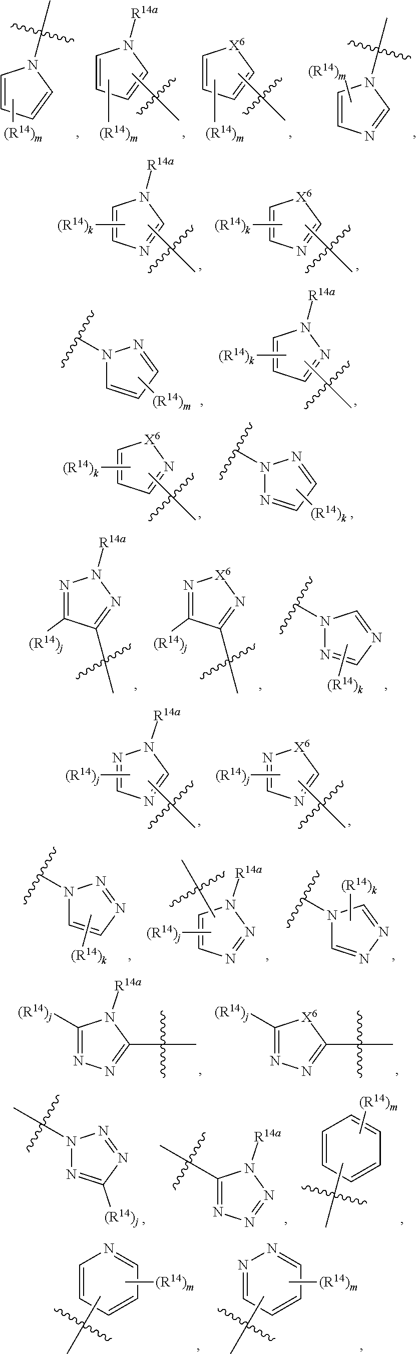

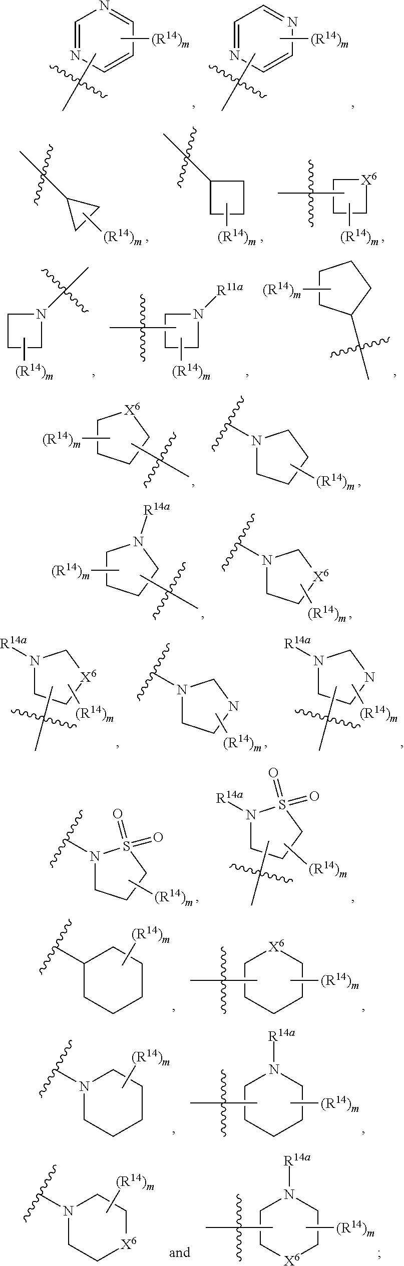

Rⁿ and R^{m} are independently selected for each occurrence from the group consisting or hydrogen and C₁₋₄alkyl;
j, p, q, r and x are independently selected from the group consisting of 0 and 1;
k, n, s, w and z are independently selected from the group consisting of 0, 1 and 2; and
m, t, u, v and y are independently selected from the group consisting of 0, 1, 2 and 3.

In another aspect, the present disclosure provides a compound of Formula Ia

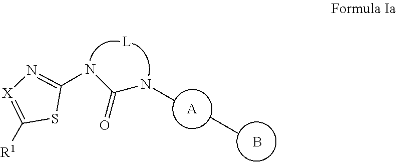

or a pharmaceutically acceptable salt thereof, wherein:

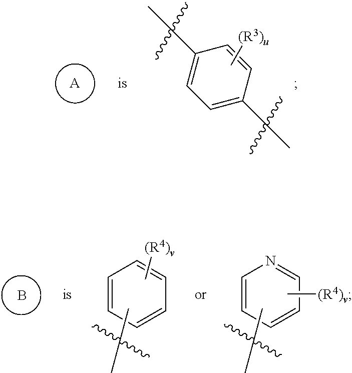

X is CR² or N;
X⁵ is CH₂, CF₂, O, S or NR^{y};
L is

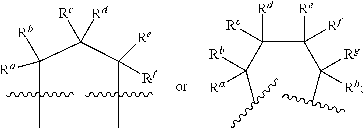

R^{a}, R^{b}, R^{c}, R^{d}, R^{e}, R^{f}, R^{g} and R^{h} are independently selected from the group consisting of hydrogen, halo, cyano, OH, NRⁿR^{m}, —C(O)OH, —C(O)OC₁₋₄alkyl, —C(O)NRⁿR^{m}, —SO₂NRⁿR^{m}, C₁₋₄alkyl, haloC₁₋₄alkyl, hydroxyC₁₋₄alkyl, C₁₋₄alkoxy, and R¹³, provided that only one of R^{a}, R^{b}, R^{c}, R^{d}, R^{e}, R^{f}, R^{g} and R^{h} can be R¹³; or two R-groups together with the carbon atom to which they are attached form a cyclopropyl, cyclobutyl, cyclopentyl, cyclohexyl,

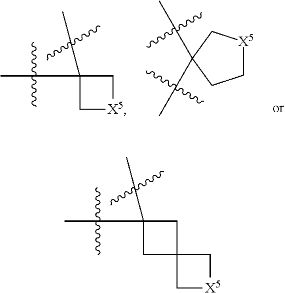

group;
R^{y} is hydrogen, C₁₋₄alkyl or acetyl;
R¹ is

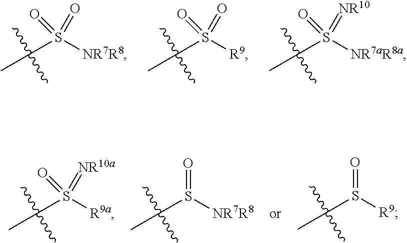

R² is hydrogen, halo, C₁₋₄alkyl, haloC₁₋₄alkyl, C₁₋₄alkoxy or haloC₁₋₄alkoxy;
R³ is independently selected for each occurrence from the group consisting of halo and cyano;
R⁴ is independently selected for each occurrence from the group consisting of halo, CN, OH, NRⁿR^{m}, C₁₋₄alkyl, haloC₁₋₄alkyl, C₂₋₄alkenyl, C₂₋₄alkynyl optionally substituted with hydroxyC₁₋₃alkyl, and cyclopropyl optionally substituted with halo or cyano;
R⁷ and R⁸ are independently selected from the group consisting of hydrogen, C₁₋₄alkyl, acetyl, C₃₋₆monocycloalkyl, phenyl, and pyridyl; or R⁷ and R⁸ together with the N atom to which they are attached form an arizidinyl, azetidinyl, pyrrolidinly, piperidinyl, morpholinyl or thiomorpholinyl group;
R^{7a} and R^{8a} are independently selected from the group consisting of hydrogen, C₁₋₄alkyl and C₃₋₆monocycloalkyl, or R⁷ and R⁸ together with the N atom to which they are attached form an arizidinyl, azetidinyl, pyrrolidinyl, or piperidinyl morpholinyl or thiomorpholinyl group;
R⁹ and R^{9a} are independently selected from the group consisting or C₁₋₄alkyl and haloC₁₋₄alkyl;
R¹⁰ and R^{10a} are independently selected from the group consisting of hydrogen and C₁₋₄alkyl;
R¹³ is selected from the group consisting of

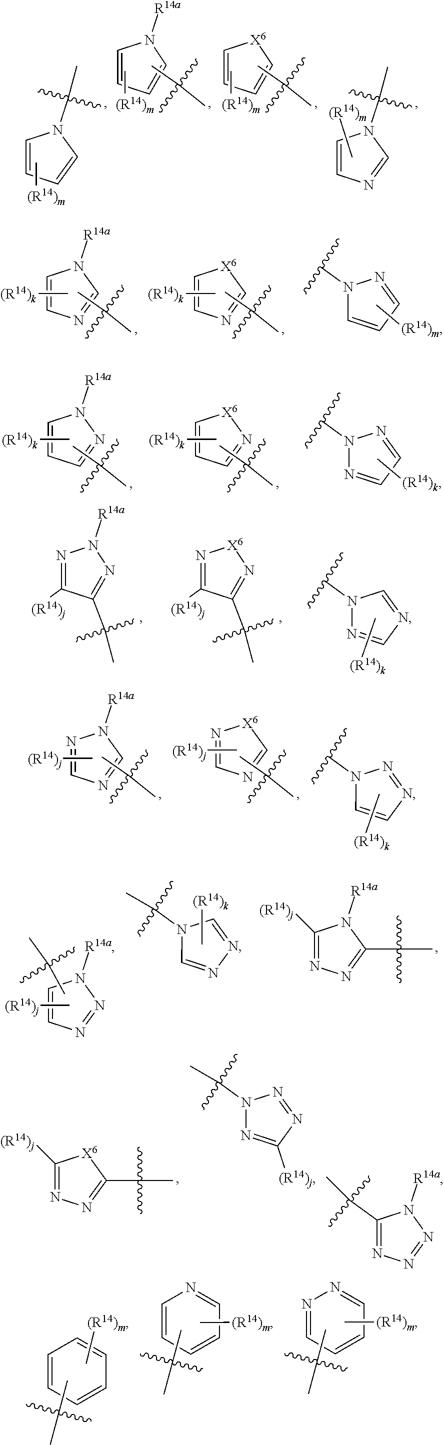

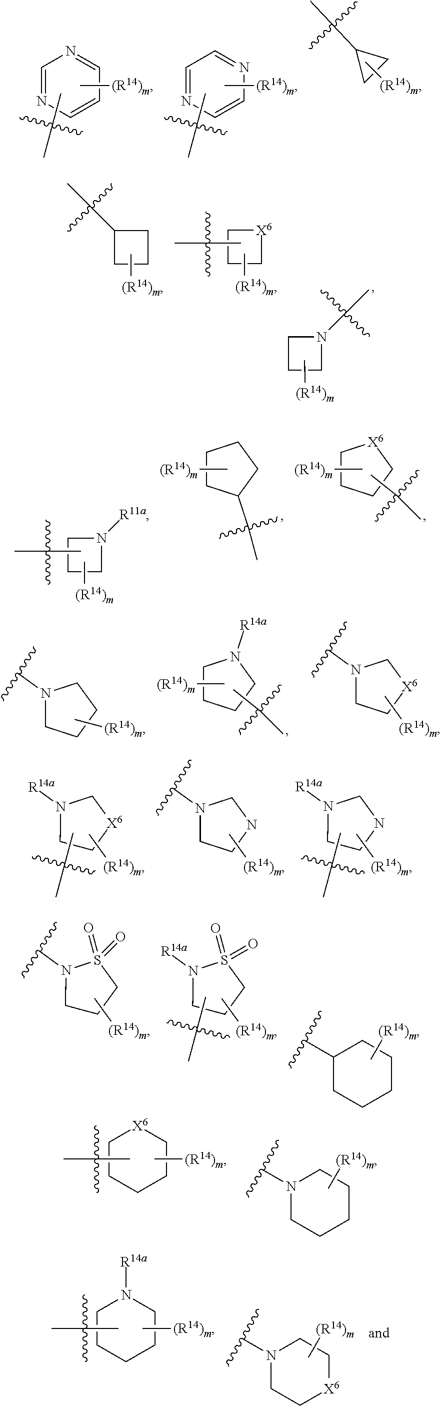

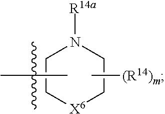

R¹⁴ is independently selected for each occurrence from the group consisting of halo, CN, OH, NRⁿR^{m}, C₁₋₄alkyl, haloC₁₋₄alkyl, C₂₋₄alkenyl, C₂₋₄alkynyl, and cyclopropyl;
R^{14a} is selected from the group consisting of hydrogen, C₁₋₄alkyl, halo C₁₋₄alkyl and hydroxyC₁₋₄alkyl;
Rⁿ and R^{m} are independently selected for each occurrence from the group consisting or hydrogen and C₁₋₄alkyl;
j is selected from the group consisting of 0 and 1;
k is selected from the group consisting of 0, 1 and 2; and
m, u and v are independently selected from the group consisting of 0, 1, 2 and 3.

The following embodiments further describe a compound of Formula I, Formula Ia, or a pharmaceutically acceptable salt thereof. It will be appreciated that all chemically allowable combinations of the embodiments described herein are envisioned as further embodiments of the invention.

In certain embodiments,

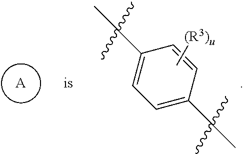

In certain embodiments,

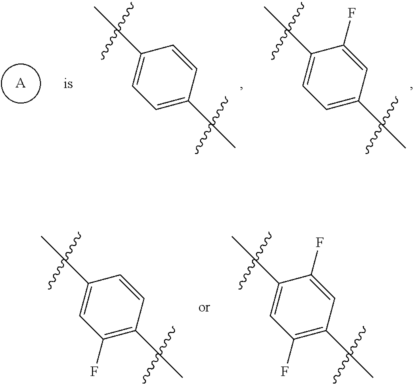

In certain embodiments,

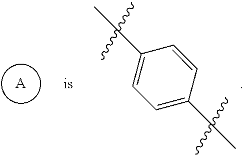

In certain embodiments,

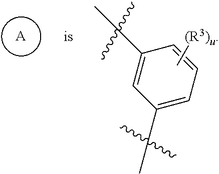

In certain embodiments,

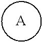

is selected from the group consisting of

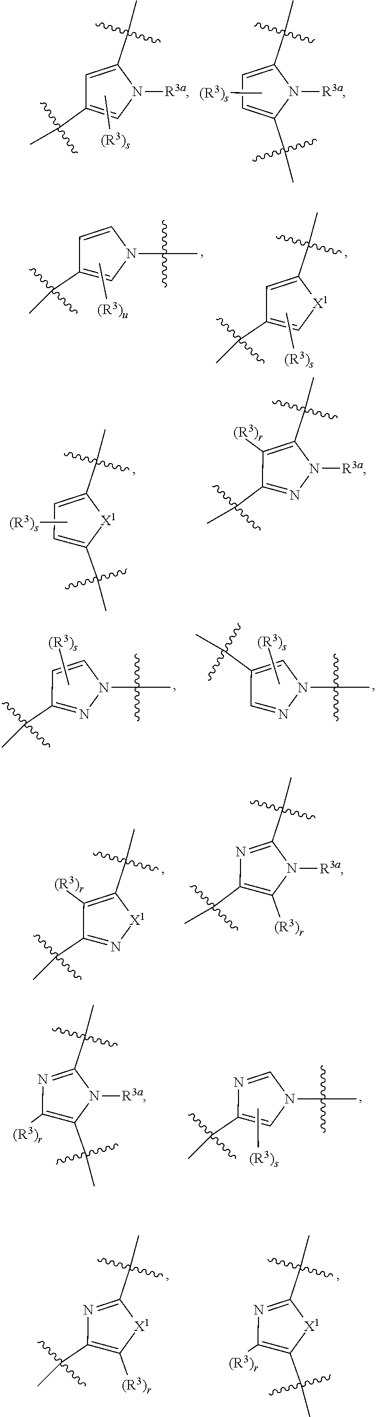

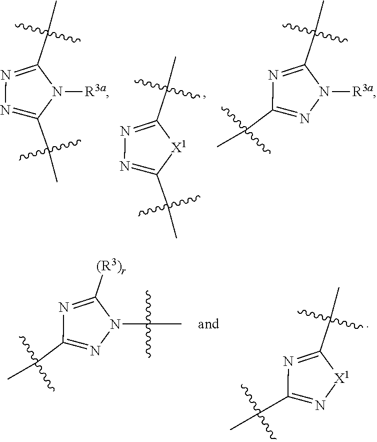

In certain embodiments,

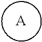

is selected from the group consisting of

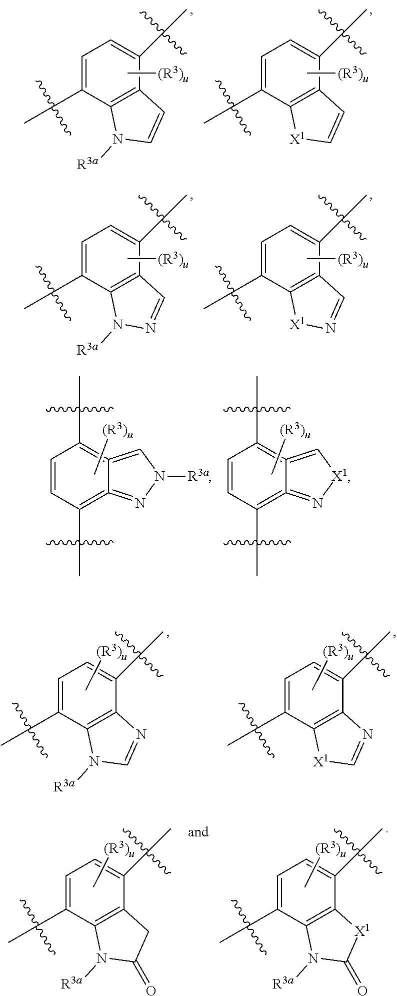

In certain embodiments,

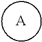

is selected from the group consisting of

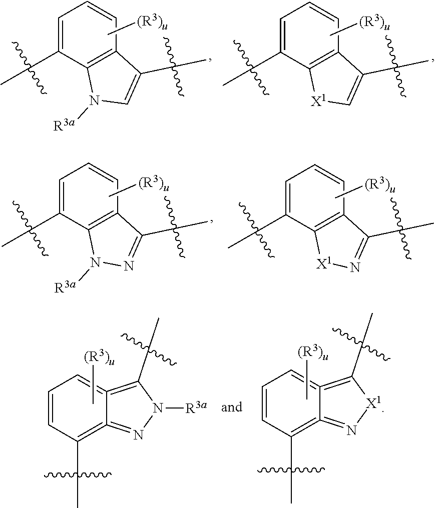

In certain embodiments,

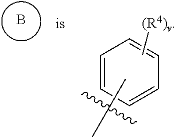

In certain embodiments,

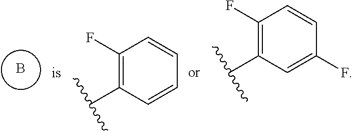

In certain embodiments,

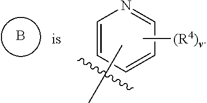

In certain embodiments,

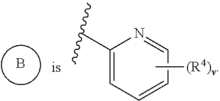

In certain embodiments,

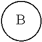

is selected from the group consisting of

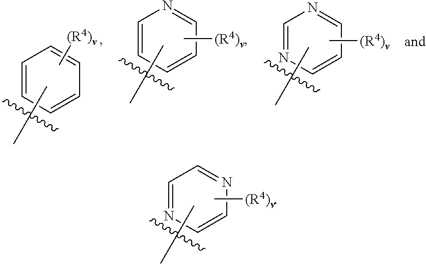

In certain embodiments,

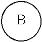

is selected from the group consisting of

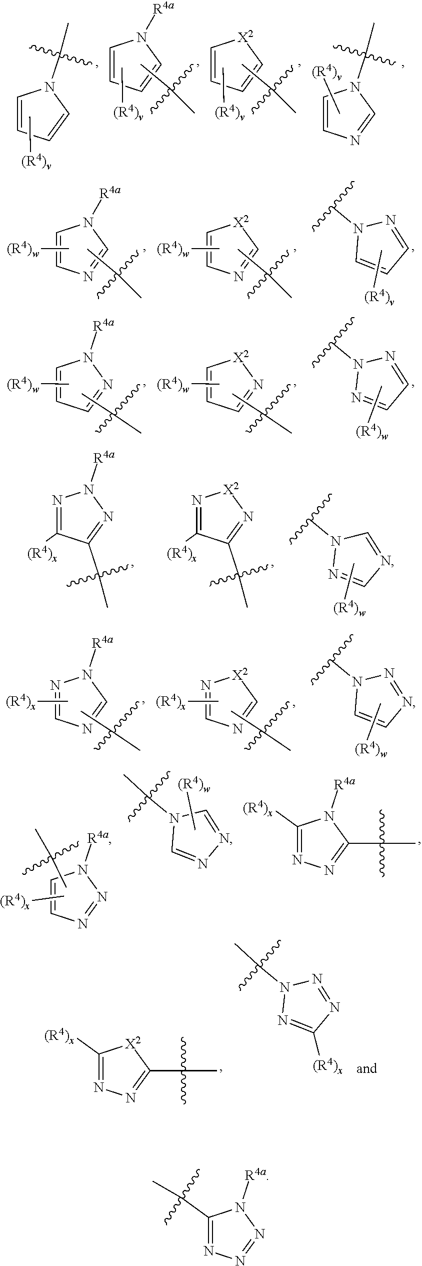

In certain embodiments,

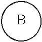

is selected from the group consisting of

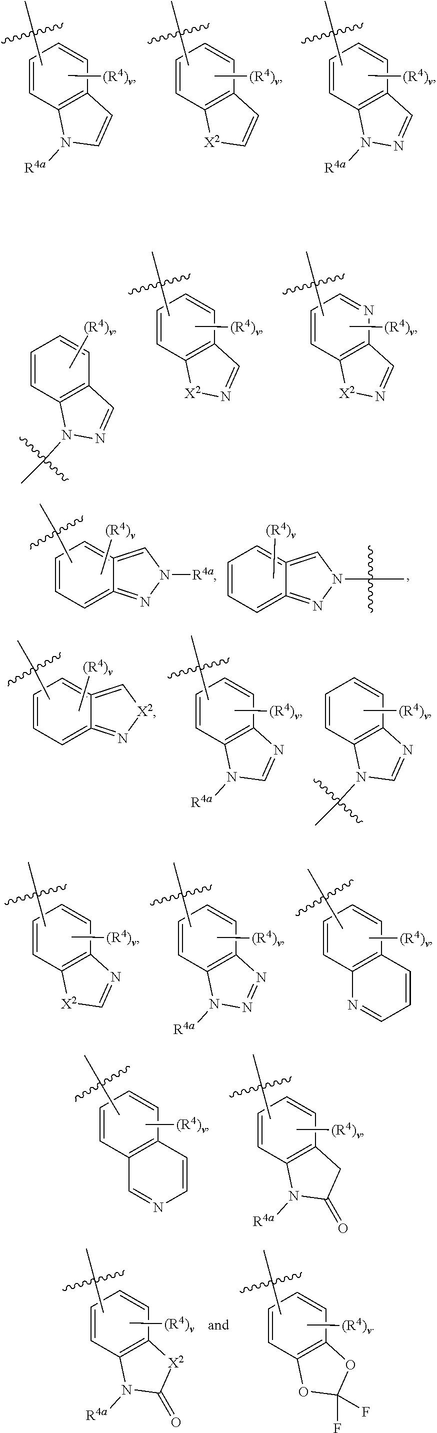

In certain embodiments, L is

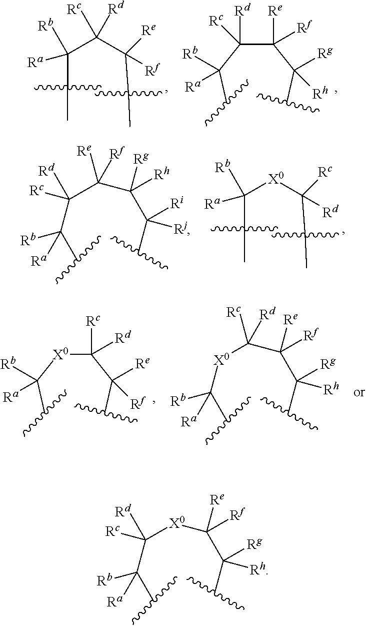

In certain embodiments, L is

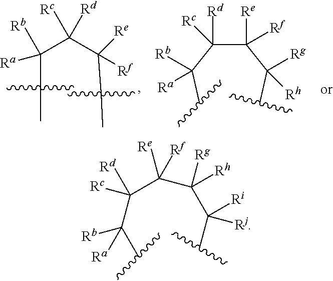

In certain embodiments, L is

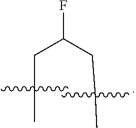 [SMILES: *CC(F)C*]

In certain embodiments, L is

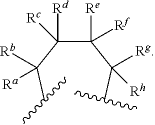

In certain embodiments, L is

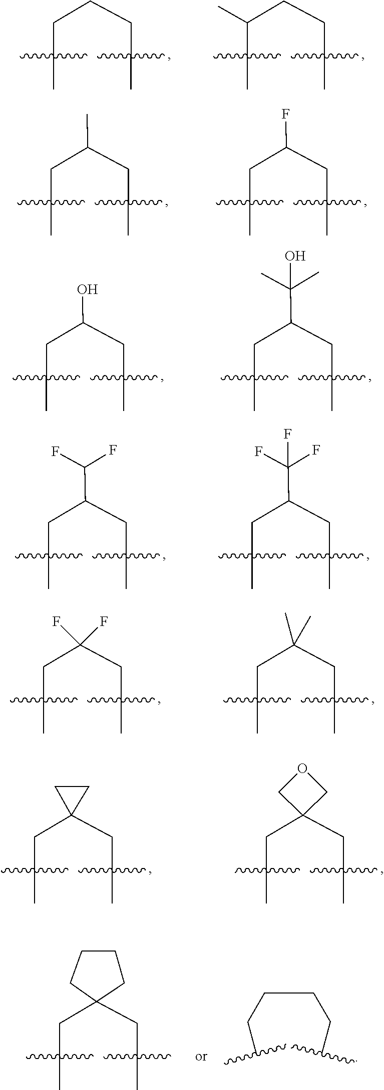

In certain embodiments, L is

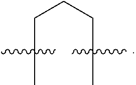 [SMILES: *CCC*]

In certain embodiments, L is

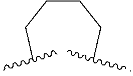 [SMILES: *CCCC*]

In certain embodiments, L is

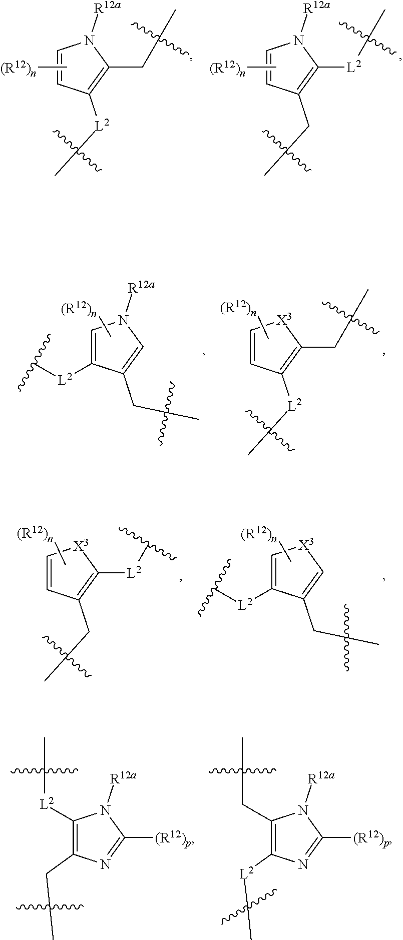

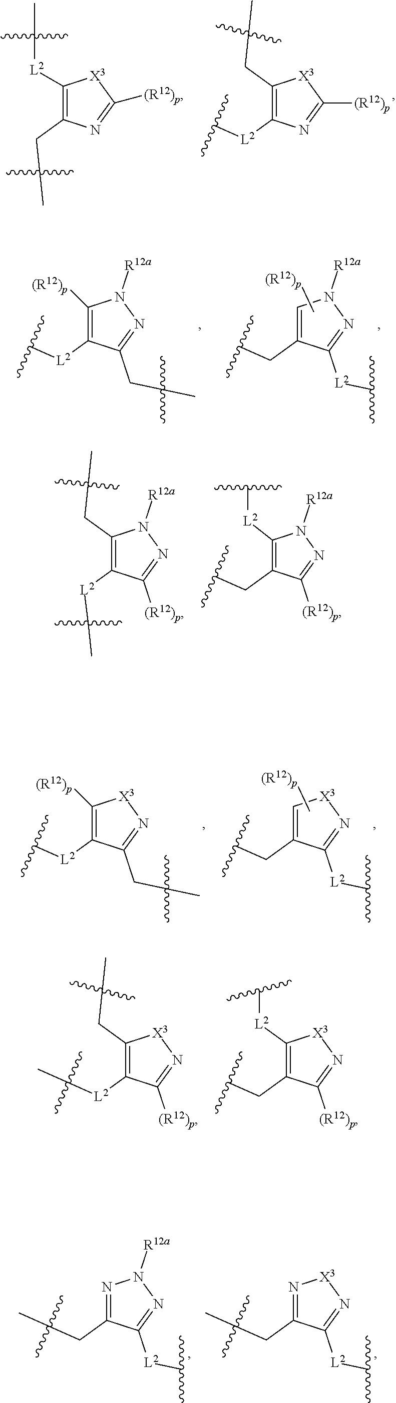

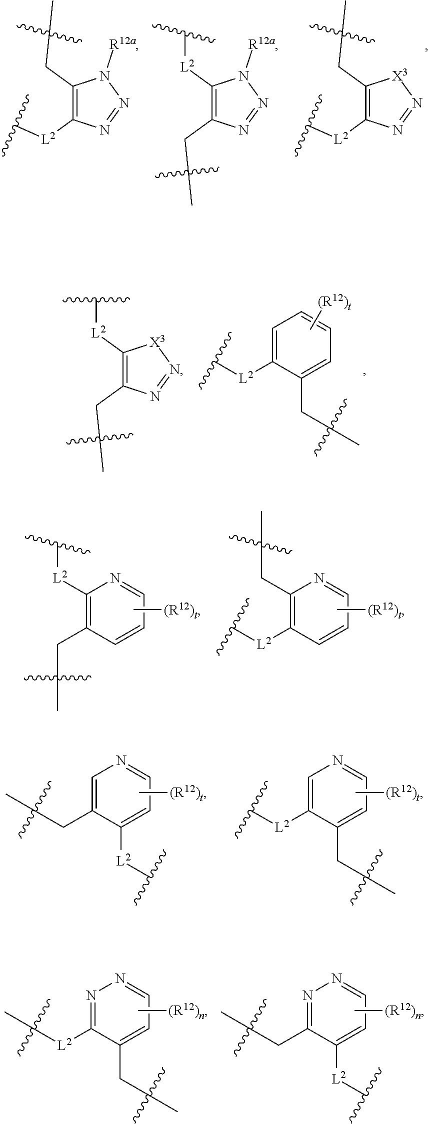

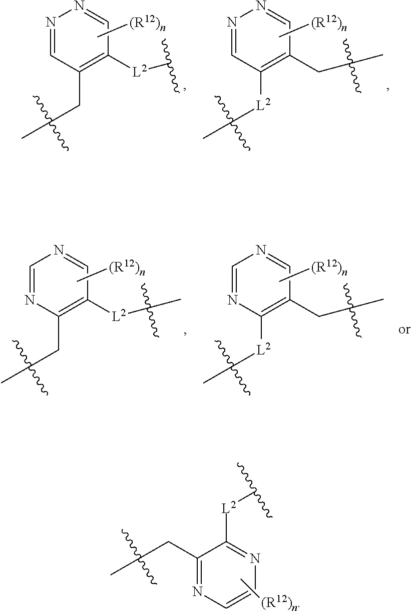

In certain embodiments. X is CR².

In certain embodiments, X is CR² and R² is Cl, F, CH₃ or CF₃.

In certain embodiments, X is CR² and R² is CH₃.

In certain embodiments, R¹ is

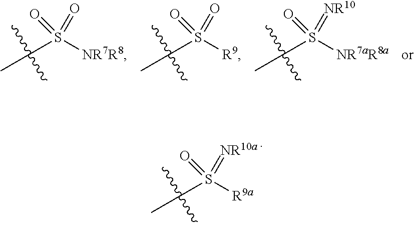

In certain embodiments, R¹ is

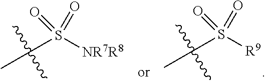

In certain embodiments, R¹ is

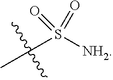 [SMILES: *S(N)(=O)=O]

In certain embodiments, R¹ is

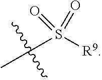

In certain embodiments, R¹ is

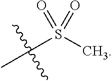 [SMILES: *S(C)(=O)=O]

In certain embodiments, R¹ is

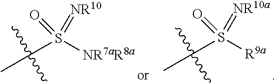

In certain embodiments, R¹ is

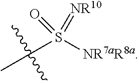

In certain embodiments, R¹ is

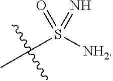 [SMILES: *S(=N)(N)=O]

In certain embodiments, R¹ is

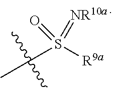

In certain embodiments, R¹ is

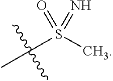 [SMILES: *S(C)(=N)=O]

In certain embodiments, R³ is halo for each occurrence and u is 1, 2 or 3.

In certain embodiments, R³ is F for each occurrence and u is 1, 2 or 3.

In certain embodiments, R⁴ is independently selected for each occurrence from the group consisting of halo, CN, methyl, CHF₂, CF₃, acetylenyl, and cyclopropyl.

In certain embodiments, R⁴ is independently selected for each occurrence from the group consisting of halo, CN, OH, NH₂, NH(CH₃), N(CH₃)₂, C₁₋₄alkyl, haloC₁₋₄alkyl, C₂₋₄alkenyl, C₂₋₄alkynyl optionally substituted with hydroxyC₁₋₃alkyl, and cyclopropyl optionally substituted with halo or cyano.

In certain embodiments, R^{4b} is selected from the group consisting of

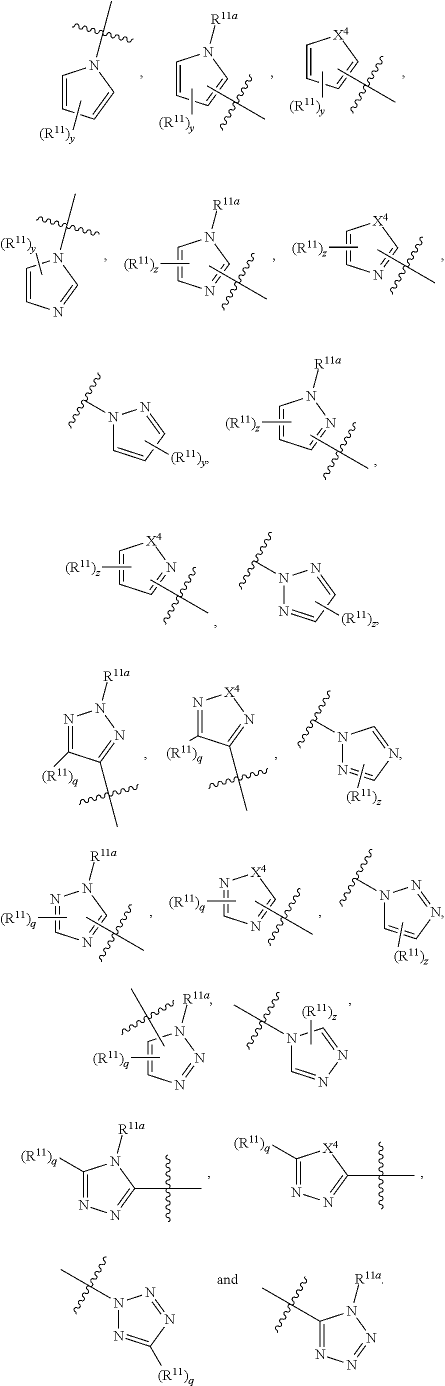

In certain embodiments, R^{4b} is selected from the group consisting of

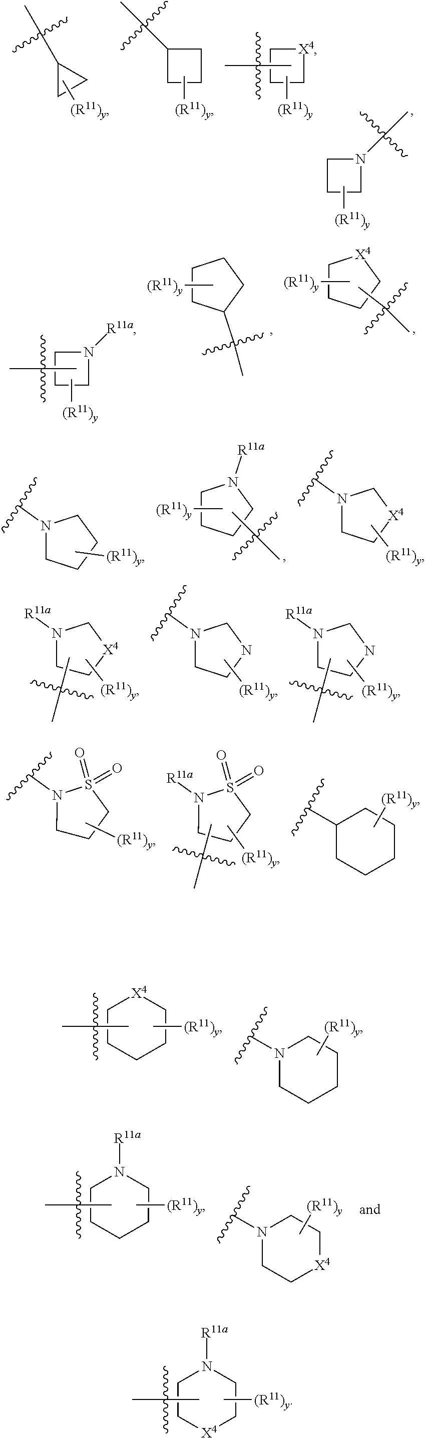

In certain embodiments, R^{a}, R^{b}, R^{c}, R^{d}, R^{e}, R^{f}, R^{g}, R^{h}, Rⁱ and R^{j} are independently selected from the group consisting of hydrogen, halo, cyano, OH, NH₂, NH(CH₃), N(CH₃)₂, —C(O)OH, —C(O)OC₁₋₄alkyl, —C(O)NH₂, —C(O)NH(CH₃), —C(O)N(CH₃)₂, —SO₂NH₂, —SO₂NH(CH₃), —SO₂N(CH₃)₂, C₁₋₄alkyl, haloC₁₋₄alkyl, hydroxyC₁₋₄alkyl and C₁₋₄alkoxy.

In certain embodiments, R³ is selected from the group consisting of

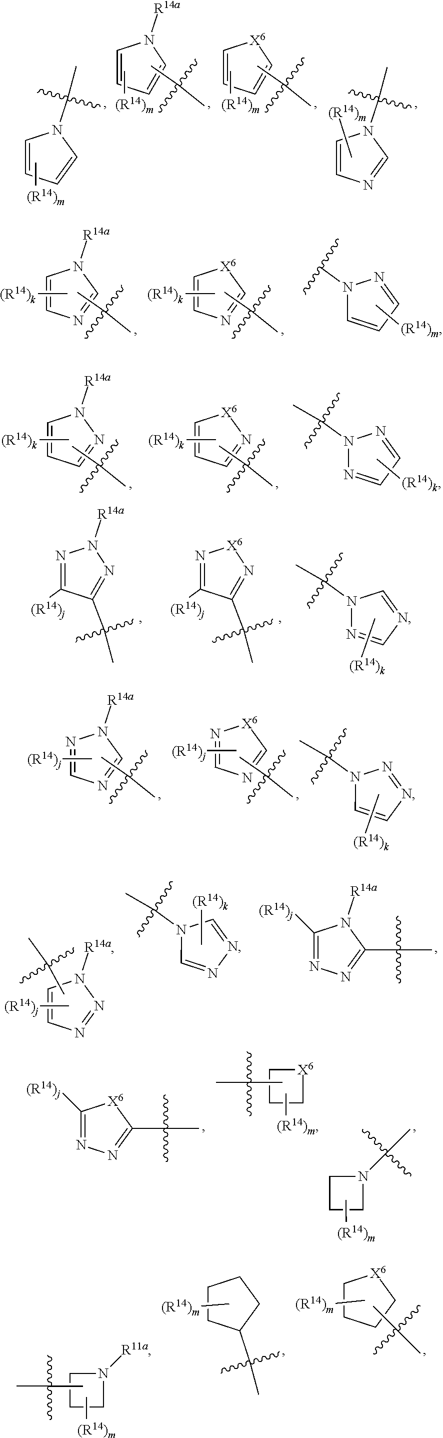

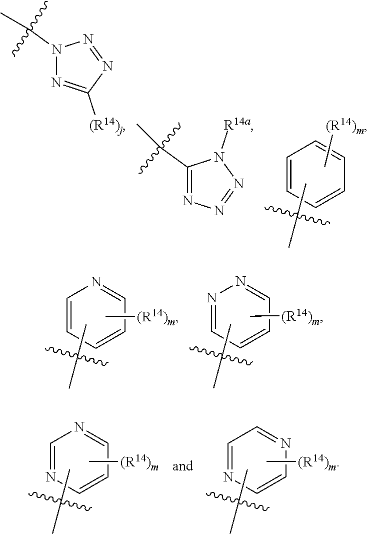

In certain embodiments, R¹³ is selected from the group consisting of

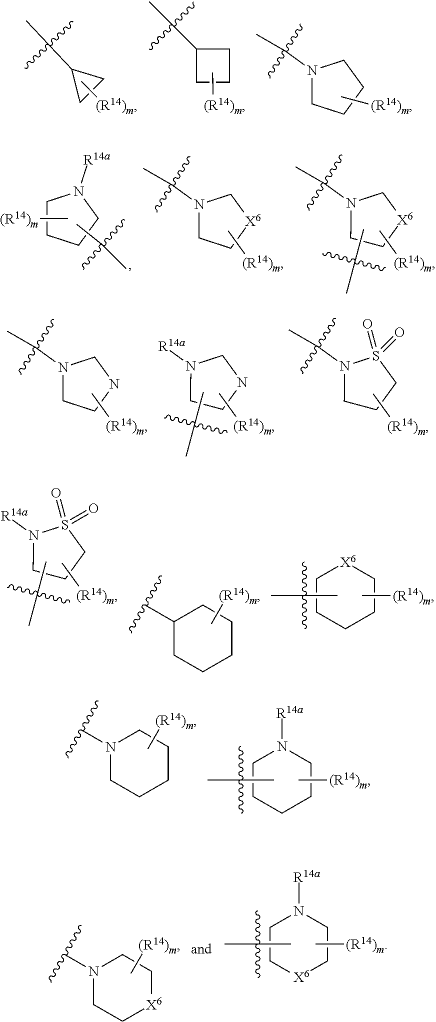

In another aspect, the present invention provides a compound of Formula II

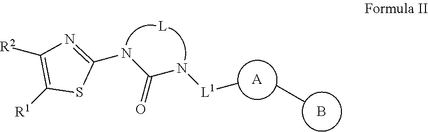

or a pharmaceutically acceptable salt thereof, wherein:

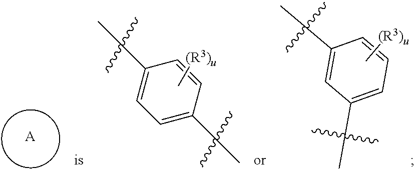

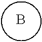

is selected from the group consisting of

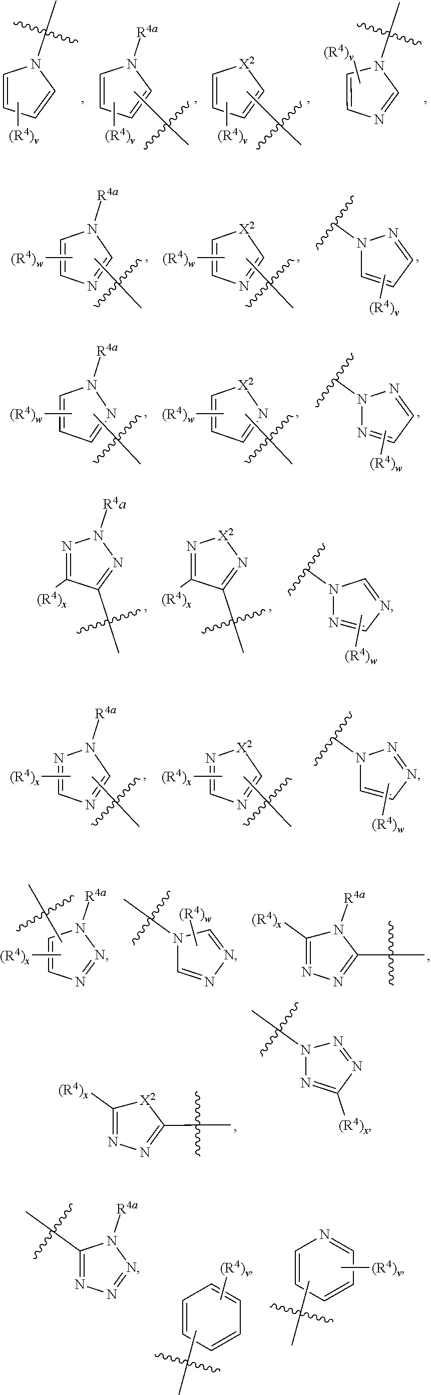

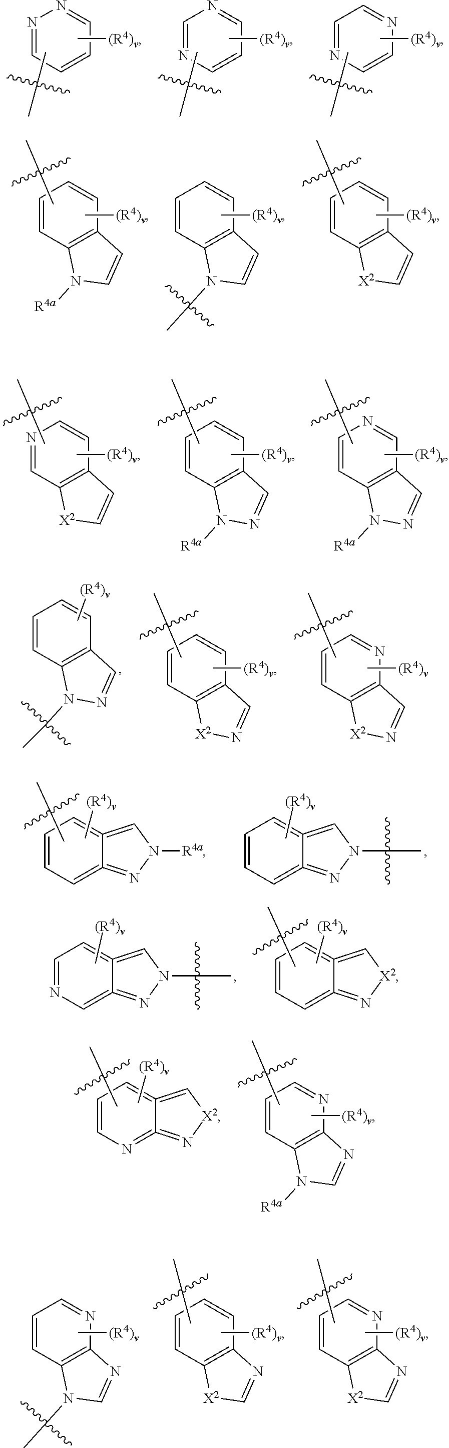

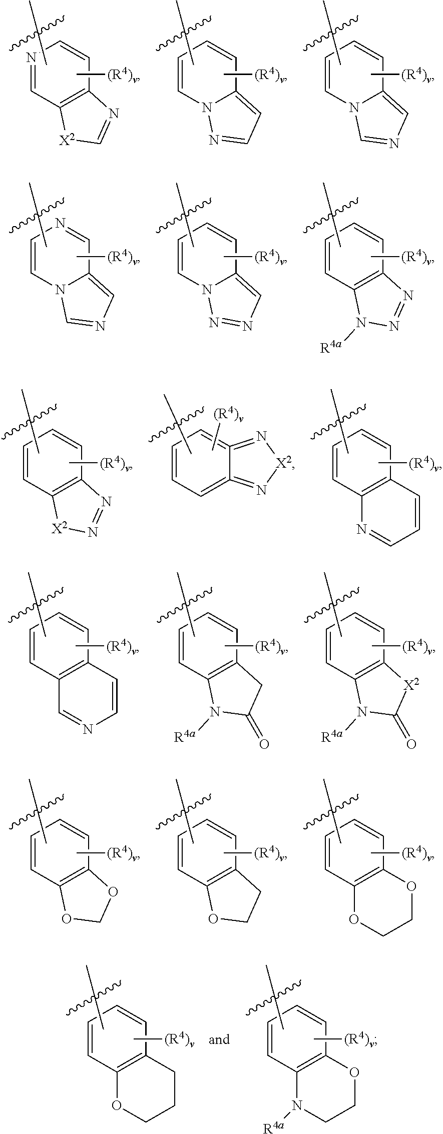

X² and X⁴ are independently selected from the group consisting of O and S;
X⁵ is CH₂, CF₂, O, S or NR,
L is

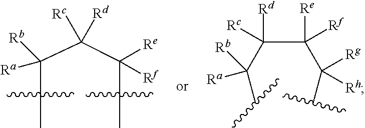

L¹ is a bond; or L¹ is —CH₂— when

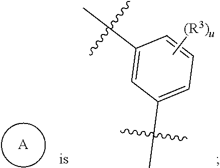

R^{a}, R^{b}, R^{c}, R^{d}, R^{e}, R^{f}, R^{g} and R^{h} are independently selected from the group consisting of hydrogen, halo, CN, OH, NRⁿR^{m}, —C(O)OH, —C(O)OC₁₋₄alkyl, —C(O)NRⁿR^{m}, —SO₂NRⁿR^{m}, C₁₋₄alkyl, C₂₋₄alkenyl, C₂₋₄alkynyl, haloC₁₋₄alkyl, hydroxyC₁₋₄alkyl, and C₁₋₄alkoxy; or two R-groups together with the carbon atom to which they are attached form a C₃₋₆monocycloalkyl,

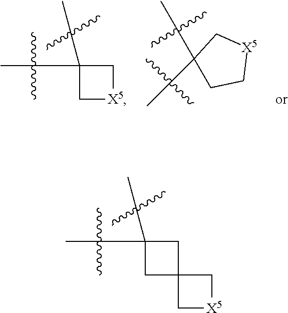

group;
Rⁿ and R^{m} are independently selected for each occurrence from the group consisting or hydrogen and C₁₋₄alkyl;
R^{y} is hydrogen, C₁₋₄alkyl or acetyl;
R is

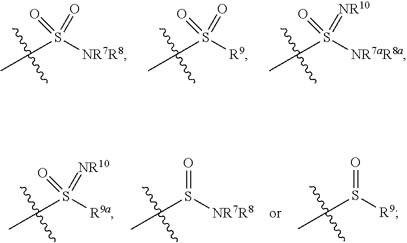

R² is hydrogen, halo, CN, OH, C₁₋₄alkyl, C₂₋₄alkenyl, C₂₋₄alkynyl, haloC₁₋₄alkyl, C₁₋₄alkoxy, hydroxyC₁₋₄alkyl or haloC₁₋₄alkoxy;
R⁴ is independently selected for each occurrence from the group consisting of halo, CN, OH, NRⁿR^{m}, C₁₋₄alkyl, haloC₁₋₄alkyl, C₂₋₄alkenyl, C₂₋₄alkynyl optionally substituted with hydroxyC₁₋₃alkyl, cyclopropyl optionally substituted with halo or cyano, and R^{4b}, provided that only one R⁴ group can be R^{4b};
R^{4a} is hydrogen, C₁₋₄alkyl, halo C₁₋₄alkyl and hydroxyC₁₋₄alkyl;
R^{4b} is selected from the group consisting of

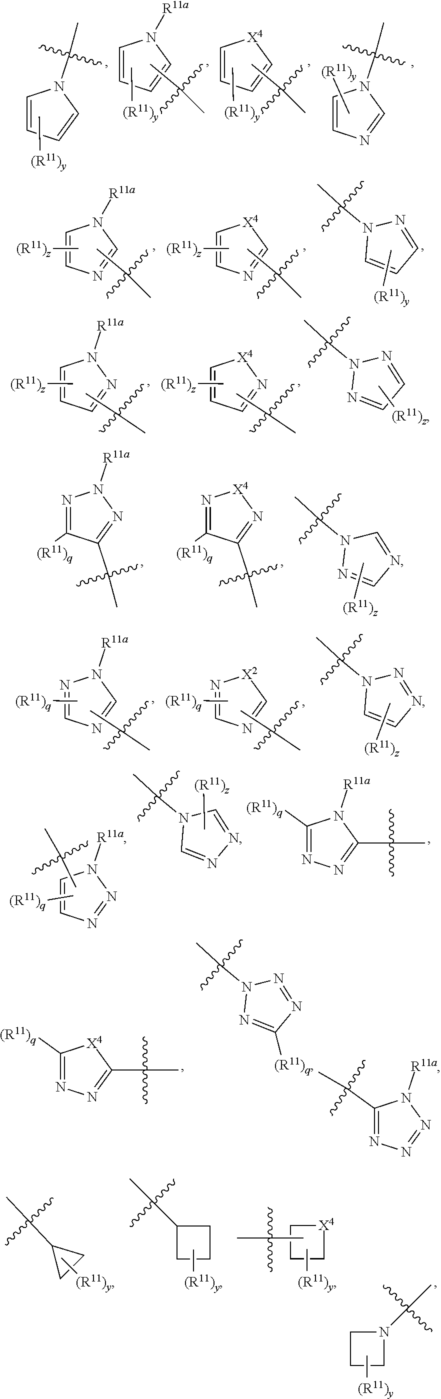

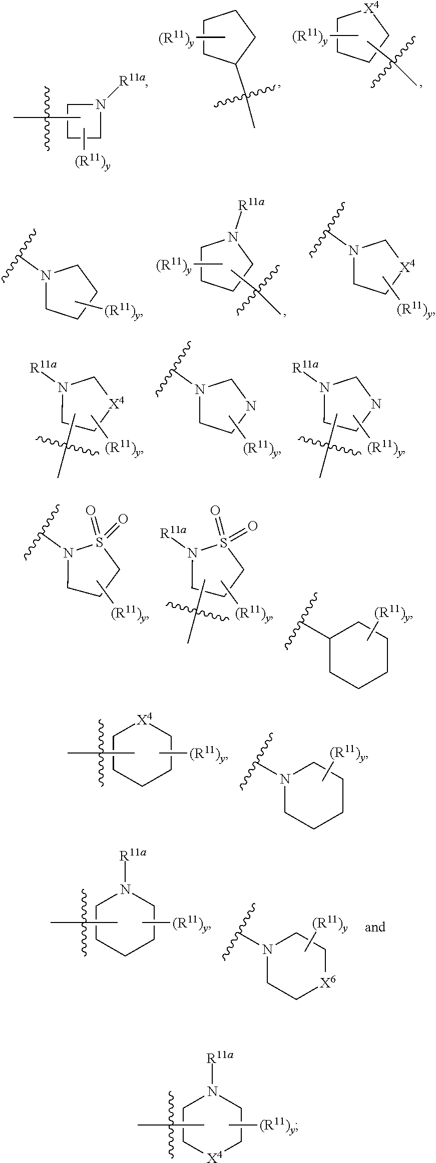

R⁷ and R⁸ are independently selected from the group consisting of hydrogen, OH, acetyl, C₁₋₁₀alkyl, haloC₁₋₁₀alkyl, hydroxyC₁₋₁₀alkyl, C₁₋₄alkoxyC₁₋₁₀alkyl, C₃₋₆monocycloalkyl, phenyl, pyridyl or indolyl; or R⁷ and R⁸ together with the N-atom to which they are attached form an arizidinyl, azetidinyl, pyrrolidinly, piperidinyl, morpholinyl or thiomorpholinyl group, wherein the arizidinyl, azetidinyl, pyrrolidinly or piperidinyl group is optionally substituted with halo, CN or OH;
R^{7a} and R^{8a} are independently selected from the group consisting of hydrogen, C₁₋₄alkyl and C₃₋₆monocycloalkyl, or R⁷ and R⁸ together with the N atom to which they are attached form an arizidinyl, azetidinyl, pyrrolidinyl, or piperidinyl morpholinyl or thiomorpholinyl group;
R⁹ and R^{9a} are independently selected from the group consisting or C₁₋₄alkyl and haloC₁₋₄alkyl;
R¹⁰ and R^{10a} are independently selected from the group consisting of hydrogen and C₁₋₄alkyl;
R¹¹ is independently selected from the group consisting of halo, CN, OH, NRⁿR^{m}, C₁₋₄alkyl, haloC₁₋₄alkyl, C₂₋₄alkenyl, C₂₋₄alkynyl, and C₃₋₆monocycloalkyl;
R^{11a} is hydrogen, C₁₋₄alkyl, halo C₁₋₄alkyl and hydroxyC₁₋₄alkyl;
q and x are independently selected from the group consisting of 0 and 1;
w and z are independently selected from the group consisting of 0, 1 and 2; and
v and y are independently selected from the group consisting of 0, 1, 2 and 3.

The following embodiments further describe a compound of Formula II, or a pharmaceutically acceptable salt thereof. It will be appreciated that all chemically allowable combinations of the embodiments described herein are envisioned as further embodiments of the invention.

In certain embodiments:

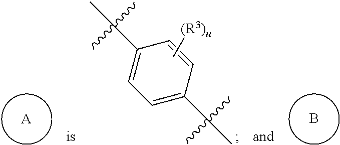

is selected from the group consisting of

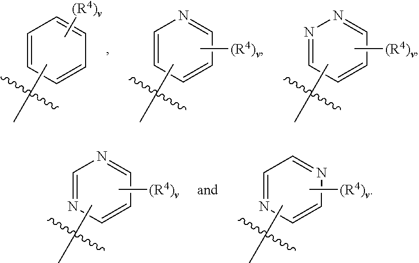

In certain embodiments:

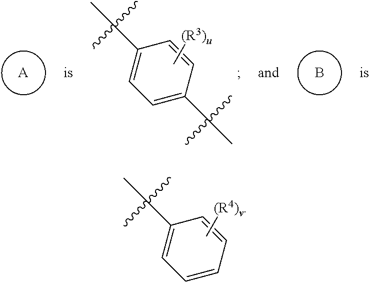

In certain embodiments:

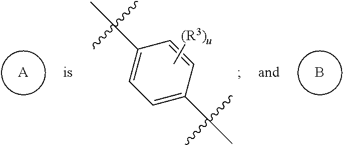

is selected from the group consisting of

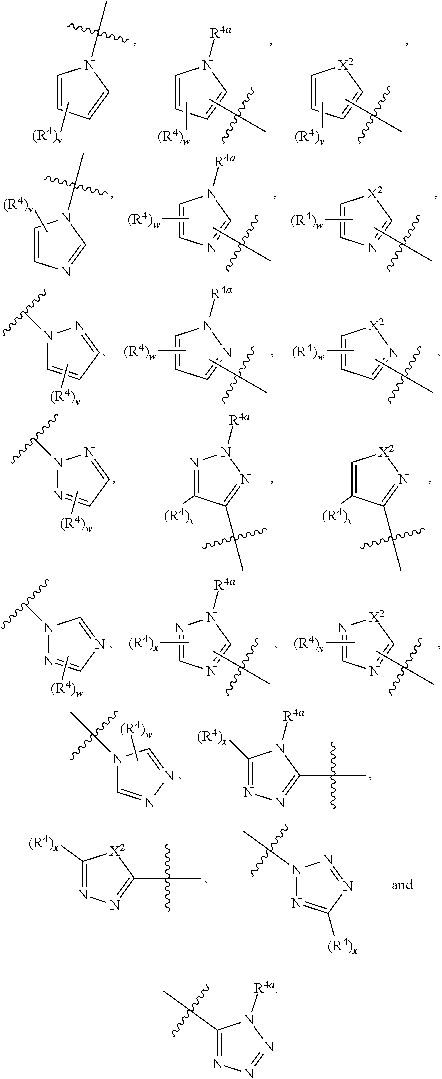

In certain embodiments:

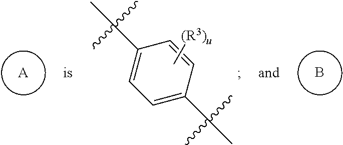

is selected from the group consisting of

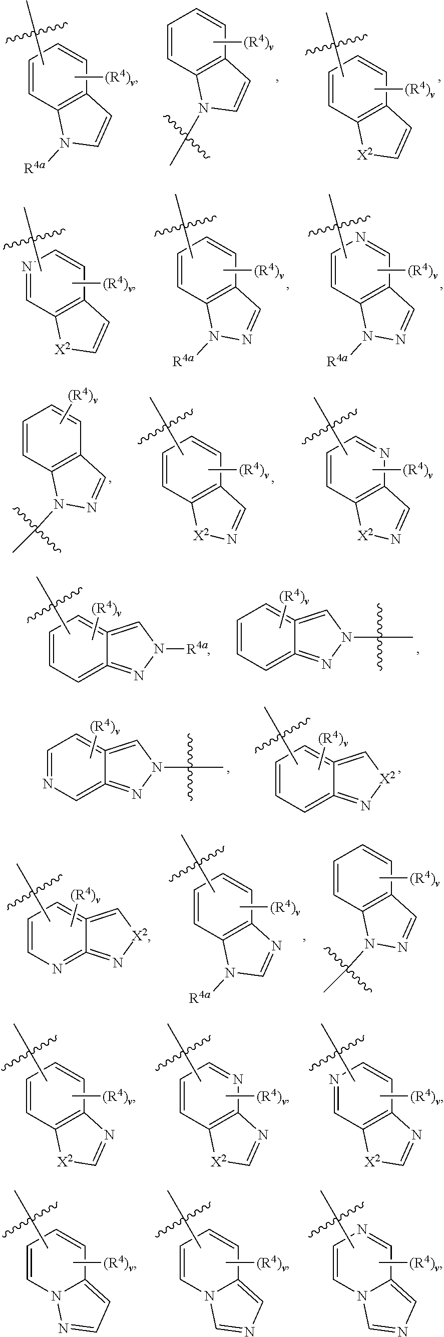

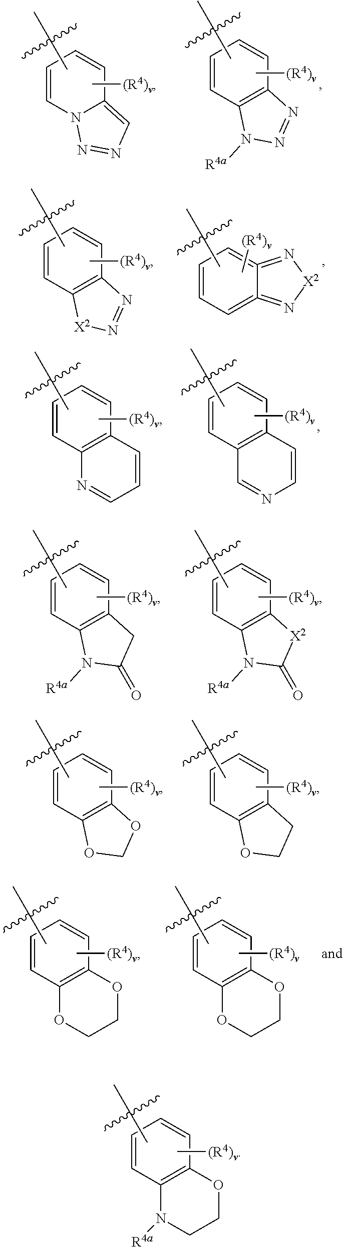

In certain embodiments:

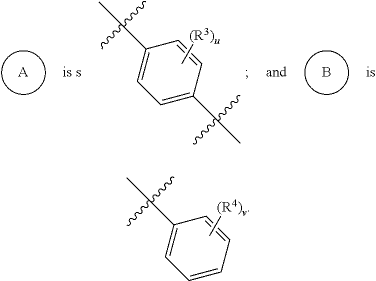

In certain embodiments: L is

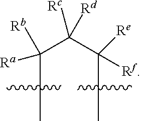

In certain embodiments, L is

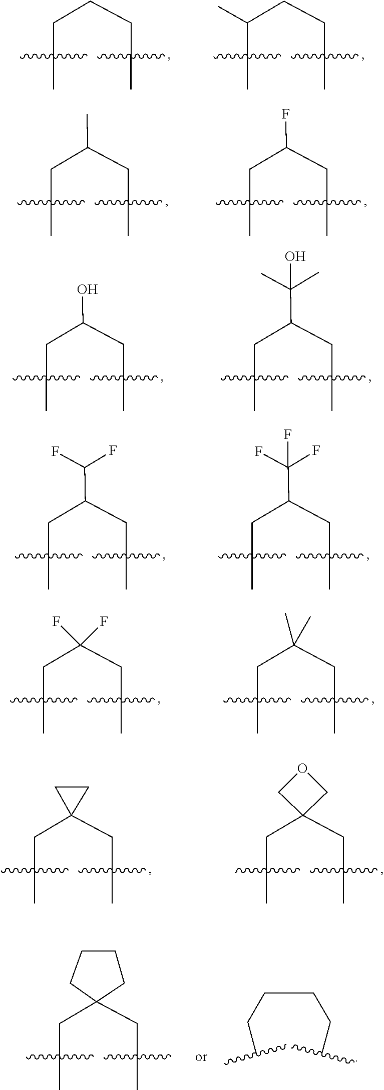

In certain embodiments, L is

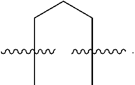 [SMILES: *CCC*]

In certain embodiments: L is

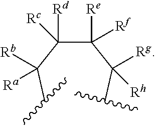

In certain embodiments, L is

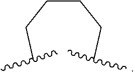 [SMILES: *CCCC*]

In certain embodiments: R² is H, Cl, F, CH₃ or CF₃.

In certain embodiments: R² is CH₃.

In certain embodiments:
R¹ is

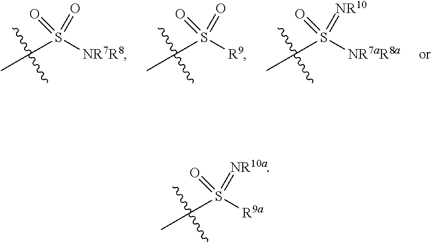

In certain embodiments: R¹ is

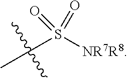

In certain embodiments: R¹ is

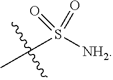 [SMILES: *S(N)(=O)=O]

In certain embodiments: R¹ is

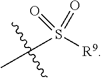

In certain embodiments: R¹ is

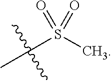 [SMILES: *S(C)(=O)=O]

In certain embodiments: R¹ is

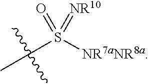

In certain embodiments: R¹ is

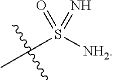 [SMILES: *S(=N)(N)=O]

In certain embodiments: R¹ is

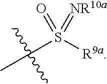

In certain embodiments: R¹ is

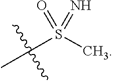 [SMILES: *S(C)(=N)=O]

In certain embodiments: R³ is halo for each occurrence and u is 0, 1, 2 or 3.

In certain embodiments: u is 0.

In certain embodiments: R⁴ is independently selected for each occurrence from the group consisting of halo, CN, methyl, CHF₂, CF₃, acetylenyl, and cyclopropyl.

In certain embodiments: R⁴ is independently selected from halo for all occurrences.

### Methods of Use

The compounds according to the present invention are useful for the treatment and prophylaxis of disorders caused by herpes viruses, in particular Herpes simplex viruses.

In one aspect, the present invention provides a method for the treatment or prophylaxis of an HSV infection in a subject in need thereof, the method comprising: administering to the subject a therapeutically effective amount of a compound of the invention, or a pharmaceutically acceptable salt thereof.

In some embodiment, the infection is a Herpes simplex infection.

In some embodiment, the infection is an HSV-1 infection.

In some embodiment, the infection is an HSV-2 infection.

In some embodiments, the infection is a Herpes simplex infection and the subject displays symptoms such as Herpes labialis, Herpes genitalis, HSV-related keratitis, encephalitis, or pneumonia.

In another embodiment, the infection is a Herpes simplex infection and the subject displays symptoms such as suppressed immune system (for example AIDS patients, cancer patients, patients having a genetic immunodeficiency, transplant patients).

In another embodiment, the infection is a Herpes simplex infection, and the subject is a new-born child or infant.

In another aspect, the present invention provides a method for suppressing recurrence of HSV symptoms or outbreaks in a subject in need thereof, the method comprising: administering to the subject a therapeutically effective amount of a compound of the invention, or a pharmaceutically acceptable salt thereof.

In some embodiment, the infection is a Herpes simplex infection.

In some embodiment, the infection is an HSV-1 infection.

In some embodiment, the infection is an HSV-2 infection.

In some embodiment, the subject is a herpes-positive patient.

In some embodiment, the subject is a herpes-simplex-positive patient.

In another aspect, the present invention provides a method for the treatment or prophylaxis of an HSV infection in a subject in need thereof, the method comprising: administering to the subject a therapeutically effective amount of a compound of the invention, or a pharmaceutically acceptable salt thereof, wherein: the infection is resistant to nucleosidic antiviral therapy.

In one embodiment, the infection is a Herpes simplex infection.

In some embodiment, the infection is a Herpes simplex infection.

In another embodiment, the subject is a herpes-positive patient.

In another embodiment, the nucleosidic antiviral therapy is selected from the group consisting of acyclovir, penciclovir, famciclovir, ganciclovir and valacyclovir.

In another aspect, the present invention provides a compound for the use as a medicament.

### Combination Therapies

The compounds according to the present invention are also useful for the treatment and prophylaxis of disorders caused by herpes viruses, in particular Herpes simplex viruses, in combination with other active ingredients.

In one aspect, the present invention provides a method for the treatment or prophylaxis of an HSV infection in a subject in need thereof, the method comprising: administering to the subject a therapeutically effective amount of a compound of the invention, or a pharmaceutically acceptable salt thereof, in combination with an antiviral agent.

In some embodiments, the antiviral agent is selected from the group consisting of acyclovir, penciclovir, famciclovir, ganciclovir and valacyclovir, foscarnet and trifluridine.

In some embodiment, the infection is a Herpes simplex infection.

In some embodiment, the infection is an HSV-1 infection.

In some embodiment, the infection is an HSV-2 infection.

In some embodiments, the infection is a Herpes simplex infection and the subject displays symptoms such as Herpes labialis, Herpes genitalis, HSV-related keratitis, encephalitis, or pneumonia.

In another embodiment, the infection is a Herpes simplex infection and the subject displays symptoms such as suppressed immune system (for example AIDS patients, cancer patients, patients having a genetic immunodeficiency, transplant patients).

In another embodiment, the infection is a Herpes simplex infection, and the subject is a new-born child or infant.

In another aspect, the present invention provides a method for suppressing recurrence of HSV symptoms or outbreaks in a subject in need thereof, the method comprising: administering to the subject a therapeutically effective amount of a compound of the invention, or a pharmaceutically acceptable salt thereof, in combination with an antiviral agent.

In some embodiments, the antiviral agent is selected from the group consisting of acyclovir, penciclovir, famciclovir, ganciclovir and valacyclovir, foscarnet and trifluridine.

In some embodiment, the infection is a Herpes simplex infection.

In some embodiment, the infection is an HSV-1 infection.

In some embodiment, the infection is an HSV-2 infection.

In some embodiment, the subject is a herpes-positive patient.

In some embodiment, the subject is a herpes-simplex-positive patient.

In another aspect, the present invention provides a compound for the use as a medicament.

In another aspect, the present invention provides a method for the treatment or prophylaxis of an HSV infection in a subject in need thereof, the method comprising: administering to the subject a therapeutically effective amount of a compound of the invention, or a pharmaceutically acceptable salt thereof, in combination with a corticosteroid.

In some embodiment, the infection is a Herpes simplex infection.

In some embodiment, the infection is an HSV-1 infection.

In some embodiment, the infection is an HSV-2 infection.

In some embodiments, the infection is a Herpes simplex infection and the subject displays symptoms such as Herpes labialis, Herpes genitalis, HSV-related keratitis, encephalitis, or pneumonia.

In another embodiment, the infection is a Herpes simplex infection and the subject displays symptoms such as suppressed immune system (for example AIDS patients, cancer patients, patients having a genetic immunodeficiency, transplant patients).

In another embodiment, the infection is a Herpes simplex infection, and the subject is a new-born child or infant.

In another aspect, the present invention provides a method for suppressing recurrence of HSV symptoms or outbreaks in a subject in need thereof, the method comprising: administering to the subject a therapeutically effective amount of a compound of the invention, or a pharmaceutically acceptable salt thereof, in combination with a corticosteroid.

In some embodiment, the infection is a Herpes simplex infection.

In some embodiment, the infection is an HSV-1 infection.

In some embodiment, the infection is an HSV-2 infection.

In some embodiment, the subject is a herpes-positive patient.

In some embodiment, the subject is a herpes-simplex-positive patient.

In another aspect, the present invention provides a compound for the use as a medicament.

### Formulations and Administration

The compounds on the invention can be converted in a known manner into the customary formulations, such as tablets, sugar-coated tablets, pills, granules, aerosols, syrups, emulsions, suspensions, and solutions, using inert, nontoxic, pharmaceutically suitable carriers and solvents.

Here, the therapeutically active compound should in each case be present in a concentration of about 0.5 to 90% by weight of the total mixture, i.e., in amounts which are sufficient to achieve the dosage range indicated.

The formulations are prepared, for example, by extending the active compounds with solvents and/or excipients, if appropriate using emulsifiers and/or dispersants, it being possible, for example, if the diluent used is water, to use, if appropriate, organic solvents as auxiliary solvents.

Administration is carried out in a customary manner, including orally, parenterally, topically, perlingually or intravenously.

In the case of parenteral administration, solutions or suspensions of the active compounds using suitable liquid carrier and excipients can be employed.

In general, it has proved advantageous in the case of intravenous administration to administer amounts of from approximately 0.001 to 20 mg/kg, preferably approximately 0.01 to 10 mg/kg, of bodyweight to achieve effective results, and in the case of oral administration the dose is approximately 0.01 to 30 mg/kg, preferably 0.1 to 20 mg/kg, of bodyweight.

In some instances, it may be necessary to depart from the amounts mentioned, namely depending on the bodyweight or on the type of administration route, on the individual response to the medicament, the manner of its formulation and the time or interval at which administration takes place. Thus, in some cases it may be adequate to manage with less than the abovementioned minimum amount, while in other cases the upper limit mentioned must be exceeded. In the case of the administration of relatively large amounts, it may be advisable to divide this into several individual administrations over the course of the day.

If appropriate, it may be useful to combine the compounds according to the invention with other active substances, in particular antiviral active substances.

The compounds used in the present invention can be in the form of a pharmaceutically acceptable salt, cocrystal or a solvate. The term “pharmaceutically acceptable salts” refers to salts prepared from pharmaceutically acceptable non-toxic bases or acids, including inorganic bases or acids and organic bases or acids. In case the compounds of the present invention contain one or more acidic or basic groups, the invention also comprises their corresponding pharmaceutically or toxicologically acceptable salts, in particular their pharmaceutically utilizable salts. Thus, the compounds of the present invention which contain acidic groups can be used according to the invention, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine or amino acids. The compounds of the present invention which contain one or more basic groups, i.e., groups which can be protonated, can be used according to the invention in the form of their addition salts with inorganic or organic acids. Examples of suitable acids include hydrogen chloride, hydrogen bromide, phosphoric acid, sulfuric acid, nitric acid, methanesulfonic acid, p-toluenesuifonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the present invention simultaneously contain acidic and basic groups in the molecule, the invention also includes, in addition to the salt forms mentioned, inner salts or betaines (zwitterions). The respective salts can be obtained by customary methods which are known to the person skilled in the art like, for example, by contacting these with an organic or inorganic acid or base in a solvent or dispersant, or by anion exchange or cation exchange with other salts. The present invention also includes all salts of the compounds of the present invention which, owing to low physiological compatibility, are not directly suitable for use in pharmaceuticals but which can be used, for example, as intermediates for chemical reactions or for the preparation of pharmaceutically acceptable salts.

Depending on the substitution pattern, the compounds according to the invention can exist in stereoisomeric forms which either behave as image and mirror image (enantiomers), or which do not behave as image and mirror image (diastereomers). The invention relates both to the enantiomers or diastereomers and their respective mixtures. Like the diastereomers, the racemic forms can be separated into the stereoisomerically uniform components in a known manner.

The scope of the invention includes those compounds which are only converted into the actual active compounds of the Formulas I and once inside the body (so-called prodrugs).

In practical use, the compounds used in the present invention can be combined as the active ingredient in intimate admixture with a pharmaceutical carrier according to conventional pharmaceutical compounding techniques. The carrier may take a wide variety of forms depending on the form of preparation desired for administration, e.g., oral, or parenteral {including intravenous). In preparing the compositions for oral dosage form, any of the usual pharmaceutical media may be employed, such as, for example, water, glycols, oils, alcohols, flavouring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or non-aqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray or as eye drops.

The tablets, pills, capsules, and the like may also contain a binder such as hydroxypropyl methylcellulose, or polyvinylpyrrolidone; diluent or fillers such as microcrystalline cellulose, dicalcium phosphate, lactose, or mannitol; a disintegrating agent such as croscarmellose sodium, polyvinylpyrrolidone, or sodium starch glycolate; a lubricant such as magnesium stearate or sodium stearyl fumarate; a glidant such as silicon dioxide; and a sweetening agent such as sucrose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

The compounds used in the present invention may also be administered parenterally. Solutions or suspensions of these active compounds can be prepared in water suitably mixed with a surfactant such as hydroxypropyl cellulose, sodium lauryl sulfate, or polysorbate. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present invention. For example, oral, rectal, topical, parenteral (including intravenous), ocular, pulmonary, nasal, and the like may be employed. Dosage forms include tablets, troches, dispersions, suspensions, solutions, capsules, creams, ointments, aerosols, and the like. Compounds of the present invention can be administered orally or as eye drop. The compounds of the present invention can also be administered orally. The effective dosage of active ingredient employed may vary depending on the particular compound employed, the mode of administration, the condition being treated, and the severity of the condition being treated. Such dosage may be ascertained readily by a person skilled in the art.

The compounds of the present invention can also be present in combination with additional active ingredients, in particular, with one or more active ingredients exhibiting advantageous effects in the treatment of any of the disorders or diseases as described herein. The compounds of the present invention can be present in a composition in combination with at least one further active substance being effective in treating a disease or disorder associated with viral infections (antiviral active compounds), preferably a disease or disorder being associated with viral infections caused by herpes viruses, such as in particular by Herpes simplex viruses (i.e., combination therapy). The at least one further active substance being effective in treating a disease or disorder associated with viral infections (antiviral active compounds) are preferably selected from the group consisting of nucleosidic drugs such as acyclovir, valacyclovir, penciclovir, ganciclovir, famciclovir and trifluridine, as well as compounds such as foscarnet and cidofovir.

Accordingly, the present invention further relates to a pharmaceutical composition comprising one or more of the compounds as described herein and at least one pharmaceutically acceptable carrier and/or excipient and/or at least one further active substance being effective in treating a disease or disorder associated with viral infections (antiviral active compounds).

The novel active compounds can be converted in a known manner into customary formulations, such as tablets, caplets, sugar-coated tablets, pills, granules, aerosols, syrups, pharmaceutically suitable carriers, and solvents. Here, the therapeutically active compound should in each case be present in a concentration of about 0.1 to 90% by weight of the total mixture, i.e., in amounts which are sufficient to achieve the dosage range indicated.

The formulations are prepared, for example, by extending the active compounds with solvents and/or excipients, if appropriate using emulsifiers and/or dispersants, if being possible, for example, if the diluent used is water, to use, if appropriate, organic solvents as auxiliary solvents.

Administration is carried out in a customary manner, preferably orally, parenterally or topically, in particular perlingually or intravenously.

In the case of parenteral administration, solutions or suspensions of the active compounds using suitable liquid carrier materials can be employed.

In general, it has proved advantageous in the case or intravenous administration to administer amounts of from approx. 0.001 to 20 mg/kg, preferably approx. 0.01 to 10 mg/kg of bodyweight to achieve effective results, and in the case of oral administration the dose is approx. 0.01 to 30 mg/kg, preferably 0.1 to 20 mg/kg of body weight.

In spite of this, it may be necessary, if appropriate, to depart from the amounts mentioned, namely depending on the bodyweight or on the type of the administration route, on the individual response to the medicament, the manner of its formulation and the time or interval at which administration takes place. Thus, in some cases it may be adequate to manage with less than the abovementioned minimum amount, while in other cases the upper limit mentioned must be exceeded. In the case of administration of relatively large amounts it may be advisable to divide this into several individual administrations over the course of the day.

### EXAMPLES

The compounds described herein can be prepared in several ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

At least some of the compounds identified as “intermediates” herein are contemplated as compounds of the disclosure.

### Abbreviations

AcOH Acetic acid
ACN Acetonitrile
Boc₂O Di-tert-butyl dicarbonate
nBuLi n-Butyllithium
DCM Dichloromethane
DIAD Diisopropyl azodicarboxylate
DIEA Diisopropyl ethylamine
DMF N, N-Dimethylformamide
DMSO Dimethyl sulfoxide
DPPF 1,1′-Bis(diphenylphosphino)ferrocene
EtOAc Ethyl acetate
Et₃N Triethylamine
HATU Hexafluorophosphate Azabenzotriazole Tetramethyl Uronium
h, hr Hour(s)
HPLC High performance liquid chromatography
LCMS Liquid chromatography-mass spectrometry
MeOH Methanol
NMO/NMMO N-Methyl morpholine-N-Oxide
NBS N-Bromosuccinimide
PE Petroleum ether
iPrOH Isopropanol
rt, r.t. Room temperature
SFC Supercritical Fluid Chromatography
TEA Triethylamine
TBAI Tetrabutylammonium iodide
TBAB Tetrabutylammonium bromide
TFA Trifluoroacetic acid
THF Tetrahydrofuran
TLC Thin-layer chromatography
XPhos 2-Dicyclohexylphosphino-2′,4′,6′-triisopropylbiphenyl
   Following LCMS Method have been Used for the Analysis of Final Compounds:

Method A: X-Bridge BEH C-18 (3×50 mm×2.5 mm); Mobile phase: A; 0.025% formic acid in H₂O; B; CH₃CN; Injection volume: 2 μL; Flow rate: 1.2 mL/min, column temperature: 50° C.; Gradient program: 2% B to 98% B in 2.2 min, held 3 min, at 3.2 min B conc. is held at 2% for 4 min.

Method B: X-select CSH 18 (3×50 mm×2.5 mm); Mobile phase: A; 0.025% formic acid in H₂O; B; CH₃CN; Injection volume: 2 VL; Flow rate: 1.2 mL/min, column temperature: 50° C.; Gradient program: 0% B to 98% B in 2 min, hold for 3 min, at 3.2 min B conc. is held at 0% for 4 min.

Method C: X-select CSH 18 (3×50 mm×2.5 mm); Mobile phase: A; 0.05% formic acid in H₂O:CH₃CN (95:5); B; 0.05% formic acid in CH₃CN; Injection volume: 2 μL; Flow rate: 1.2 mL/min, column temperature: 50° C.; Gradient program: 0% B to 98% B in 2 min, hold for 3 min, at 3.2 min B conc. is held at 0% for 4 min.

Method D: X-select CSH C₁₈ (3×50 mm×2.5 μm); Mobile phase: A; 2 mM in Ammonium Bicarbonate; B; CH₃CN; Injection volume: 2 μL Flow rate: 1.2 mL/min, column temperature: 50° C.; Gradient program: 0% B to 98% B in 2 min, hold for 3 min, at 3.2 min B conc. is held at 0% for 4 min.

Method E: X-select CSH 18 (3×50 mm×2.5 mm); Mobile phase: A; 0.05% formic acid in H₂O; B; CH₃CN; Injection volume: 2p L; Flow rate: 1.5 mL/min, column temperature: 50° C.; Gradient program: 0% B to 100% B in 1.5 min, hold 2.2 min, at 2.6 min B conc. is held at 0% for 3 min.

### Example 1. 4-Methyl-2-(2-oxo-3-(4-(pyridin-2-yl) phenyl) tetrahydropyrimidin-1(2H)-yl) thiazole-5-sulfonamide (1)

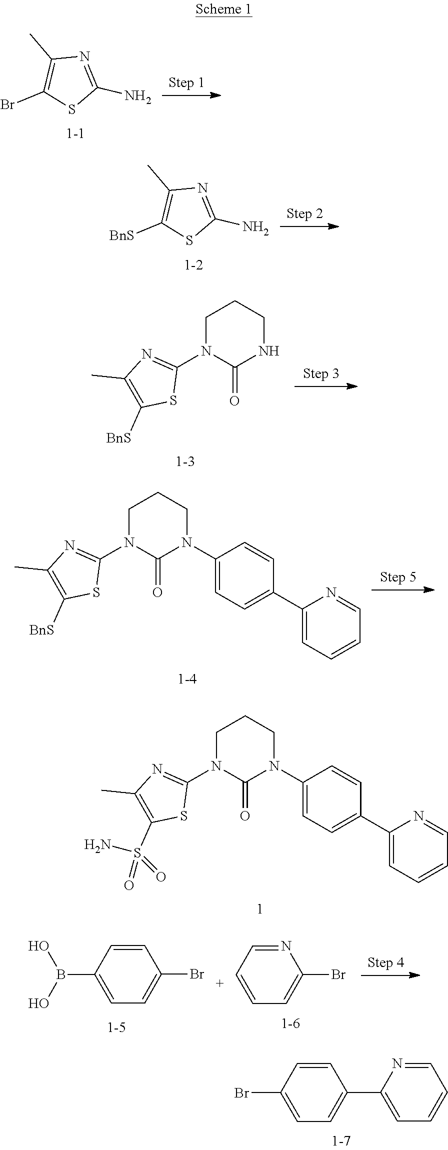

### Step 1. Synthesis of 5-(benzylthio)-4-methylthiazol-2-amine (1-2)

Benzyl mercaptan (15.8 mL, 134.70 mmol) was added dropwise to a stirred solution of 1-1 (20 g, 103.62 mmol) in ethanol (200 mL) at 0° C. The resulting reaction mixture was slowly warmed to room temperature and stirred at 80° C. for 3 h. The reaction mixture was concentrated under reduced pressure to dryness. The resulting residue was dissolved in EtOAc and the organic layer washed with water. The combined organic layers were dried over anhydrous Na₂SO₄, filtered, and concentrated under reduced pressure. The crude product was purified by CombiFlash chromatography (eluting with 10-20% EtOAc in heptane) to afford 1-2 (25 g, 93.9%) as a dark brown colored sticky solid. TLC: 50% EtOAc/Heptane (R_{f}: 0.5). MS (ESI): calcd. for Cn H₁₂N₂S₂: 236.04; Found: 236.95 [M+1]⁺. ¹H NMR (400 MHz, DMSO-d₆): δ 7.35-7.17 (m, 3H), 7.12 (dd, J=1.6, 7.8 Hz, 2H), 7.06 (s, 2H), 3.77 (s, 2H), 1.73 (s, 3H) ppm.

### Step 2. Synthesis of 1-(5-(benzylthio)-4-methylthiazol-2-yl) tetrahydropyrimidin-2(1H)-one (1-3)

A mixture of 1-chloro-3-isocyanatepropane (1.29 g, 10.847 mmol) and compound 1-2 (2 g, 8.474 mmol) in THF (100 mL) was heated at 65° C. for 7 h. TBAI (0.15 g, 0.423 mmol) and K₂CO₃ (1.43 g, 11.016 mmol) were added portion wise to the resulting solution while maintaining the same temperature and stirring continued at 65° C. for 16 h. The reaction mixture was concentrated under reduced pressure to dryness. The crude compound was purified by CombiFlash chromatography (eluting with 60-70% EtOAc in heptane) to afford 1-3 (1.3 g, 48.1%) as an off-white solid. TLC: 70% EtOAc/heptane (R^{f} 0.5). MS (ESI): calcd. for C₁₅H₁₇N₃OS₂: 319.08; Found: 320.10 [M+1]⁺. ¹H NMR (400 MHz, DMSO-d₆): δ 7.45 (br s, 1H), 7.30-7.20 (m, 3H), 7.19-7.03 (m, 2H), 3.93 (t, J=5.6 Hz, 2H), 3.84 (s, 2H), 3.23-3.17 (m, 2H), 1.89 (s, 3H) ppm.

### Step 3. Synthesis of 1-(5-(benzylthio)-4-methylthiazol-2-yl)-3-(4-(pyridin-2-yl) phenyl) tetrahydropyrimidin-2(1H)-one (1-4)

To a stirred solution of 1-3 (0.8 g, 2.50 mmol) in 1,4-dioxane (12 mL) were added compound 1-7 (0.6 g, 2.75 mmol), K₂CO₃ (0.7 g, 5.00 mmol) and 1,2-dimethylethylenediamine (0.11 g, 1.25 mmol). The reaction mixture was purged under nitrogen for 10 min. CuI (0.095 g, 0.50 mmol) was then added under a nitrogen atmosphere. The reaction mixture was heated at 100° C. for 16 h. The reaction mixture was filtered through a Celite bed and washed with ethyl acetate. The filtrate was diluted with water and extracted with EtOAc which was washed with brine. The combined organic layers were dried over anhydrous Na₂SO₄, filtered, and concentrated under 30 reduced pressure. The crude compound was purified by reverse phase CombiFlash chromatography (eluting with 25% ACN in 0.01 M HCOOH in water) to afford 1-4 (0.5 g, 42.3%) as an off-white solid. TLC: 80% EtOAc/Heptane (R_{f}: 0.5). MS (ESI): calcd. for C₂₆H₂₄N₄OS₂: 472.14; Found: 473.2 [M+1]⁺.

### Step 4. Synthesis of 2-(4-bromophenyl) pyridine (1-7)

To a stirred solution of 2-bromopyridine, 1-6 (3 g, 18.987 mmol) in toluene: H₂O:EtOH (1:1:0.2, 150 mL) were added (4-bromophenyl) boronic acid, 1-5 (4.9 g, 24.683 mmol) and Na₂CO₃ (14.90 g, 140.50 mmol) and the reaction mixture was purged under nitrogen for 10 min. Pd (PPh₃)₄ (0.66 g, 0.576 mmol) was the added under a nitrogen atmosphere. The reaction mixture was heated at 100° C. for 16 h. After completion of the reaction (monitored by TLC), the reaction mixture was cooled to room temperature, filtered through a pad of Celite and washed with ethyl acetate. The filtrate was diluted with water and extracted with EtOAc. The combined organic layers were dried over anhydrous Na₂SO₄, filtered, and concentrated under reduced pressure. The crude compound was purified by CombiFlash chromatography (eluting with 20-30% EtOAc in heptane) to afford 1-7 (2 g, 45%) as an off-white solid. TLC: 30% EtOAc/heptane (R_{f}: 0.5). MS (ESI): calcd. for C₁₁H₈BrN: 232.98; Found: 235.98 [M+2]⁺. ¹H NMR (400 MHz, CDCl₃): δ 8.69 (d, J=4.8 Hz, 1H), 7.92-7.84 (m, 2H), 7.80-7.73 (m, 1H), 7.73-7.69 (m, 1H), 7.63-7.57 (m, 2H), 7.30-7.23 (m, 1H) ppm.

### Step 5. Synthesis of 4-methyl-2-(2-oxo-3-(4-(pyridin-2-yl) phenyl) tetrahydropyrimidin-1(2H)-yl) thiazole-5-sulfonamide (1)

To a stirred solution of 1-4 (0.3 g, 0.635 mmol) in AcOH/H₂O (2.8/0.15 mL), NCS (0.31 g, 2.38 mmol) was added, and the reaction mixture was stirred at room temperature for 15 min. After completion of the reaction (monitored by TLC) the reaction mixture was concentrated under reduced pressure to dryness. The residue was dissolved in THF (5 mL) and aqueous ammonia (3 mL) was added while stirring was continued at room temperature for another 4 h. The reaction mixture was concentrated under reduced pressure to dryness. The residue was dissolved in DCM and organic layer was washed with water. The combined organic layers were dried over anhydrous Na₂SO₄, filtered, and concentrated under reduced pressure. The crude compound obtained was purified by reverse phase CombiFlash chromatography (eluting with 10% ACN in 0.01M HCOOH in water) to afford 1 (26 mg, 9.5%) as an off-white solid. TLC: 100% EtOAc (R_{f} 0.5).

### Example 2. 2-(3-(5′-Ethynyl-2′-fluoro-[1,1′-biphenyl]-4-yl)-2-oxotetrahydropyrimidin-1(2H)-yl)-4-methylthiazole-5-sulfonamide (17)

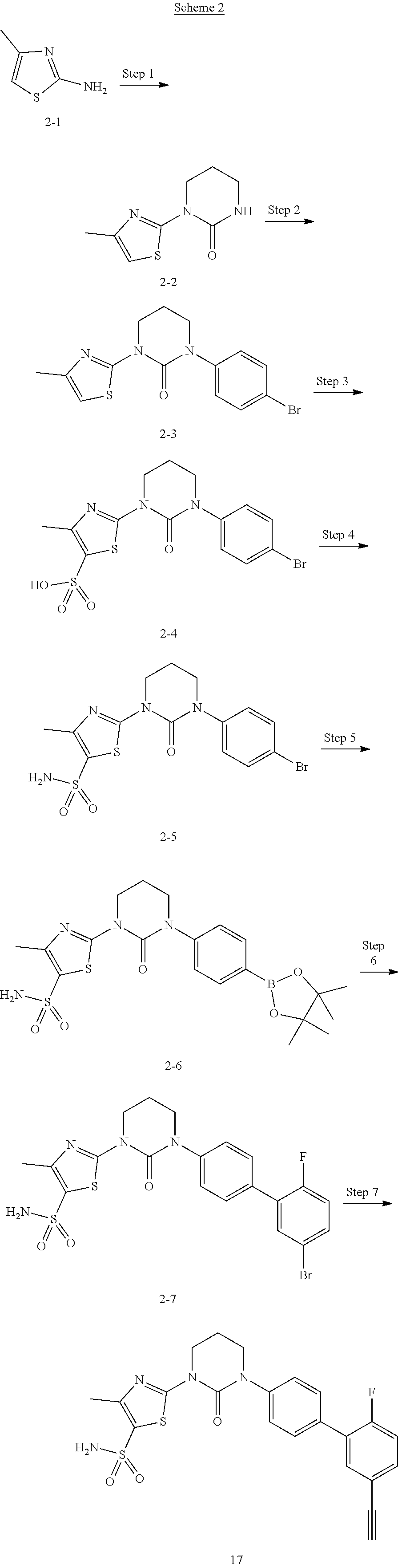

### Step 1. Synthesis of 1-(4-methylthiazol-2-yl) tetrahydropyrimidin-2(1H)-one (2-2)

A mixture of 2-1 (10 g, 87.71 mmol) and 1-chloro-3-isocyanatopropane (15.5 g, 131.57 mmol) in THE (150 mL) was heated at 70° C. for 6 h. To the resulting solution, TBAB (1.4 g, 4.38 mmol) and K₂CO₃ (15.73 g, 114.02 mmol) were added portion wise maintaining the same temperature and stirring continued at 70° C. for 16 h. After completion of the reaction (monitored by TLC), the reaction mixture was diluted with water and extracted with EtOAc. The combined organic layers were dried over anhydrous Na₂SO₄, filtered, and concentrated under reduced pressure. The crude compound was purified by CombiFlash chromatography (eluting with 60-70% EtOAc in heptane) to afford 2-2 (10 g, 57.6%) as an off-white solid. TLC: 70% EtOAc/heptane (R_{f}: 0.5). MS (ESI): calcd. for C₈H₁₁N₃OS: 197.06; Found: 198.17 [M+1]⁺. ¹H NMR (400 MHz, DMSO-d₆): δ 7.30 (s, 1H), 6.60 (s, 1H), 3.99 (t, J=5.4 Hz, 2H), 3.20-3.19 (m, 2H), 2.28 (s, 3H), 1.99-1.89 (m, 2H) ppm.

### Step 2. Synthesis of 1-(4-bromophenyl)-3-(4-methylthiazol-2-yl) tetrahydropyrimidin-2(1H)-one (2-3)

To a stirred solution of 2-2 (5 g, 25.25 mmol) in 1, 4-dioxane (200 mL) were added 1-bromo-4-iodobenzene (14 g, 50.5 mmol), K₂CO₃ (6.9 g, 50.5 mmol) and 1,2-dimethylethylenediamine (1 g, 12.62 mmol) the reaction mixture was purged under nitrogen for 10 min., CuI (0.95 g, 5.05 mmol) was added under a nitrogen atmosphere. The reaction mixture was heated at 100° C. for 16 h. The reaction mixture was filtered through Celite bed and washed with ethyl acetate. The filtrate was diluted with water and extracted with EtOAc then washed with brine. The combined organic layers were dried over anhydrous Na₂SO₄, filtered, and concentrated under reduced pressure. The crude compound obtained was purified by CombiFlash chromatography (eluting with 50-70% EtOAc in heptane) to afford 2-3 (2.5 g, 28.4%) as an off-white solid. TLC: 60% EtOAc/heptane (R_: 0.5). MS (ESI): calcd. for C₁₄H₁₄BrN₃OS: 351.00; Found: 354.09 [M+2]⁺.

### Step 3. Synthesis of 2-(3-(4-bromophenyl)-2-oxotetrahydropyrimidin-1(2H)-yl)-4-methylthiazole-5-sulfonic acid (2-4)

To a stirred solution of 2-3 (2 g, 5.68 mmol) in dry DCM (20 mL) at 0° C. in an inert atmosphere, was added chlorosulfuric acid (1.3 g, 11.363 mmol) and the resulting reaction mixture was slowly warmed to room temperature and stirred for 16 h. After completion of the reaction (monitored by TLC), the reaction mixture was quenched with ice cold water and stirred for 5-10 min. The precipitated solid out was collected by filtration and dried in vacuo to afford 2-4 (2 g, 81.9%) as an off-white solid. TLC: 5% MeOH/DCM (R_{f}: 0.5) which was used in the next step without further purification. MS (ESI): calcd. for C₁₄H₁₄BrN₃O₄S₂: 430.96; Found: 434.00 [M+2]⁺

### Step 4. Synthesis of 2-(3-(4-bromophenyl)-2-oxotetrahydropyrimidin-1(2H)-yl)-4-methylthiazole-5-sulfonamide (2-5)

A mixture of 2-4 (1 g, 2.315 mmol) and POCl₃ (10 mL) was allowed to stir at 100° C. for 12 h. The reaction mixture was concentrated under reduced pressure to dryness. The residue was dissolved in THF (10 mL) and aqueous ammonia (20 mL) was added at 0° C. while stirring was continued at room temperature for another 4 h. The reaction mixture was diluted with water and extracted with EtOAc. The combined organic layers were dried over anhydrous Na₂SO₄ filtered, and concentrated under reduced pressure. The crude compound was purified by CombiFlash chromatography (eluting with 30-40% EtOAc in heptane) to afford 2-5 (0.8 g, 80.8%) as an off-white solid. TLC: 50% EtOAc/heptane (R_{f}: 0.5). MS (ESI): calcd. for C₁₄H₁₅BrN₄O₃S₂: 429.98; Found: 431.1 [M+1]⁺.

### Step 5. Synthesis of 4-methyl-2-(2-oxo-3-(4-(4,4,5,5-tetramethyl-1,3,2-dioxaborolan-2-yl) phenyl) tetrahydropyrimidin-1(2H)-yl) thiazole-5-sulfonamide (2-6)

To a stirred solution of 2-5 (0.6 g, 1.392 mmol) in 1, 4-dioxane (20 mL) were added bis(pinacolato)diborane (0.53 g, 2.088 mmol) and KOAc (0.27 g, 2.784 mmol) and the reaction mixture was purged under nitrogen for 10 min. PdCl₂(dppf) (0.1 g, 0.14 mmol) was added under a nitrogen atmosphere. The reaction mixture was heated at 120° C. for 16 h. After completion of the reaction (monitored by TLC), the reaction mixture was cooled to room temperature, diluted with water and extracted with EtOAc then washed with brine. The combined organic layers were dried over anhydrous Na₂SO₄, filtered, and concentrated under reduced pressure. The crude compound was purified by CombiFlash chromatography (eluting with 30-40% EtOAc in heptane) to afford 2-6 (0.8 g, 80.8%) as an off-white solid. TLC: 50% EtOAc/heptane (R_{f}: 0.5). MS (ESI): calcd. for C₂₀H₂₇BN₄O₅S₂: 478.15; Found: 479.2 [M+1]⁺. ¹H NMR (400 MHz, DMSO-d₆): δ 7.70 (d, J=7.8 Hz, 2H), 7.55 (s, 2H), 7.42-7.38 (m, 2H), 4.17-4.09 (m, 2H), 3.82-3.74 (m, 2H), 2.44 (s, 3H), 2.26-2.15 (m, 2H), 1.30 (s, 9H) ppm.

### Step 6. Synthesis of 2-(3-(5′-bromo-2′-fluoro-[1,1′-biphenyl]-4-yl)-2-oxotetrahydropyrimidin-1(2H)-yl)-4-methylthiazole-5-sulfonamide (2-7)

To a stirred solution of 2-6 (0.4 g, 0.836 mmol) in 1, 4-dioxane and water (16:4 mL) were added 4-bromo-1-fluoro-2-iodobenzene (0.25 g, 0.836 mmol) and Na₂CO₃ (0.17 g, 1.673 mmol) and the reaction mixture was purged under nitrogen for 10 min. Pd(dppf) Cl₂ (61 mg, 0.083 mmol) was added under nitrogen atmosphere and the reaction mixture heated at 100° C. for 12 h. After completion of the reaction (monitored by TLC), the reaction mixture was cooled to room temperature, diluted with water and extracted with EtOAc then washed with brine. The combined organic layers were dried over anhydrous Na₂SO₄, filtered, and concentrated under reduced pressure. The crude compound was purified by CombiFlash chromatography (eluting with 30-40% EtOAc in heptane) to afford 2-7 (0.2 g, 45.5%) as an off-white solid. TLC: 50% EtOAc/heptane (R_{f} 0.5). MS (ESI): calcd. for C₂₀H₁₈BrFN₄O₃S₂: 524.00; Found: 525.00 [M+1]⁺.

### Step 7. Synthesis of 2-(3-(5′-ethynyl-2′-fluoro-[1,1′-biphenyl]-4-yl)-2-oxotetrahydropyrimidin-1(2H)-yl)-4-methylthiazole-5-sulfonamide (17)

To a stirred solution of 2-7 (0.1 g, 0.190 mmol) in DMF (5 mL) was added triethylamine (0.1 mL, 0.571 mmol) and CuI (4 mg, 0.019 mmol) and the reaction mixture purged under nitrogen for 10 min. Pd (PPh₃)₂Cl₂ (27 mg, 0.038 mmol) and ethynyltrimethylsilane (56 mg, 0.571 mmol) were added under a nitrogen atmosphere and then the reaction mixture was heated at 120° C. for 12 h. After completion of the reaction (monitored by TLC), the reaction mixture was cooled to room temperature, diluted with water and extracted with EtOAc then washed with brine. The 15 combined organic layers were dried over anhydrous Na₂SO₄, filtered, and concentrated under reduced pressure. The crude compound obtained was purified by preparative HPLC to afford 17 (5 mg, 5.61%) as an off-white solid. TLC: 50% EtOAc/heptane (R_{f} 0.5).

### Example 3. 2-(3-(5′-Fluoro-2′-(3-hydroxy-3-methylbut-1-yn-1-yl)-[1,1′-biphenyl]-4-yl)-2-oxotetrahydropyrimidin-1(2H)-yl)-4-methylthiazole-5-sulfonamide (19)

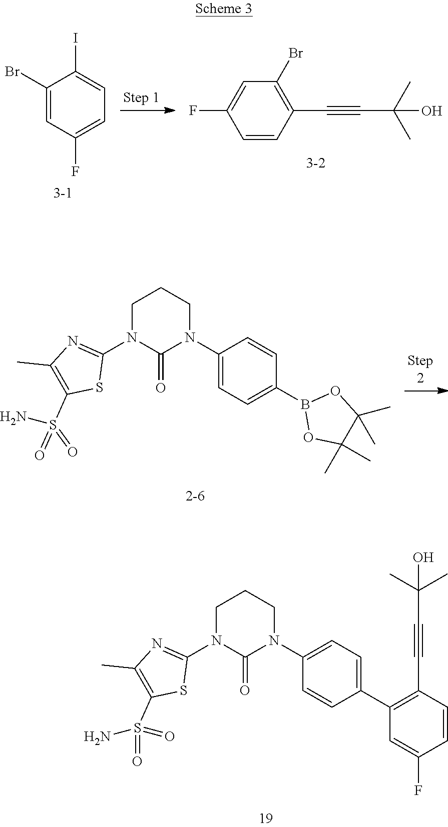

### Step 1. Synthesis of 4-(2-bromo-4-fluorophenyl)-2-methylbut-3-yn-2-ol (3-2)

To a stirred solution of compound 3-1 (1 g, 3.323 mmol) in DIPEA (10 mL) were added 2-methylbut-3-yn-2-ol (0.49 g, 4.984 mmol) and the reaction mixture was purged under nitrogen for 10 min. To this resulting reaction mixture, CuI (63 mg, 0.332 mmol) and Pd(dppf)Cl₂ (0.11 g, 0.166 mmol) were added under a nitrogen atmosphere. The reaction mixture was stirred at room temperature for 16 h. The reaction mixture was filtered through Celite bed and washed with ethyl acetate. The filtrate was diluted with water and extracted with EtOAc then washed with brine. The combined organic layers were dried over anhydrous Na₂SO₄, filtered, and concentrated under reduced pressure. The crude compound obtained was purified by CombiFlash chromatography (eluting with 5-10% EtOAc in heptane) to afford 3-2 (0.8 g, 94.1%) as a brown oil. TLC: 10% EtOAc/Heptane (R_{f}: 0.5).

### Step 2. Synthesis of 2-(3-(5′-fluoro-2′-(3-hydroxy-3-methylbut-1-yn-1-yl)-[1,1′-biphenyl]-4-yl)-2-oxotetrahydropyrimidin-1(2H)-yl)-4-methylthiazole-5-sulfonamide (19)

To a stirred solution of 2-6 (0.2 g, 0.418 mmol) in 1, 4-dioxane:water (4:1, 10 mL) was added 4-(2-bromo-4-fluorophenyl)-2-methylbut-3-yn-2-ol (0.1 g, 0.418 mmol) followed by Na₂CO₃ (88 mg, 0.836 mmol) and the resulting reaction mixture was purged under nitrogen for 20 min. To this resulting reaction mixture, Pd(dppf)Cl₂ (30 mg, 0.0418 mmol) was added under a nitrogen atmosphere and the reaction mixture was heated at 100° C. for 16 h. The reaction mixture was filtered through Celite bed and washed with ethyl acetate. The filtrate was diluted with water and extracted with EtOAc then washed with brine. The combined organic layers were dried over anhydrous Na₂SO₄, filtered, and concentrated under reduced pressure. The crude compound obtained was purified by preparative HPLC to afford 19 (10 mg, 4.5%) as an off-white solid. TLC: 50% EtOAc/Heptane (R_{f}: 0.5).

### Example 4. 4-Methyl-2-(2-oxo-3-(3′-(thiazol-5-yl)-[1,1′-biphenyl]-4-yl)tetrahydropyrimidin-1(2H)-yl)thiazole-5-sulfonamide (30)

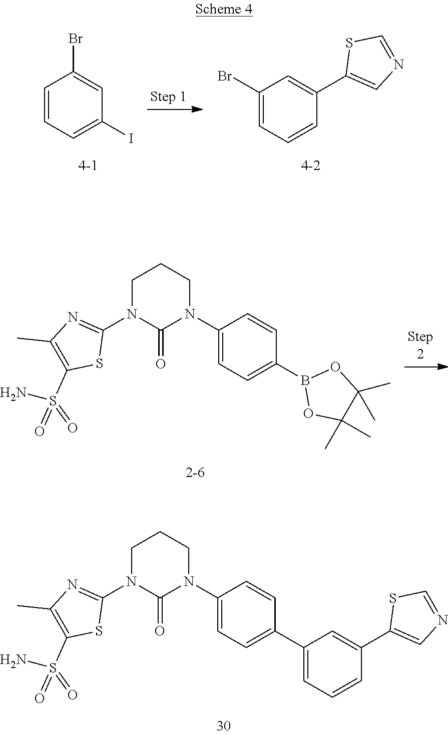

### Step 1. Synthesis of 2-(3-bromophenyl)thiazole (4-2)

To a stirred solution of 4-1 (1 g, 6.097 mmol) in 1, 4-dioxane:water (4:1, 15 mL) was added (3-bromophenyl)boronic acid (1.34 g, 6.707 mmol) followed by K₃PO₄ (3.18 g, 15.242 mmol) and the resulting reaction mixture was purged under nitrogen for 20 min. To this resulting reaction mixture, Pd(dppf)Cl₂ (0.445 g, 0.6097 mmol) was added under nitrogen atmosphere and the reaction mixture was heated at 100° C. for 16 h. The reaction mixture was filtered through Celite bed and washed with ethyl acetate. The filtrate was diluted with water and extracted with EtOAc then washed with brine. The combined organic layers were dried over anhydrous Na₂SO₄, filtered, and concentrated under reduced pressure. The crude product was purified by CombiFlash chromatography (eluting with 2-3% EtOAc in heptane) to afford 4-2 (0.2 g, 13.7%) as an off-white solid. TLC: 10% EtOAc/Heptane (R_{f}: 0.5).

### Step 2. Synthesis of 4-methyl-2-(2-oxo-3-(3′-(thiazol-2-yl)-[1,1′-biphenyl]-4-yl)tetrahydropyrimidin-1(2H)-yl)thiazole-5-sulfonamide (30)

To a stirred solution of 8 (0.2 g, 0.418 mmol) in 1, 4-dioxane:water (4:1, 10 mL) was added 2-(3-bromophenyl)thiazole (0.1 g, 0.418 mmol) followed by K₃PO₄ (0.221 g, 1.04 mmol) and the resulting reaction mixture was purged under nitrogen for 20 min. To this resulting reaction mixture, Pd(dppf)Cl₂ (30.6 mg, 0.041 mmol) was added under a nitrogen atmosphere and the reaction mixture was heated at 100° C. for 16 h. The reaction mixture was filtered through Celite bed and washed with ethyl acetate. The filtrate was diluted with water and extracted with EtOAc then washed with brine. The combined organic layers were dried over anhydrous Na₂SO₄, filtered, and concentrated under reduced pressure. The crude compound obtained was purified by preparative HPLC to afford 30 (14 mg, 6.5%) as an off-white solid. TLC: 80% EtOAc/Heptane (R_{f}: 0.5).

### Example 5. 4-Methyl-2-(2-oxo-3-(4-(thiazol-2-yl) phenyl) tetrahydropyrimidin-1(2H)-yl) thiazole-5-sulfonamide (42)

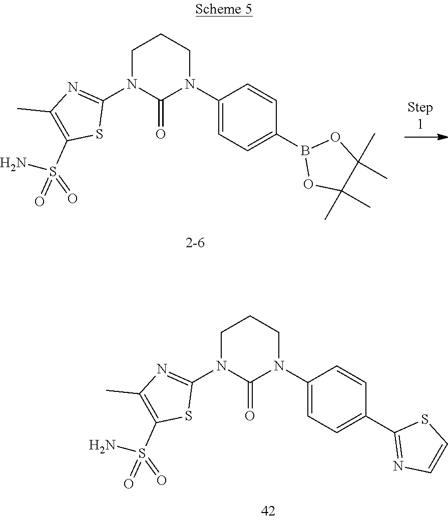

### Step 1. Synthesis of 4-methyl-2-(2-oxo-3-(4-(thiazol-2-yl) phenyl) tetrahydropyrimidin-1(2H)-yl) thiazole-5-sulfonamide (42)

To a stirred solution of 2-6 (0.15 g, 0.313 mmol) in 1, 4-dioxane and water (8:2 mL) were added 2-bromothiazole (0.1 g, 0.626 mmol) and K₃PO₄ (0.13 g, 0.626 mmol) and the reaction mixture was purged under nitrogen for 10 min. Pd(dppf)Cl₂ (23 mg, 0.0313 mmol) was added under a nitrogen atmosphere. The reaction mixture was heated at 100° C. for 16 h. After completion of the reaction (monitored by TLC), the reaction mixture was cooled to room temperature, diluted with water and extracted with EtOAc then washed with brine. The combined organic layers were dried over anhydrous Na₂SO₄, filtered, and concentrated under reduced pressure. The crude compound was purified by preparative HPLC to afford 42 (20 mg, 15.4%) as an off-white solid. TLC: 50% EtOAc/heptane (R_{f}: 0.5).

### Example 6. 2-(3-(4-(Benzo[d]thiazol-4-yl) phenyl)-2-oxotetrahydropyrimidin-1(2H)-yl)-4-methylthiazole-5-sulfonamide (51)

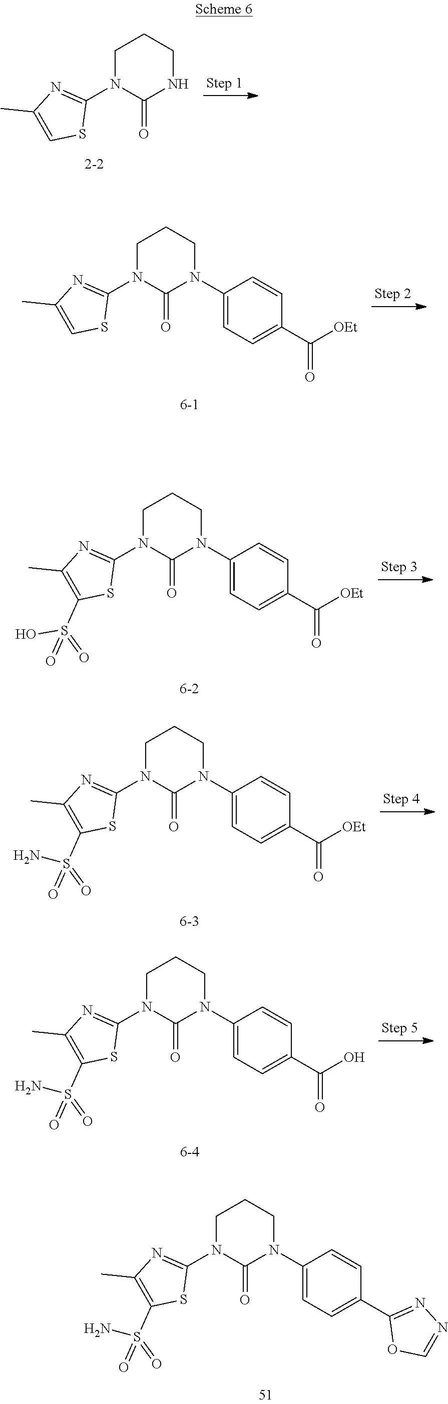 [SMILES: Cc1csc(N2CCCNC2=O)n1 ; CCOC(=O)c1ccc(N2CCCN(c3nc(C)cs3)C2=O)cc1 ; CCOC(=O)c1ccc(N2CCCN(c3nc(C)c(S(=O)(=O)O)s3)C2=O)cc1 ; CCOC(=O)c1ccc(N2CCCN(c3nc(C)c(S(N)(=O)=O)s3)C2=O)cc1 ; Cc1nc(N2CCCN(c3ccc(C(=O)O)cc3)C2=O)sc1S(N)(=O)=O ; Cc1nc(N2CCCN(c3ccc(-c4nnco4)cc3)C2=O)sc1S(N)(=O)=O]

### Step 1. Synthesis of ethyl 4-(3-(4-methylthiazol-2-yl)-2-oxotetrahydropyrimidin-1(2H)-yl) benzoate (6-1)

To a stirred solution of 2-2 (3.1 g, 15.656 mmol) in 1, 4-dioxane (100 mL) were added ethyl 4-iodobenzoate (4.3 g, 15.656 mmol), K₂CO₃ (4.3 g, 3.131 mmol) and 1,2-dimethylethylenediamine (0.7 g, 1.269 mmol) and the reaction mixture was purged under nitrogen for 10 min. To this resulting reaction mixture, CuI (0.6 g, 3.131 mmol) was subsequently added under a nitrogen atmosphere and the resulting reaction mixture was heated at 100° C. for 24 h. After completion of the reaction, the reaction mixture was filtered through Celite bed and washed with ethyl acetate. The filtrate was diluted with water and extracted with EtOAc then washed with brine. The combined organic layers were dried over anhydrous Na₂SO₄, filtered, and concentrated under reduced pressure. The crude compound obtained was purified by CombiFlash chromatography (eluting with 50-70% EtOAc in heptane) to afford the 6-1 (3 g, 57.6%) as an off-white solid. TLC: 50% EtOAc/Heptane (R_{f}: 0.5). MS (ESI): calcd. for C₁₇H₁₉N₃O₃S: 345.1; Found: 346.1 [M+1]⁺.

### Step 2. Synthesis of 2-(3-(4-(ethoxycarbonyl)phenyl)-2-oxotetrahydropyrimidin-1(2H)-yl)-4-methylthiazole-5-sulfonic acid (6-2)

To a stirred solution of 6-1 (3 g, 8.695 mmol) in dry DCM (50 mL) at 0° C. in an inert atmosphere, chlorosulfuric acid (2 g, 17.39 mmol) was added and the resulting reaction mixture was slowly warmed to room temperature and stirred for 16 h. After completion of the reaction (monitored by TLC), the reaction mixture was quenched with ice cold water and stirred for 5-10 min. The precipitated solid obtained out was collected by filtration and dried in vacuo to afford 6-2 (3 g, crude) as an off-white solid. TLC: 50% EtOAc/heptane (R_{f}: 0.5) which was used in the next step without further purification. MS (ESI): calcd. for C₁₇H₁₉N₃O₆S₂: 425.1; Found: 426.1 [M+1]⁺.

### Step 3. Synthesis of ethyl 4-(3-(4-methyl-5-sulfamoylthiazol-2-yl)-2-oxotetrahydropyrimidin-1(2H)-yl) benzoate (6-3)

A mixture of 6-2 (0.5 g, 1.176 mmol) and POCl₃ (5 mL) was allowed to stir at 100° C. for 16 h. The reaction mixture was concentrated under reduced pressure to dryness. The residue was dissolved in THF (5 mL) and aqueous ammonia (8 mL) was added at 0° C. while stirring was continued at room temperature for another 4 h. After completion of the reaction (monitored by TLC), the reaction mixture was quenched with ice cold water and stirred for 5-10 min. The precipitated solid obtained out was collected by filtration and dried in vacuo to afford 6-3 (0.3 g, crude) as an off-white solid. TLC: 50% EtOAc/heptane (R_{f}: 0.5) which was used in the next step without further purification. MS (ESI): calcd. for C₁₇H₂₉N₄O₅S₂: 424.09; Found: 424.92 [M+1]⁺.

### Step 4. Synthesis of 4-(3-(4-methyl-5-sulfamoylthiazol-2-yl)-2-oxotetrahydropyrimidin-1(2H)-yl) benzoic acid (6-4)

To a stirred solution of 6-3 (0.2 g, 0.471 mmol) in THF:MeOH:H₂O (8:1:1 mL) at 0° C., Lithium hydroxide monohydrate (39 mg, 0.943 mmol) was added portion wise. The resulting reaction mixture was slowly warmed to room temperature and allowed to stir for 3 h. The reaction mixture was concentrated under reduced pressure to dryness. The residue was treated with saturated KHSO₄ solution and the precipitated solid obtained out was collected by filtration and dried in vacuo to afford 6-4 (0.2 g, crude) as an off-white solid, which was used in the next step without further purification. TLC: 50% EtOAc/heptane (R_{f}: 0.5); MS (ESI): calcd. for C₁₅H₁₆N₄O₅S₂: 396.06; Found: 397.2 [M+1]⁺.

### Step 5. Synthesis of 2-(3-(4-(1,3,4-oxadiazol-2-yl)phenyl)-2-oxotetrahydropyrimidin-1(2H)-yl)-4-methylthiazole-5-sulfonamide (51)

To a stirred solution of 6-4 (0.2 g, 0.505 mmol) in DCM/EtOH (10/10 mL) was added (N-isocyanoimino)triphenylphosphorane (0.30 g, 1.010 mmol) and the resulting reaction mixture was allowed to stir at room temperature for 16 h. After completion of the reaction (monitored by TLC), the resulting reaction mixture was diluted with water and extracted with ethyl acetate. The combined organic layers were dried over anhydrous Na₂SO₄, filtered, and concentrated under reduced pressure. The crude was purified by preparative HPLC to afford 51 (36 mg, 16.9%) as an off-white solid.

### Example 7. 2-(3-(4-(Benzo[d]thiazol-7-yl) phenyl)-2-oxotetrahydropyrimidin-1(2H)-yl)-4-methylthiazole-5-sulfonamide (67)

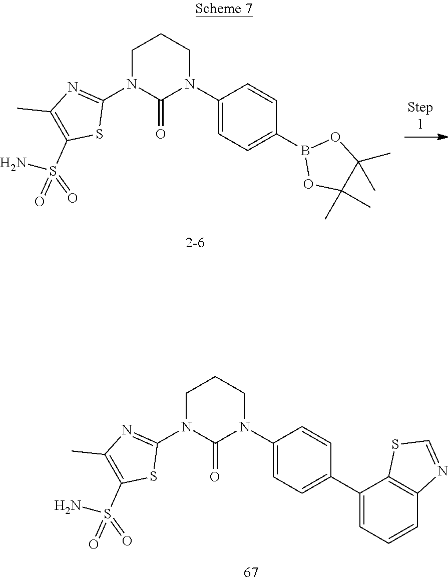

### Step 1. Synthesis of 2-(3-(4-(benzo[d]thiazol-7-yl) phenyl)-2-oxotetrahydropyrimidin-1(2H)-yl)-4-methylthiazole-5-sulfonamide (67)

To a stirred solution of 2-6 (0.2 g, 0.418 mmol) in 1, 4-dioxane and water (5/0.5 mL) was added 7-bromobenzo[d]thiazole (0.18 g, 0.836 mmol) followed by K₂CO₃ (0.17 g, 0.836 mmol) and the reaction mixture was purged under nitrogen for 10 min. To this resulting solution, Pd(dppf)Cl₂ (30 mg, 0.041 mmol) was added under a nitrogen atmosphere and the reaction mixture was heated at 120° C. for 16 h. After completion of the reaction (monitored by TLC), the reaction mixture was cooled to room temperature, diluted with water and extracted with EtOAc then washed with brine. The combined organic layers were dried over anhydrous Na₂SO₄, filtered, and concentrated under reduced pressure. The crude compound was purified by preparative HPLC to afford 67 (10 mg, 5%) as an off-white solid. TLC: 60% EtOAc/heptane (R_{f} 0.5).

### Example 8. 2-(3-(4-(Benzo[d]thiazol-4-yl) phenyl)-2-oxotetrahydropyrimidin-1(2H)-yl)-4-methylthiazole-5-sulfonamide (71)

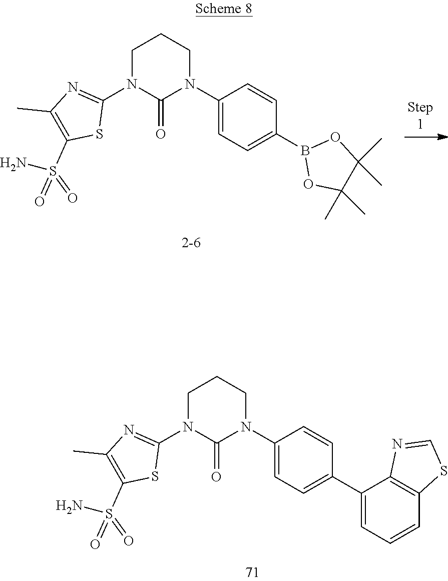

### Step 1. Synthesis of 2-(3-(4-(benzo[d]thiazol-4-yl) phenyl)-2-oxotetrahydropyrimidin-1(2H)-yl)-4-methylthiazole-5-sulfonamide (71)

To a stirred solution of 2-6 (0.2 g, 0.418 mmol) in 1, 4-dioxane and water (4:1, 10 mL) were added 4-bromobenzo[d]thiazole (0.13 g, 0.627 mmol) and Na₂CO₃ (88 mg, 0.836 mmol) and the reaction mixture was purged under nitrogen for 10 min. To this resulting solution, Pd(dppf)Cl₂ (30 mg, 0.0406 mmol) was added under a nitrogen atmosphere and the reaction mixture was heated at 100° C. for 12 h. After completion of the reaction (monitored by TLC), the reaction mixture was cooled to room temperature, diluted with water and extracted with EtOAc then washed with brine. The combined organic layers were dried over anhydrous Na₂SO₄, filtered, and concentrated under reduced pressure. The crude compound was purified by preparative HPLC to afford 71 (30 mg, 15%) as an off-white solid. TLC: 50% EtOAc/heptane (R_{f}: 0.5).

### Example 9. 2-(3-(2,5-Difluoro-[1,1′-biphenyl]-4-yl)-2-oxotetrahydropyrimidin-1(2H)-yl)-4-methylthiazole-5-sulfonamide (99)

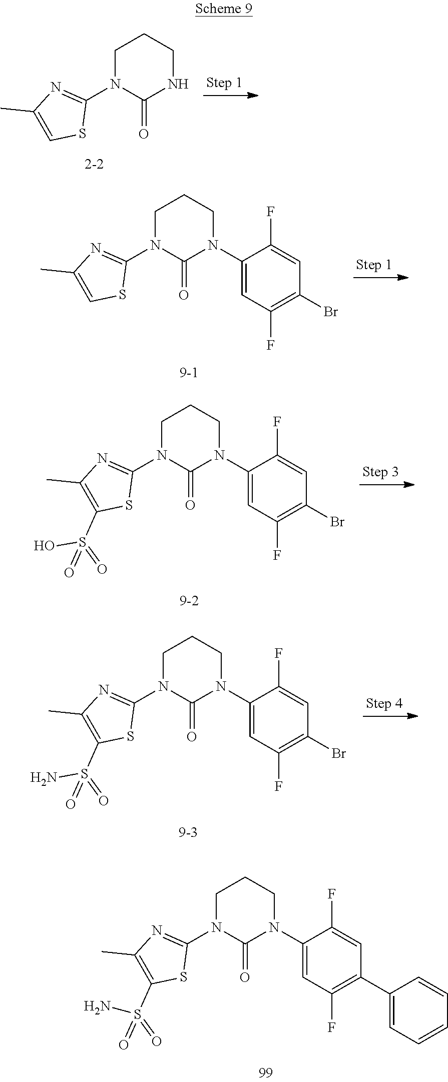 [SMILES: Cc1csc(N2CCCNC2=O)n1 ; Cc1csc(N2CCCN(c3cc(F)c(Br)cc3F)C2=O)n1 ; Cc1nc(N2CCCN(c3cc(F)c(Br)cc3F)C2=O)sc1S(=O)(=O)O ; Cc1nc(N2CCCN(c3cc(F)c(Br)cc3F)C2=O)sc1S(N)(=O)=O ; Cc1nc(N2CCCN(c3cc(F)c(-c4ccccc4)cc3F)C2=O)sc1S(N)(=O)=O]

### Step 1. Synthesis of 1-(4-bromo-2,5-difluorophenyl)-3-(4-methylthiazol-2-yl) tetrahydropyrimidin-2(1H)-one (9-1)

To a stirred solution of 2-2 (1.5 g, 7.614 mmol) in 1, 4-dioxane (30 mL) were added 1,4-dibromo-2,5-difluorobenzene (3.10 g, 11.42 mmol), K₂CO₃ (2.1 g, 15.228 mmol) and 1,2-Dimethylethylenediamine (0.27 g, 3.045 mmol) the reaction mixture was purged under nitrogen for 10 min. To this resulting reaction mixture, CuI (0.3 g, 1.522 mmol) was added under a nitrogen atmosphere. The reaction mixture was heated at 120° C. for 24 h. The reaction mixture was filtered through Celite*545 bed and washed with ethyl acetate. The filtrate was diluted with water and extracted with EtOAc then washed with brine. The combined organic layers were dried over anhydrous Na₂SO₄, filtered, and concentrated under reduced pressure. The crude compound obtained was purified by CombiFlash chromatography (eluting with 50-70% EtOAc in heptane) to afford 9-1 (0.8 g, 27.1%) as an off-white solid. TLC: 50% EtOAc/Heptane (R_{f}: 0.5). MS (ESI): calcd. for C₁₄H₁₂BrF₂N₃OS: 386.99; Found: 390.0 [M+2]⁺. ¹H NMR (400 MHz, DMSO-d₆): δ 7.86 (dd, J=6.4, 9.3 Hz, 1H), 7.67 (d, J=2.4 Hz, 1H), 6.72 (s, 1H), 4.16 (t, J=5.9 Hz, 2H), 3.69 (t, J=5.4 Hz, 2H), 2.25 (s, 3H), 2.23-2.17 (m, 2H) ppm.

### Step 2. Synthesis of 2-(3-(4-bromo-2,5-difluorophenyl)-2-oxotetrahydropyrimidin-1(2K)-yl)-4-methylthiazole-5-sulfonic acid (9-2)

To a stirred solution of 9-1 (0.89 g, 2.294 mmol) in dry DCM (20 mL) at 0° C. in an inert atmosphere, chlorosulfuric acid (0.38 g, 5.734 mmol) was added and the resulting reaction mixture was slowly warmed to room temperature and stirred for 12 h. After completion of the reaction (monitored by TLC), the reaction mixture was quenched with ice cold water and stirred for 5-10 min. The precipitated solid obtained out was collected by filtration and dried in vacuo to afford 9-2 (0.75 g, crude) as an off-white solid. TLC: 50% EtOAc/heptane (R_{f}: 0.5), which was used in the next step without further purification. MS (ESI): calcd. for C₁₄H₁₂BrF₂N₃O₄S₂: 466.94; Found: 469.85 [M+2]⁺. ¹H NMR (400 MHz, DMSO-d₆): δ 7.87 (t, J=7.1 Hz, 1H), 7.69 (t, J=7.1 Hz, 1H), 5.79 (br.s, 1H), 4.22-3.98 (m, 2H), 3.73-3.59 (m. 2H). 2.32 (s, 3H), 2.27-2.09 (m, 2H) ppm.

### Step 3. Synthesis of 2-(3-(4-bromo-2,5-difluorophenyl)-2-oxotetrahydropyrimidin-1(2H)-yl)-4-methylthiazole-5-sulfonamide (9-3)

A mixture of 9-2 (1 g, 2.136 mmol) and POCl₃ (10 mL) was allowed to stir at 100° C. for 12 h. The reaction mixture was concentrated under reduced pressure to dryness. The residue was dissolved in THF (5 mL) and aqueous ammonia (5 mL) was added at 0° C. while stirring was continued at room temperature for another 2 h. The reaction mixture was diluted with water and extracted with EtOAc. The combined organic layers were dried over anhydrous Na₂SO₄ filtered, and concentrated under reduced pressure. The crude compound was purified by CombiFlash chromatography (eluting with 30-40% EtOAc in heptane) to afford 9-3 (0.8 g, 80.8%) as an off-white solid. TLC: 50% EtOAc/heptane (R_{f}: 0.5). MS (ESI): calcd. for C₁₄H₁₃BrF₂N₄O₃S₂: 465.96; Found: 467.0 [M+1]⁺. ¹H NMR (400 MHz, DMSO-d₆): δ 7.93-7.85 (m, 1H), 7.70 (t, J=7.8 Hz, 1H), 7.57 (br.s, 2H), 4.21-4.12 (m, 2H), 3.77-3.67 (m, 2H), 2.44 (s, 3H), 2.27-2.17 (m, 2H) ppm.

### Step 4. Synthesis of 2-(3-(2,5-difluoro-[1,1′-biphenyl]-4-yl)-2-oxotetrahydropyrimidin-1(2H)-yl)-4-methylthiazole-5-sulfonamide (99)

To a stirred solution of 9-3 (0.2 g, 0.428 mmol) in 1, 4-dioxane and water (4:1 mL) were added phenylboronic acid (78 mg, 0.642 mmol) and Na₂CO₃ (90 mg, 0.856 mmol) and the reaction mixture was purged under nitrogen for 10 min. Pd(dppf)Cl₂ (31 mg, 0.043 mmol) was added under a nitrogen atmosphere. The reaction mixture was heated at 110° C. for 12 h. The reaction mixture was filtered through Celite bed and washed with ethyl acetate, the resulting filtrate was concentrated under reduced pressure. The crude compound obtained was purified by CombiFlash chromatography (eluting with 80-90% EtOAc in heptane) to afford 99 (0.12 g, 60.6%) as an off-white solid. TLC: 30% EtOAc/heptane (R_{f}: 0.5).

### Example 10. 2-(3-(2′,5′-Difluoro-[1,1′-biphenyl]-4-yl)-2-oxo-1,3-diazepan-1-yl)-4-methylthiazole-5-sulfonamide (101)

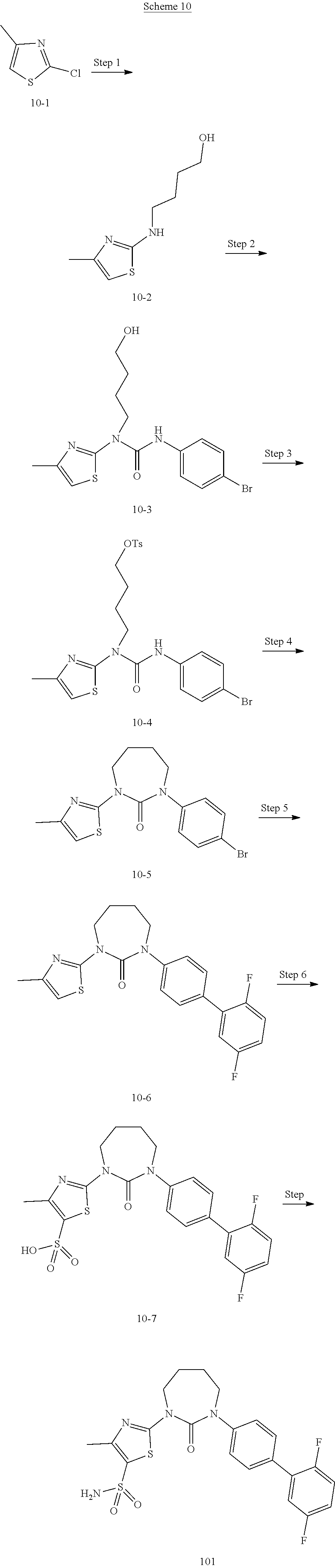

### Step 1. Synthesis of 4-((4-methylthiazol-2-yl) amino) butan-1-ol (10-2)

To a stirred solution of 10-1 (5 g, 37.593 mmol) in DMSO (50 mL) were added K₂CO₃ (7.78 g, 56.390 mmol), 4-aminobutan-1-ol (6.66 g, 75.186 mmol) followed by CuI (0.71 g, 3.759 mmol). The resulting reaction mixture was heated at 100° C. for 12 h. After completion of the reaction (monitored by TLC), the reaction mixture was cooled to room temperature, filtered through a pad of Celite*545 and washed with ethyl acetate. The filtrate was diluted with water and extracted with EtOAc then washed with brine. The combined organic layers were dried over anhydrous Na₂SO₄, filtered, and concentrated under reduced pressure. The crude product was purified by CombiFlash column chromatography [eluting with 20-30% EtOAc in heptane] to afford 10-2 (4 g, 28.7%) as a pale brown colored oil. TLC: 20% EtOAc/heptane (R_{f}: 0.2). MS (ESI): calcd. for C₈H₁₄N₂OS: 186.27; Found: 187.0 [M+1]⁺. ¹H NMR (400 MHz, DMSO-d₆): δ 7.37 (br s, 1H), 6.10 (s, 1H), 4.40 (t, J=4.6 Hz, 1H), 3.45-3.35 (m, 2H), 3.21-3.09 (m, 2H), 2.07 (s, 3H), 1.60-1.41 (m, 4H) ppm.

### Step 2. Synthesis of 3-(4-bromophenyl)-1-(4-hydroxybutyl)-1-(4-methylthiazol-2-yl) urea (10-3)

To a stirred solution of 10-2 (1.5 g, 8.064 mmol) in 1,4 dioxane (30 mL) were added compound 1-bromo-4-isocyanatobenzene (1.6 g, 8.064 mmol). The resulting reaction mixture was allowed to stir at room temperature for 12 h. After completion of the reaction (monitored by TLC), the reaction mixture was diluted with water and extracted with EtOAc then washed with brine. The combined organic layers were dried over anhydrous Na₂SO₄, filtered, and concentrated under reduced pressure. The crude product was purified by CombiFlash column chromatography [eluting with 30-40% EtOAc in heptane] to afford 10-3 (1.1 g, 38.8%) as an off-white solid. TLC: 50% EtOAc/heptane (R_{f}: 0.2). MS (ESI): calcd. for C₁₅H₁₈BrN₃O₂S: 383.03; Found: 386 [M+2]⁺.

### Step 3. Synthesis of 4-(3-(4-bromophenyl)-1-(4-methylthiazol-2-yl) ureido) butyl 4-methylbenzenesulfonate (10-4)

To a stirred solution of 10-3 (1.0 g, 2.604 mmol) in DCM (20 mL) were added triethyl amine (0.55 mL, 3.906 mmol) followed by tosyl chloride (0.6 g, 3.125 mmol) at 0° C. The resulting reaction mixture was slowly warmed to room temperature and allowed to stir for 12 h. After completion of the reaction (monitored by TLC), the reaction mixture was diluted with water and extracted with DCM. The combined organic layers were dried over anhydrous Na₂SO₄, filtered and concentrated under reduced pressure. The crude product was purified by CombiFlash column chromatography [eluting with 30-40% EtOAc in heptane] to afford 10-4 (1 g, 71.4%) as an off-white solid. TLC: 50% EtOAc/heptane (R_{f}: 0.5). MS (ESI): calcd. for C₂₂H₂BrN₃O₄S₂: 537.04; Found: 538.14 [M+1]⁺. 1H NMR (400 MHz, DMSO-d₆): δ 10.05 (s, 1H), 7.75 (d, J=8.3 Hz, 2H), 7.55-7.46 (m, 4H), 7.46-7.41 (m, 2H), 6.78 (s, 1H), 4.15-4.03 (m, 4H), 2.39 (s, 3H), 2.27 (s, 3H), 1.66-1.58 (m, 4H) ppm.

### Step 4. Synthesis of 1-(4-bromophenyl)-3-(4-methylthiazol-2-yl)-1,3-diazepan-2-one (10-5)

To a stirred solution of 10-4 (1 g, 1.858 mmol) in dry DMF (10 mL) at 0° C. under a nitrogen atmosphere, NaH (60% w/w in mineral oil, 0.134 g, 2.788 mmol) was added in small portions and The resulting reaction mixture was slowly warmed to room temperature and stirred for 4 h. After completion of the reaction (monitored by TLC), the reaction mixture was quenched with ice cold water and stirred for 15 min. The precipitated solid obtained was collected by filtration and dried in vacuo to afford 10-5 (0.6 g, 88.2%) as an off-white solid. TLC: 30% EtOAc/heptane (R_{f}: 0.5) which was used in the next step without further purification. MS (ESI): calcd. for C₁₅H₁₆BrN₃OS: 365.02; Found: 368 [M+2]⁺. ¹H NMR (400 MHz, DMSO-d₆): δ 7.55 (d, J=8.8 Hz, 2H), 7.31 (d, J=8.8 Hz, 2H), 6.66 (s, 1H), 4.24-4.16 (m, 2H), 3.73-3.64 (m, 2H), 2.24 (s, 3H), 1.96-1.86 (m, 2H), 1.84-1.74 (m, 2H) ppm.

### Step 5. Synthesis of 1-(2′,5′-difluoro-[1,1′-biphenyl]-4-yl)-3-(4-methylthiazol-2-yl)-1,3-diazepan-2-one (10-6)

To a stirred solution of 10-5 (0.6 g, 1.639 mmol) in 1,4 dioxane:H2O (4:1, 20 mL) were added (2,5-difluorophenyl) boronic acid (0.38 g, 2.46 mmol) and Cs₂CO₃ (1 g, 3.278 mmol) and the reaction mixture was purged under nitrogen for 10 min. Pd (PPh₃)₂Cl₂ (0.12 g, 3.278 mmol) was added under a nitrogen atmosphere. The reaction mixture was heated at 100° C. for 12 h. After completion of the reaction (monitored by TLC), the reaction mixture was cooled to room temperature, filtered through a pad of Celite and washed with ethyl acetate. The filtrate was diluted with water and extracted with EtOAc. The combined organic layers were dried over anhydrous Na₂SO₄, filtered, and concentrated under reduced pressure. The crude compound was purified by CombiFlash chromatography (eluting with 0-30% EtOAc in heptane) to afford 10-6 (0.6 g, 92.3%) as a yellow solid. TLC: 30% EtOAc/heptane (R_{f}: 0.5). MS (ESI): calcd. for C₂₁H₁₉F₂N₃OS: 399.12; Found: 400.05 [M+1]⁺. ¹H NMR (400 MHz, DMSO-d₆): δ 7.60 (d, J=7.8 Hz, 2H), 7.47 (d, J=7.8 Hz, 3H), 7.44-7.34 (m, 1H), 7.34-7.19 (m, 1H), 6.69 (s, 1H), 4.28-4.20 (m, 2H), 3.82-3.75 (m, 2H), 2.26 (s, 3H), 2.00-1.90 (m, 2H), 1.87-1.77 (m, 2H) ppm.

### Step 6. Synthesis of 2-(3-(2′,5′-difluoro-[1,1′-biphenyl]-4-yl)-2-oxo-1,3-diazepan-1-yl)-4-methylthiazole-5-sulfonic acid (10-7)

To a stirred solution of 10-6 (0.3 g, 0.751 mmol) in dry DCM (10 mL) at −5° C. in an inert atmosphere, chlorosulfuric acid (0.15 mL, 2.255 mmol) was added and the resulting reaction mixture was slowly warmed to room temperature and stirred for 12 h. After completion of the reaction (monitored by TLC), the reaction mixture was quenched with ice cold water and stirred for 5-10 min. The precipitated solid obtained out was collected by filtration and dried in vacuo to afford 10-7 (0.6 g, 88.2%) as an off-white solid, which was used in the next step without further purification. TLC: 30% EtOAc/heptane (R_{f}: 0.5). MS (ESI): calcd. for C₂₁H₁₉F₂N₃O₄S₂: 479.08; Found: 480.21 [M+1]⁺. ¹H NMR (400 MHz, DMSO-d₆): δ 7.60 (d, J=7.8 Hz, 2H), 7.48 (d, J=8.3 Hz, 3H), 7.44-7.33 (m, 1H), 7.33-7.19 (m, 1H), 6.10 (br s, 1H), 4.23-4.13 (m, 2H), 3.82-3.73 (m, 2H), 2.34 (s, 3H), 2.00-1.89 (m, 2H), 1.88-1.75 (m, 2H) ppm.

### Step 7. Synthesis of 2-(3-(2′,5′-difluoro-[1,1′-biphenyl]-4-yl)-2-oxo-1,3-diazepan-1-yl)-4-methylthiazole-5-sulfonamide (101)

To a stirred solution of 2-7 (0.2 g, 0.417 mmol) in DCM (10 mL) at −10° C. in an inert atmosphere, oxalyl chloride (0.1 mL, 1.252 mmol) followed by DMF (cat.) were added. The resulting reaction mixture was slowly warmed to room temperature and allowed to stir at 60° C. for 2 h. The reaction mixture was concentrated under reduced pressure to dryness. The resulting residue was dissolved in THF (5 mL) and aqueous ammonia (5 mL) was added at 0° C. while stirring was continued at room temperature for another 1 h. The reaction mixture was diluted with water and extracted with EtOAc. The combined organic layers were dried over anhydrous Na₂SO₄, filtered, and concentrated under reduced pressure. The crude compound was purified by CombiFlash chromatography (eluting with 30-40% EtOAc in heptane) followed by preparative HPLC to afford 101 (40 mg, 20%) as an off-white solid. TLC: 50% EtOAc/heptane (R_{f}: 0.5).

### Example 11. 2-(3-(2′,5′-Difluoro-[1,1′-biphenyl]-4-yl)-2-oxoimidazolidin-1-yl)-4-methylthiazole-5-sulfonamide (104)

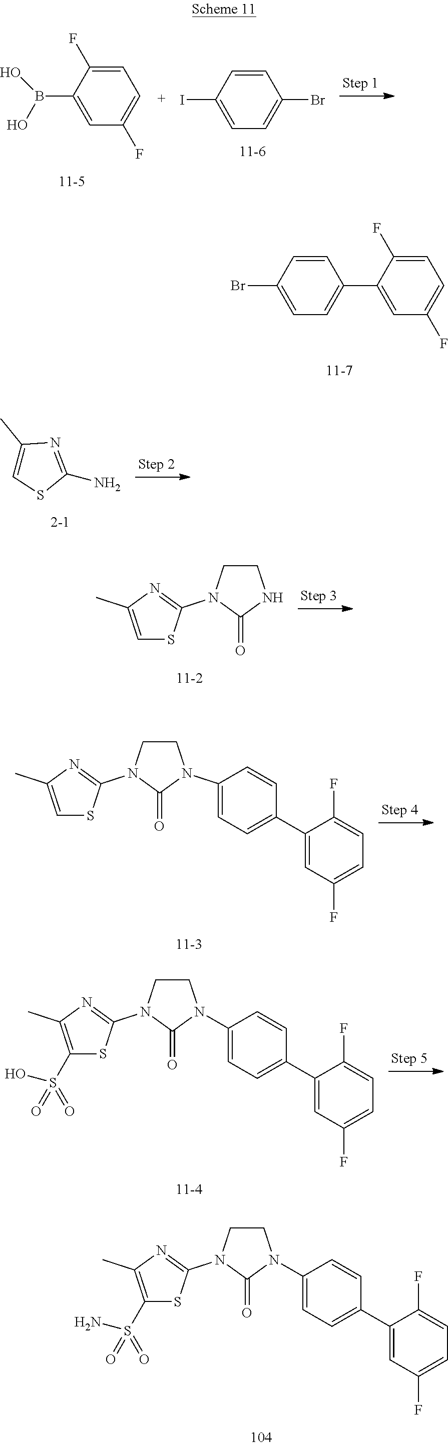

### Step 1. Synthesis of 4′-bromo-2,5-difluoro-1,1′-biphenyl (11-7)

To a stirred solution of 11-6 (5 g, 17.674 mmol) in 1,4 dioxane/H₂O (50 mL/5 mL) were added (2,5-difluorophenyl) boronic acid (11-5) (3.07 g, 19.441 mmol) and K₃PO₄ (7.5 g, 35.348 mmol) and the reaction mixture was purged under nitrogen for 10 min. PdCl₂(dppf) (1.29 g, 1.767 mmol) was added under a nitrogen atmosphere. The reaction mixture was heated at 80° C. for 1 h. After completion of the reaction (monitored by TLC), the reaction mixture was cooled to room temperature, filtered through a pad of Celite®545 and washed with ethyl acetate. The filtrate was diluted with water and extracted with EtOAc. The combined organic layers were dried over anhydrous Na₂SO₄, filtered, and concentrated under reduced pressure. The crude compound was purified by CombiFlash chromatography (eluting with 100% heptane) to afford 11-7 (2.3 g, 48.6%) as an off-white solid. TLC: 100% heptane (R_{f}: 0.5). ¹H NMR (400 MHz, CDCl₃) δ 7.58 (d, J=8.3 Hz, 2H), 7.40 (d, J=7.3 Hz, 2H), 7.15-7.06 (m, 2H), 7.05-6.97 (m, 1H) ppm.

### Step 2. Synthesis of 1-(4-methylthiazol-2-yl) imidazolidin-2-one (11-2)

A mixture of 2-1 (5 g, 43.859 mmol) and 1-chloro-2-isocyanatoethane (6.9 g, 65.79 mmol) in THF (100 mL) was heated at 65° C. for 5 h. To this resulting solution, TBAB (0.7 g, 2.192 mmol) and K₂CO₃ (12.10 g, 87.718 mmol) were added portion wise maintaining the same temperature and stirring continued at 65° C. for 16 h. The reaction mixture was diluted with water and extracted with EtOAc. The combined organic layers were dried over anhydrous Na₂SO₄, filtered, and concentrated under reduced pressure. The crude compound was purified by CombiFlash chromatography (eluting with 60-70% EtOAc in heptane) to afford 11-2 (6 g, 75%) as an off-white solid. TLC: 70% EtOAc/heptane (R_{f}: 0.5). MS (ESI): calcd. for C₇H₉N₃OS: 183.05; Found: 184.00 [M+1]⁺. ¹H NMR (400 MHz, DMSO-d₆): δ 7.52 (br s, 1H), 6.65 (s, 1H), 4.00 (t, J=7.8 Hz, 2H), 3.48 (t, J=8.1 Hz, 2H), 2.21 (s, 3H) ppm.

### Step 3. Synthesis of 1-(2′,5′-difluoro-[1,1′-biphenyl]-4-yl)-3-(4-methylthiazol-2-yl) imidazolidin-2-one (11-3)

To a stirred solution of 11-2 (2 g, 10.752 mmol) in 1, 4-dioxane (50 mL) were added 11-7 (5.7 g, 21.505 mmol), K₂CO₃ (2.96 g, 21.505 mmol) and 1,2-Dimethylethylenediamine (0.47 g, 5.376 mmol) the reaction mixture was purged under nitrogen for 10 min. To this resulting reaction mixture, CuI (0.4 g, 2.150 mmol) was added under a nitrogen atmosphere. The reaction mixture was heated at 100° C. for 16 h. The reaction mixture was filtered through Celite bed and washed with ethyl acetate. The filtrate was diluted with water and extracted with EtOAc then washed with brine. The combined organic layers were dried over anhydrous Na₂SO₄, filtered, and concentrated under reduced pressure. The crude compound obtained was purified by CombiFlash chromatography (eluting with 50-70% EtOAc in heptane) to afford 11-3 (0.35 g, 17.5%) as an off-white solid. TLC: 60% EtOAc/Heptane (R_{f}: 0.5). MS (ESI): calcd. for C₁₉H₁₅F₂N₃OS: 371.09; Found: 372.35 [M+1]⁺. ¹H NMR (400 MHz, DMSO-d₆): δ 7.77 (d, J=8.3 Hz, 2H), 7.69-7.57 (m, 2H), 7.49-7.30 (m, 2H), 7.30-7.19 (m, 1H), 6.79 (s, 1H), 4.21-4.05 (m, 4H), 2.28 (s, 3H) ppm.

### Step 4. Synthesis of 2-(3-(2′,5′-difluoro-[1,1′-biphenyl]-4-yl)-2-oxoimidazolidin-1-yl)-4-methylthiazole-5-sulfonic acid (11-4)

To a stirred solution of 11-3 (0.3 g, 0.808 mmol) in dry DCM (5 mL) at 0° C. in an inert atmosphere, chlorosulfuric acid (0.16 mL, 2.425 mmol) was added and the resulting reaction mixture was slowly warmed to room temperature and stirred for 16 h. After completion of the reaction (monitored by TLC), the reaction mixture was quenched with ice cold water and stirred for 5-10 min. The precipitated solid obtained out was collected by filtration and dried in vacuo to afford 11-4 (0.2 g, 66.6%) as an off-white solid. TLC: 5% MeOH/DCM (R_{f}: 0.5) which was used in the next step without further purification.

### Step 5. Synthesis of 2-(3-(2′,5′-difluoro-[1,1′-biphenyl]-4-yl)-2-oxoimidazolidin-1-yl)-4-methylthiazole-5-sulfonamide (104)

To a stirred solution of 11-4 (0.2 g, 0.442 mmol) in DCM (5 mL) at 0° C. in an inert atmosphere, oxalyl chloride (0.1 mL, 1.327 mmol) followed by DMF (cat.) were added. The resulting reaction mixture was slowly warmed to room temperature and allowed to stir at 50° C. for 2 h. The reaction mixture was concentrated under reduced pressure to dryness. The resulting residue was dissolved in THE (2 mL) and aqueous ammonia (5 mL) was added at 0° C. while stirring was continued at room temperature for another 1 h. The reaction mixture was diluted with water and extracted with EtOAc. The combined organic layers were dried over anhydrous Na₂SO₄, filtered, and concentrated under reduced pressure. The crude compound was purified by CombiFlash chromatography (eluting with 0-1% MeOH in DCM) followed by preparative HPLC to afford 104 (8 mg, 4.2%) as an off-white solid.

### Example 12. 2-(3-(2′,5′-Difluoro-[1,1′-biphenyl]-4-yl)-6-methyl-2-oxotetrahydropyrimidin-1(2H)-yl)-4-methylthiazole-5-sulfonamide (107)

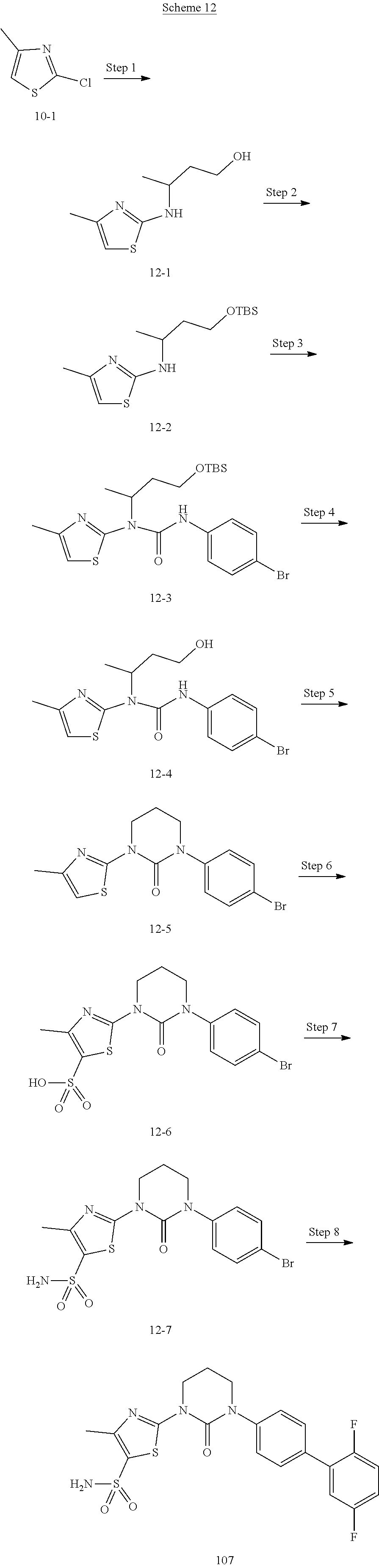

### Step 1. Synthesis of 3-((4-methylthiazol-2-yl)amino)butan-1-ol (12-1)

To a stirred solution of 10-1 (1 g, 7.518 mmol) in DMSO (10 mL) was added 3-aminobutan-1-ol (1.0 g, 11.278 mmol mmol) followed by K₂CO₃ (2.5 g, 18.795 mmol) and the reaction mixture was purged under nitrogen for 10 min. To this resulting reaction mixture, CuI (0.142 g, 0.751 mmol) was added under a nitrogen atmosphere and the reaction mixture was heated at 120° C. for 16 h. The reaction mixture was filtered through Celite bed and washed with ethyl acetate. The filtrate was concentrated under reduced pressure. The crude compound obtained was purified by CombiFlash chromatography (eluting with 30-70% EtOAc in heptane) to afford 12-1 (0.35 g, 25%) as an off-white solid. TLC: 50% EtOAc/Heptane (R_{f}: 0.5).

### Step 2. Synthesis of N-(4-((tert-butyldimethylsilyl)oxy)butan-2-yl)-4-methylthiazol-2-amine (12-2)

To a stirred solution of 12-1 (1.5 g, 8.064 mmol) in dry DCM (20 mL) at 0° C. under a nitrogen atmosphere, Imidazole (1.09 g, 16.128 mmol) was added in small portions and the resulting reaction mixture was stirred at the same temperature for 10-15 min. To this reaction mixture, tert-butyldimethylsilyl chloride (1.38 g, 8.870 mmol) was added at 0° C. and then the reaction mixture was slowly warmed to room temperature and stirred for 16 h. After completion of the reaction (monitored by TLC), the reaction mixture was concentrated under reduced pressure. The crude compound was purified by CombiFlash chromatography (eluting with 30-40% EtOAc in heptane) to afford 12-2 (1 g, 41.6%) as a yellow-colored oil. TLC: 30% EtOAc/heptane (R_{f}: 0.5).

### Step 3. Synthesis of 3-(4-bromophenyl)-1-(4-((tert-butyldimethylsilyl)oxy)butan-2-yl)-1-(4-methylthiazol-2-yl)urea (12-3)

To a stirred solution of 12-2 (1 g, 3.333 mmol) in 1,4 dioxane (10 mL) at 0° C. in an inert atmosphere, 1-bromo-4-isocyanatobenzene (0.78 g, 4.00 mmol) was added and the resulting reaction mixture was slowly warmed to room temperature and stirred for 4 h. After completion of the reaction (monitored by TLC), the reaction mixture was concentrated under reduced pressure. The crude product was purified by CombiFlash column chromatography [eluting with 30-40% EtOAc in heptane] to afford 12-3 (1 g, 62.5%) as an off-white solid. TLC: 30% EtOAc/heptane (R_{f}: 0.5).

### Step 4. Synthesis of 3-(4-bromophenyl)-1-(4-hydroxybutan-2-yl)-1-(4-methylthiazol-2-yl)urea (12-4)

To a stirred solution of 12-3 (1 g, 2.012 mmol) in dry THF (5 mL) at 0° C. under a nitrogen atmosphere, Tetrabutylammonium fluoride (1M solution in THF, 4.02 mL, 4.024 mmol) was added dropwise. The resulting reaction mixture was slowly warmed to room temperature and stirred for 4 h. After completion of the reaction (monitored by TLC), the reaction mixture was quenched with ice cold water and extracted with EtOAc. The combined organic layers were dried over anhydrous Na₂SO₄, filtered, and concentrated under reduced pressure. The crude compound was purified by CombiFlash chromatography (eluting with 20-30% EtOAc in heptane) to afford 12-4 (0.5 g, crude) as a pale-yellow oil. TLC: 50% EtOAc/heptane (R_{f}: 0.5).

### Step 5. Synthesis of 1-(4-bromophenyl)-4-methyl-3-(4-methylthiazol-2-yl)tetrahydropyrimidin-2(1H)-one (12-5)

To a stirred solution of 12-4 (0.5 g, 1.305 mmol) in Toluene (5 mL) at 0° C., was added triphenylphosphine (0.68 g, 2.610 mmol) followed by DEAD (0.52 g, 2.61 mmol). The resulting reaction mixture was slowly warmed to room temperature and stirred at 100° C. for 16 h. After completion of the reaction (monitored by TLC), the reaction mixture was concentrated under reduced pressure. The crude compound was purified by CombiFlash chromatography (eluting with 20-30% EtOAc in heptane) to afford 12-5 (0.3 g, 62.8) as a pale-yellow oil. TLC: 50% EtOAc/heptane (R_{f}: 0.5).

### Step 6. Synthesis of 2-(3-(4-bromophenyl)-6-methyl-2-oxotetrahydropyrimidin-1(2H)-yl)-4-methylthiazole-5-sulfonic acid (12-6)

To a stirred solution of 12-5 (0.3 g, 0.821 mmol) in dry DCM (5 mL) at 0° C. in an inert atmosphere, chlorosulfonic acid (0.19 g, 1.643 mmol) was added, and the resulting reaction mixture was slowly warmed to room temperature and stirred for 16 h. After completion of the reaction (monitored by TLC), the reaction mixture was concentrated under reduced pressure to dryness. The crude residue obtained was purified by trituration with diethyl ether. The obtained solid was filtered off and dried in vacuo to afford 12-6 (0.25 g, crude) as an off-white solid, TLC: 40% EtOAc/Heptane (R_{f}: 0.3), which was used in the next step without further purification.

### Step 7. Synthesis of 2-(3-(4-bromophenyl)-6-methyl-2-oxotetrahydropyrimidin-1(2H)-yl)-4-methylthiazole-5-sulfonamide (12-7)

A mixture of 12-6 (0.25 g, 0.327 mmol) and POCl₃ (3 mL) was allowed to stir at 80° C. for 12 h. The reaction mixture was concentrated under reduced pressure to dryness. The resulting residue was dissolved in THE (3 mL) and aqueous ammonia (5 mL) was added at 0° C. while stirring was continued at room temperature for another 16 h. After completion of the reaction (monitored by TLC), the reaction mixture was concentrated under reduced. The crude compound was purified by CombiFlash chromatography (eluting with 40-50% EtOAc in heptane) to afford 12-7 (0.15 g, 78%) as an off-white solid. TLC: 50% EtOAc/heptane (R_{f}: 0.5).

### Step 8. Synthesis of 2-(3-(2′,5′-difluoro-[1,1′-biphenyl]-4-yl)-6-methyl-2-oxotetrahydropyrimidin-1(2H)-yl)-4-methylthiazole-5-sulfonamide (107)

To a stirred solution of 12-7 (0.15 g, 0.337 mmol) in 1,4 dioxane:H₂O (4:1, 10 mL) were added (2,5-difluorophenyl) boronic acid (0.8 g, 0.506 mmol) and K₃PO₄ (0.143 g, 0.674 mmol) and the reaction mixture was purged under nitrogen for 10 min. Pd(dppf)Cl₂ (24.7 mg, 0.0337 mmol) was added under a nitrogen atmosphere and the reaction mixture was heated at 100° C. for 4 h. After completion of the reaction (monitored by TLC), the reaction mixture was cooled to room temperature, filtered through a pad of Celite*545 and washed with ethyl acetate. The filtrate was concentrated under reduced pressure. The crude compound was purified by CombiFlash chromatography followed by preparative HPLC to afford 107 (90 mg, 55.9%) as an off-white solid. TLC: 50% EtOAc/heptane (R_{f}: 0.5).

### Example 13. 2-(7-(2′,5′-Difluoro-[1,1′-biphenyl]-4-yl)-6-oxo-5,7-diazaspiro[2.5]octan-5-yl)-4-methylthiazole-5-sulfonamide (110)

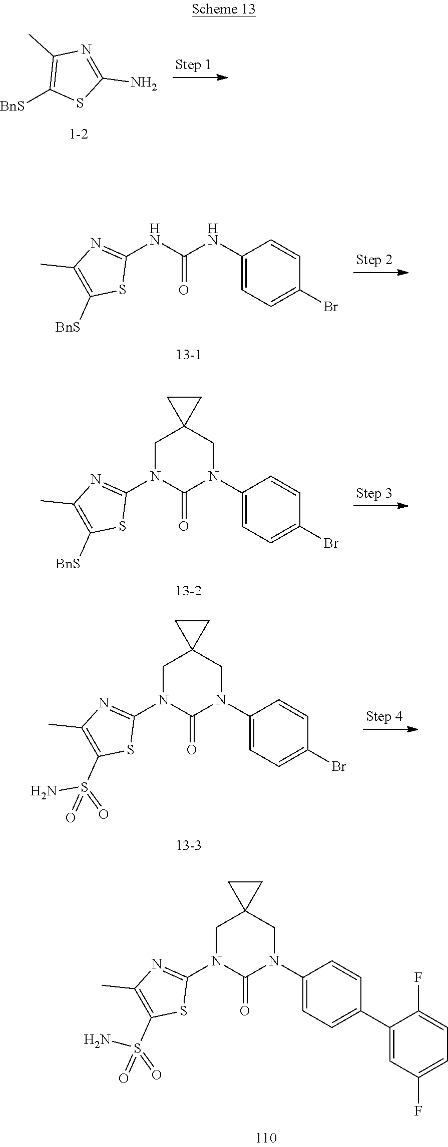

### Step 1. Synthesis of 1-(5-(benzylthio)-4-methylthiazol-2-yl)-3-(4-bromophenyl) urea (13-1)

To a stirred solution of 1-2 (2.5 g, 10.593 mmol) in 1,4 dioxane (25 mL) at 0° C. in an inert atmosphere, 1-bromo-4-isocyanatobenzene (2.08 g, 10.593 mmol) was added and the resulting reaction mixture was slowly warmed to room temperature and stirred for 4 h. After completion of the reaction (monitored by TLC), the reaction mixture was quenched with ice cold water and stirred for 5-10 min. The precipitated solid obtained out was collected by filtration and dried in vacuo to afford 13-1 (2 g, 44.7%) as an off-white solid. TLC: 50% EtOAc/heptane (R_{f}: 0.5), which was used in the next step without further purification. ¹H NMR (400 MHz, DMSO-d₆): δ 10.56 (br s, 1H), 9.11 (s, 1H), 7.47 (s, 4H), 7.31-7.20 (m, 3H), 7.11 (d, J=6.8 Hz, 2H), 3.87 (s, 2H), 1.86 (s, 3H) ppm.

### Step 2. Synthesis of 5-(5-(benzylthio)-4-methylthiazol-2-yl)-7-(4-bromophenyl)-5,7-diazaspiro[2.5]octan-6-one (13-2)

To a stirred solution of 13-1 (1.5 g, 3.464 mmol) in dry DMF (30 mL) at 0° C. under a nitrogen atmosphere, Cs₂CO₃ (3.37 g, 10.392 mmol) was added in small portions and the resulting reaction mixture was stirred at room temperature for 30 min. To this reaction mixture, 1,1-bis(bromomethyl)cyclopropane (0.79 g, 3.464 mmol) was added at 0° C. and then the reaction mixture was slowly warmed to room temperature and stirred at 100° C. for 12 h. After completion of the reaction (monitored by TLC), the reaction mixture was quenched with ice cold water and extracted with EtOAc. The combined organic layers were dried over anhydrous Na₂SO₄, filtered, and concentrated under reduced pressure. The crude compound was purified by CombiFlash chromatography (eluting with 30-40% EtOAc in heptane) to afford 13-2 (0.4 g, 23.5%) as an off-white solid. TLC: 30% EtOAc/heptane (R_{f}: 0.5). MS (ESI): calcd. for C₂₃H₂₂BrN₃OS₂: 499.04; Found: 500.1 [M+1]. ¹H NMR (400 MHz, DMSO-d₆): δ 7.58 (d, J=7.8 Hz, 2H), 7.32 (d, J=8.3 Hz, 2H), 7.28-7.21 (m, 3H), 7.16-7.07 (m, 2H), 3.93 (s, 2H), 3.85 (s, 2H), 3.59 (s, 2H), 1.91 (s, 3H), 0.80-0.71 (m, 4H) ppm.

### Step 3. Synthesis of 2-(7-(4-bromophenyl)-6-oxo-5,7-diazaspiro [2.5]octan-5-yl)-4-methylthiazole-5-sulfonamide (13-3)

To a stirred solution of 13-2 (0.1 g, 0.200 mmol) in AcOH/H₂O (2 mL/0.2 mL), NCS (0.1 g, 0.751 mmol) was added, and the reaction mixture was stirred at room temperature for 30 min. After completion of the reaction (monitored by TLC), the reaction mixture was concentrated under reduced pressure to dryness. The resulting residue was dissolved in THF (1 mL) and aqueous ammonia (2 mL) was added at 0° C. while stirring was continued at the same temperature for another 16 h. The reaction mixture was concentrated under reduced pressure to dryness. The resulting reaction mixture was diluted with water and extracted with ethyl acetate. The combined organic layers were dried over anhydrous Na₂SO₄, filtered, and concentrated under reduced pressure. The crude compound obtained was purified by CombiFlash chromatography (eluting with 1-2% MeOH in DCM) to afford 13-3 (0.1 g, 55%) as an off-white solid. TLC: 5% MeOH/DCM (R_{f}: 0.3). ¹H NMR (400 MHz, DMSO-d₆): δ 7.62-7.54 (m, 4H), 7.34 (d, J=7.8 Hz, 2H), 3.98 (s, 2H), 3.62 (s, 2H), 2.41 (s, 3H), 0.78 (d, J=6.4 Hz, 4H) ppm.

### Step 4. Synthesis of 2-(7-(2′,5′-difluoro-[1,1′-biphenyl]-4-yl)-6-oxo-5,7-diazaspiro [2.5]octan-5-yl)-4-methylthiazole-5-sulfonamide (110)

To a stirred solution of 13-3 (90 mg, 0.197 mmol) in 1, 4-dioxane and water (2:0.4 mL) were added (2,5-difluorophenyl) boronic acid (46.74 mg, 0.296 mmol) and K₃PO₄ (0.10 g, 0.492 mmol) and the reaction mixture was purged under nitrogen for 10 min. Pd(dppf)Cl₂ (14 mg, 0.0197 mmol) was added under a nitrogen atmosphere. The reaction mixture was heated at 80° C. for 12 h. After completion of the reaction (monitored by TLC), the reaction mixture was cooled to room temperature, diluted with water and extracted with EtOAc then washed with brine. The combined organic layers were dried over anhydrous Na₂SO₄, filtered, and concentrated under reduced pressure. The crude compound obtained was purified by preparative HPLC to afford 110 (10 mg, 10.3%) as an off-white solid. TLC: 5% MeOH/DCM (R_{f}: 0.3).

### Example 14. 4-Methyl-2-(7-(4-(1-methyl-1H-pyrazol-5-yl)phenyl)-6-oxo-5,7-diazaspiro[2.5]octan-5-yl)thiazole-5-sulfonamide (113)

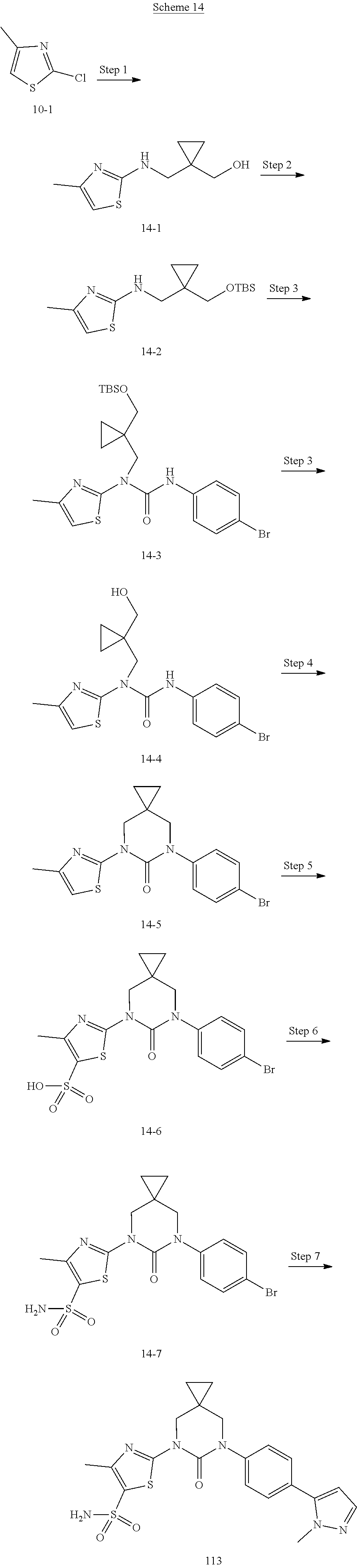

### Step 1. Synthesis of (1-(((4-methylthiazol-2-yl)amino)methyl)cyclopropyl)methanol (14-1)

To a stirred solution of 10-1 (3 g, 22.455 mmol) in DMSO (50 mL) was added compound 2 (3.4 g, 33.682 mmol mmol) followed by K₂CO₃ (4.65 g, 33.682 mmol) and the reaction mixture was purged under nitrogen for 10 min. To this resulting reaction mixture, CuI (0.43 g, 2.245 mmol) was added under a nitrogen atmosphere and the reaction mixture was heated at 120° C. for 16 h. The reaction mixture was filtered through Celite bed and washed with ethyl acetate. The filtrate was concentrated under reduced pressure. The crude compound obtained was purified by CombiFlash chromatography (eluting with 10-20-70% EtOAc in heptane) to afford 14-1 (0.8 g, 18%) as an off-white solid. TLC: 20% EtOAc/Heptane (R_{f}: 0.5). MS (ESI): calcd. for C₉H₁₄N₂OS: 198.08; Found: 199.1 [M+1]⁺.

### Step 2. Synthesis of N-((1-(((tert-butyldimethylsilyl)oxy)methyl)cyclopropyl)methyl)-4-methylthiazol-2-amine (14-2)

To a stirred solution of 14-1 (0.4 g, 2.020 mmol) in dry DCM (20 mL) at 0° C. under a nitrogen atmosphere, Imidazole (0.20 g, 3.030 mmol) was added in small portions and the resulting reaction mixture was stirred at the same temperature for 10-15 min. To this reaction mixture, tert-Butyldimethylsilyl chloride (0.36 g, 2.424 mmol) was added at 0° C. and then the reaction mixture was slowly warmed to room temperature and stirred for 16 h. After completion of the reaction (monitored by TLC), the reaction mixture was concentrated under reduced pressure. The crude compound was purified by CombiFlash chromatography (eluting with 30-40% EtOAc in heptane) to afford 14-2 (0.7 g, 55.5%) as a yellow-colored oil. TLC: 20% EtOAc/heptane (R_{f}: 0.5).

### Step 3. Synthesis of 3-(4-bromophenyl)-1-((1-(((tert-butyldimethylsilyl)oxy)methyl)cyclopropyl)methyl)-1-(4-methylthiazol-2-yl)urea (14-3)

To a stirred solution of 14-2 (0.35 g, 1.121 mmol) in 1,4 dioxane (10 mL) at 0° C. in an inert atmosphere, 1-bromo-4-isocyanatobenzene (0.22 g, 1.121 mmol) was added and the resulting reaction mixture was slowly warmed to room temperature and stirred for 12 h. After completion of the reaction (monitored by TLC), the reaction mixture was concentrated under reduced pressure. The crude product was purified by CombiFlash column chromatography [eluting with 30-40% EtOAc in heptane] to afford 14-3 (0.5 g, 43.8%) as an off-white solid. TLC: 20-30% EtOAc/heptane (R_{f}: 0.5). MS (ESI): calcd. for C₂₂H₃₂BrN₃O₂SSi: 509.12; Found: 512.13 [M+2]⁺.

### Step 4. Synthesis of 3-(4-bromophenyl)-1-((1-(hydroxymethyl)cyclopropyl)methyl)-1-(4-methylthiazol-2-yl)urea (14-4)

To a stirred solution of 14-3 (0.25 g, 0.490 mmol) in dry THF (5 mL) at 0° C. under a nitrogen atmosphere, Tetrabutylammonium fluoride (1M solution in THF, 1 mL, 0.980 mmol) was added dropwise. The resulting reaction mixture was slowly warmed to room temperature and stirred for 4 h. After completion of the reaction (monitored by TLC), the reaction mixture was quenched with ice cold water and extracted with EtOAc. The combined organic layers were dried over anhydrous Na₂SO₄, filtered, and concentrated under reduced pressure. The crude compound was purified by CombiFlash chromatography (eluting with 20-30% EtOAc in heptane) to afford 14-4 (0.4 g, crude) as an off-white solid. TLC: 30% EtOAc/heptane (R_{f}: 0.5). MS (ESI): calcd. for C₂₂H₃₂BrN₃O₂SSi: 395.03; Found: 396.1 [M+1]⁺.

### Step 5. Synthesis of 5-(4-bromophenyl)-7-(4-methylthiazol-2-yl)-5,7-diazaspiro[2.5]octan-6-one (14-5)

To a stirred solution of 14-4 (0.2 g, 0.506 mmol) in Toluene (5 mL) at 0° C., was added triphenylphosphine (0.2 g, 0.759 mmol) followed by DEAD (0.13 g, 0.759 mmol). The resulting reaction mixture was slowly warmed to room temperature and stirred at 100° C. for 16 h. After completion of the reaction (monitored by TLC), the reaction mixture was concentrated under reduced pressure. The crude compound was purified by CombiFlash chromatography (eluting with 20-30% EtOAc in heptane) to afford 14-5 (0.24 g, 63.1%) as an off-white solid. TLC: 30% EtOAc/heptane (R_{f}: 0.5). MS (ESI): calcd. for C₁₆H₁₆BrN₃OS: 377.02; Found: 377.90 [M+1]⁺.

### Step 6. Synthesis of 2-(7-(4-bromophenyl)-6-oxo-5,7-diazaspiro[2.5]octan-5-yl)-4-methylthiazole-5-sulfonic acid (14-6)

To a stirred solution of 14-5 (0.12 g, 0.317 mmol) in dry DCM (10 mL) at 0° C. in an inert atmosphere, Chlorosulfonic acid (0.11 g, 0.952 mmol) was added, and the resulting reaction mixture was slowly warmed to room temperature and stirred for 16 h. The reaction mixture was concentrated under reduced pressure to dryness. The crude residue obtained was purified by trituration with diethyl ether. The obtained solid was filtered off and dried in vacuo to afford 14-6 (0.15 g, crude) as an off-white solid. TLC: 30% EtOAc/Heptane (R_{f}: 0.3) which was used in the next step without further purification. MS (ESI): calcd. for C₁₆H₁₆BrN₃O₄S₂: 456.98; Found: 459.7 [M+2]⁺.

### Step 7. Synthesis of 2-(7-(4-bromophenyl)-6-oxo-5,7-diazaspiro[2.5]octan-5-yl)-4-methylthiazole-5-sulfonamide (14-7)

A mixture of 14-6 (0.15 g, 0.327 mmol) and POCl₃ (1.5 mL) was allowed to stir at 100° C. for 12 h. The reaction mixture was concentrated under reduced pressure to dryness. The resulting residue was dissolved in THF (3 mL) and aqueous ammonia (5 mL) was added at 0° C. while stirring was continued at room temperature for another 1 h. The reaction mixture was concentrated under reduced pressure. The crude residue obtained was purified by trituration with diethyl ether. The obtained solid was filtered off and dried in vacuo to afford 14-7 (0.1 g, crude) as a pale brown colored solid. TLC: 30% EtOAc/Heptane (R_{f}: 0.3) which was used in the next step without further purification. MS (ESI): calcd. for C₁₆H₁₇BrN₄O₃S₂: 455.99; Found: 458.7 [M+2]⁺.

### Step 8. Synthesis of 4-methyl-2-(7-(4-(1-methyl-1H-pyrazol-5-yl)phenyl)-6-oxo-5,7-diazaspiro[2.5]octan-5-yl)thiazole-5-sulfonamide (113)

To a stirred solution of 14-7 (0.1 g, 0.218 mmol) in 1,4-dioxane:H₂O (4:1, 10 mL) were added 1-methyl-5-(4,4,5,5-tetramethyl-1,3,2-dioxaborolan-2-yl)-1H-pyrazole (75 mg, 0.327 mmol) and Na₂CO₃ (47 mg, 0.436 mmol) and the reaction mixture was purged under nitrogen for 10 min. Pd (dppf)Cl₂ (16 mg, 0.0218 mmol) was added under a nitrogen atmosphere and the reaction 15 mixture was heated at 100° C. for 12 h. After completion of the reaction (monitored by TLC), the reaction mixture was cooled to room temperature, filtered through a pad of Celite and washed with ethyl acetate. The filtrate was concentrated under reduced pressure. The crude compound was purified by CombiFlash chromatography followed by preparative HPLC to afford 113 (20 mg, 20%) as an off-white solid. TLC: 30% EtOAc/heptane (R_{f}: 0.3).

### Example 15. 2-(3-(2′,5′-Difluoro-[1,1′-biphenyl]-4-yl)-2-oxotetrahydropyrimidin-1(2H)-yl)-N,4-dimethylthiazole-5-sulfonamide (116)

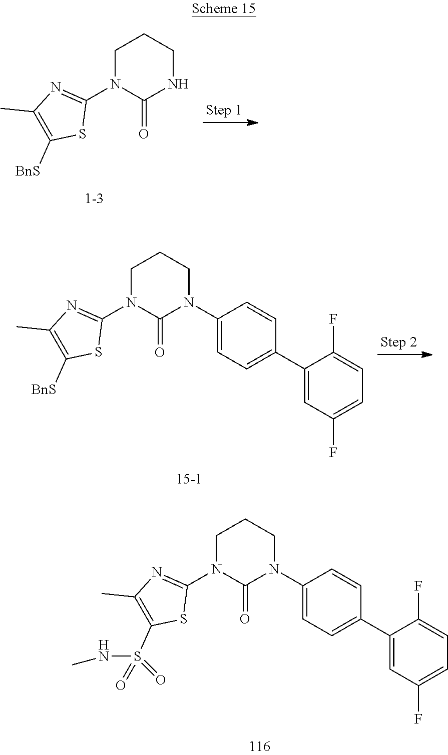

### Step 1. Synthesis of 1-(5-(benzylthio)-4-methylthiazol-2-yl)-3-(2′,5′-difluoro-[1,1′-biphenyl]-4-yl) tetrahydropyrimidin-2(1H)-one (15-1)

To a stirred solution of 1-3 (0.5 g, 1.567 mmol) in 1, 4-dioxane (5 mL) were added compound 11-7 (0.46 g, 1.724 mmol), K₂CO₃ (0.43 g, 3.134 mmol) and 1,2-dimethylethylenediamine (69 mg, 0.783 mmol) the reaction mixture was purged under nitrogen for 10 min. To this resulting reaction mixture, CuI (60 mg, 0.313 mmol) was added under a nitrogen atmosphere. The reaction mixture was heated at 100° C. for 16 h. The reaction mixture was filtered through Celite bed and washed with ethyl acetate. The filtrate was diluted with water and extracted with EtOAc then washed with brine. The combined organic layers were dried over anhydrous Na₂SO₄, filtered, and concentrated under reduced pressure. The crude compound obtained was purified by CombiFlash chromatography (eluting with 50-70% EtOAc in heptane) to afford 15-1 (0.25 g, 31.6%) as an off-white solid. TLC: 60% EtOAc/Heptane (R_{f}: 0.5). MS (ESI): calcd. for Chemical Formula C₂₇H₂₃F₂N₃OS₂: 507.13; Found: 508.44 [M+1]⁺.

### Step 2. Synthesis of 2-(3-(2′,5′-difluoro-[1,1′-biphenyl]-4-yl)-2-oxotetrahydropyrimidin-1(2H)-yl)-N,4-dimethylthiazole-5-sulfonamide (116)

To a stirred solution of 15-1 (0.15 g, 0.295 mmol) in AcOH:H₂O (1.5:0.1 mL), NCS (0.15 g, 1.10 mmol) was added, and the reaction mixture was stirred at room temperature for 30 min. After completion of the reaction (monitored by TLC), the reaction mixture was concentrated under reduced pressure to dryness. The resulting residue was dissolved in THF (2 mL), to this resulting solution, Methyl amine (33 wt. % solution in methanol, 3 mL) was added at 0° C. and stirring continued at room temperature for another 1 h. The reaction mixture was concentrated under reduced pressure to dryness. The resulting residue was dissolved in ethyl acetate and the organic layer was washed with water. The combined organic layers were dried over anhydrous Na₂SO₄, filtered, and concentrated under reduced pressure. The crude compound was purified by preparative HPLC to afford 116 (15 mg, 10.6%) as an off-white solid. TLC: 5% MeOH/DCM (R_{f}: 0.5)

### Example 16. 1-(2′,5′-Difluoro-[1,1′-biphenyl]-4-yl)-3-(4-methyl-5-(S-methylsulfonimidoyl) thiazol-2-yl) tetrahydropyrimidin-2(1H)-one (139)

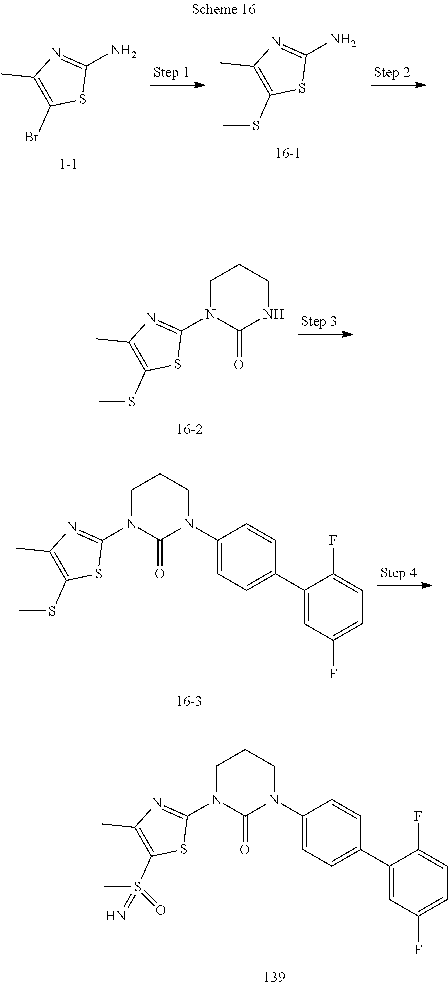

### Step 1. Synthesis of 4-methyl-5-(methylthio) thiazol-2-amine (16-1)

To a stirred solution of 1-1 (1 g, 5.181 mmol) in methanol (10 mL) at 0° C., Sodium thiomethoxide (0.544 g, 7.77 mmol) was added drop wise. The resulting reaction mixture was slowly warmed to room temperature and stirred for 16 h. The reaction mixture was concentrated under reduced pressure to dryness. The resulting residue was dissolved in EtOAc and the organic layer was washed with water. The combined organic layers were dried over anhydrous Na₂SO₄, filtered, and concentrated under reduced pressure. The crude product was purified by CombiFlash chromatography (eluting with 10-20% EtOAc in heptane) to afford 16-1 (0.6 g, 72.3%) as an off-white solid. TLC: 50% EtOAc/Heptane (R_{f}: 0.5). MS (ESI): calcd. for C₅H₈N₂S₂: 160.01; Found: 160.75 [M+1]⁺. ¹H NMR (400 MHz, DMSO-d₆) b 7.08 (br s, 2H), 2.21 (s, 3H), 2.12 (s, 3H).

### Step 2. Synthesis of 1-(4-methyl-5-(methylthio) thiazol-2-yl) tetrahydropyrimidin-2(1H)-one (16-2)

A mixture of 16-1 (0.6 g, 3.75 mmol) and 1-chloro-3-isocyanatopropane (0.58 g, 4.87 mmol) in THF (12 mL) was heated at 65° C. for 5 h. To this resulting solution, TBAI (69 mg, 0.187 mmol) and K₂CO₃ (0.77 g, 5.62 mmol) were added portion wise maintaining the same temperature and stirring continued at 65° C. for 16 h. The reaction mixture was diluted with water and extracted with EtOAc. The combined organic layers were dried over anhydrous Na₂SO₄, filtered, and concentrated under reduced pressure. The crude compound was purified by CombiFlash chromatography (eluting with 60-70% EtOAc in heptane) to afford 16-2 (0.5 g, 54.8%) as an off-white solid. TLC: 50% EtOAc/heptane (R_{f}: 0.5). MS (ESI): calcd. for C₉H₁₃N₃OS₂: 174.03; Found: 174.77 [M+1]⁺. ¹H NMR (400 MHz, DMSO-d₆): δ 7.44 (s, 1H), 4.00-3.92 (m, 2H), 3.25-3.17 (m, 2H), 2.29 (s, 3H), 2.28 (s, 3H), 2.00-1.89 (m, 2H) ppm.

### Step 3. Synthesis of 1-(2′,5′-difluoro-[1,1′-biphenyl]-4-yl)-3-(4-methyl-5-(methylthio) thiazol-2-yl) tetrahydropyrimidin-2(1H)-one (16-3)

To a stirred solution of 16-2 (0.5 g, 2.057 mmol) in 1, 4-dioxane (6 mL) were added 11-7 (0.66 g, 2.47 mmol), K₂CO₃ (0.57 g, 4.11 mmol) and 1,2-Dimethylethylenediamine (90 mg, 1.028 mmol) the reaction mixture was purged under nitrogen for 10 min. To this resulting reaction mixture, CuI (78 mg, 0.41 mmol) was added under a nitrogen atmosphere. The reaction mixture was heated at 110° C. for 16 h. The reaction mixture was filtered through Celite bed and washed with ethyl acetate. The filtrate was diluted with water and extracted with EtOAc then washed with brine. The combined organic layers were dried over anhydrous Na₂SO₄, filtered, and concentrated under reduced pressure. The crude compound obtained was purified by CombiFlash chromatography (eluting with 50-70% EtOAc in heptane) to afford 16-3 (0.4 g, 45%) as an off-white solid. TLC: 50% EtOAc/Heptane (R_{f}: 0.5). MS (ESI): calcd. for C₂₁H₁₉F₂N₃OS₂: 431.09; Found: 432.1 [M+1]⁺. ¹H NMR (400 MHz, DMSO-d₆): δ 7.63-7.59 (m, 2H), 7.51-7.34 (m, 4H), 7.30-7.24 (m, 1H), 4.17-4.08 (m, 2H), 3.86-3.78 (m, 2H), 2.34-2.26 (m, 6H), 2.26-2.17 (m, 2H) ppm.

### Step 4. Synthesis of 1-(2′,5′-difluoro-[1,1′-biphenyl]-4-yl)-3-(4-methyl-5-(S-methylsulfonimidoyl) thiazol-2-yl) tetrahydropyrimidin-2(1H)-one (139)

To a stirred solution of 16-3 (0.1 g, 0.23 mmol) in ACN:MeOH (2:2 mL) were added Ammonium carbamate (0.054 g, 0.69 mmol) and PhI(OAc)₂ (0.34 g, 0.92 mmol). The resulting reaction mixture was allowed to stir at room temperature for 2 h. After completion of the reaction (monitored by TLC), the reaction mixture was concentrated under reduced pressure. The crude residue obtained was dissolved in ethyl acetate and washed with water. The combined organic layers were dried over anhydrous Na₂SO₄, filtered, and concentrated under reduced pressure. The crude compound was purified by CombiFlash chromatography (eluting with 0-70% EtOAc in heptane) followed by preparative HPLC to afford 139 (15 mg, 14%) as an off-white solid. TLC: 80% EtOAc/heptane (R_{f}: 0.5).

### Example 17. (R)-1-(2′,5′-Difluoro-[1,1′-biphenyl]-4-yl)-3-(4-methyl-5-(S-methylsulfonimidoyl) thiazol-2-yl) tetrahydropyrimidin-2(1H)-one (140) and (S)-1-(2′,5′-difluoro-[1,1′-biphenyl]-4-yl)-3-(4-methyl-5-(S-methylsulfonimidoyl)thiazol-2-yl)tetrahydropyrimidin-2(1H)-one (141)

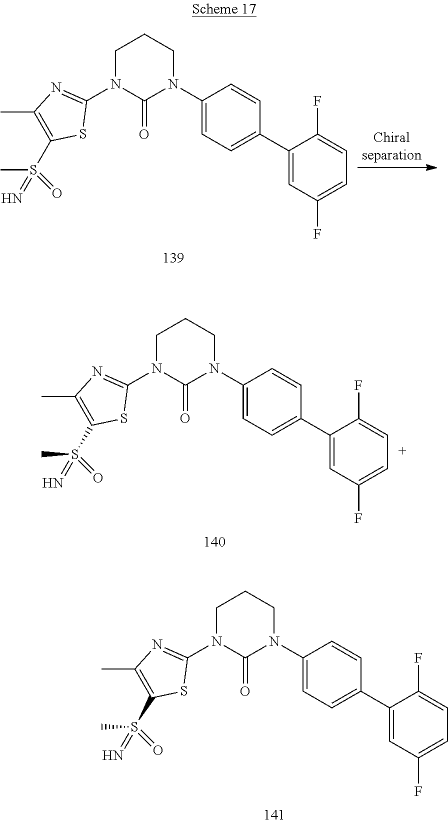 [SMILES: Cc1nc(N2CCCN(c3ccc(-c4cc(F)ccc4F)cc3)C2=O)sc1S(C)(=N)=O ; Cc1nc(N2CCCN(c3ccc(-c4cc(F)ccc4F)cc3)C2=O)sc1[S@@](C)(=N)=O ; Cc1nc(N2CCCN(c3ccc(-c4cc(F)ccc4F)cc3)C2=O)sc1[S@](C)(=N)=O]

Compound 139 chiral separation under the condition below afforded 140 and 141 as an off-white solid.

### Chiral Separation Condition for 140

Chiral HPLC: Rt 14.02 min, 100%
CHIRAL PAK IC (150×4.6 mm, 3 μm); MOBILE PHASE A: n-Hexane; MOBILE PHASE B: DCM:MEOH (50:50); PROGRAM-AB 40:60; FLOW RATE: 1.0 mL/min.

### Chiral Separation Condition for 141

Chiral HPLC: Rt 16.02 min, 99.05%
CHIRAL PAK IC (150×4.6 mm, 3 m); MOBILE PHASE A: n-Hexane; MOBILE PHASE B: DCM:MEOH (50:50); PROGRAM-AB 40:60; FLOW RATE: 1.0 mL/min.

The stereochemistry of 140 and 141 was arbitrarily assigned, respectively.

### Example 18. 1-(2′,5′-Difluoro-[1,1′-biphenyl]-4-yl)-3-(5-(isopropylsulfonyl)-4-methylthiazol-2-yl) tetrahydropyrimidin-2(1H)-one (162)

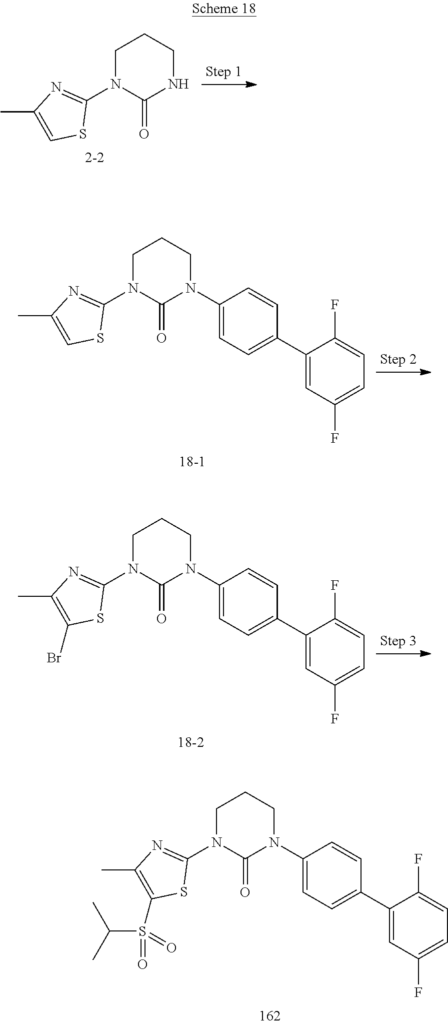 [SMILES: Cc1csc(N2CCCNC2=O)n1 ; Cc1csc(N2CCCN(c3ccc(-c4cc(F)ccc4F)cc3)C2=O)n1 ; Cc1nc(N2CCCN(c3ccc(-c4cc(F)ccc4F)cc3)C2=O)sc1Br ; Cc1nc(N2CCCN(c3ccc(-c4cc(F)ccc4F)cc3)C2=O)sc1S(=O)(=O)C(C)C]

### Step 1. Synthesis of 1-(2′,5′-difluoro-[1,1′-biphenyl]-4-yl)-3-(4-methylthiazol-2-yl) tetrahydropyrimidin-2(1H)-one (18-1)

To a stirred solution of 2-2 (5 g, 25.380 mmol) in 1, 4-dioxane (100 mL) were added 4′-bromo-2,5-difluoro-1,1′-biphenyl (8.16 g, 30.456 mmol), K₂CO₃ (8.75 g, 63.45 mmol) followed by CuI (0.96 g, 5.076 mmol) and the resulting reaction mixture was purged under nitrogen for 20 min. To this resulting reaction mixture, 1,2-dimethylethylenediamine (0.9 g, 10.152 mmol) was added under a nitrogen atmosphere. The reaction mixture was heated at 120° C. for 16 h in a sealed tube. The reaction mixture was filtered through Celite bed and washed with ethyl acetate. The filtrate was diluted with water and extracted with EtOAc then washed with brine. The combined organic layers were dried over anhydrous Na₂SO₄, filtered, and concentrated under reduced pressure. The crude compound obtained was purified by CombiFlash chromatography (eluting with 30-40% EtOAc in heptane) to afford 18-1 (4.1 g, 41.9%) as an off-white solid. TLC: 50% 15 EtOAc/Heptane (R_{f}: 0.5). MS (ESI): calcd. for C₂₀H₁₇F₂N₃OS: 385.11; Found: 385.90 [M+1]⁺. ¹H NMR (400 MHz, DMSO-d₆): δ 7.61 (d, J=7.8 Hz, 2H), 7.54-7.35 (m, 4H), 7.35-7.21 (m, 1H), 6.70 (s, 1H), 4.17 (t, J=5.6 Hz, 2H), 3.81 (t, J=4.9 Hz, 2H), 2.26 (s, 3H), 2.24-2.21 (m, 2H) ppm.

### Step 2. Synthesis of 1-(5-bromo-4-methylthiazol-2-yl)-3-(2′,5′-difluoro-[1,1′-biphenyl]-4-yl) tetrahydropyrimidin-2(1H)-one (18-2)

To a stirred solution of 18-1 (1 g, 2.597 mmol) in DCM (10 mL) was added NBS (0.6 g, 3.37 mmol) and the resulting reaction mixture was stirred at room temperature for 16 h. After completion of the reaction (monitored by TLC), the reaction mixture was diluted with water and extracted with EtOAc then washed with brine. The combined organic layers were dried over anhydrous Na₂SO₄, filtered, and concentrated under reduced pressure. The crude compound obtained was purified by CombiFlash chromatography (eluting with 30-40% EtOAc in heptane) to afford 18-2 (0.7 g, 58.3%) as an off-white solid. TLC: 50% EtOAc/Heptane (R_{f}: 0.5). MS (ESI): calcd. for C₂₀H₁₆BrF₂N₃OS: 463.02; Found: 466.0 [M+2]⁺.

### Step 3. Synthesis of 1-(2′,5′-difluoro-[1,1′-biphenyl]-4-yl)-3-(5-(isopropylsulfonyl)-4-methylthiazol-2-yl) tetrahydropyrimidin-2(1H)-one (162)

To a stirred solution of 18-2 (0.5 g, 1.079 mmol) in DMF (10 mL) were added propane-2-sulfinate sodium salt (0.56 g, 4.319 mmol) and CS₂CO₃ (0.87 g, 2.69 mmol) and then was subsequently added CuI (21 mg, 0.1079 mmol) followed by L-proline (25 mg, 0.216 mmol). The resulting reaction mixture was heated at 80° C. for 12 h. The reaction mixture was diluted with water and extracted with EtOAc then washed with brine. The combined organic layers were dried over anhydrous Na₂SO₄, filtered, and concentrated under reduced pressure. The crude compound obtained was purified by preparative HPLC to afford 162 (8 mg, 1.5%) as an off-white solid.

### Example 19. 2-(3-(2′,5′-Difluoro-[1,1′-biphenyl]-4-yl)-2-oxotetrahydropyrimidin-1(2H)-yl)-4-(trifluoromethyl) thiazole-5-sulfonamide (169)

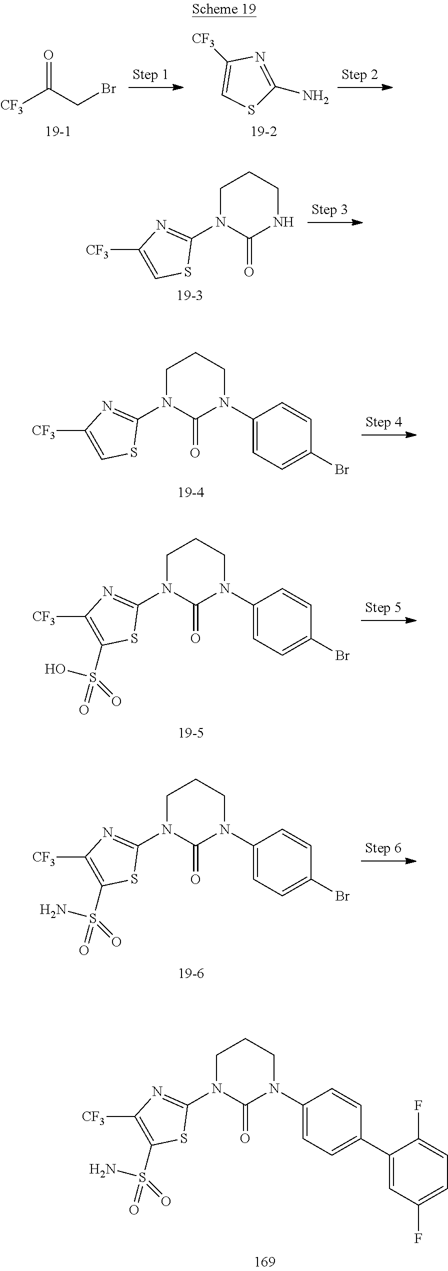

### Step 1. Synthesis of 4-(trifluoromethyl) thiazol-2-amine (19-2)

To a stirred solution of 19-1 (2.7 g, 14.210 mmol) in ethanol (50 mL) was added thiourea (2.16 g, 28.42 mmol) and the resulting reaction mixture was stirred at 60° C. for 2 h. The reaction mixture was concentrated under reduced pressure to dryness. The crude compound was purified by CombiFlash chromatography (eluting with 20-30% EtOAc in heptane) to afford 19-2 (3 g, crude) as a sticky liquid. TLC: 20% EtOAc/heptane (R_{f}: 0.2). MS (ESI): calcd. for C₄H₃F₃N₂S: 168.00; Found: 169.00 [M+1]⁺.

### Step 2. Synthesis of 1-(4-(trifluoromethyl) thiazol-2-yl) tetrahydropyrimidin-2(1H)-one (19-3)

To a mixture of 19-2 (4 g, 23.809 mmol) and 1-chloro-3-isocyanatopropane (4.2 g, 35.714 mmol) in THF (60 mL) were added TBAI (0.38 g, 1.190 mmol) and K₂CO₃ (4.28 g, 30.951 mmol) portion wise the resulting reaction mixture was stirred at 70° C. for 16 h. After completion of the reaction (monitored by TLC), the reaction mixture was diluted with water and extracted with EtOAc. The combined organic layers were dried over anhydrous Na₂SO₄, filtered, and concentrated under reduced pressure. The crude compound was purified by CombiFlash chromatography (eluting with 30-40% EtOAc in heptane) to afford 19-3 (2.7 g, 45.2%) as an off-white solid. TLC: 40% EtOAc/heptane (R_{f}: 0.5). MS (ESI): calcd. for C₈H₈F₃N₃OS: 251.03; Found: 251.80 [M+1]⁺.

### Step 3. Synthesis of 1-(4-bromophenyl)-3-(4-(trifluoromethyl) thiazol-2-yl) tetrahydropyrimidin-2(1H)-one (19-4)

To a stirred solution of 19-3 (2.7 g, 10.756 mmol) in ACN (50 mL) were added 1-bromo-4-iodobenzene (3.65 g, 12.908 mmol), CS₂CO₃ (6.99 g, 21.512 mmol) followed by CuI (0.81 g, 4.302 mmol) and the resulting reaction mixture was purged under nitrogen for 10 min. To this resulting reaction mixture, 1,2-Dimethylethylenediamine (0.378 g, 4.302 mmol) was added under a nitrogen atmosphere. The reaction mixture was heated at 100° C. for 16 h in a sealed tube. The reaction mixture was filtered through Celite bed and washed with ethyl acetate. The filtrate was diluted with water and extracted with EtOAc then washed with brine. The combined organic layers were dried over anhydrous Na₂SO₄, filtered, and concentrated under reduced pressure. The crude compound obtained was purified by CombiFlash chromatography (eluting with 30-40% EtOAc in heptane) to afford 19-4 (2 g, 45.9%) as an off-white solid. TLC: 50% EtOAc/heptane (R_{f}: 0.5). MS (ESI): calcd. for C₁₄H₁₁BrF₃N₃OS: 404.98; Found: 405.80 [M+1]⁺.

### Step 4. Synthesis of 2-(3-(4-bromophenyl)-2-oxotetrahydropyrimidin-1(2H)-yl)-4-(trifluoromethyl) thiazole-5-sulfonic acid (19-5)

To a stirred solution of 19-4 (0.5 g, 1.234 mmol) in dry DCM (8 mL) at 0° C. in an inert atmosphere, chlorosulfuric acid (0.4 mL, 4.938 mmol) was added and the resulting reaction mixture was slowly warmed to room temperature and stirred for 48 h. The reaction mixture was concentrated under reduced pressure to dryness. The crude residue obtained was purified by trituration with diethyl ether. The obtained solid was filtered off and dried in vacuo to afford 19-5 (0.5 g, crude) as an off-white solid. TLC: 30% EtOAc/Heptane (R_{f}: 0.3). MS (ESI): calcd. for C₁₄H₁₁BrF₃N₃O₄S₂: 484.93; Found: 485.65 [M+1]⁺.

### Step 5. Synthesis of 2-(3-(4-bromophenyl)-2-oxotetrahydropyrimidin-1(2H)-yl)-4-(trifluoromethyl) thiazole-5-sulfonamide (19-6)

A stirred solution of 19-5 (0.5 g, 1.030 mmol) in POCl₃ (6 mL) was allowed to stir at 100° C. for 2 h. The reaction mixture was concentrated under reduced pressure to dryness. The resulting residue was dissolved in THF (3 mL) and aqueous ammonia (6 mL) was added at 0° C. while stirring was continued at room temperature for another 16 h. The reaction mixture was diluted with water and extracted with EtOAc. The combined organic layers were dried over anhydrous Na₂SO₄, filtered, and concentrated under reduced pressure. The crude compound was purified by CombiFlash chromatography (eluting with 30-40% EtOAc in heptane) to afford 19-6 (0.2 g, 40.1%) as an off-white solid. MS (ESI): calcd. for C₁₄H₁₂BrF₃N₄O₃S₂: 483.95; Found: 484.85 [M+1]⁺.

### Step 6. Synthesis of 2-(3-(2′,5′-difluoro-[1,1′-biphenyl]-4-yl)-2-oxotetrahydropyrimidin-1(2H)-yl)-4-(trifluoromethyl) thiazole-5-sulfonamide (169)

To a stirred solution of 19-6 (0.2 g, 0.413 mmol) in 1,4 dioxane:H₂O (5:1 mL) were added (2,5-difluorophenyl) boronic acid (0.1 g, 0.619 mmol) and K₃PO₄ (0.21 g, 1.032 mmol) and the reaction mixture was purged under nitrogen for 10 min. Pd(dppf) Cl₂ (30 mg, 0.041 mmol) was added under a nitrogen atmosphere. The reaction mixture was heated at 100° C. for 4 h. After completion of the reaction (monitored by TLC), the reaction mixture was cooled to room temperature, filtered through a pad of Celite and washed with ethyl acetate. The filtrate was diluted with water and extracted with EtOAc. The combined organic layers were dried over anhydrous Na₂SO₄, filtered and concentrated under reduced pressure. The crude compound was purified by CombiFlash chromatography (eluting with 10-30% EtOAc in heptane) to afford 169 (25 mg, 11.6%) as an off-white solid. TLC: 30% EtOAc/heptane (R_{f}: 0.3).

### Example 20. 2-(3-((2′,5′-Difluoro-[1,1′-biphenyl]-3-yl) methyl)-2-oxotetrahydropyrimidin-1(2H)-yl)-4-methylthiazole-5-sulfonamide (172)

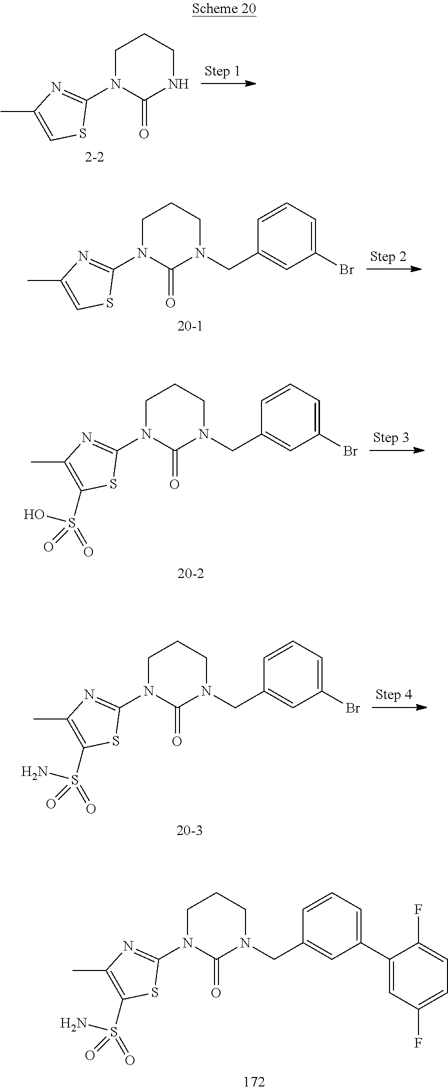 [SMILES: Cc1csc(N2CCCNC2=O)n1 ; Cc1csc(N2CCCN(Cc3cccc(Br)c3)C2=O)n1 ; Cc1nc(N2CCCN(Cc3cccc(Br)c3)C2=O)sc1S(=O)(=O)O ; Cc1nc(N2CCCN(Cc3cccc(Br)c3)C2=O)sc1S(N)(=O)=O ; Cc1nc(N2CCCN(Cc3cccc(-c4cc(F)ccc4F)c3)C2=O)sc1S(N)(=O)=O]

### Step 1. Synthesis of 1-(3-bromobenzyl)-3-(4-methylthiazol-2-yl) tetrahydropyrimidin-2(1H)-one (20-1)

To a stirred solution of 2-2 (4 g, 20.304 mmol) in dry THF (80 mL) at 0° C. under a nitrogen atmosphere, NaH (60% w/w in mineral oil, 1.21 g, 30.456 mmol) was added in small portion and the resulting reaction mixture was stirred at the same temperature for 30 min. To this reaction mixture, 1-bromo-4-(bromomethyl) benzene (6 g, 24.36 mmol) was added at 0° C. and The resulting reaction mixture was stirred at the same temperature for 2 h. After completion of the reaction (monitored by TLC), the reaction mixture was quenched with ice cold water and extracted with EtOAc. The combined organic layers were dried over anhydrous Na₂SO₄, filtered and concentrated under reduced pressure. The crude compound was purified by CombiFlash chromatography (using a gradient method of 50-60% EtOAc in heptane) to afford 20-1 (5.21 g, 70.3%) as an off-white solid. TLC: 50% EtOAc/heptane (R_{f}: 0.5). MS (ESI): calcd. for C₁₅H₁₆BrN₃OS: 365.02; Found: 368 [M+2]⁺. ¹H NMR (400 MHz, DMSO-d₆): δ 7.51-7.46 (m, 2H), 7.36-7.27 (m, 2H), 6.66 (s, 1H), 4.57 (s, 2H), 4.07 (t, J=5.6 Hz, 2H), 3.36-3.33 (m, 2H), 2.23 (s, 3H), 2.06-1.99 (m, 2H) ppm

### Step 2. Synthesis of 2-(3-(3-bromobenzyl)-2-oxotetrahydropyrimidin-1(2H)-yl)-4-methylthiazole-5-sulfonic acid (20-2)

To a stirred solution of 20-1 (1 g, 2.739 mmol) in dry DCM (10 mL) at 0° C. in an inert atmosphere, chlorosulfuric acid (0.63 g, 5.48 mmol) was added and the resulting reaction mixture was slowly warmed to room temperature and stirred for 16 h. After completion of the reaction (monitored by TLC), the reaction mixture was quenched with ice cold water and stirred for 5-10 min. The precipitated solid obtained out was collected by filtration and dried in vacuo to afford 20-2 (1.02 g, crude) as an off-white solid. TLC: 50% EtOAc/heptane (R_{f}: 0.2) which was used in the next step without further purification. MS (ESI): calcd. for C₁₅H₁₆BrN₃O₄S₂: 444.98; Found: 446 [M+1]. ¹H NMR (400 MHz, DMSO-d₆): δ 7.52-7.45 (m, 2H), 7.35-7.28 (m, 2H), 4.57 (s, 2H), 4.04-3.97 (m, 2H), 3.41-3.25 (m, 2H), 2.32 (s, 3H), 2.08-1.96 (m, 2H) ppm.

### Step 3. Synthesis of 2-(3-(3-bromobenzyl)-2-oxotetrahydropyrimidin-1(2H)-yl)-4-methylthiazole-5-sulfonamide (20-3)

To a stirred solution of 20-2 (1 g, 2.242 mmol) in POCl₃ (10 mL) was allowed to stir at 80° C. for 12 h. The reaction mixture was concentrated under reduced pressure to dryness. The resulting residue was dissolved in THF (5 mL) and aqueous ammonia (10 mL) was added at 0° C. while stirring was continued at room temperature for another 12 h. The reaction mixture was diluted with water and extracted with EtOAc. The combined organic layers were dried over anhydrous Na₂SO₄, filtered, and concentrated under reduced pressure. The crude compound was purified by CombiFlash chromatography (using a gradient method of 1-2% MeOH in DCM) to afford 20-3 (0.60 g, 60.6%) as an off-white solid. TLC: 50% EtOAc/heptane (R_{f}: 0.5). MS (ESI): calcd. for C₁₅H₁₇BrN₄O₃S₂: 443.99; Found: 447.1 [M+2]⁺. ¹H NMR (400 MHz, DMSO-d₆): δ 7.55-7.50 (m, 4H), 7.31 (s, 2H), 4.58 (s, 2H), 4.11-4.00 (m, 2H), 3.39-3.33 (m, 2H), 2.42 (s, 3H), 2.10-2.01 (m, 2H) ppm.

### Step 4. Synthesis of 2-(3-((2′,5′-difluoro-[1,1′-biphenyl]-3-yl) methyl)-2-oxotetrahydropyrimidin-1(2H)-yl)-4-methylthiazole-5-sulfonamide (172)

To a stirred solution of 20-3 (0.3 g, 0.677 mmol) in 1, 4-dioxane and water (4:1.5 mL) were added (2,5-difluorophenyl) boronic acid (0.16 g, 1.015 mmol) and K₃PO₄ (0.36 g, 1.692 mmol) and the reaction mixture was purged under nitrogen for 10 min. Pd(dppf)Cl₂ (50 mg, 0.0677 mmol) was added under a nitrogen atmosphere. The reaction mixture was heated at 80° C. for 2 h. After completion of the reaction (monitored by TLC), the reaction mixture was cooled to room temperature, diluted with water and, extracted with EtOAc then washed with brine. The combined organic layers were dried over anhydrous Na₂SO₄, filtered, and concentrated under reduced pressure. The crude compound obtained was purified by CombiFlash chromatography (eluting with 1-2% MeOH in DCM) to afford 172 (0.14 g, 45.6%) as an off-white solid. TLC: 5% MeOH/DCM (R_{f}: 0.5).

### Example 21. 2-(3-(1-(2′,5′-Difluoro-[1,1′-biphenyl]-3-yl) ethyl)-2-oxotetrahydropyrimidin-1(2H)-yl)-4-methylthiazole-5-sulfonamide (174)

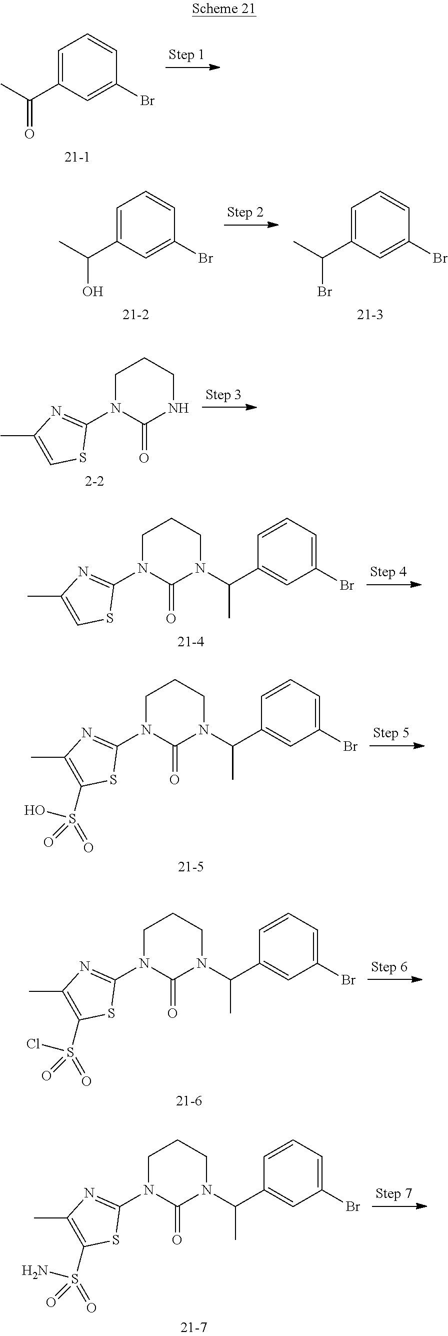

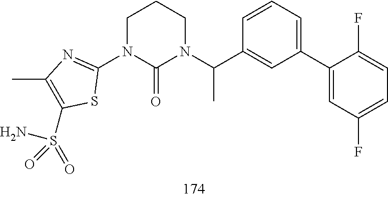 [SMILES: Cc1nc(N2CCCN(C(C)c3cccc(-c4cc(F)ccc4F)c3)C2=O)sc1S(N)(=O)=O]

### Step 2. Synthesis of 1-bromo-3-(1-bromoethyl) benzene (21-3)

To a stirred solution of 21-2 (4.5 g, 22.38 mmol) in DCM (20 mL) at 0° C. in an inert atmosphere, Phosphorus tribromide (2.13 mL, 22.38 mmol) was added dropwise and the reaction mixture was stirred at the same temperature for 4 h. After completion of the reaction (monitored by TLC), the reaction mixture was quenched with saturated NaHCO₃ solution and extracted with EtOAc. The combined organic layers were dried over anhydrous Na₂SO₄, filtered, and concentrated under reduced pressure to afford 21-3 (4 g, crude) as a pale green solid, which was used in the next step without further purification. TLC: 10% EtOAc/heptane (R_{f}: 0.5).

### Step 3. Synthesis of 1-(1-(3-bromophenyl) ethyl)-3-(4-methylthiazol-2-yl) tetrahydropyrimidin-2(1H)-one (21-4)

To a stirred solution of 2-2 (0.8 g, 4.060 mmol) in dry DMF (8 mL) at 0° C. under a nitrogen atmosphere, NaH (60% w/w in mineral oil, 0.24 g, 6.091 mmol) was added in small portions and the resulting reaction mixture was stirred at the same temperature for 15 min. To this reaction mixture, 21-3 (1.6 g, 6.091 mmol) was added at 0° C. and then the reaction mixture was slowly warmed to room temperature and stirred at 50° C. for 16 h. After completion of the reaction (monitored by TLC), the reaction mixture was quenched with ice cold water and extracted with EtOAc. The combined organic layers were dried over anhydrous Na₂SO₄, filtered, and concentrated under reduced pressure. The crude compound was purified by CombiFlash chromatography (eluting with 20-30% EtOAc in heptane) to afford 21-4 (1.2 g, 77.9%) as a yellow solid. TLC: 40% EtOAc/heptane (R_{f}: 0.5). MS (ESI): calcd. for C₁₆H₁₈BrN₃OS: 379.04; Found: 379.9 [M+1]⁺.

### Step 4. Synthesis of 2-(3-(1-(3-bromophenyl) ethyl)-2-oxotetrahydropyrimidin-1(2H)-yl)-4-methylthiazole-5-sulfonic acid (21-5)

To a stirred solution of 21-4 (1.2 g, 3.165 mmol) in dry DCM (12 mL) at 0° C. in an inert atmosphere, chlorosulfuric acid (0.42 mL, 6.331 mmol) was added and the resulting reaction mixture was slowly warmed to room temperature and stirred for 16 h. The reaction mixture was concentrated under reduced pressure to dryness. The crude residue obtained was purified by trituration with diethyl ether. The obtained solid was filtered off and dried in vacuo to afford 21-5 (0.8 g, crude) as a pale brown colored solid. TLC: 50% EtOAc/heptane (R_{f}: 0.3). MS (ESI): calcd. for C₁₆H₁₈BrN₃O₄S₂: 458.99; Found: 461.7 [M+2]⁺.

### Step 5. Synthesis of 2-(3-(1-(3-bromophenyl) ethyl)-2-oxotetrahydropyrimidin-1(2H)-yl)-4-methylthiazole-5-sulfonyl chloride (21-6)

A stirred solution of 21-5 (0.8 g, 1.742 mmol) in POCl₃ (8 mL) was allowed to stir at 80° C. for 16 h. After completion of the reaction (monitored by TLC), the reaction mixture was cooled to room temperature and concentrated under reduced pressure to afford 21-6 (0.7 g, crude) as an orange colored solid, which was used without further purification.

### Step 6. Synthesis of 2-(3-(1-(3-bromophenyl) ethyl)-2-oxotetrahydropyrimidin-1(2H)-yl)-4-methylthiazole-5-sulfonamide (21-7)

To a stirred solution of 21-6 (0.6 g 1.257 mmol) in THF (6 mL) at 0° C., aqueous ammonia (1 mL) was added dropwise. The resulting reaction mixture was slowly warmed to room temperature and allowed to stir for 16 h. After completion of the reaction (monitored by TLC), the reaction mixture was diluted with water and extracted, with EtOAc. The combined organic layers were dried over anhydrous Na₂SO₄, filtered, and concentrated under reduced pressure. The crude product was purified by CombiFlash chromatography (eluting with 20-50% EtOAc in heptane) to afford the title compound 21-7 (0.16 g, 29.8%) as an off-white solid. TLC: 50% EtOAc/heptane (R_{f}: 0.3). MS (ESI): calcd. for C₁₆H₁₉BrN₄O₃S₂: 458.01; Found: 460.7 [M+2]⁺.

### Step 7. Synthesis of 2-(3-(1-(2′,5′-difluoro-[1,1′-biphenyl]-3-yl) ethyl)-2-oxotetrahydropyrimidin-1(2H)-yl)-4-methylthiazole-5-sulfonamide (174)

To a stirred solution of 21-7 (0.15 g, 0.327 mmol) in 1, 4-dioxane and water (2.5:0.5 mL) were added (2,5-difluorophenyl) boronic acid (0.13 g, 0.818 mmol) and K₃PO₄ (0.17 g, 0.818 mmol) and the reaction mixture was purged under nitrogen for 10 min. Pd(dppf)Cl₂ (25 mg, 0.038 mmol) was added under a nitrogen atmosphere. The reaction mixture was heated at 80° C. for 10 h. After completion of the reaction (monitored by TLC), the reaction mixture was cooled to room temperature, diluted with water, and extracted with EtOAc then washed with brine. The combined organic layers were dried over anhydrous Na₂SO₄, filtered, and concentrated under reduced pressure. The crude compound was purified by preparative HPLC to afford 174 (80 mg) as an off-white solid. TLC: 50% EtOAc/heptane (R_{f}: 0.5).

### Example 22. Synthesis of 2-(3-(2′,5′-difluoro-[1,1′-biphenyl]-4-yl)-2-oxotetrahydropyrimidin-1(2H)-yl)-4-methylthiazole-5-sulfonamide (5)

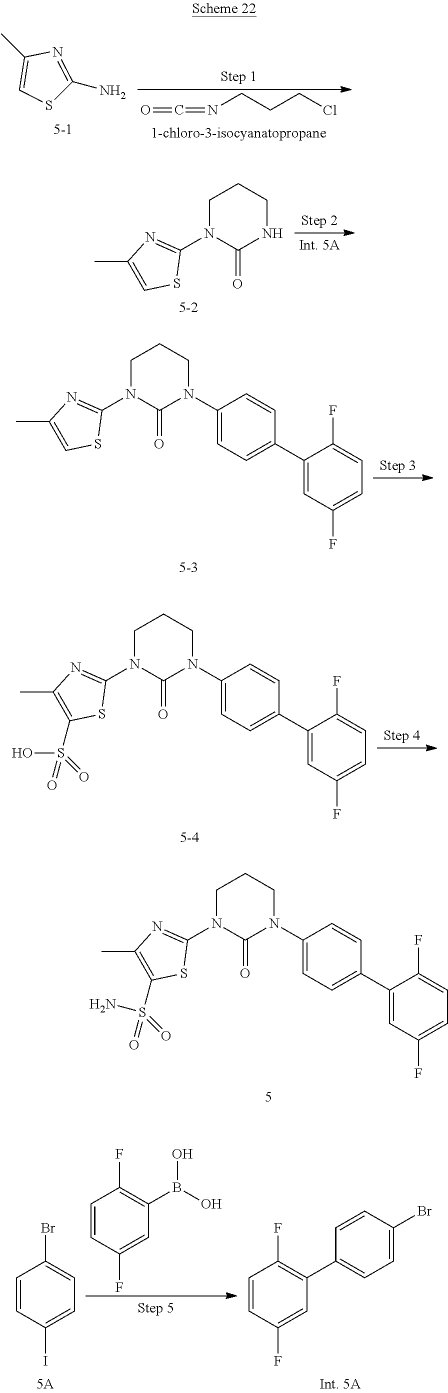

### Step 1. Synthesis of 1-(4-methylthiazol-2-yl) tetrahydropyrimidin-2(1H)-one (5-2)

A mixture of compound 5-1 (6 g, 52.632 mmol) and 1-chloro-3-isocyanatopropane (6.26 g, 52.632 mmol) in THF (60 mL) was heated at 70° C. for 6 h. To the resulting solution, TBAB (1.7 g, 5.263 mmol) and K₂CO₃ (18.15 g, 131.58 mmol) were added portion wise maintaining the same temperature and stirring continued at 70° C. for 16 h. After completion of the reaction (monitored by TLC), the reaction mixture was diluted with water and extracted with EtOAc. The combined organic layers were dried over anhydrous Na₂SO₄, filtered and concentrated under reduced pressure. The crude compound was purified by CombiFlash chromatography (eluting with 60-70% EtOAc in heptane) to afford the title compound 5-2 (5.1 g, 49.2%) as an off-white solid. TLC: 70% EtOAc/heptane (R_{f}: 0.5). MS (ESI): calcd. for C₈H₁₁N₃OS: 197.06; Found: 198.17 [M+1]⁺. ¹H NMR (400 MHz, DMSO-d₆): δ 7.30 (s, 1H), 6.60 (s, 1H), 3.99 (t, J=5.4 Hz, 2H), 3.20-3.19 (m, 2H), 2.28 (s, 3H), 1.99-1.89 (m, 2H) ppm.

### Step 2. Synthesis of 1-(2′,5′-difluoro-[1,1′-biphenyl]-4-yl)-3-(4-methylthiazol-2-yl) tetrahydropyrimidin-2(1H)-one (5-3)

To a stirred solution of compound 5-2 (5 g, 25.380 mmol) in 1, 4-dioxane (100 mL) were added 20 Int #5A (8.16 g, 30.456 mmol), K₂CO₃ (8.75 g, 63.45 mmol) followed by CuI (0.96 g, 5.076 mmol) and the resulting reaction mixture was purged under nitrogen for 20 min. To this resulting reaction mixture, 1,2-Dimethylethylenediamine (0.9 g, 10.152 mmol) was added under a nitrogen atmosphere. The reaction mixture was heated at 120° C. for 24 h in a sealed tube. The reaction mixture was filtered through Celite bed and washed with ethyl acetate. The filtrate was diluted with water and, extracted with EtOAc then washed with brine. The combined organic layers were dried over anhydrous Na₂SO₄, filtered and concentrated under reduced pressure. The crude compound obtained was purified by CombiFlash chromatography (eluting with 30-40% EtOAc in heptane) to afford the title compound 5-3 (4.1 g, 41.9%) as an off-white solid. TLC: 50% EtOAc/Heptane (R_{f}: 0.5). MS (ESI): calcd. for C₂₀H₁₇F₂N₃OS: 385.11; Found: 385.90 [M+1]⁺. ¹H NMR (400 MHz, DMSO-d₆): δ 7.61 (d, J=7.8 Hz, 2H), 7.54-7.35 (m, 4H), 7.35-7.21 (m, 1H), 6.70 (s, 1H), 4.17 (t, J=5.6 Hz, 2H), 3.81 (t, J=4.9 Hz, 2H), 2.26 (s, 3H), 2.24-2.21 (m, 2H) ppm.

### Step 3. Synthesis of 2-(3-(2′,5′-difluoro-[1,1′-biphenyl]-4-yl)-2-oxotetrahydropyrimidin-1(2H)-yl)-4-methylthiazole-5-sulfonic acid (5-4)

To a stirred solution of compound 5-3 (4 g, 10.389 mmol) in dry DCM (40 mL) at 0° C. in an inert atmosphere, chlorosulfuric acid (2.07 mL, 31.168 mmol) was added and the resulting reaction mixture was slowly warmed to room temperature and stirred for 12 h. The reaction mixture was concentrated under reduced pressure to dryness. The crude residue obtained was purified by trituration with diethyl ether. The obtained solid was filtered off and dried in vacuo to afford the title compound 5-4 (3.35 g, crude) as an off-white solid. TLC: 100% EtOAc (R_{f}: 0.2). MS (EST): calcd. for C₂₀H₁₇F₂N₃O₄S₂: 465.06; Found: 466 [M+1]⁺.

### Step 4. Synthesis of 2-(3-(2′,5′-difluoro-[1,1′-biphenyl]-4-yl)-2-oxotetrahydropyrimidin-1(2H)-yl)-4-methylthiazole-5-sulfonamide (5)

A stirred solution of compound 5-4 (3.3 g, 7.096 mmol) in POCl₃ (33 mL) was allowed to stir at 90° C. for 5 h. The reaction mixture was concentrated under reduced pressure to dryness. The resulting residue was dissolved in THF (66 mL), and aqueous ammonia (33 mL) was added at −5° C. while stirring was continued at room temperature for another 12 h. The reaction mixture was diluted with water and extracted with EtOAc. The combined organic layers were dried over anhydrous Na₂SO₄, filtered and concentrated under reduced pressure. The crude compound was purified by CombiFlash chromatography (eluting with 100% EtOAc) to afford the desired product as a white solid 5 (1.1 g, 44.6%) as a white solid. ¹H NMR (400 MHz, DMSO-d₆): δ 7.65-7.59 (m, 2H), 7.55 (br s, 2H), 7.53-7.48 (m, 2H), 7.48-7.36 (m, 2H), 7.31-7.25 (m, 1H), 4.17 (t, J=6.1 Hz, 2H), 3.82 (t, J=5.6 Hz, 2H), 2.45 (s, 3H), 2.29-2.18 (m, 2H) ppm.

### Step 5. Synthesis of 4′-bromo-2,5-difluoro-1,1′-biphenyl (Int. 5A)

To a stirred solution of compound 5A (5 g, 17.674 mmol) in 1,4 dioxane:H₂O (50:5 mL) were added (2,5-difluorophenyl) boronic acid (3.07 g, 19.441 mmol) and K₃PO₄ (7.5 g, 35.348 mmol) and the reaction mixture was purged under nitrogen for 10 min. Pd(dppf)Cl₂ (1.29 g, 1.767 mmol) was added under a nitrogen atmosphere. The reaction mixture was heated at 80° C. for 1 h. After completion of the reaction (monitored by TLC), the reaction mixture was cooled to room temperature, filtered through a pad of Celite and washed with ethyl acetate. The filtrate was diluted with water and extracted with EtOAc. The combined organic layers were dried over anhydrous Na₂SO₄, filtered, and concentrated under reduced pressure. The crude compound was purified by CombiFlash chromatography (eluting with 100% heptane) to afford the title compound Int. 5A (2.3 g, 48.6%) as an off-white solid. TLC: 100% heptane (R_{f}: 0.5). ¹H NMR (400 MHz, CDCl₃): δ 7.58 (d, J=8.3 Hz, 2H), 7.40 (d, J=7.3 Hz, 2H), 7.15-7.06 (m, 2H), 7.05-6.97 (m, 1H) ppm.

### Example 23. Synthesis of 2-(3-(3′-(1-(difluoromethyl)-1H-pyrazol-4-yl)-2′,5′-difluoro-[1,1′-biphenyl]-4-yl)-2-oxotetrahydropyrimidin-1(2H)-yl)-4-methylthiazole-5-sulfonamide (228)

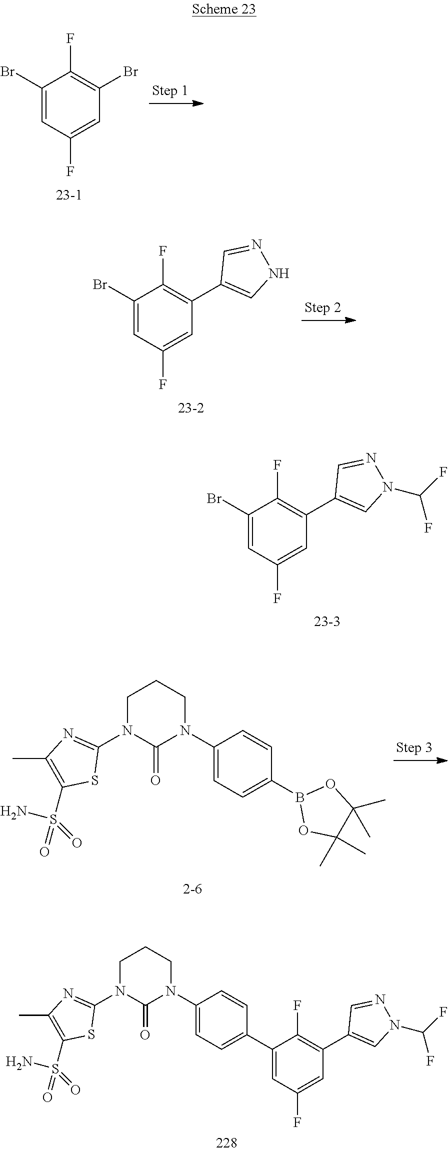

### Synthesis of 4-(3-bromo-2,5-difluorophenyl)-1H-pyrazole (23-2)

To a stirred solution of 1,3-dibromo-2,5-difluorobenzene (23-1) (1215 mg, 4.47 mmol) and (1H-pyrazol-4-yl) boronic acid (500 mg, 4.47 mmol) in DME (10 mL), water (4.00 mL) was added K₂CO₃ (1.853 g, 13.41 mmol) and Pd(dppfcl2)DCM (365 mg, 0.447 mmol) and purged with nitrogen for 10 min. The reaction mixture was heated at 80° C. for 3 h. The reaction was monitored by UPLC. After completion, the reaction mixture was diluted with cold water (10 mL) and extracted with ethyl acetate (2×15 mL). The combined organic layer was washed with brine (1×15 mL), dried over anhydrous sodium sulfate, and concentrated on a rotary evaporator (bath temperature 45° C.) under reduced pressure to afford 4-(3-bromo-2,5-difluorophenyl)-1H-pyrazole (23-2) (200 mg, 0.749 mmol, 16.7% yield) as a crude compound. LCMS: 1.704 min, 97.65%, 259.0 (M+H)⁺, (Column: Atlantis dC18 (50*4.6) 5 μm), Mobile phase A: 0.1% Formic acid in H₂O, Mobile phase B: ACN, Flow Rate: 1.5 ml/min)

### Synthesis of 4-(3-bromo-2,5-difluorophenyl)-1-(difluoromethyl)-1H-pyrazole (23-3)

To a stirred solution of 4-(3-bromo-2,5-difluorophenyl)-1H-pyrazole (23-2) (200 mg, 0.772 mmol) and potassium fluoride (90 mg, 1.544 mmol) in Acetonitrile (10 mL), were added Diethyl (bromodifluoromethyl)phosphonate (0.275 mL, 1.544 mmol) and the reaction mixture was stirred at RT for 16 h. After completion, the reaction mixture was diluted with cold water (10 mL) and extracted with ethyl acetate (2×15 mL). The combined organic layer was washed with brine (1×15 mL), dried over anhydrous sodium sulfate, and concentrated on a rotary evaporator (bath temperature 45° C.) under reduced pressure to afford crude. The obtained crude was purified by Isolera column chromatography Technique using 230-400 silica gel, eluted in 20% Ethyl acetate: Pet ether. The combined pure fractions are concentrated on a rota evaporator under reduced pressure to afford pure 4-(3-bromo-2,5-difluorophenyl)-1-(difluoromethyl)-1H-pyrazole (23-3) (20 mg, 0.062 mmol, 8% yield) as a pale-yellow liquid. LCMS: 2.044 min, 97.65%, 310.9 (M+H)⁺, (Column: XBridge C18 (50×4.6 mm) 3.5 μm, Mobile phase A: 0.1% TFA in H2O, Mobile phase B: ACN, Flow Rate: 2.0 ml/min).

### Synthesis of 2-(3-(3′-(1-(difluoromethyl)-1H-pyrazol-4-yl)-2′,5′-difluoro-[1,1′-biphenyl]-4-yl)-2-oxotetrahydropyrimidin-1(2H)-yl)-4-methylthiazole-5-sulfonamide (228)

To a stirred solution of 4-(3-bromo-2,5-difluorophenyl)-1-(difluoromethyl)-1H-pyrazole (23-3) (20 mg, 0.065 mmol) and 4-methyl-2-(2-oxo-3-(4-(4,4,5,5-tetramethyl-1,3,2-dioxaborolan-2-yl)phenyl)tetrahydropyrimidin-1(2H)-yl)thiazole-5-sulfonamide (2-6) (31.0 mg, 0.065 mmol) in THF (10 mL), water (4.00 mL) was added K₂CO₃ (26.8 mg, 0.194 mmol) and XPhos Pd G2 (5.09 mg, 6.47 μmol) and purged with nitrogen for 10 min. The reaction mixture was heated at 80° C. for 3 h. The reaction was monitored by LCMS. The reaction mixture was diluted with water (5 mL) and extracted with Ethyl acetate (2×5 mL). The combined organic layer was washed with water (5 mL) followed by brine solution, dried over sodium sulfate, and concentrated. The resulting crude compound was purified by Prep HPLC (FA method) to afford 2-(3-(3′-(1-(difluoromethyl)-1H-pyrazol-4-yl)-2′,5′-difluoro-[1,1′-biphenyl]-4-yl)-2-oxotetrahydropyrimidin-1(2H)-yl)-4-methylthiazole-5-sulfonamide (228) (4.25 mg, 7.25 μmol, 11.2% yield) as an off white solid.

### Example 24: Synthesis of 2-(3-(4-(4,6-difluoropyridin-2-yl)phenyl)-2-oxotetrahydropyrimidin-1(2H)-yl)-4-methylthiazole-5-sulfonamide (235)

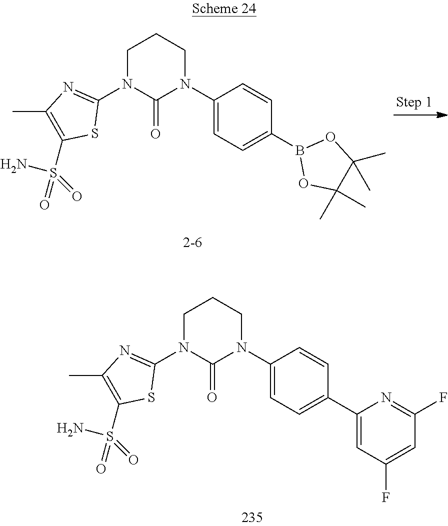

### Synthesis of Synthesis of 2-(3-(4-(4,6-difluoropyridin-2-yl)phenyl)-2-oxotetrahydropyrimidin-1(2H)-yl)-4-methylthiazole-5-sulfonamide (235)

To a stirred solution of compound 2-6 (0.18 g, 0.386 mmol) in 1, 4-dioxane and water (4:1 mL) was added 2-bromo-4,6-difluoropyridine (50 mg, 0.257 mmol) followed by K₃PO₄ (0.10 g, 0.515 mmol) and the reaction mixture was purged under nitrogen for 10 min. To this resulting solution, PdCl₂(dppf) (19 mg, 0.0257 mmol) was added under a nitrogen atmosphere and the reaction mixture was heated at 110° C. for 16 h. After completion of the reaction (monitored by TLC), the reaction mixture was cooled to room temperature, diluted with water, and extracted with EtOAc then washed with brine. The combined organic layers were dried over anhydrous Na₂SO₄, filtered, and concentrated under reduced pressure. The crude compound was purified by preparative HPLC to afford the title compound 235 (20 mg, 16.5%) as an off-white solid.

### Example 25: Synthesis of 2-(8-(2′,5′-difluoro-[1,1′-biphenyl]-4-yl)-7-oxo-2-oxa-6,8-diazaspiro[3.5]nonan-6-yl)-4-methylthiazole-5-sulfonamide (339)

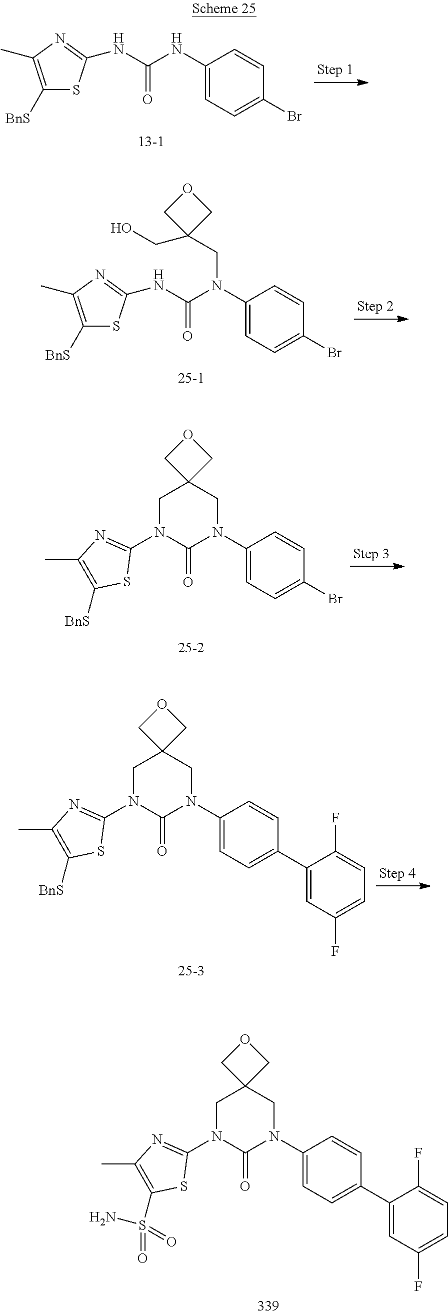

### Synthesis of 3-(5-(benzylthio)-4-methylthiazol-2-yl)-1-(4-bromophenyl)-1-((3-(hydroxymethyl)oxetan-3-yl)methyl)urea (25-1)

To a stirred solution of compound 13-1 (4 g, 9.259 mmol) in DMF (40 mL) at 0° C., was added CS₂CO₃ (7.54 g, 23.147 mmol) followed by (3-(bromomethyl)oxetan-3-yl)methanol (2.51 g, 13.888 mmol). The resulting reaction mixture was slowly warmed to room temperature and stirred for 16 h. After completion of the reaction (monitored by TLC), the reaction mixture was diluted with water and extracted with EtOAc. The combined organic layers were dried over anhydrous Na₂SO₄, filtered, and concentrated under reduced pressure. The crude compound was purified by Combi Flash chromatography (eluting with 40-50% EtOAc in heptane) to afford the title compound 25-1 (1 g, 20.2%) as a pale-yellow solid.

### Synthesis of 6-(5-(benzylthio)-4-methylthiazol-2-yl)-8-(4-bromophenyl)-2-oxa-6,8-diazaspiro[3.5]nonan-7-one (25-2)

To a stirred solution of compound 25-1 (0.9 g, 1.70 mmol) in Toluene (10 mL) at 0° C., was added triphenylphosphine (0.66 g, 2.50 mmol) and DEAD (0.44 g, 2.50 mmol). The resulting reaction mixture was slowly warmed to room temperature and stirred at 100° C. for 16 h. After completion of the reaction (monitored by TLC), the reaction mixture was diluted with water and extracted with EtOAc. The combined organic layers were dried over anhydrous Na₂SO₄, filtered, and concentrated under reduced pressure. The crude compound was purified by Combi Flash chromatography (eluting with 40-50% EtOAc in heptane) to afford the title compound 25-2 (0.55 g, 63%) as a pale-yellow solid.

### Synthesis of 6-(5-(benzylthio)-4-methylthiazol-2-yl)-8-(2′,5′-difluoro-[1,1′-biphenyl]-4-yl)-2-oxa-6,8-diazaspiro[3.5]nonan-7-one (25-3)

To a stirred solution of compound 25-2 (0.5 g., 0.968 mmol) in 1,4 dioxane:H₂O (7:2 mL) was added (2,5-difluorophenyl)boronic acid (0.23 g, 1.45 mmol) followed by K₃PO₄ (0.4 g, 1.94 mmol) and the reaction mixture was purged under nitrogen for 10 min. PdCl₂(dppf) (35 mg., 0.0484 mmol) was added under a nitrogen atmosphere. The reaction mixture was heated at 100° C. for 16 h. The reaction mixture was filtered through Celite bed and washed with ethyl acetate. The filtrate was diluted with water and extracted with EtOAc then washed with brine. The combined organic layers were dried over anhydrous Na₂SO₄, filtered, and concentrated under reduced pressure. The crude compound obtained was purified by Combi Flash chromatography (eluting with 50-60% EtOAc in heptane) to afford the title compound 25-3 (0.11 g, 21.6%) as an off-white solid.

### Synthesis of 2-(8-(2′,5′-difluoro-[1,1′-biphenyl]-4-yl)-7-oxo-2-oxa-6,8-diazaspiro[3.5]nonan-6-yl)-4-methylthiazole-5-sulfonamide (339)

To a stirred solution of compound 25-3 (0.110 g, 0.200 mmol) in AcOH:H₂O (3:0.1 mL) was added N-chlorosuccinimide (93 mg, 0.701 mmol mmol) and then the reaction mixture was stirred at room temperature for 30 min. After completion of the reaction (monitored by TLC), the reaction mixture was concentrated under reduced pressure to dryness. The resulting residue was dissolved in THF (1 mL) and aqueous ammonia (2 mL) was added at 0° C. while stirring was continued at room temperature for another 1 h. The reaction mixture was diluted with water and extracted with ethyl acetate. The combined organic layers were dried over anhydrous Na₂SO₄, filtered. The crude compound obtained was purified by Combi Flash chromatography (eluting with 70-80% EtOAc in heptane) followed by preparative HPLC to afford the title compound 339 (13 mg, 13%) as an off-white solid.

### Example 26. Synthesis of (S)-2-(3-(2′,5′-difluoro-[1,1′-biphenyl]-4-yl)-5-(2-hydroxypropan-2-yl)-2-oxotetrahydropyrimidin-1(2H)-yl)-4-methylthiazole-5-sulfonamide (343) and (R)-2-(3-(2′,5′-difluoro-[1,1′-biphenyl]-4-yl)-5-(2-hydroxypropan-2-yl)-2-oxotetrahydropyrimidin-1(2H)-yl)-4-methylthiazole-5-sulfonamide (344)

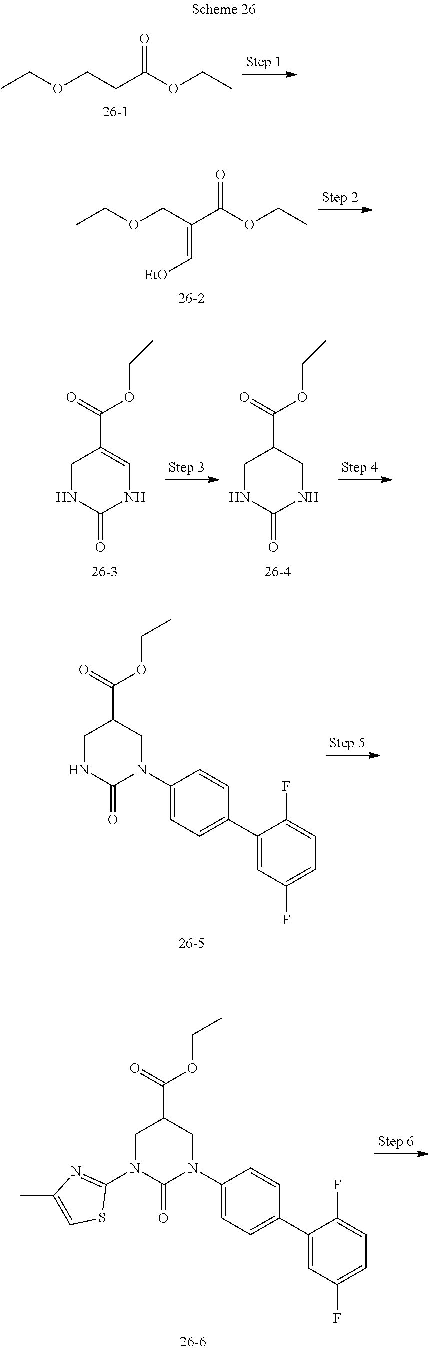

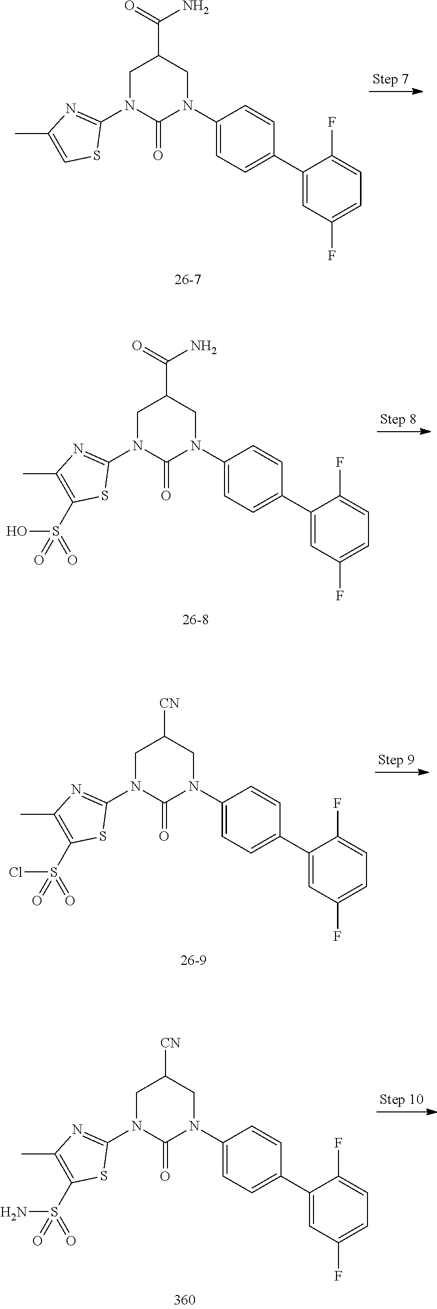 [SMILES: Cc1csc(N2CC(C(N)=O)CN(c3ccc(-c4cc(F)ccc4F)cc3)C2=O)n1 ; Cc1nc(N2CC(C(N)=O)CN(c3ccc(-c4cc(F)ccc4F)cc3)C2=O)sc1S(=O)(=O)O ; Cc1nc(N2CC(C#N)CN(c3ccc(-c4cc(F)ccc4F)cc3)C2=O)sc1S(=O)(=O)Cl ; Cc1nc(N2CC(C#N)CN(c3ccc(-c4cc(F)ccc4F)cc3)C2=O)sc1S(N)(=O)=O]

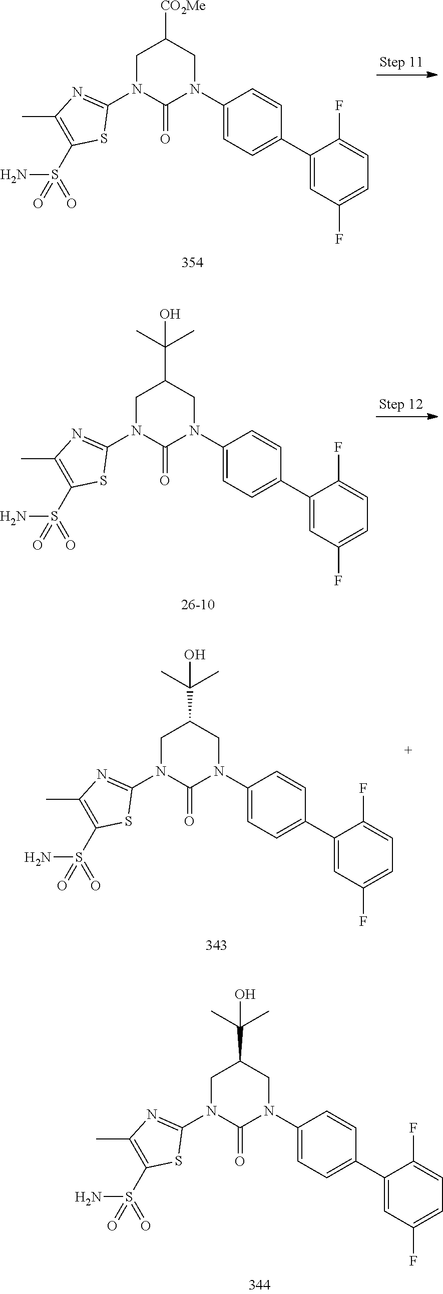 [SMILES: COC(=O)C1CN(c2ccc(-c3cc(F)ccc3F)cc2)C(=O)N(c2nc(C)c(S(N)(=O)=O)s2)C1 ; Cc1nc(N2CC(C(C)(C)O)CN(c3ccc(-c4cc(F)ccc4F)cc3)C2=O)sc1S(N)(=O)=O ; Cc1nc(N2C[C@H](C(C)(C)O)CN(c3ccc(-c4cc(F)ccc4F)cc3)C2=O)sc1S(N)(=O)=O ; Cc1nc(N2C[C@@H](C(C)(C)O)CN(c3ccc(-c4cc(F)ccc4F)cc3)C2=O)sc1S(N)(=O)=O]

### Synthesis of ethyl (E)-3-ethoxy-2-(ethoxymethyl)acrylate (26-2)

To a stirred solution of compound 26-1 (10 g, 68.493 mmol) in Toluene (120 mL) at 0° C. under a nitrogen atmosphere, Sodium ethoxide (9.3 g, 136.98 mmol) and ethyl formate (10 g, 136.98 mmol) were added, and the resulting reaction mixture was stirred at the same temperature for 2 h. To this reaction mixture, dimethyl sulfate (13 mL, 136.98 mmol) was added at 0° C. and then the reaction mixture was slowly warmed to room temperature and stirred at 50° C. for 16 h. After completion of the reaction (monitored by TLC), the reaction mixture was quenched with water and extracted with EtOAc. The combined organic layers were dried over anhydrous Na₂SO₄, filtered, and concentrated under reduced pressure to afford the title compound 26-2 (11 g, crude) as yellow oil. TLC: 20% EtOAc/heptane (R_{f} 0.2).

### Synthesis of ethyl 2-oxo-1,2,3,4-tetrahydropyrimidine-5-carboxylate (26-3)

To a stirred solution of compound 26-2 (11 g, 54.455 mmol) in ethanol (120 mL) was added urea (3.2 g, 54.455 mmol mmol) followed by conc.HCl (2.4 mL) and the resulting reaction mixture was heated at 80° C. for 16 h. The reaction mixture was concentrated under reduced pressure to dryness. The crude residue obtained was purified by trituration with ethanol. The obtained solid was filtered off and dried in vacuo to afford the title compound 26-3 (2.0 g, crude) as an off-white solid. TLC: 50% EtOAc/heptane (R_{f} 0.3) which was used in the next step without further purification.

### Synthesis of ethyl 2-oxohexahydropyrimidine-5-carboxylate (26-4)

An autoclave was charged with a solution of compound 26-3 (1.6 g, 9.090 mmol) in ethanol (50 mL) and the solution was purged under a nitrogen atmosphere for 10 min. 10% Pd/C (0.6 g) was added at room temperature under an inert atmosphere. The resulting reaction mixture was stirred at room temperature for 16 h under hydrogen atmosphere at 100 Psi. After completion of the reaction (monitored by TLC), the reaction mixture was filtered through a pad of Celite and washed with EtOAc. The filtrate was concentrated under reduced pressure to dryness to afford the title compound 26-4 (1.2 g, crude) as an off-white solid. TLC: 70% EtOAc/heptane (R_{f}: 0.5).

### Synthesis of ethyl 1-(2′,5′-difluoro-[1,1′-biphenyl]-4-yl)-2-oxohexahydropyrimidine-5-carboxylate (26-5)

To a stirred solution of compound 26-4 (1.2 g, 6.976 mmol) in toluene (20 mL) was added 4′-bromo-2,5-difluoro-1,1′-biphenyl (1.5 g, 5.581 mmol) and K₂CO₃ (2.4 g, 17.44 mmol) and the reaction mixture was purged under nitrogen for 10 min. XPhos (0.66 g, 1.395 mmol) and Pd₂(dba)₃ (0.63 g, 0.697 mmol) were added under a nitrogen atmosphere. The reaction mixture was heated at 110° C. for 16 h. After completion of the reaction (monitored by TLC), the reaction mixture was cooled to room temperature, diluted with water, and extracted with EtOAc then washed with brine. The combined organic layers were dried over anhydrous Na₂SO₄, filtered, and concentrated under reduced pressure. The crude compound obtained was purified by CombiFlash chromatography (eluting with 60-80% EtOAc in heptane) to afford the title compound 26-5 (0.7 g, 28%) as an off white solid. TLC: 70% EtOAc/heptane (R_{f} 0.5).

### Synthesis of ethyl 1-(2′,5′-difluoro-[1,1′-biphenyl]-4-yl)-3-(4-methylthiazol-2-yl)-2-oxohexahydropyrimidine-5-carboxylate (26-6)

To a stirred solution of compound 26-5 (0.7 g, 1.944 mmol) in toluene (12 mL) was added 2-bromo-4-methylthiazole (0.27 g, 1.555 mmol) and K₂CO₃ (0.64 g, 4.86 mmol) and the reaction mixture was purged under nitrogen for 10 min. XPhos (0.37 g, 0.777 mmol) and Pd₂(dba)₃ (0.35 g, 0.388 mmol) were added under a nitrogen atmosphere. The reaction mixture was heated at 110° C. for 16 h. After completion of the reaction (monitored by TLC), the reaction mixture was cooled to room temperature, diluted with water, and extracted with EtOAc then washed with brine. The combined organic layers were dried over anhydrous Na₂SO₄, filtered, and concentrated under reduced pressure. The crude compound obtained was purified by CombiFlash chromatography (eluting with 10-40% EtOAc in heptane) to afford the title compound 26-6 (0.5 g, 62.5%) as an off white solid. TLC: 50% EtOAc/heptane (R_{f} 0.5).

### Synthesis of 1-(2′,5′-difluoro-[1,1′-biphenyl]-4-yl)-3-(4-methylthiazol-2-yl)-2-oxohexahydropyrimidine-5-carboxamide (26-7)

A solution of compound 26-6 (0.2 g, 0.437 mmol) in 7M ammonia in methanol (10 mL) was heated at 70° C. for 16 h. After completion of the reaction (monitored by TLC), the reaction mixture was concentrated under reduced pressure. The crude compound obtained was purified by CombiFlash chromatography (eluting with 60-80% EtOAc in heptane) to afford the title compound 26-7 (0.14 g, 74.8%) as an off white solid. TLC: 70% EtOAc/heptane (R_{f} 0.5).

### Synthesis of 2-(5-carbamoyl-3-(2′,5′-difluoro-[1,1′-biphenyl]-4-yl)-2-oxotetrahydropyrimidin-1(2H)-yl)-4-methylthiazole-5-sulfonic acid (26-8)

To a stirred solution of compound 26-7 (0.2 g, 0.467 mmol) in dry DCM (5 mL) at 0° C. in an inert atmosphere, Chlorosulfonic acid (0.1 g, 0.934 mmol) was added, and the resulting reaction mixture was slowly warmed to room temperature and stirred for 16 h. The reaction mixture was concentrated under reduced pressure to dryness. The crude residue obtained was purified by trituration with diethyl ether. The obtained solid was filtered off and dried in vacuo to afford the title compound 26-8 (0.13 g, crude) as an off-white solid. TLC: 70% EtOAc/heptane (R_{f} 0.3) which was used in the next step without further purification.

### Synthesis of 2-(5-cyano-3-(2′,5′-difluoro-[1,1′-biphenyl]-4-yl)-2-oxotetrahydropyrimidin-1(2H)-yl)-4-methylthiazole-5-sulfonyl chloride (26-9)

To a stirred solution of compound 26-8 (0.1 g, 0.196 mmol) in 1,2-dichloroethane (2 mL) was added POCl₃ (1 mL) and the resulting reaction mixture was allowed to stir at 80° C. for 16 h. After completion of the reaction (monitored by TLC), the reaction mixture was concentrated under reduced pressure to dryness to afford the title compound 26-9 (0.1 g, crude) as a brown colored oil. TLC: 70% EtOAc in heptane (R_{f} 0.5). This compound was used as such without further purification.

### Synthesis of 2-(5-cyano-3-(2′,5′-difluoro-[1,1′-biphenyl]-4-yl)-2-oxotetrahydropyrimidin-1(2H)-yl)-4-methylthiazole-5-sulfonamide (360)

To a stirred solution of compound 26-9 (0.1 g 0.196 mmol) in THF (2 mL) at 0° C., aqueous ammonia (1 mL) was added dropwise. The resulting reaction mixture was slowly warmed to room temperature and allowed to stir for 2 h. After completion of the reaction (monitored by TLC), the reaction mixture was diluted with water and extracted with EtOAc. The combined organic layers were dried over anhydrous Na₂SO₄, filtered, and concentrated under reduced pressure. The crude product was purified by preparative HPLC to afford the title 360 (15 mg, 15.6%) as an off-white solid. TLC: 50% EtOAc/heptane (R_{f} 0.5).

### Synthesis of methyl 1-(2′,5′-difluoro-[1,1′-biphenyl]-4-yl)-3-(4-methyl-5-(methylsulfonyl)thiazol-2-yl)-2-oxohexahydropyrimidine-5-carboxylate (384)

To a stirred solution of compound 360 (0.2 g, 0.409 mmol) in MeOH:H₂O (4:1 mL) was added H₂SO₄ (2 mL) and the resulting reaction mixture was heated at 100° C. for 16 h. After completion of the reaction (monitored by TLC), the reaction mixture was cooled to room temperature, diluted with water, and extracted with EtOAc then washed with brine. The combined organic layers were dried over anhydrous Na₂SO₄, filtered, and concentrated under reduced pressure. The crude compound obtained was purified by CombiFlash chromatography (eluting with 0-80% EtOAc in heptane) followed by preparative HPLC to afford the title compound 384 (0.15 g, 70.4%) as an off white solid. TLC: 50% EtOAc/heptane (R_{f} 0.5).

### Synthesis of (R)-2-(3-(2′,5′-difluoro-[1,1′-biphenyl]-4-yl)-5-(2-hydroxypropan-2-yl)-2-oxotetrahydropyrimidin-1(2H)-yl)-4-methylthiazole-5-sulfonamide (343) and (R)-2-(3-(2′,5′-difluoro-[1,1′-biphenyl]-4-yl)-5-(2-hydroxypropan-2-yl)-2-oxotetrahydropyrimidin-1(2H)-yl)-4-methylthiazole-5-sulfonamide (344)

To a stirred solution of compound 384 (0.15 g, 0.287 mmol) in dry THF (5 mL) at −78° C. under an Argon atmosphere, Methyllithium solution (1.6 M in diethyl ether, 0.89 mL, 1.436 mmol) was added dropwise and the reaction mixture was stirred at the same temperature for 1 h. After completion of the reaction (monitored by TLC), the reaction mixture was quenched with saturated NH₄Cl solution and extracted with EtOAc. The combined organic layers were dried over anhydrous Na₂SO₄. filtered, and concentrated under reduced pressure. The crude compound was purified by CombiFlash chromatography (eluting with 0-70% EtOAc in heptane) to give 26-10. Preparative HPLC and SFC chiral separation of 26-10 afforded compound 343 (0.13 g, 86.6%) and compound 344 as an off-white solid, respectively. The stereochemistry was arbitrarily assigned. TLC: 70% EtOAc/heptane (R_{f}: 0.5).

### Example 27. Synthesis of 2-(3-(2′,5′-difluoro-[1,1′-biphenyl]-4-yl)-5-hydroxy-2-oxotetrahydropyrimidin-1(2H)-yl)-4-methylthiazole-5-sulfonamide (353)

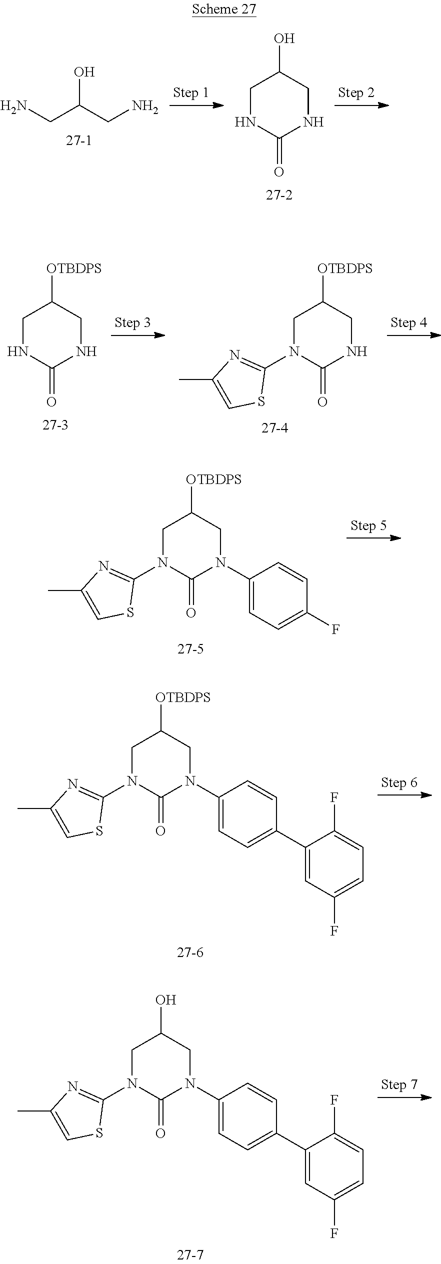

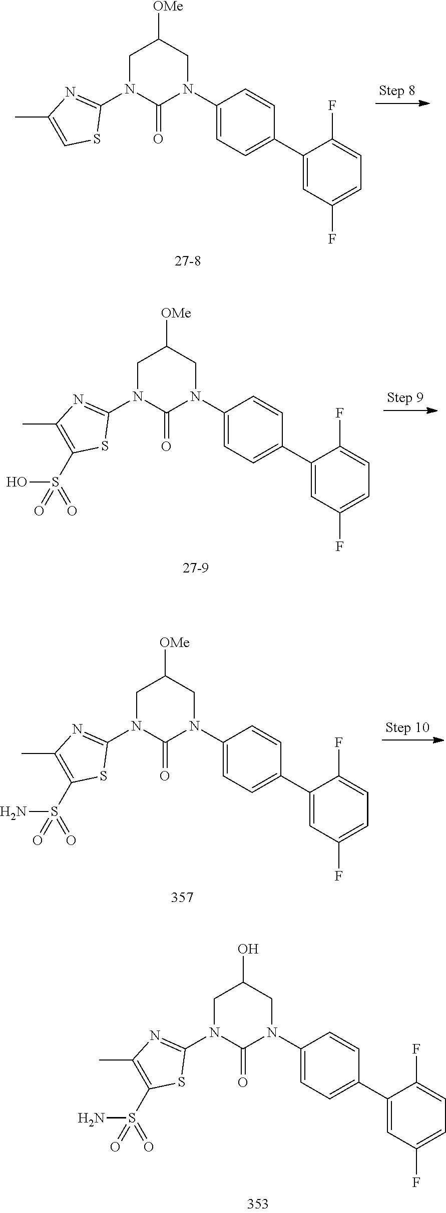 [SMILES: COC1CN(c2ccc(-c3cc(F)ccc3F)cc2)C(=O)N(c2nc(C)cs2)C1 ; COC1CN(c2ccc(-c3cc(F)ccc3F)cc2)C(=O)N(c2nc(C)c(S(=O)(=O)O)s2)C1 ; COC1CN(c2ccc(-c3cc(F)ccc3F)cc2)C(=O)N(c2nc(C)c(S(N)(=O)=O)s2)C1 ; Cc1nc(N2CC(O)CN(c3ccc(-c4cc(F)ccc4F)cc3)C2=O)sc1S(N)(=O)=O]

### Synthesis of 5-hydroxytetrahydropyrimidin-2(1H)-one (27-2)

To a stirred solution of compound 27-1 (10 g, 111.11 mmol) in 1,5,7-Triazabicyclodec-5-ene (0.77 g, 5.555 mmol) at 0° C. in an inert atmosphere, diethyl carbonate (1.57 g, 133.33 mmol) was added, and the resulting reaction mixture was slowly warmed to room temperature and stirred at 130° C. for 1 h. The reaction mixture was concentrated under reduced pressure to dryness. The crude residue obtained was purified by trituration with DCM and stirred for 1 h. The obtained solid was filtered off and dried in vacuo to afford the title compound 27-2 (5 g, crude) as an off-white solid. TLC: 100% EtOAc (R_{f} 0.2).

### Synthesis of 5-((tert-butyldiphenylsilyl)oxy)tetrahydropyrimidin-2(1H)-one (27-3)

To a stirred solution of compound 27-2 (5 g, 43.103 mmol) in DMF (80 mL) at 0° C. under a nitrogen atmosphere, Imidazole (5.8 g, 86.206 mmol) was added in small portions and the resulting reaction mixture was stirred at the same temperature for 10-15 min. To this reaction mixture, tert-butyl(chloro)diphenylsilane (17.7 g, 64.654 mmol) was added at 0° C. and then the reaction mixture was slowly warmed to room temperature and stirred for 16 h. After completion of the reaction (monitored by TLC), the reaction mixture was concentrated under reduced pressure. The crude compound obtained was purified by CombiFlash chromatography (eluting with 40-50% EtOAc in heptane) to afford the title compound 27-3 (10 g, 65.5%) as an off white solid. TLC: 50% EtOAc/heptane (R_{f} 0.5).

### Synthesis of 5-((tert-butyldimethylsilyl)oxy)-1-(4-methylthiazol-2-yl)tetrahydropyrimidin-2(1H)-one (27-4)

To a stirred solution of compound 27-3 (3.7 g, 10.451 mmol) in Toluene (10 mL) was added 2-bromo-4-methylthiazole (1.67 g, 9.406 mmol) followed by K₂CO₃ (3.6 g, 26.127 mmol) and the reaction mixture was purged under nitrogen for 10 min. Pd₂(dba)₃ (0.95 g, 1.045 mmol) and Xphos (0.24 g, 0.522 mmol) were added under a nitrogen atmosphere and the reaction mixture was heated at 110° C. for 24 h. After completion of the reaction (monitored by TLC), the reaction mixture was cooled to room temperature, filtered through a pad of Celite and washed with ethyl acetate. The filtrate was concentrated under reduced pressure. The crude product was purified by CombiFlash chromatography (eluting with 40-50% EtOAc in heptane) to afford the title compound 27-4 (1 g, 21.2%) as an off-white solid. TLC: 30% EtOAc/heptane (R_{f} 0.5).

### Synthesis of 5-((tert-butyldimethylsilyl)oxy)-1-(4-iodophenyl)-3-(4-methylthiazol-2-yl)tetrahydropyrimidin-2(1H)-one (27-5)

To a stirred solution of compound 27-41.0 g, 2.217 mmol) in 1, 4-dioxane (15 mL) were added 1-bromo-4-iodobenzene (0.941 g, 3.325 mmol), K₂CO₃ (0.61 g, 4.434 mmol) and 1,2-Dimethylethylenediamine (0.039 g, 0.443 mmol) the reaction mixture was purged under nitrogen for 10 min. To this resulting reaction mixture, CuI (42 mg, 0.221 mmol) was added under a nitrogen atmosphere. The reaction mixture was heated at 100° C. for 16 h. The reaction mixture was filtered through Celite bed and washed with ethyl acetate. The filtrate was diluted with water and extracted with EtOAc then washed with brine. The combined organic layers were dried over anhydrous Na₂SO₄, filtered, and concentrated under reduced pressure. The crude compound obtained was purified by CombiFlash chromatography (eluting with 20-30% EtOAc in heptane) to afford the title compound 27-5 (1.0 g, 68.96%) as an off white solid. TLC: 30% EtOAc/heptane (R_{f} 0.5).

### Synthesis of 5-((tert-butyldiphenylsilyl)oxy)-1-(2′,5′-difluoro-[1,1′-biphenyl]-4-yl)-3-(4-methylthiazol-2-yl)tetrahydropyrimidin-2(1H)-one (27-6)

To a stirred solution of compound 27-5 (1 g, 1.531 mmol) in 1, 4-dioxane and water (15:4 mL) were added (2,5-difluorophenyl)boronic acid (0.36 g, 2.296 mmol) and K₃PO₄ (0.811 g, 3.827 mmol) and the reaction mixture was purged under nitrogen for 10 min. PdCl₂(dppf) (0.11 g, 0.153 mmol) was added under a nitrogen atmosphere. The reaction mixture was heated at 110° C. for 16 h. After completion of the reaction (monitored by TLC), the reaction mixture was cooled to room temperature, diluted with water and extracted with EtOAc then washed with brine. The combined organic layers were dried over anhydrous Na₂SO₄, filtered, and concentrated under reduced pressure. The crude compound obtained was purified by CombiFlash chromatography (eluting with 20-30% EtOAc in heptane) to afford the title compound 27-6 (0.7 g, 71.5%) as an off white solid. TLC: 30% EtOAc/heptane (R_{f}: 0.5).

### Synthesis of 1-(2′,5′-difluoro-[1,1′-biphenyl]-4-yl)-5-hydroxy-3-(4-methylthiazol-2-yl)tetrahydropyrimidin-2(1H)-one (27-7)

To a stirred solution of compound 27-6 (0.7 g, 1.095 mmol) in dry THF (10 mL) at 0° C. under a nitrogen atmosphere, Tetrabutylammonium fluoride (1M solution in THF, 0.572 mL, 2.190 mmol) was added dropwise. The resulting reaction mixture was slowly warmed to room temperature and stirred for 2 h. After completion of the reaction (monitored by TLC), the reaction mixture was quenched with ice cold water and extracted with EtOAc. The combined organic layers were dried over anhydrous Na₂SO₄, filtered, and concentrated under reduced pressure. The crude compound was purified by CombiFlash chromatography (eluting with 20-30% EtOAc in heptane) to afford the title compound 27-7 (0.4 g, 91.1%) as an off white solid. TLC: 30% EtOAc/heptane (R_{f} 0.5).

### Synthesis of 1-(2′,5′-difluoro-[1,1′-biphenyl]-4-yl)-5-methoxy-3-(4-methylthiazol-2-yl)tetrahydropyrimidin-2(1H)-one (27-8)

To a stirred solution of compound 27-7 (0.4 g, 0.997 mmol) in THF (5 mL) at 0° C. under a nitrogen atmosphere, NaH (60% w/w in mineral oil, 79.8 mg, 1.995 mmol) was added in small portions and the resulting reaction mixture was stirred at the same temperature for 5-10 min. To this reaction mixture, Methyl iodide (0.28 g, 1.995 mmol) was added at 0° C. and then the reaction mixture was slowly warmed to room temperature and stirred for 2 h. After completion of the reaction (monitored by TLC), the reaction mixture was quenched with water and extracted with EtOAc. The combined organic layers were dried over anhydrous Na₂SO₄, filtered, and concentrated under reduced pressure. The crude compound was purified by CombiFlash chromatography (eluting with 20-30% EtOAc in heptane) to afford the title compound 27-8 (0.2 g, 48.3%) as an off white solid. TLC: 30% EtOAc/heptane (R_{f} 0.5).

### Synthesis of 2-(3-(2′,5′-difluoro-[1,1′-biphenyl]-4-yl)-5-methoxy-2-oxotetrahydropyrimidin-1(2H)-yl)-4-methylthiazole-5-sulfonic acid (27-9)

To a stirred solution of compound 27-8 (0.15 g, 0.361 mmol) in dry DCM (4 mL) at 0° C. in an inert atmosphere, Chlorosulfonic acid (84 mg, 0.722 mmol) was added, and the resulting reaction mixture was slowly warmed to room temperature and stirred for 16 h. After completion of the reaction (monitored by TLC), the reaction mixture was concentrated under reduced pressure to dryness. The crude residue obtained was purified by trituration with diethyl ether. The obtained solid was filtered off and dried in vacuo to afford the title compound 27-9 (0.15 g, crude) as an off-white solid. TLC: 50% EtOAc/heptane (R_{f} 0.3) which was used in the next step without further purification.

### Synthesis of 2-(3-(2′,5′-difluoro-[1,1′-biphenyl]-4-yl)-5-methoxy-2-oxotetrahydropyrimidin-1(2H)-yl)-4-methylthiazole-5-sulfonamide (357)

A mixture of compound 27-9 (0.15 g, 0.303 mmol) and POCl₃ (2 mL) was allowed to stir at 90° C. for 6 h. The reaction mixture was concentrated under reduced pressure to dryness. The resulting residue was dissolved in THF (1 mL) and aqueous ammonia (2 mL) was added at 0° C. while stirring was continued at room temperature for another 16 h. The reaction mixture was diluted with water and extracted with EtOAc. The combined organic layers were dried over anhydrous Na₂SO₄, filtered, and concentrated under reduced pressure. The crude compound was purified by preparative HPLC to afford the title compound 357 (12 mg, 8%) as an off-white solid. TLC: 50% EtOAc/heptane (R_{f} 0.5).

### Synthesis of 2-(3-(2′,5′-difluoro-[1,1′-biphenyl]-4-yl)-5-hydroxy-2-oxotetrahydropyrimidin-1(2H)-yl)-4-methylthiazole-5-sulfonamide (353)

To a stirred solution of 357 (0.32 g, 0.646 mmol) in DMF (1 mL) at 0° C. under a nitrogen atmosphere, Boron tribromide solution (1.0 M in DCM, 10 mL 10.343 mmol) was added dropwise. The resulting reaction mixture was slowly warmed to room temperature and stirred at 100° C. for 48 h. After completion of the reaction (monitored by TLC), the reaction mixture was concentrated under reduced pressure. The crude compound was purified by CombiFlash chromatography (eluting with 2-5% MeOH in DCM) followed by preparative HPLC to afford the title compound 353 (10 mg, 3.2%) as an off white solid. TLC: 5% MeOH/DCM (R_{f}: 0.3).

### Example 28: Synthesis of 2-(3-(2′,5′-difluoro-[1,1′-biphenyl]-4-yl)-2-oxotetrahydropyrimidin-1(2H)-yl)-N-(2-hydroxyethyl)-4-methylthiazole-5-sulfonamide (388)

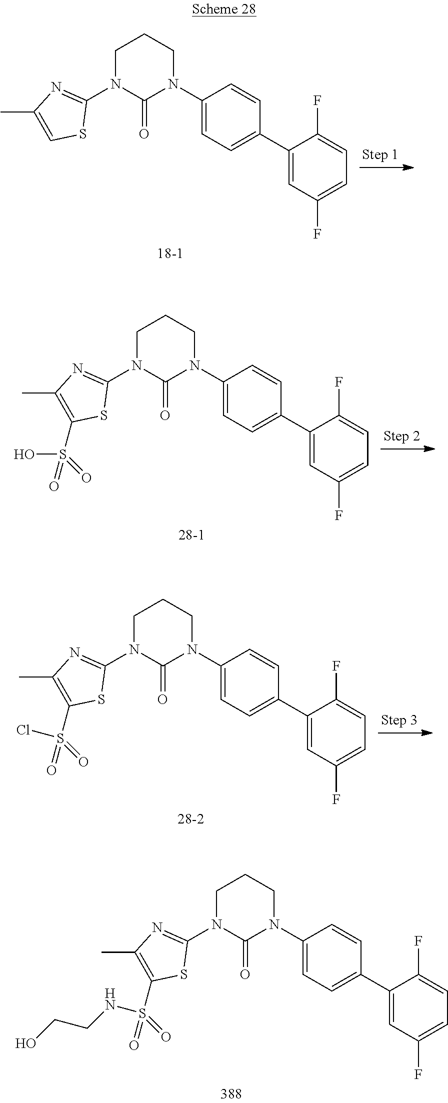 [SMILES: Cc1csc(N2CCCN(c3ccc(-c4cc(F)ccc4F)cc3)C2=O)n1 ; Cc1nc(N2CCCN(c3ccc(-c4cc(F)ccc4F)cc3)C2=O)sc1S(=O)(=O)O ; Cc1nc(N2CCCN(c3ccc(-c4cc(F)ccc4F)cc3)C2=O)sc1S(=O)(=O)Cl ; Cc1nc(N2CCCN(c3ccc(-c4cc(F)ccc4F)cc3)C2=O)sc1S(=O)(=O)NCCO]

### Synthesis of 2-(3-(2′,5′-difluoro-[1,1′-biphenyl]-4-yl)-2-oxotetrahydropyrimidin-1(2H)-yl)-4-methylthiazole-5-sulfonic acid (28-1)

To a stirred solution of compound 18-1 (4 g, 10.389 mmol) in dry DCM (40 mL) at 0° C. in an inert atmosphere, chlorosulfuric acid (2.07 mL, 31.168 mmol) was added and the resulting reaction mixture was slowly warmed to room temperature and stirred for 12 h. The reaction mixture was concentrated under reduced pressure to dryness. The crude residue obtained was purified by trituration with diethyl ether. The obtained solid was filtered off and dried in vacuo to afford the title compound 28-1 (3.35 g, crude) as an off-white solid. TLC: 100% EtOAc (R_{f} 0.2).

### Synthesis of 2-(3-(2′,5′-difluoro-[1,1′-biphenyl]-4-yl)-2-oxotetrahydropyrimidin-1(2H)-yl)-4-methylthiazole-5-sulfonyl chloride (28-2)

A stirred solution of compound 28-1 (4 g, 8.602 mmol) in POCl₃ (20 mL) was allowed to stir at 100° C. for 12 h. After completion of the reaction (monitored by TLC), the reaction mixture was concentrated under reduced pressure to dryness to afford the title compound 28-2 (3.5 g, crude) as a brown colored solid. TLC: 50% EtOAc in heptane (R_{f}. 0.5). This compound was used as such for the next step without further purification.

### Synthesis of 2-(3-(2′,5′-difluoro-[1,1′-biphenyl]-4-yl)-2-oxotetrahydropyrimidin-1(2H)-yl)-N-(2-hydroxyethyl)-4-methylthiazole-5-sulfonamide (388)

To a stirred solution of compound 28-2 (0.6 g, 1.242 mmol) in THF (6 mL) at 0° C. was added DIPEA (0.65 mL, 3.726 mmol) followed by 2-aminoethan-1-ol (0.14 g, 1.863 mmol). The resulting reaction mixture was slowly warmed to room temperature and stirred for 1 h. After completion of the reaction (monitored by TLC), the reaction mixture was quenched with water and extracted with EtOAc. The combined organic layers were dried over anhydrous Na₂SO₄, filtered, and concentrated under reduced pressure. The crude compound obtained was purified by CombiFlash chromatography (eluting with 50-60% EtOAc in heptane) followed by preparative HPLC to afford the title compound 388 (16 mg, 2.5%) as an off-white solid. TLC: 50% EtOAc/Heptane (R_{f}: 0.5).

### Example 29: Synthesis of 1-(5-((difluoromethyl)sulfonyl)-4-methylthiazol-2-yl)-3-(2′-fluoro-[1,1′-biphenyl]-4-yl)tetrahydropyrimidin-2(1H)-one (412)

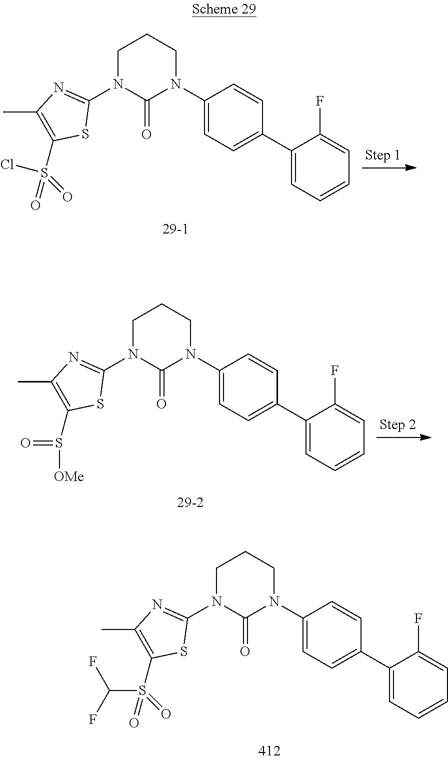 [SMILES: Cc1nc(N2CCCN(c3ccc(-c4ccccc4F)cc3)C2=O)sc1S(=O)(=O)Cl ; COS(=O)c1sc(N2CCCN(c3ccc(-c4ccccc4F)cc3)C2=O)nc1C ; Cc1nc(N2CCCN(c3ccc(-c4ccccc4F)cc3)C2=O)sc1S(=O)(=O)C(F)F]

### Synthesis of sodium 2-(3-(2′-fluoro-[1,1′-biphenyl]-4-yl)-2-oxotetrahydropyrimidin-1(2H)-yl)-4-methylthiazole-5-sulfinate (29-2)

A stirred solution of sodium sulfite (62.1 mg, 0.483 mmol) in water (10 ml) was stirred at RT for 10 minutes. sodium bicarbonate (83 mg, 0.966 mmol) was added, and the resulting solution was stirred at 50° C. for 1 h. Compound 29-1 (300 mg, 0.483 mmol) was added portion wise to the solution and the reaction mixture was continued to stir at 50° C. for 16 h. After completion of the reaction, as monitored by TLC, the reaction mixture was concentrated under a vacuum to remove the solvent. To the residue was added Methanol (5 mL) and stirred for 30 minutes. The solids formed were removed through filtration, and the filtrate was concentrated under a vacuum to afford sodium salt of compound 29-2 (300 mg, 90%) as a yellow solid.

LCMS: 1.26 min, 66.06%, 433.0 [M+H]⁺, (Column: Acquity BEH C₁₈ (50×2.1 mm) 1.7 μm), Mobile phase A: 0.1% Formic acid in H₂O, Mobile phase B: 0.05% Formic acid in ACN, Flow Rate: 1.0 mL/min.

Note: Sulfonic acid mass was observed by LCMS.

### Synthesis of 1-(5-((difluoromethyl)sulfonyl)-4-methylthiazol-2-yl)-3-(2′-fluoro-[1,1′-biphenyl]-4-yl)tetrahydropyrimidin-2(1H)-one (412)

To a stirred solution of KOH (247 mg, 4.41 mmol) in water (3.00 mL) was added compound 29-2 (100 mg, 0.221 mmol) in Acetonitrile (3 mL). Then the reaction mixture was cooled to 0-5° C. and stirred for 10 minutes vigorously and diethyl (bromodifluoromethyl)phosphonate (177 mg, 0.662 mmol) and the reaction mixture was allowed to stir at RT for 16 h. The reaction was monitored by TLC. The reaction mixture was diluted with water (10 mL) and extracted with EtOAc (2×20 mL). The combined organic extract was washed with brine solution (20 mL), dried over anhydrous sodium sulfate, filtered, and concentrated. The crude was purified by preparative HPLC (ABC method) to afford compound 412 (1.9 mg, 1.8%) as an off white solid. LCMS: 2.22 min, 98.02%, 482.0 (M+H)⁺, (Column: XBridge C8 (50×4.6 mm) 3.5 m, Mobile phase: A: 0.1% TFA in H₂O, Mobile phase: B: Acetonitrile, Flow Rate: 2.0 ml/min)

HPLC: 6.86 min, 98.90%, (Column: XBridge C8 (50×4.6 mm) 3.5 μm, Mobile phase: A: 5 mM Ammonium acetate in H₂O, Mobile phase: B: Acetonitrile, Flow Rate: 2.0 ml/min)

### Example 30: Synthesis of Synthesis of 2-(3-(2′,5′-difluoro-[1,1′-biphenyl]-4-yl)-2-oxotetrahydropyrimidin-1(2H)-yl)-4-(fluoromethyl)thiazole-5-sulfonamide (441)

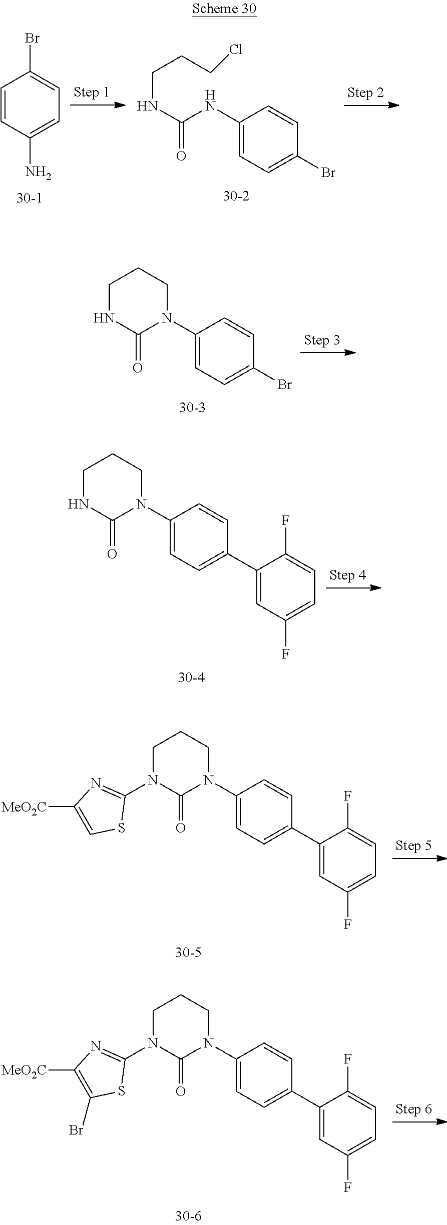 [SMILES: Nc1ccc(Br)cc1 ; O=C(NCCCCl)Nc1ccc(Br)cc1 ; O=C1NCCCN1c1ccc(Br)cc1 ; O=C1NCCCN1c1ccc(-c2cc(F)ccc2F)cc1 ; COC(=O)c1csc(N2CCCN(c3ccc(-c4cc(F)ccc4F)cc3)C2=O)n1 ; COC(=O)c1nc(N2CCCN(c3ccc(-c4cc(F)ccc4F)cc3)C2=O)sc1Br]

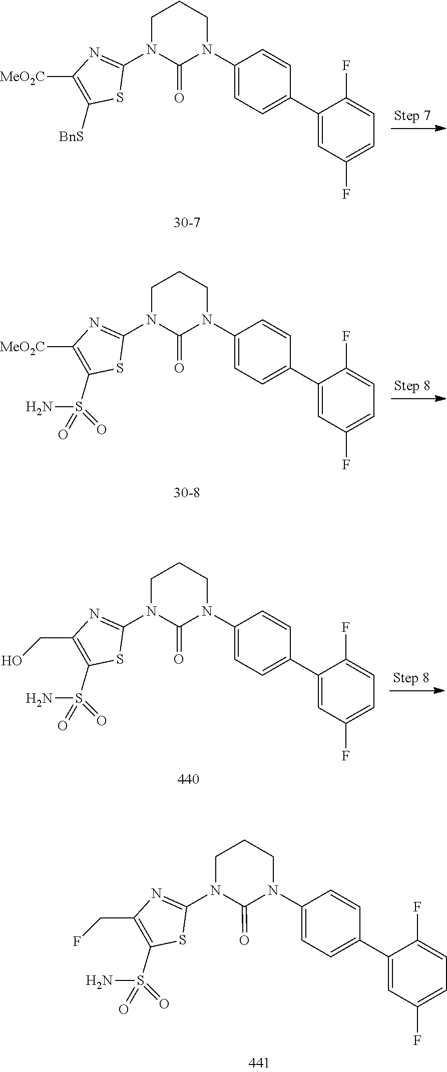

### Synthesis of 1-(4-bromophenyl)-3-(3-chloropropyl)urea (30-2)

To a stirred solution of compound 30-1 (5 g, 28.089 mmol) in THE (80 mL) at 0° C. under a nitrogen atmosphere, 1-chloro-3-isocyanatopropane (5 g, 42.134 mmol) was added and the resulting reaction mixture was slowly warmed to room temperature and stirred at 85° C. for 16 h. The reaction mixture was diluted with water and extracted with ethyl acetate. The combined organic layers were dried over anhydrous Na₂SO₄, filtered, and concentrated under reduced pressure to afford the title compound 30-2 (6 g, crude) as an off-white solid, which was used in the next step without further purification.

### Synthesis of 1-(4-bromophenyl)tetrahydropyrimidin-2(1H)-one (30-3)

To a stirred solution of compound 30-2 (6 g, 20.689 mmol) in acetonitrile (90 mL) was added K₂CO₃ (5.7 g, 41.379 mmol) followed by TBAB (3.3 g, 10.344 mmol). The resulting reaction mixture was heated at 90° C. for 16 h. After completion of the reaction (monitored by TLC), the reaction mixture was cooled to room temperature, diluted with water, and extracted with EtOAc. The combined organic layers were dried over anhydrous Na₂SO₄, filtered, and concentrated under reduced pressure. The crude product was purified by CombiFlash chromatography (eluting with 70-80% EtOAc in heptane) to afford the title compound 30-3 (4 g, 76.9%) as an off-white solid.

### Synthesis of 1-(2′,5′-difluoro-[1,1′-biphenyl]-4-yl)tetrahydropyrimidin-2(1H)-one (30-4)

To a stirred solution of compound 30-3 (2.7 g, 11.00 mmol) in 1,4 dioxane:H₂O (20:5 mL) was added (2,5-difluorophenyl)boronic acid (2 g, 13.00 mmol) followed by K₃PO₄ (6.7 g, 32 mmol) and the reaction mixture was purged under nitrogen for 10 min. PdCl₂(dppf) (0.81 g, 1.1 mmol) was added under a nitrogen atmosphere. The reaction mixture was heated at 100° C. for 12 h. After completion of the reaction (monitored by TLC), the reaction mixture was cooled to room temperature, filtered through a pad of Celite and washed with ethyl acetate. The filtrate was diluted with water and extracted with EtOAc. The combined organic layers were dried over anhydrous Na₂SO₄, filtered, and concentrated under reduced pressure. The crude product was purified by CombiFlash column chromatography [eluting with 70-80% EtOAc in heptane] to afford the title compound 30-4 (1 g, 33%) as an off-white solid.

### Synthesis of methyl 2-(3-(2′,5′-difluoro-[1,1′-biphenyl]-4-yl)-2-oxotetrahydropyrimidin-1(2H)-yl)thiazole-4-carboxylate (30-5)

To a stirred solution of compound 30-4 (2 g, 6.937 mmol) in 1,4-dioxane (40 mL) was added compound 7 (2.31 g, 10.41 mmol) followed by CS₂CO₃ (5.65 g, 17.34 mmol) and the reaction mixture was purged under nitrogen for 10 min. Xantphos (0.4 g, 0.693 mmol) and Pd₂(dba)₃ (0.6 g, 0.693 mmol) were added under a nitrogen atmosphere. The reaction mixture was heated at 120° C. for 16 h. After completion of the reaction (monitored by TLC), the reaction mixture was cooled to room temperature, filtered through a pad of Celite and washed with ethyl acetate. The filtrate was diluted with water and extracted with EtOAc then washed with brine. The combined organic layers were dried over anhydrous Na₂SO₄, filtered, and concentrated under reduced pressure. The crude compound obtained was purified by CombiFlash chromatography (eluting with 50-100% EtOAc in heptane) to afford the title compound 30-5 (1.55 g, 52%) as an off-white solid.

### Synthesis of methyl 5-bromo-2-(3-(2′,5′-difluoro-[1,1′-biphenyl]-4-yl)-2-oxotetrahydropyrimidin-1(2H)-yl)thiazole-4-carboxylate (30-6)

To a stirred solution of compound 30-5 (1.5 g, 3.50 mmol) in DCM (30 mL) was added N-Bromosuccinimide (1.3 g, 7.00 mmol). The resulting reaction mixture was heated at 55° C. for 2 h. After completion of the reaction (monitored by TLC), the reaction mixture was cooled to room temperature, diluted with water, and extracted with DCM. The combined organic layers were dried over anhydrous Na₂SO₄, filtered, and concentrated under reduced pressure. The crude product was purified by CombiFlash chromatography (eluting with 40-50% EtOAc in heptane) to afford the title compound 30-6 (1.60 g, 90%) as an off-white solid.

### Synthesis of methyl 5-(benzylthio)-2-(3-(2′,5′-difluoro-[1,1′-biphenyl]-4-yl)-2-oxotetrahydropyrimidin-1(2H)-yl)thiazole-4-carboxylate (30-7)

To a stirred solution of compound 30-6 (1.5 g, 3.00 mmol) in 1,4-dioxane (25 mL) was added phenylmethanethiol (0.92 g, 7.4 mmol) followed by DIPEA (1.2 g, 8.90 mmol) and the reaction mixture was purged under nitrogen for 10 min. Xantphos (0.18 g, 0.30 mmol) and Pd₂(dba)₃ (0.14 g, 0.15 mmol) were added under a nitrogen atmosphere. The reaction mixture was heated at 110° C. for 36 h. After completion of the reaction (monitored by TLC), the reaction mixture was cooled to room temperature, filtered through a pad of Celite and washed with ethyl acetate. The filtrate was diluted with water and extracted with EtOAc then washed with brine. The combined organic layers were dried over anhydrous Na₂SO₄, filtered, and concentrated under reduced pressure. The crude compound obtained was purified by CombiFlash chromatography (eluting with 60-70% EtOAc in heptane) to afford the title compound 30-7 (1.4 g, 86%) as a yellow solid.

### Synthesis of methyl 2-(3-(2′,5′-difluoro-[1,1′-biphenyl]-4-yl)-2-oxotetrahydropyrimidin-1(2H)-yl)-5-sulfamoylthiazole-4-carboxylate (30-8)

To a stirred solution of compound 30-7 (1 g, 1.80 mmol) in AcOH:H₂O (8:0.1 mL) was added N-chlorosuccinimide (0.75 g, 5.40 mmol) and then the reaction mixture was stirred at room temperature for 30 min. After completion of the reaction (monitored by TLC), the reaction mixture was concentrated under reduced pressure to dryness. The resulting residue was dissolved in THE (20 mL) and aqueous ammonia (10 mL) was added at 0° C. while stirring was continued at room temperature for another 2 h. The reaction mixture was diluted with water and extracted with ethyl acetate. The combined organic layers were dried over anhydrous Na₂SO₄, filtered to afford the title compound 30-8 (0.5 g, 54%) as an off-white solid.

### Synthesis of 2-(3-(2′,5′-difluoro-[1,1′-biphenyl]-4-yl)-2-oxotetrahydropyrimidin-1(2H)-yl)-4-(hydroxymethyl)thiazole-5-sulfonamide (440)

To a stirred solution of compound 30-8 (0.15 g, 0.295 mmol) in THE (4 mL) at 0° C. under a nitrogen atmosphere, Lithium borohydride (14 mg, 0.590 mmol) was added. The resulting reaction mixture was stirred at the same temperature for 5-10 min and then the reaction mixture was slowly warmed to room temperature and stirred for 2 h. After completion of the reaction (monitored by TLC), the reaction mixture was quenched with water and extracted with EtOAc. The combined organic layers were dried over anhydrous Na₂SO₄, filtered, and concentrated under reduced pressure. The crude compound was purified by preparative HPLC to afford the title compound 440 (10 mg, 7%) as an off-white solid.

### Synthesis of 2-(3-(2′,5′-difluoro-[1,1′-biphenyl]-4-yl)-2-oxotetrahydropyrimidin-1(2H)-yl)-4-(fluoromethyl)thiazole-5-sulfonamide (441)

To a stirred solution of compound 440 (0.2 g, 0.416 mmol) in DCM (7 mL) at 0° C. under a nitrogen atmosphere, DAST (0.21 g, 1.249 mmol) was added. The resulting reaction mixture was stirred at the same temperature for 5-10 min and then the reaction mixture was slowly warmed to room temperature and stirred for 2 h. After completion of the reaction (monitored by TLC), the reaction mixture was quenched with water and extracted with EtOAc. The combined organic layers were dried over anhydrous Na₂SO₄, filtered, and concentrated under reduced pressure. The crude compound was purified by preparative HPLC to afford the title compound 441 (10 mg, 4.9%) as an off-white solid.

Table 1 shows structures and analytical data of representative exemplified compounds of the present invention. These compounds can be prepared according to the synthetic schemes described above and using procedures known to those of ordinary skill in the art.

TABLE 1
Representative exemplified compounds of the present invention

| Compound | | MS | |
|---|---|---|---|
| Number | Structure | [M + 1]⁺ | ¹H NMR |
| 1 | 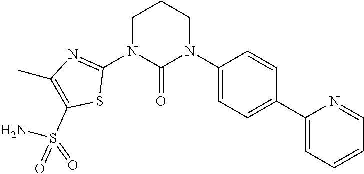 [SMILES: Cc1nc(N2CCCN(c3ccc(-c4ccccn4)cc3)C2=O)sc1S(N)(=O)=O] | 429.9 | ¹H NMR (400 MHz, DMSO-d₆): δ 8.65-8.64 (m, 1H), 8.12 (d, J = 8.0 Hz, 2H), 7.97-7.83 (m, 2H), 7.56 (s, 2H), 7.50 (d, J = 8.4 Hz, 2H), 7.40- 7.31 (m, 1H), 4.15-4.14 (m, 2H), 3.80-3.70 (m, 2H), 2.45 (s, 3H), 2.30- 2.2-0 (m, 2 H) ppm |
| 2 | 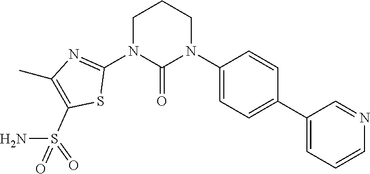 [SMILES: Cc1nc(N2CCCN(c3ccc(-c4cccnc4)cc3)C2=O)sc1S(N)(=O)=O] | 430.0 | ¹H NMR (400 MHz, DMSO-d₆): δ 8.92 (s, 1H), 8.58 (d, J = 4.9 Hz, 1H), 8.10 (d, J = 7.8 Hz, 1H), 7.78 (d, J = 8.3 Hz, 2H), 7.56 (s, 2H), 7.54- 7.49 (m, 3H), 4.17 (t, J = 5.6 Hz, 2H), 3.85-3.79 (m, 2H), 2.45 (s, 3H), 2.28-2.20 (m, 2H) ppm |
| 3 | 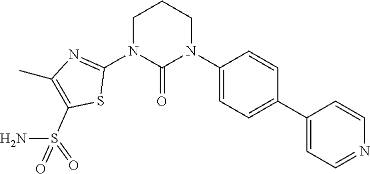 [SMILES: Cc1nc(N2CCCN(c3ccc(-c4ccncc4)cc3)C2=O)sc1S(N)(=O)=O] | 429.9 | ¹H NMR (400 MHz, DMSO-d₆): δ 8.69-8.60 (m, 2H), 7.86 (d, J = 8.6 Hz, 2H), 7.79-7.68 (m, 2H), 7.61-7.49 (m, 4H), 4.17 (t, J = 5.9 Hz, 2H), 3.83 (t, J = 5.6 Hz, 2H), 2.45 (s, 3H), 2.28-2.19 (m, 2H) ppm |
| 4 | 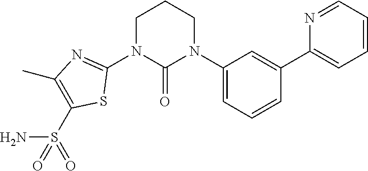 [SMILES: Cc1nc(N2CCCN(c3cccc(-c4ccccn4)c3)C2=O)sc1S(N)(=O)=O] | 430.1 | ¹H NMR (400 MHz, DMSO-d₆): δ 8.68 (dd, J = 0.8, 4.8 Hz, 1H), 8.12 (t, J = 1.8 Hz, 1H), 8.02- 7.96 (m, 2H), 7.93-7.88 (m, 1H), 7.57-7.51 (m, 3H), 7.47-7.42 (m, 1H), 7.42-7.34 (m, 1H), 4.18 (t, J = 5.9 Hz, 2H), 3.84 (t, J = 5.6 Hz, 2H), 2.45 (s, 3H), 2.30-2.20 (m, 2H) ppm |
| 5 | 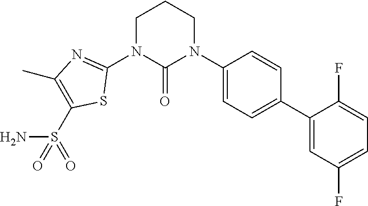 [SMILES: Cc1nc(N2CCCN(c3ccc(-c4cc(F)ccc4F)cc3)C2=O)sc1S(N)(=O)=O] | 464.9 | ¹H NMR (400 MHz, DMSO-d₆): δ 7.65-7.59 (m, 2H), 7.55 (br s, 2H), 7.53-7.48 (m, 2H), 7.48- 7.36 (m, 2H), 7.31-7.25 (m, 1H), 4.17 (t, J = 6.1 Hz, 2H), 3.82 (t, J = 5.6 Hz, 2H), 2.45 (s, 3H), 2.29-2.18 (m, 2H) ppm |
| 6 | 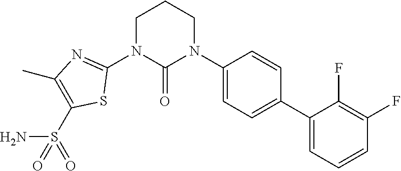 [SMILES: Cc1nc(N2CCCN(c3ccc(-c4cccc(F)c4F)cc3)C2=O)sc1S(N)(=O)=O] | 465.4 | ¹H NMR (400 MHz, DMSO-d₆): δ 7.62 (d, J = 7.5 Hz, 2H), 7.58-7.50 (m, 4H), 7.48-7.29 (m, 3H), 4.23-4.09 (m, 2H), 3.86-3.79 (m, 2H), 2.45 (s, 3H), 2.28-2.20 (m, 2H) ppm |
| 7 | 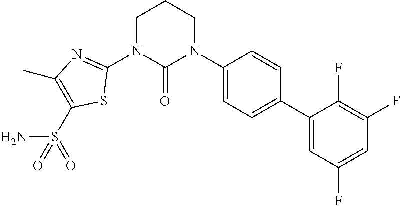 [SMILES: Cc1nc(N2CCCN(c3ccc(-c4cc(F)cc(F)c4F)cc3)C2=O)sc1S(N)(=O)=O] | 483.2 | ¹H NMR (400 MHz, DMSO-d₆): δ 7.68-7.63 (m, 2H), 7.63-7.49 (m, 5H), 7.40-7.32 (m, 1H), 4.17 (t, J = 6.0 Hz, 2H), 3.84 (t, J = 5.6 Hz, 2H), 2.45 (s, 3H), 2.29-2.20 (m, 2H) ppm |
| 8 | 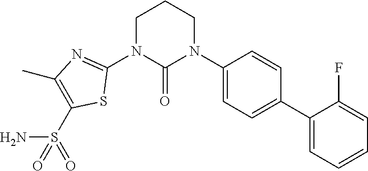 [SMILES: Cc1nc(N2CCCN(c3ccc(-c4ccccc4F)cc3)C2=O)sc1S(N)(=O)=O] | 446.9 | ¹H NMR (400 MHz, DMSO-d₆): δ 7.62-7.53 (m, 5H), 7.52-7.41 (m, 3H), 7.36-7.29 (m, 2H), 4.17 (t, J = 5.9 Hz, 2H), 3.82 (t, J = 5.6 Hz, 2H), 2.45 (s, 3H), 2.29-2.19 (m, 2H) ppm |
| 9 | 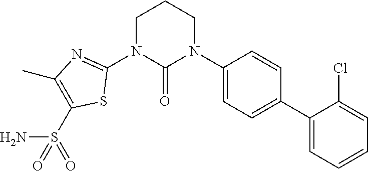 [SMILES: Cc1nc(N2CCCN(c3ccc(-c4ccccc4Cl)cc3)C2=O)sc1S(N)(=O)=O] | 463.0 | ¹H NMR (400 MHz, DMSO-d₆): δ 7.60-7.55 (m, 3H), 7.52-7.37 (m, 7H), 4.17 (t, J = 5.9 Hz, 2H), 3.86-3.79 (m, 2H), 2.45 (s, 3H), 2.30-2.16 (m, 2H) ppm |
| 10 | 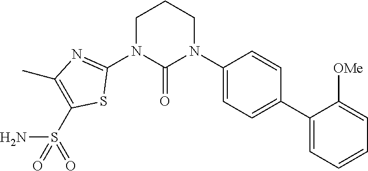 [SMILES: COc1ccccc1-c1ccc(N2CCCN(c3nc(C)c(S(N)(=O)=O)s3)C2=O)cc1] | 459.4 | ¹H NMR (400 MHz, DMSO-d₆): δ 7.57-7.54 (m, 2H), 7.54-7.47 (m, 2H), 7.42-7.30 (m, 4H), 7.13 (d, J = 7.6 Hz, 1H), 7.07-7.01 (m, 1H), 4.17 (t, J = 6.0 Hz, 2H), 3.83- 3.80 (m, 2H), 3.79 (s, 3H), 2.45 (s, 3H), 2.26- 2.19 (m, 2H) ppm |
| 11 | 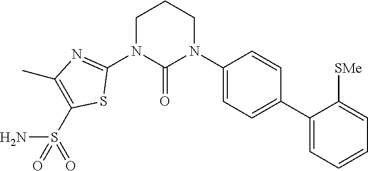 [SMILES: CSc1ccccc1-c1ccc(N2CCCN(c3nc(C)c(S(N)(=O)=O)s3)C2=O)cc1] | 475.5 | ¹H NMR (400 MHz, DMSO-d₆): δ 7.57 (s, 2H), 7.47-7.35 (m, 6H), 7.26-7.20 (m, 2H), 4.17 (t, J = 6.1 Hz, 2H), 3.82 (t, J = 5.6 Hz, 2H), 2.45 (s, 3H), 2.39 (s, 3H), 2.27- 2.19 (m, 2H) ppm |
| 12 | 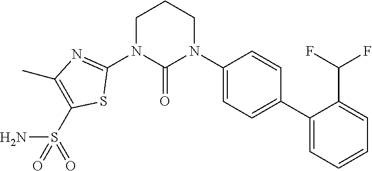 [SMILES: Cc1nc(N2CCCN(c3ccc(-c4ccccc4C(F)F)cc3)C2=O)sc1S(N)(=O)=O] | 479.2 | ¹H NMR (400 MHz, DMSO-d₆): δ 7.78-7.70 (m, 1H), 7.66-7.52 (m, 4H), 7.52-7.44 (m, 2H), 7.43-7.34 (m, 3H), 6.98- 6.64 (t, J = 54 Hz, 1H), 4.19-4.10 (m, 2H), 3.86- 3.77 (m, 2H), 2.43 (s, 3H), 2.27-2.18 (m, 2H) ppm |
| 13 | 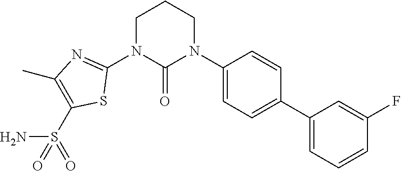 [SMILES: Cc1nc(N2CCCN(c3ccc(-c4cccc(F)c4)cc3)C2=O)sc1S(N)(=O)=O] | 446.9 | ¹H NMR (400 MHz, DMSO-d₆): δ 7.75 (d, J = 8.6 Hz, 2H), 7.61-7.44 (m, 7H), 7.25-7.16 (m, 1H), 4.16 (t, J = 6.0 Hz, 2H), 3.81 (t, J = 5.6 Hz, 2H), 2.45 (s, 3H), 2.29- 2.17 (m, 2H) ppm |
| 14 | 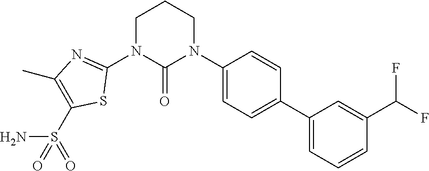 [SMILES: Cc1nc(N2CCCN(c3ccc(-c4cccc(C(F)F)c4)cc3)C2=O)sc1S(N)(=O)=O] | 479.1 | ¹H NMR (400 MHz, DMSO-d₆): δ 7.92-7.83 (m, 2H), 7.75 (d, J = 8.5 Hz, 2H), 7.67-7.61 (m, 1H), 7.60-7.54 (m, 3H), 7.54-7.48 (m, 2H), 7.10 (t, J = 56 Hz, 1H), 4.17 (t, J = 5.9 Hz, 2H), 3.82 (t, J = 5.5 Hz, 2H), 2.45 (s, 3H), 2.27-2.20 (m, 2H) ppm |
| 15 | 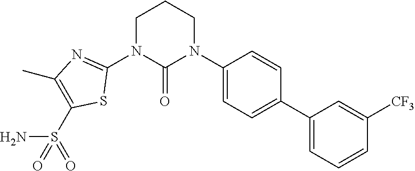 [SMILES: Cc1nc(N2CCCN(c3ccc(-c4cccc(C(F)(F)F)c4)cc3)C2=O)sc1S(N)(=O)=O] | 497.2 | ¹H NMR (400 MHz, DMSO-d₆): δ 8.04-7.97 (m, 2H), 7.82-7.78 (m, 2H), 7.78-7.68 (m, 2H), 7.56 (s, 2H), 7.54-7.49 (m, 2H), 4.17 (t, J = 6.0 Hz., 2H), 3.82 (t, J = 5.6 Hz, 2H), 2.45 (s, 3H), 2.27-2.21 (m, 2H) ppm |
| 16 | 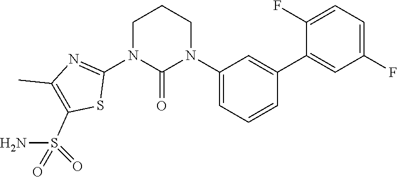 [SMILES: Cc1nc(N2CCCN(c3cccc(-c4cc(F)ccc4F)c3)C2=O)sc1S(N)(=O)=O] | 464.9 | ¹H NMR (400 MHz, DMSO-d₆): δ 7.61 (s, 1H), 7.58-7.36 (m, 7H), 7.36-7.22 (m, 1H), 4.16 (t, J = 5.6 Hz, 2H), 3.92- 3.75 (m, 2H), 2.44 (s, 3H), 2.29-2.17 (m, 2H) ppm |
| 17 | 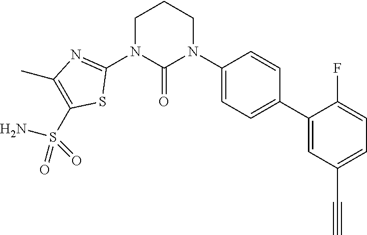 [SMILES: C#Cc1ccc(F)c(-c2ccc(N3CCCN(c4nc(C)c(S(N)(=O)=O)s4)C3=O)cc2)c1] | 471.2 | ¹H NMR (400 MHz, DMSO-d₆): δ 7.67-7.58 (m, 3H), 7.57-7.48 (m, 5H), 7.37 (dd, J = 8.5, 10.6 Hz, 1H), 4.23 (s, 1H), 4.16 (t, J = 6.0 Hz, 2H), 3.83 (t, J = 5.6 Hz, 2H), 2.45 (s, 3H), 2.29- 2.18 (m, 2H) ppm |
| 18 | 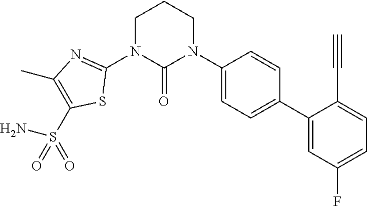 [SMILES: C#Cc1ccc(F)cc1-c1ccc(N2CCCN(c3nc(C)c(S(N)(=O)=O)s3)C2=O)cc1] | 471.2 | ¹H NMR (400 MHz, DMSO-d₆): δ 7.70-7.61 (m, 3H), 7.56 (s, 2H), 7.53-7.44 (m, 2H), 7.36- 7.30 (m, 1H), 7.29- 7.22 (m, 1H), 4.22-4.11 (m, 3H), 3.83 (t, J = 5.5 Hz, 2H), 2.45 (s, 3H), 2.28-2.19 (m, 2H) ppm |
| 19 | 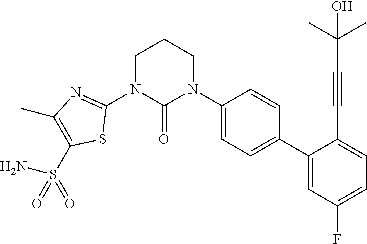 [SMILES: Cc1nc(N2CCCN(c3ccc(-c4cc(F)ccc4C#CC(C)(C)O)cc3)C2=O)sc1S(N)(=O)=O] | 527.1 | ¹H NMR (400 MHz, DMSO-d₆): δ 7.76-7.61 (m, J = 8.0 Hz, 2H), 7.55 (s, 3H), 7.52-7.43 (m, 2H), 7.40-7.30 (m, 1H), 7.29-7.15 (m, 1H), 5.34 (s, 1H), 4.23-4.11 (m, 2H), 3.88-3.76 (m, 2H), 2.45 (s, 3H), 2.29-2.15 (m, 2H), 1.36 (s, 6H) ppm |
| 20 | 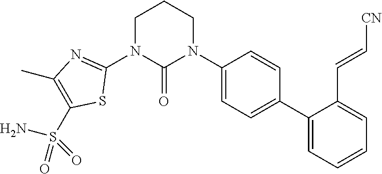 [SMILES: Cc1nc(N2CCCN(c3ccc(-c4ccccc4/C=C/C#N)cc3)C2=O)sc1S(N)(=O)=O] | 480.2 | ¹H NMR (400 MHz, DMSO-d₆): δ 7.87 (d, J = 7.8 Hz, 1H), 7.62-7.42 (m, 7H), 7.42-7.32 (m, 3H), 6.48 (d, J = 16.6 Hz, 1H), 4.23-4.13 (m, 2H), 3.91-3.79 (m, 2H), 2.45 (s, 3H), 2.32-2.16 (m, 2H) ppm |
| 21 | 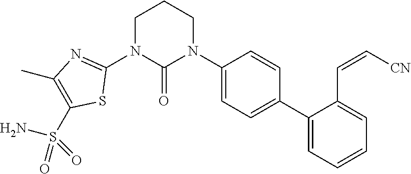 [SMILES: Cc1nc(N2CCCN(c3ccc(-c4ccccc4/C=C\C#N)cc3)C2=O)sc1S(N)(=O)=O] | 480.2 | ¹H NMR (400 MHz, DMSO-d₆): δ 7.96 (d, J = 7.3 Hz, 1H), 7.62-7.52 (m, 4H), 7.52-7.47 (m, 3H), 7.42-7.33 (m, 2H), 7.26-7.20 (m, 1H), 5.94 (d, J = 11.7 Hz, 1H), 4.20- 4.14 (m, 2H), 3.87- 3.79 (m, 2H), 2.45 (s, 3H), 2.28-2.20 (m, 2H) ppm |
| 22 | 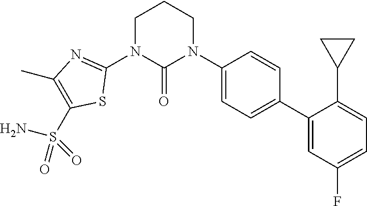 [SMILES: Cc1nc(N2CCCN(c3ccc(-c4cc(F)ccc4C4CC4)cc3)C2=O)sc1S(N)(=O)=O] | 487.3 | ¹H NMR (400 MHz, DMSO-d₆): δ 7.55 (s, 2H), 7.52-7.45 (m, 4H), 7.14-7.00 (m, 3H), 4.17 (t, J = 6.0 Hz, 2H), 3.83 (t, J = 5.6 Hz, 2H), 2.45 (s, 3H), 2.28-2.19 (m, 2H), 1.86-1.76 (m, 1H), 0.88-0.81 (m, 2H), 0.69- 0.64 (m, 2H) ppm |
| 23 | 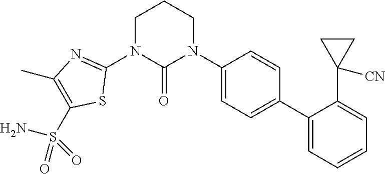 [SMILES: Cc1nc(N2CCCN(c3ccc(-c4ccccc4C4(C#N)CC4)cc3)C2=O)sc1S(N)(=O)=O] | 494.5 | ¹H NMR (400 MHz, DMSO-d₆): δ 7.60-7.30 (m, 10H), 4.17 (t, J = 5.9 Hz, 2H), 3.86 (t, J = 5.4 Hz, 2H), 2.45 (s, 3H), 2.29-2.21 (m, 2H), 1.50- 1.39 (m, 2H), 1.24- 1.10 (m, 2H) ppm |
| 24 | 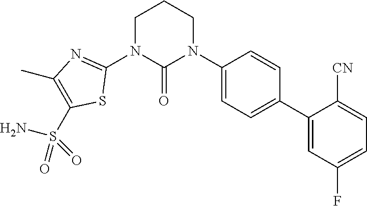 [SMILES: Cc1nc(N2CCCN(c3ccc(-c4cc(F)ccc4C#N)cc3)C2=O)sc1S(N)(=O)=O] | 472.3 | ¹H NMR (400 MHz, DMSO-d₆): δ 8.08 (dd, J = 5.6, 8.6 Hz, 1H), 7.71- 7.64 (m, 2H), 7.61-7.54 (m, 5H), 7.53-7.43 (m, 1H), 4.17 (t, J = 6.0 Hz, 2H), 3.85 (t, J = 5.6 Hz, 2H), 2.45 (s, 3H), 2.28- 2.20 (m, 2H) ppm |
| 25 | 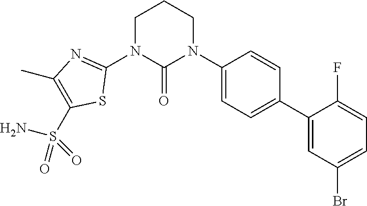 [SMILES: Cc1nc(N2CCCN(c3ccc(-c4cc(Br)ccc4F)cc3)C2=O)sc1S(N)(=O)=O] | 526.6 | ¹H NMR (400 MHz, DMSO-d₆): δ 7.74 (dd, J = 2.5, 6.9 Hz, 1H), 7.65- 7.58 (m, 3H), 7.56 (s, 2H), 7.54-7.47 (m, 2H), 7.34 (dd, J = 8.9, 10.4 Hz, 1H), 4.16 (t, J = 5.9 Hz, 2H), 3.82 (t, J = 5.6 Hz, 2H), 2.45 (s, 3H), 2.32-2.15 (m, 2H) ppm |
| 26 | 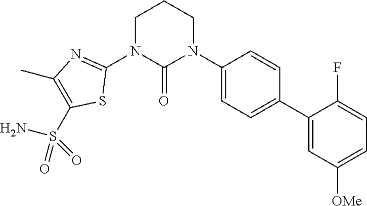 [SMILES: COc1ccc(F)c(-c2ccc(N3CCCN(c4nc(C)c(S(N)(=O)=O)s4)C3=O)cc2)c1] | 477.2 | ¹H NMR (400 MHz, DMSO-d₆): δ 7.64-7.58 (m, 2H), 7.56 (s, 2H), 7.51-7.46 (m, 2H), 7.29- 7.21 (m, 1H), 7.10- 7.03 (m, 1H), 7.00-6.92 (m, 1H), 4.20-4.14 (m, 2H), 3.86-3.81 (m, 2H), 3.80 (s, 3H), 2.45 (s, 3H), 2.28-2.18 (m, 2H) ppm |
| 27 | 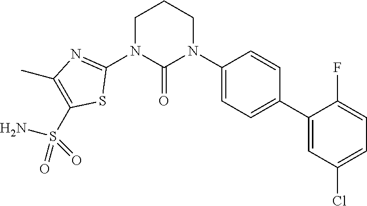 [SMILES: Cc1nc(N2CCCN(c3ccc(-c4cc(Cl)ccc4F)cc3)C2=O)sc1S(N)(=O)=O] | 481.2 | ¹H NMR (400 MHz, DMSO-d₆): δ 7.62 (d, J = 7.3 Hz, 3H), 7.56 (brs, 2H), 7.53-7.45 (m, 3H), 7.44-7.35 (m, 1H), 4.21- 4.12 (m, 2H), 3.86- 3.78 (m, 2H), 2.45 (s, 3H), 2.28-2.18 (m, 2H) ppm |
| 28 | 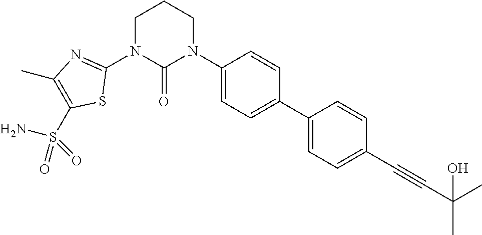 [SMILES: Cc1nc(N2CCCN(c3ccc(-c4ccc(C#CC(C)(C)O)cc4)cc3)C2=O)sc1S(N)(=O)=O] | 510.7 | ¹H NMR (400 MHz, DMSO-d₆): δ 7.75-7.68 (m, 4H), 7.55 (s, 2H), 7.52-7.44 (m, 4H), 5.48 (s, 1H), 4.16 (t, J = 5.9 Hz, 2H), 3.81 (t, J = 5.6 Hz, 2H), 2.44 (s, 3H), 2.26-2.19 (m, 2H), 1.48 (s, 6H) ppm |
| 29 | 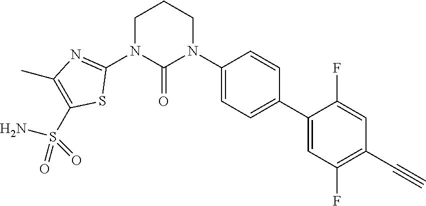 [SMILES: C#Cc1cc(F)c(-c2ccc(N3CCCN(c4nc(C)c(S(N)(=O)=O)s4)C3=O)cc2)cc1F] | 489.2 | ¹H NMR (400 MHz, DMSO-d₆): δ 7.67-7.62 (m, 2H), 7.62-7.55 (m, 3H), 7.55-7.49 (m, 3H), 4.68 (s, 1H), 4.16 (t, J = 6.0 Hz, 2H), 3.82 (t, J = 5.6 Hz, 2H), 2.44 (s, 3H), 2.26-2.21 (m, 2H) ppm |
| 30 | 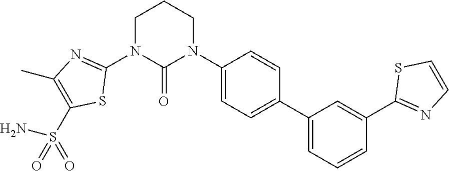 [SMILES: Cc1nc(N2CCCN(c3ccc(-c4cccc(-c5nccs5)c4)cc3)C2=O)sc1S(N)(=O)=O] | 512.0 | ¹H NMR (400 MHz, DMSO-d₆): δ 8.20 (s, 1H), 8.00-7.92 (m, 2H), 7.86-7.77 (m, 4H), 7.66- 7.59 (m, 1H), 7.58- 7.49 (m, 4H), 4.21-4.13 (m, 2H), 3.86-3.80 (m, 2H), 2.45 (s, 3H), 2.29- 2.20 (m, 2H) ppm |
| 31 | 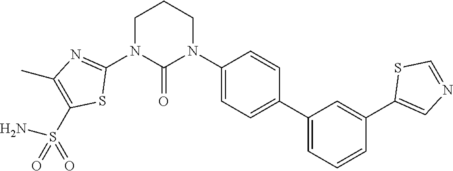 [SMILES: Cc1nc(N2CCCN(c3ccc(-c4cccc(-c5cncs5)c4)cc3)C2=O)sc1S(N)(=O)=O] | 512.2 | ¹H NMR (400 MHz, DMSO-d₆): δ 9.13 (s, 1H), 8.46 (s, 1H), 7.97 (s, 1H), 7.80 (d, J = 8.6 Hz, 2H), 7.74-7.63 (m, 2H), 7.60-7.47 (m, 5H), 4.17 (t, J = 5.9 Hz, 2H), 3.83 (t, J = 5.6 Hz, 2H), 2.45 (s, 3H), 2.28-2.21 (m, 2H) ppm |
| 32 | 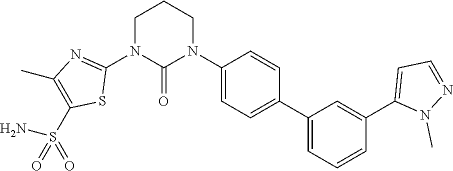 [SMILES: Cc1nc(N2CCCN(c3ccc(-c4cccc(-c5ccnn5C)c4)cc3)C2=O)sc1S(N)(=O)=O] | 509.2 | ¹H NMR (400 MHz, DMSO-d₆): δ 7.81-7.73 (m, 4H), 7.64-7.58 (m, 1H), 7.58-7.52 (m, 3H), 7.52-7.46 (m, 3H), 6.51 (d, J = 1.9 Hz, 1H), 4.17 (t, J = 6.0 Hz, 2H), 3.91 (s, 3H), 3.82 (t, J = 5.7 Hz, 2H), 2.45 (s, 3H), 2.28-2.19 (m, 2H) ppm |
| 33 | 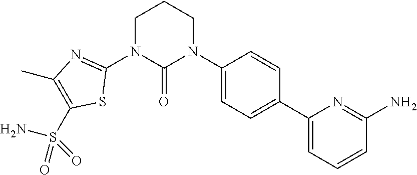 [SMILES: Cc1nc(N2CCCN(c3ccc(-c4cccc(N)n4)cc3)C2=O)sc1S(N)(=O)=O] | 445.2 | ¹H NMR (400 MHz, CD₃OD-d₄): δ 7.99 (dd, J = 7.4, 8.8 Hz, 1H), 7.84 (d, J = 8.8 Hz, 2H), 7.64 (d, J = 8.6 Hz, 2H), 7.22- 7.17 (m, 1H), 6.99 (d, J = 8.9 Hz, 1H), 4.29 (t, J = 6.1 Hz, 2H), 3.91 (t, J = 5.7 H7, 2H), 2.53 (s, 3H), 2.40-2.28 (m, 2H) ppm |
| 34 | 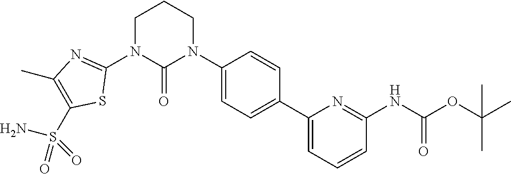 [SMILES: Cc1nc(N2CCCN(c3ccc(-c4cccc(NC(=O)OC(C)(C)C)n4)cc3)C2=O)sc1S(N)(=O)=O] | 545.1 | ¹H NMR (400 MHz, DMSO-d₆): δ 9.76 (s, 1H), 8.11 (d, J = 8.8 Hz, 2H), 7.87-7.78 (m, 1H), 7.76-7.70 (m, 1H), 7.62 (d, J = 7.8 Hz, 1H), 7.56 (s, 2H), 7.53-7.45 (m, 2H), 4.16 (t, J = 5.9 Hz, 2H), 3.83 (t, J = 5.1 Hz, 2H), 2.45 (s, 3H), 2.28- 2.18 (m, 2H), 1.49 (s, 9H) ppm |
| 35 | 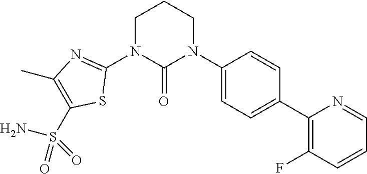 [SMILES: Cc1nc(N2CCCN(c3ccc(-c4ncccc4F)cc3)C2=O)sc1S(N)(=O)=O] | 448.0 | ¹H NMR (400 MHz, DMSO-d₆): δ 8.57-8.55 (m, 1H), 7.96 (dd, J = 1.4, 8.6 Hz, 2H), 7.92- 7.78 (m, 1H), 7.61-7.46 (m, 5H), 4.17 (t, J = 5.9 Hz, 2H), 3.85 (t, J = 5.6 Hz, 2H), 2.45 (s, 3H), 2.30-2.19 (m, 2H) ppm |
| 36 | 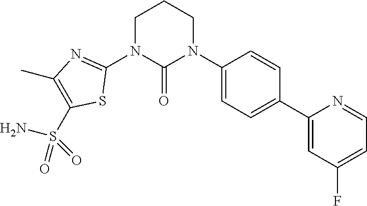 [SMILES: Cc1nc(N2CCCN(c3ccc(-c4cc(F)ccn4)cc3)C2=O)sc1S(N)(=O)=O] | 448.2 | ¹H NMR (400 MHz, DMSO-d₆): δ 8.70 (dd, J = 5.6, 9.1 Hz, 1H), 8.20- 8.14 (m, 2H), 7.95 (dd, J = 2.4, 11.1 Hz, 1H), 7.58- 7.48 (m, 4H), 7.32-7.27 (m, 1H), 4.16 (t, J = 6.1 Hz, 2H), 3.84 (t, J = 5.6 Hz, 2H), 2.45 (s, 3H), 2.23 (quin, J = 5.8 Hz, 2H) ppm |
| 37 | 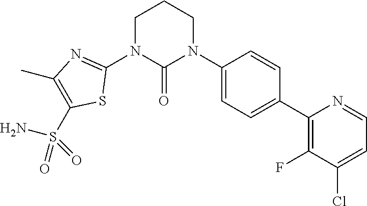 [SMILES: Cc1nc(N2CCCN(c3ccc(-c4nccc(Cl)c4F)cc3)C2=O)sc1S(N)(=O)=O] | 481.9 | ¹H NMR (400 MHz, DMSO-d₆): δ 8.52 (d, J = 4.9 Hz, 1H), 7.95 (d, J = 7.3 Hz, 2H), 7.77-7.72 (m, 1H), 7.57 (d, J = 8.3 Hz, 2H), 4.17 (t, J = 5.4 Hz, 2H), 3.85 (t, J = 5.1 Hz, 2H), 2.45 (s, 3H), 2.28-2.20 (m, 2H) ppm(Note: -NH₂ not observed) |
| 38 | 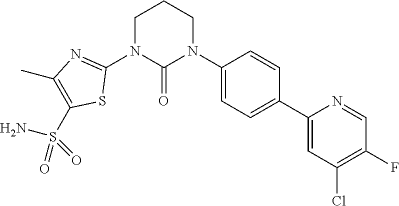 [SMILES: Cc1nc(N2CCCN(c3ccc(-c4cc(Cl)c(F)cn4)cc3)C2=O)sc1S(N)(=O)=O] | 481.9 | ¹H NMR (400 MHz, DMSO-d₆): δ 8.79 (s, 1H), 8.35 (d, J = 5.9 Hz, 1H), 8.13 (d, J = 8.3 Hz, 2H), 7.52 (d, J = 8.3 Hz, 2H), 4.21-4.12 (m, 2H), 3.88-3.79 (m, 2H), 2.45 (s, 3H), 2.28-2.18 (m, 2H) ppm. (Note: -NH₂ not observed) |
| 39 | 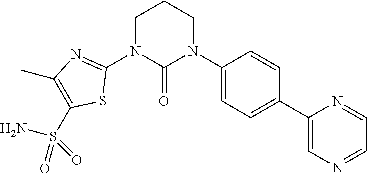 [SMILES: Cc1nc(N2CCCN(c3ccc(-c4cnccn4)cc3)C2=O)sc1S(N)(=O)=O] | 431.0 | ¹H NMR (400 MHz, DMSO-d₆): δ 9.28 (s, 1H), 8.73 (s, 1H), 8.62 (s, 1H), 8.19 (d, J = 8.8 Hz, 2H), 7.57 (s, 4H), 4.22- 4.13 (m, 2H), 3.93-3.80 (m, 2H), 2.45 (s, 3H), 2.29-2.19 (m, 2H) ppm |
| 40 | 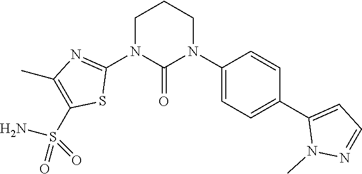 [SMILES: Cc1nc(N2CCCN(c3ccc(-c4ccnn4C)cc3)C2=O)sc1S(N)(=O)=O] | 433.4 | ¹H NMR (400 MHz, DMSO-d₆): δ 7.60-7.55 (m, 4H), 7.53-7.45 (m, 3H), 6.42 (d, J = 1.9 Hz, 1H), 4.16 (t, J = 5.6 Hz, 2H), 3.88 (s, 3H), 3.86- 3.79 (m, 2H), 2.45 (s, 3H), 2.29-2.18 (m, 2H) ppm |
| 41 | 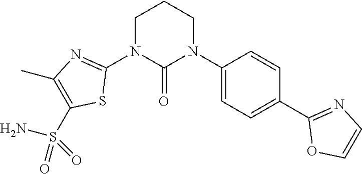 [SMILES: Cc1nc(N2CCCN(c3ccc(-c4ncco4)cc3)C2=O)sc1S(N)(=O)=O] | 420.2 | ¹H NMR (400 MHz, DMSO-d₆): δ 8.23 (d, J = 0.6 Hz, 1H), 8.04-7.98 (m, 2H), 7.61-7.52 (m, 4H), 7.42-7.36 (m, 1H), 4.16 (t, J = 5.9 Hz, 2H), 3.83 (t, J = 5.6 Hz, 2H), 2.44 (s, 3H), 2.29-2.18 (m, 2H) ppm |
| 42 | 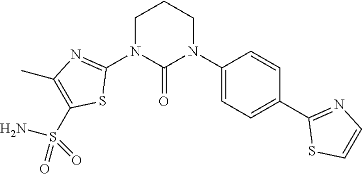 [SMILES: Cc1nc(N2CCCN(c3ccc(-c4nccs4)cc3)C2=O)sc1S(N)(=O)=O] | 435.9 | ¹H NMR (400 MHz, DMSO-d₆): δ 8.00-7.97 (m, 2H), 7.94 (d, J = 3.3 Hz, 1H), 7.80 (d, J = 3.1 Hz, 1H), 7.59-7.49 (m, 4H), 4.16 (t, J = 6.0 Hz, 2H), 3.83 (t, J = 5.6 Hz, 2H), 2.45 (s, 3H), 2.29- 2.18 (m, 2H) ppm |
| 43 | 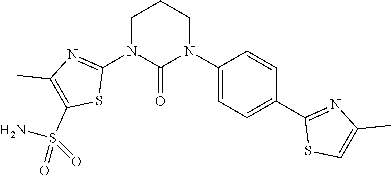 | 450.5 | ¹H NMR (400 MHz, DMSO-d₆): δ 7.97-7.91 (m, 2H), 7.56 (s, 2H), 7.54-7.47 (m, 2H), 7.33 (d, J = 1.0 Hz, 1H), 4.16 (t, J = 6.0 Hz, 2H), 3.82 (t, J = 5.6 Hz, 2H), 2.45 (s, 3H), 2.43 (s, 3H), 2.28- 2.17 (m, 2H) ppm |
| 44 | 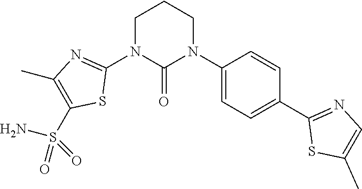 [SMILES: Cc1cnc(-c2ccc(N3CCCN(c4nc(C)c(S(N)(=O)=O)s4)C3=O)cc2)s1] | 450.1 | ¹H NMR (400 MHz, DMSO-d₆): δ 7.95-7.84 (m, 2H), 7.64-7.58 (m, 1H), 7.56 (s, 2H), 7.52- 7.48 (m, 2H), 4.16 (t, J = 6.0 Hz, 2H), 3.82 (t, J = 5.6 Hz, 2H), 2.45 (s, 3H), 2.27-2.18 (m, 2H) ppm (Note: -3H merged in solvent peak) |
| 45 | 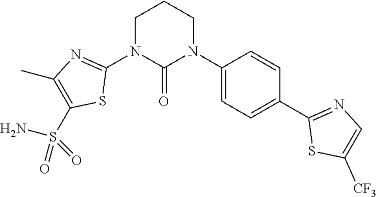 [SMILES: Cc1nc(N2CCCN(c3ccc(-c4ncc(C(F)(F)F)s4)cc3)C2=O)sc1S(N)(=O)=O] | 503.9 | ¹H NMR (400 MHz, DMSO-d₆): δ 8.54 (d, J = 1.3 Hz, 1H), 8.09-8.03 (m, 2H), 7.63-7.54 (m, 4H), 4.16 (t, J = 5.9 Hz, 2H), 3.85 (t, J = 5.6 Hz, 2H), 2.45 (s, 3H), 2.29- 2.19 (m, 2H) ppm |
| 46 | 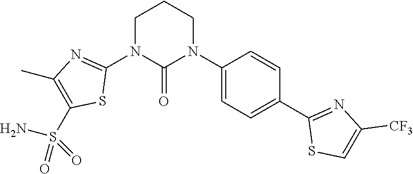 [SMILES: Cc1nc(N2CCCN(c3ccc(-c4nc(C(F)(F)F)cs4)cc3)C2=O)sc1S(N)(=O)=O] | 504.2 | ¹H NMR (400 MHz, DMSO-d₆): δ 8.56 (d, J = 0.9 Hz, 1H), 8.06-8.00 (m, 2H), 7.61-7.54 (m, 4H), 4.16 (t, J = 6.0 Hz, 2H), 3.84 (t, J = 5.6 Hz, 2H), 2.45 (s, 3H), 2.30- 2.18 (m, 2H) ppm |
| 47 | 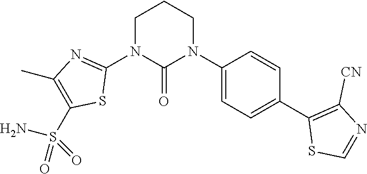 [SMILES: Cc1nc(N2CCCN(c3ccc(-c4scnc4C#N)cc3)C2=O)sc1S(N)(=O)=O] | 460.9 | ¹H NMR (400 MHz, DMSO-d₆): δ 9.28 (s, 1H), 7.82 (d, J = 8.3 Hz, 2H), 7.62 (d, J = 8.3 Hz, 2H), 7.57 (brs, 2H), 4.23- 4.11 (m, 2H), 3.91- 3.80 (m, 2H), 2.45 (s, 3H), 2.27-2.20 (m, 2H) ppm |
| 48 | 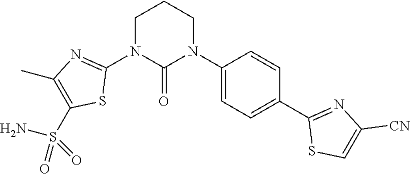 [SMILES: Cc1nc(N2CCCN(c3ccc(-c4nc(C#N)cs4)cc3)C2=O)sc1S(N)(=O)=O] | 460.9 | ¹H NMR (400 MHz, DMSO-d₆): δ 8.91 (s, 1H), 8.04 (d, J = 8.3 Hz, 2H), 7.58 (d, J = 8.8 Hz, 2H), 4.19-4.12 (m, 2H), 3.89-3.80 (m, 2H), 2.45 (s, 3H), 2.29-2.18 (m, 2H) ppm (Note: -NH₂ not observed) |
| 49 | 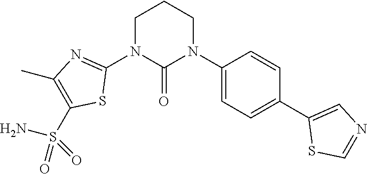 [SMILES: Cc1nc(N2CCCN(c3ccc(-c4cncs4)cc3)C2=O)sc1S(N)(=O)=O] | 436.0 | ¹H NMR (400 MHz, DMSO-d₆): δ 9.09 (s, 1H), 8.33 (s, 1H), 7.73 (d, J = 8.3 Hz, 2H), 7.56 (s, 2H), 7.51-7.42 (m, 2H), 4.19-4.12 (m, 2H), 3.84- 3.76 (m, 2H), 2.44 (s, 3H), 2.28-2.17 (m, 2H) ppm |
| 50 | 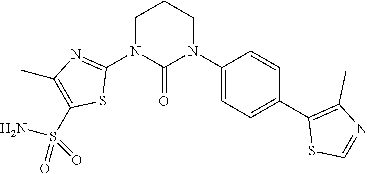 [SMILES: Cc1ncsc1-c1ccc(N2CCCN(c3nc(C)c(S(N)(=O)=O)s3)C2=O)cc1] | 450.2 | ¹H NMR (400 MHz, DMSO-d₆): δ 9.01 (s, 1H), 7.58-7.52 (m, 4H), 7.51-7.47 (m, 2H), 4.19- 4.13 (m, 2H), 3.85- 3.79 (m, 2H), 2.45 (s, 3H), 2.27-2.20 (m, 2H) ppm |
| 51 | 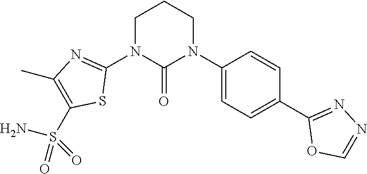 [SMILES: Cc1nc(N2CCCN(c3ccc(-c4nnco4)cc3)C2=O)sc1S(N)(=O)=O] | 421.2 | ¹H NMR (400 MHz, DMSO-d₆): δ 9.35 (s, 1H), 8.08-8.04 (m, 2H), 7.66-7.62 (m, 2H), 7.57 (s, 2H), 4.16 (t, J = 6.1 Hz, 2H), 3.86 (t, J = 5.6 Hz, 2H), 2.45 (s, 3H), 2.28-2.20 (m, 2H) ppm |
| 52 | 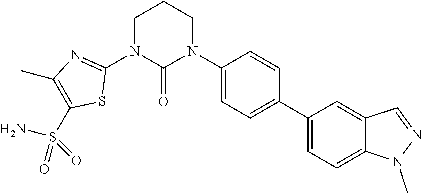 [SMILES: Cc1nc(N2CCCN(c3ccc(-c4ccc5c(cnn5C)c4)cc3)C2=O)sc1S(N)(=O)=O] | 483.0 | ¹H NMR (400 MHz, DMSO-d₆): δ 8.17 (s, 1H), 7.78 (d, J = 8.5 Hz, 2H), 7.66 (d, J = 8.4 Hz, 1H), 7.59-7.47 (m, 5H), 7.35-7.24 (m, 1H), 4.18 (t, J = 5.9 Hz, 2H), 4.10 (s, 3H), 3.85 (t, J = 5.5 Hz, 2H), 2.45 (s, 3H), 2.32-2.20 (m, 2H) ppm |
| 53 | 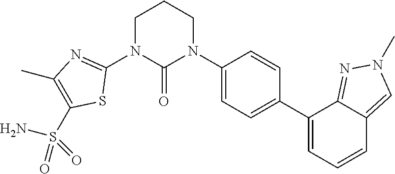 [SMILES: Cc1nc(N2CCCN(c3ccc(-c4cccc5cn(C)nc45)cc3)C2=O)sc1S(N)(=O)=O] | 483.2 | ¹H NMR (400 MHz, DMSO-d₆): δ 8.44 (s, 1H), 8.09 (d, J = 8.3 Hz, 2H), 7.74-7.68 (m, 1H), 7.55 (s, 2H), 7.53-7.43 (m, 3H), 7.15 (t, J = 7.6 Hz, 1H), 4.21 (s, 3H), 4.20-4.15 (m, 2H), 3.87- 3.80 (m, 2H), 2.45 (s, 3H), 2.32-2.17 (m, 2H) ppm |
| 54 | 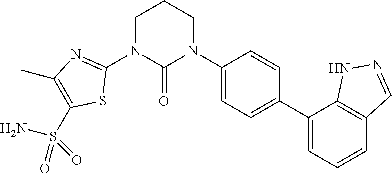 [SMILES: Cc1nc(N2CCCN(c3ccc(-c4cccc5cn[nH]c45)cc3)C2=O)sc1S(N)(=O)=O] | 469.1 | ¹H NMR (400 MHz, DMSO-d₆): δ 13.24 (br s, 1H), 8.19 (s, 1H), 7.82- 7.71 (m, 3H), 7.59-7.51 (m, 4H), 7.48-7.40 (m, 1H), 7.29-7.19 (m, 1H), 4.18 (t, J = 6.0 Hz, 2H), 3.85 (t, J = 5.6 Hz, 2H), 2.44 (s, 3H), 2.31-2.19 (m, 2H) ppm |
| 55 | 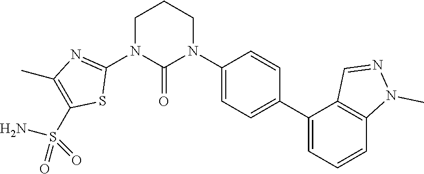 [SMILES: Cc1nc(N2CCCN(c3ccc(-c4cccc5c4cnn5C)cc3)C2=O)sc1S(N)(=O)=O] | 483.2 | ¹H NMR (400 MHz, DMSO-d₆): δ 8.17 (d, J = 0.9 Hz, 1H), 7.81-7.76 (m, 2H), 7.66 (d, J = 8.5 Hz, 1H), 7.59-7.47 (m, 5H), 7.29 (d, J = 6.6 Hz, 1H), 4.18 (t, J = 6.0 Hz, 2H), 4.10 (s, 3H), 3.85 (t, J = 5.6 Hz, 2H), 2.45 (s, 3H), 2.29-2.21 (m, 2H) ppm |
| 56 | 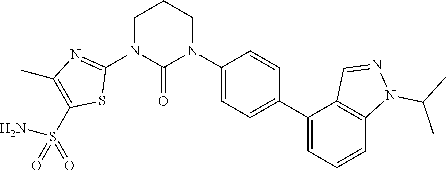 [SMILES: Cc1nc(N2CCCN(c3ccc(-c4cccc5c4cnn5C(C)C)cc3)C2=O)sc1S(N)(=O)=O] | 511.2 | ¹H NMR (400 MHz, DMSO-d₆): δ 8.18 (s, 1H), 7.77 (d, J = 8.6 Hz, 2H), 7.74-7.70 (m, 1H), 7.59-7.52 (m, 4H), 7.50- 7.45 (m, 1H), 7.27 (d, J = 7.2 Hz, 1H), 5.09-5.03 (m, 1H), 4.18 (t, J = 6.2 Hz, 2H), 3.85 (t, J = 5.3 Hz, 2H), 2.45 (s, 3H), 2.29-2.22 (m, 2H), 1.52 (d, J = 6.6 Hz, 6H) ppm |
| 57 | 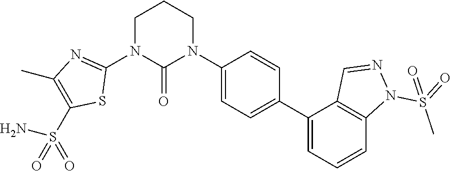 [SMILES: Cc1nc(N2CCCN(c3ccc(-c4cccc5c4cnn5S(C)(=O)=O)cc3)C2=O)sc1S(N)(=O)=O] | 547.0 | ¹H NMR (400 MHz, DMSO-d₆): δ 8.72 (s, 1H), 8.07-7.96 (m, 1H), 7.83-7.70 (m, 3H), 7.62- 7.52 (m, 5H), 4.24- 4.12 (m, 2H), 3.90-3.81 (m, 2H), 3.51 (s, 3H), 2.46 (s, 3H), 2.30-2.20 (m, 2H) ppm |
| 58 | 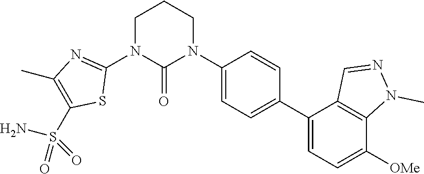 [SMILES: COc1ccc(-c2ccc(N3CCCN(c4nc(C)c(S(N)(=O)=O)s4)C3=O)cc2)c2cnn(C)c12] | 513.2 | ¹H NMR (400 MHz, DMSO-d₆): δ 8.09 (s, 1H), 7.73-7.66 (m, 2H), 7.55 (br.s, 2H), 7.52- 7.46 (m, 2H), 7.19-7.12 (m, 1H), 6.98-6.91 (m, 1H), 4.27 (s, 3H), 4.21- 4.14 (m, 2H), 3.99 (s, 3H), 3.88-3.78 (m, 2H), 2.45 (s, 3H), 2.29-2.18 (m, 2H) ppm |
| 59 | 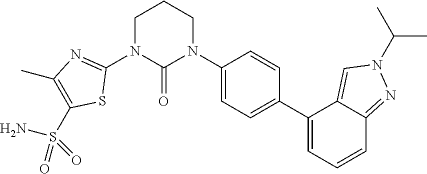 [SMILES: Cc1nc(N2CCCN(c3ccc(-c4cccc5nn(C(C)C)cc45)cc3)C2=O)sc1S(N)(=O)=O] | 511.2 | ¹H NMR (400 MHz, DMSO-d₆): δ 8.56 (s, 1H), 7.77 (d, J = 8.6 Hz, 2H), 7.63 (d, J = 8.7 Hz, 1H), 7.57 (s, 2H), 7.55- 7.49 (m, 2H), 7.36-7.30 (m, 1H), 7.18 (d, J = 6.6 Hz, 1H), 4.91-4.84 (m, 1H), 4.18 (t, J = 5.9 Hz, 2H), 3.84 (t, J = 5.6 Hz, 2H), 2.45 (s, 3H), 2.29- 2.22 (m, 2H), 1.57 (d, J = 6.6 Hz, 6H) ppm |
| 60 | 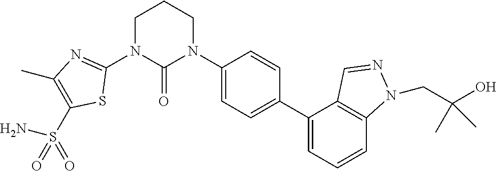 [SMILES: Cc1nc(N2CCCN(c3ccc(-c4cccc5c4cnn5CC(C)(C)O)cc3)C2=O)sc1S(N)(=O)=O] | 541.1 | ¹H NMR (400 MHz, DMSO-d₆): δ 8.20-8.16 (m, 1H), 7.78 (d, J = 8.5 Hz, 2H), 7.76-7.69 (m, 1H), 7.60-7.51 (m, 4H), 7.50-7.41 (m, 1H), 7.26 (d, J = 6.8 Hz, 1H), 4.68 (s, 1H), 4.36 (s, 2H), 4.18 (t, J = 6.0 Hz, 2H), 3.85 (t, J = 5.4 Hz, 2H), 2.45 (s, 3H), 2.31-2.19 (m, 2H), 1.16 (s, 6H) ppm |
| 61 | 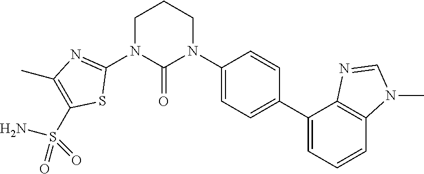 [SMILES: Cc1nc(N2CCCN(c3ccc(-c4cccc5c4ncn5C)cc3)C2=O)sc1S(N)(=O)=O] | 483.2 | ¹H NMR (400 MHz, DMSO-d₆): δ 8.26 (s, 1H), 8.15 (d, J = 8.4 Hz, 2H), 7.61-7.52 (m, 3H), 7.52-7.44 (m, 3H), 7.44- 7.32 (m, 1H), 4.23- 4.13 (m, 2H), 3.89 (s, 3H), 3.86-3.81 (m, 2H), 2.45 (s, 3H), 2.31-2.18 (m, 2H) ppm |
| 62 | 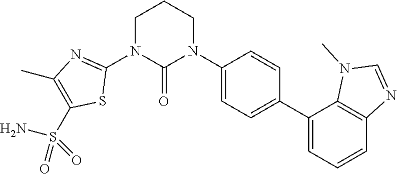 [SMILES: Cc1nc(N2CCCN(c3ccc(-c4cccc5ncn(C)c45)cc3)C2=O)sc1S(N)(=O)=O] | 483.3 | ¹H NMR (400 MHz, DMSO-d₆): δ 8.92 (s, 1H), 7.81 (d, J = 8.0 Hz, 1H), 7.57 (s, 2H), 7.55- 7.52 (m, 4H), 7.52-7.44 (m, 1H), 7.32-7.26 (m, 1H), 4.18 (t, J = 5.8 Hz, 2H), 3.86 (t, J = 5.2 Hz, 2H), 3.52 (s, 3H), 2.45 (s, 3H), 2.29-2.22 (m, 2H) ppm |
| 63 | 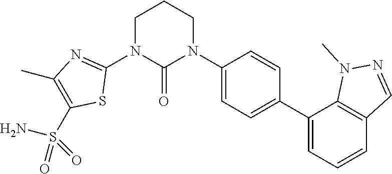 [SMILES: Cc1nc(N2CCCN(c3ccc(-c4cccc5cnn(C)c45)cc3)C2=O)sc1S(N)(=O)=O] | 483.3 | ¹H NMR (400 MHz, DMSO-d₆): δ 8.15 (s, 1H), 7.80 (dd, J = 1.8, 7.3 Hz, 1H), 7.56 (s, 2H), 7.54-7.50 (m, 4H), 7.27- 7.17 (m, 2H), 4.18 (t, J = 5.9 Hz, 2H), 3.87 (t, J = 5.5 Hz, 2H), 3.63 (s, 3H), 2.45 (s, 3H), 2.32-2.20 (m, 2H) ppm |
| 64 | 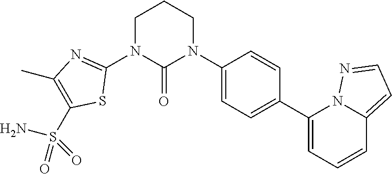 [SMILES: Cc1nc(N2CCCN(c3ccc(-c4cccc5ccnn45)cc3)C2=O)sc1S(N)(=O)=O] | 469.3 | ¹H NMR (400 MHz, DMSO-d₆): δ 8.09-7.92 (m, 3H), 7.75 (d, J = 8.8 Hz, 1H), 7.62-7.49 (m, 4H), 7.40-7.25 (m, 1H), 7.05 (d, J = 6.8 Hz, 1H), 6.74 (s, 1H), 4.22-4.14 (m, 2H), 3.91-3.83 (m, 2H), 2.46 (s, 3H), 2.34- 2.17 (m, 2H) ppm |
| 65 | 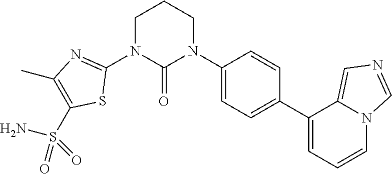 [SMILES: Cc1nc(N2CCCN(c3ccc(-c4cccn5cncc45)cc3)C2=O)sc1S(N)(=O)=O] | 469.2 | ¹H NMR (400 MHz, DMSO-d₆): δ 8.51 (s, 1H), 8.38 (d, J = 7.1 Hz, 1H), 7.80-7.76 (m, 2H), 7.59-7.49 (m, 5H), 6.92 (d, J = 6.1 Hz, 1H), 6.82- 6.77 (m, 1H), 4.18 (t, J = 5.9 Hz, 2H), 3.85 (t, J = 5.7 Hz, 2H), 2.45 (s, 3H), 2.29-2.21 (m, 2H) ppm |
| 66 | 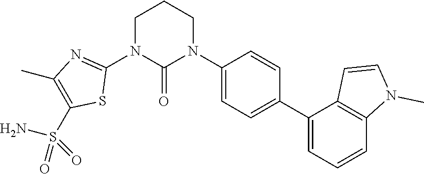 [SMILES: Cc1nc(N2CCCN(c3ccc(-c4cccc5c4ccn5C)cc3)C2=O)sc1S(N)(=O)=O] | 482.2 | ¹H NMR (400 MHz, DMSO-d₆): δ 7.72-7.67 (m, 2H), 7.56 (s, 2H), 7.53-7.45 (m, 3H), 7.45- 7.39 (m, 1H), 7.26 (t, J = 7.7 Hz, 1H), 7.16-7.13 (m, 1H), 6.58-6.55 (m, 1H), 4.18 (t, J = 6.0 Hz, 2H), 3.88-3.80 (m, 5H), 2.45 (s, 3H), 2.28-2.21 (m, 2H) ppm |
| 67 | 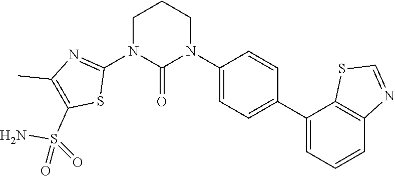 [SMILES: Cc1nc(N2CCCN(c3ccc(-c4cccc5ncsc45)cc3)C2=O)sc1S(N)(=O)=O] | 486.0 | ¹H NMR (400 MHz, DMSO-d₆): δ 9.48 (s, 1H), 8.13 (d, J = 7.3 Hz, 1H), 7.83-7.74 (m, 2H), 7.73-7.65 (m, 1H), 7.65- 7.53 (m, 5H), 4.23- 4.14 (m, 2H), 3.91-3.82 (m, 2H), 2.45 (s, 3H), 2.30-2.20 (m, 2H) ppm |
| 68 | 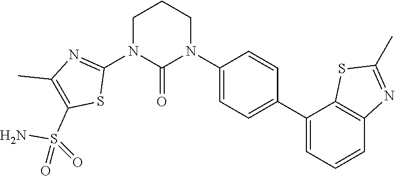 | 500.3 | ¹H NMR (400 MHz, DMSO-d₆): δ 7.94 (dd, J = 1.0, 8.0 Hz, 1H), 7.77- 7.72 (m, 2H), 7.64-7.49 (m, 6H), 4.18 (t, J = 6.0 Hz, 2H), 3.85 (t, J = 5.6 Hz, 2H), 2.82 (s, 3H), 2.45 (s, 3H), 2.30-2.20 (m, 2H) ppm |
| 69 | 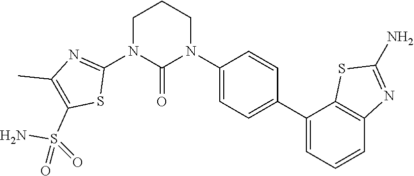 [SMILES: Cc1nc(N2CCCN(c3ccc(-c4cccc5nc(N)sc45)cc3)C2=O)sc1S(N)(=O)=O] | 501.2 | ¹H NMR (400 MHz, DMSO-d₆): δ 7.67 (d, J = 8.5 Hz, 2H), 7.56 (s, 2H), 7.55-7.49 (m, 4H), 7.37- 7.31 (m, 2H), 7.12 (dd, J = 2.6, 6.1 Hz, 1H), 4.17 (t, J = 5.9 Hz, 2H), 3.84 (t, J = 5.6 Hz, 2H), 2.48- 2.45 (s, 3H), 2.28-2.20 (m, 2H) ppm |
| 70 | 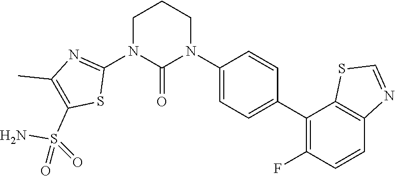 [SMILES: Cc1nc(N2CCCN(c3ccc(-c4c(F)ccc5ncsc45)cc3)C2=O)sc1S(N)(=O)=O] | 504.3 | ¹H NMR (400 MHz, DMSO-d₆): δ 9.41 (s, 1H), 8.11 (dd, J = 2.5, 8.4 Hz, 1H), 7.94 (d, J = 8.4 Hz, 2H), 7.63-7.48 (m, 5H), 4.18 (t, J = 5.9 Hz, 2H), 3.85 (t, J = 5.5 Hz, 2H), 2.45 (s, 3H), 2.29- 2.22 (m, 2H) ppm |
| 71 | 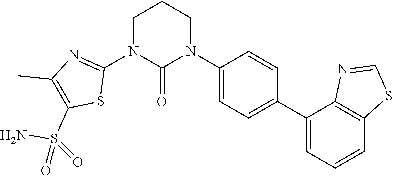 [SMILES: Cc1nc(N2CCCN(c3ccc(-c4cccc5scnc45)cc3)C2=O)sc1S(N)(=O)=O] | 484.3 | ¹H NMR (400 MHz, DMSO-d₆): δ 9.44 (s, 1H), 8.20 (d, J = 7.8 Hz, 1H), 7.90 (d, J = 8.3 Hz, 2H), 7.70-7.66 (m, 1H), 7.63-7.54 (m, 3H), 7.54- 7.48 (m, 2H), 4.18 (t, J = 5.9 Hz, 2H), 3.85 (t, J = 5.6 Hz, 2H), 2.45 (s, 3H), 2.28-2.22 (m, 2H) ppm |
| 72 | 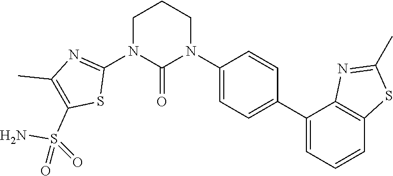 | 499.9 | ¹H NMR (400 MHz, DMSO-d₆): δ 8.08-8.02 (m, 1H), 7.88-7.81 (m, 2H), 7.59-7.54 (m, 3H), 7.53-7.45 (m, 3H), 4.22- 4.15 (m, 2H), 3.88- 3.81 (m, 2H), 2.82 (s, 3H), 2.45 (s, 3H), 2.29- 2.21 (m, 2H) ppm |
| 73 | 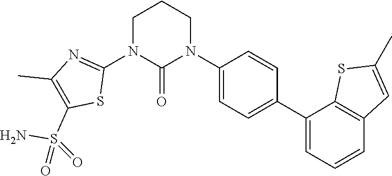 | 499.3 | ¹H NMR (400 MHz, DMSO-d₆): δ 7.74 (d, J = 7.8 Hz, 3H), 7.59-7.52 (m, 4H), 7.50-7.41 (m, 1H), 7.40-7.30 (m, 1H), 7.24 (s, 1H), 4.21-4.14 (m, 2H), 3.89-3.80 (m, 2H), 2.56 (s, 3H), 2.45 (s, 3H), 2.29-2.20 (m, 2H) ppm |
| 74 | 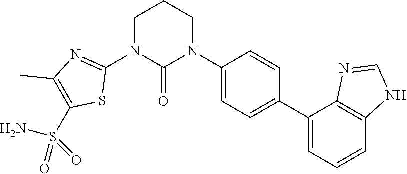 [SMILES: Cc1nc(N2CCCN(c3ccc(-c4cccc5[nH]cnc45)cc3)C2=O)sc1S(N)(=O)=O] | 469.1 | ¹H NMR (400 MHz, DMSO-d₆): δ 13.25 (s, 1H), 8.20 (s, 1H), 7.79 (d, J = 8.5 Hz, 2H), 7.59- 7.50 (m, 5H), 7.50-7.40 (m, 1H), 7.26 (d, J = 6.9 Hz, 1H), 4.18 (t, J = 5.9 Hz, 2H), 3.85 (t, J = 5.5 Hz, 2H), 2.45 (s, 3H), 2.30-2.21 (m, 2H) ppm |
| 75 | 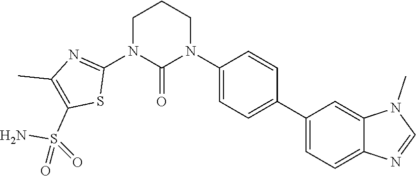 [SMILES: Cc1nc(N2CCCN(c3ccc(-c4ccc5ncn(C)c5c4)cc3)C2=O)sc1S(N)(=O)=O] | 483.3 | ¹H NMR (400 MHz, DMSO-d₆): δ 8.17 (d, J = 0.9 Hz, 1H), 7.81-7.75 (m, 2H), 7.66 (d, J = 8.5 Hz, 1H), 7.59-7.46 (m, 5H), 7.29 (d, J = 6.6 Hz, 1H), 4.18 (t, J = 6.0 Hz, 2H), 4.10 (s, 3H), 3.85 (t, J = 5.6 Hz, 2H), 2.45 (s, 3H), 2.29-2.21 (m, 2H) ppm |
| 76 | 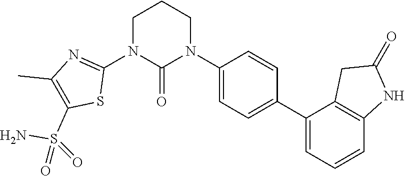 [SMILES: Cc1nc(N2CCCN(c3ccc(-c4cccc5c4CC(=O)N5)cc3)C2=O)sc1S(N)(=O)=O] | 484.1 | ¹H NMR (400 MHz, DMSO-d₆): δ 10.47 (s, 1H), 7.61 (d, J = 7.8 Hz, 2H), 7.57-7.53 (m, 2H), 7.51-7.38 (m, 2H), 7.31- 7.24 (m, 1H), 7.03 (d, J = 7.8 Hz, 1H), 6.82 (d, J = 7.3 Hz, 1H), 4.19-4.11 (m, 2H), 3.84-3.76 (m, 2H), 3.63 (s, 2H), 2.43 (s, 3H), 2.26-2.17 (m, 2H) ppm |
| 77 | 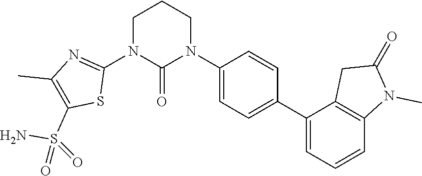 | 498.3 | ¹H NMR (400 MHz, DMSO-d₆): δ 7.66-7.61 (m, J = 8.5 Hz, 2H), 7.56 (s, 2H), 7.47 (d, J = 8.5 Hz, 2H), 7.45-7.36 (m, 1H), 7.13 (d, J = 7.4 Hz, 1H), 7.00 (d, J = 7.6 Hz, 1H), 4.17 (t, J = 6.0 Hz, 2H), 3.82 (t, J = 5.6 Hz, 2H), 3.72 (s, 2H), 3.16 (s, 3H), 2.45 (s, 3H), 2.28- 2.18 (m, 2H) ppm |
| 78 | 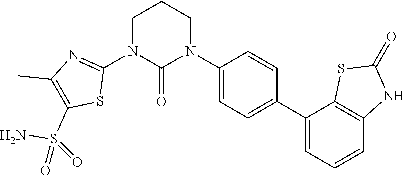 [SMILES: Cc1nc(N2CCCN(c3ccc(-c4cccc5[nH]c(=O)sc45)cc3)C2=O)sc1S(N)(=O)=O] | 502.2 | ¹H NMR (400 MHz, DMSO-d₆): δ 12.04 (s, 1H), 7.65-7.59 (m, 2H), 7.58-7.51 (m, 4H), 7.44- 7.38 (m, 1H), 7.25 (dd, J = 0.9, 7.7 Hz, 1H), 7.15 (dd, J = 0.9, 7.9 Hz, 1H), 4.17 (t, J = 5.9 Hz, 2H), 3.83 (t, J = 5.6 Hz, 2H), 2.45 (s, 3H), 2.28-2.18 (m, 2H) ppm |
| 79 | 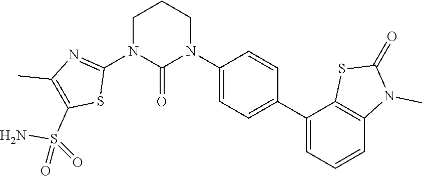 | 516.0 | ¹H NMR (400 MHz, DMSO-d₆): δ 7.64-7.59 (m, 2H), 7.58-7.46 (m, 5H), 7.37-7.29 (m, 2H), 4.19-4.10 (m, 2H), 3.86- 3.76 (m, 2H), 3.44 (s, 3H), 2.43 (s, 3H), 2.28- 2.18 (m, 2H) ppm |
| 80 | 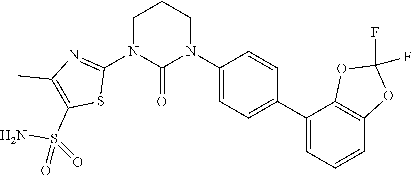 [SMILES: Cc1nc(N2CCCN(c3ccc(-c4cccc5c4OC(F)(F)O5)cc3)C2=O)sc1S(N)(=O)=O] | 508.9 | ¹H NMR (400 MHz, DMSO-d₆): δ 7.77 (d, J = 8.6 Hz, 2H), 7.61-7.48 (m, 5H), 7.45-7.39 (m, 1H), 7.37-7.30 (m, 1H), 4.17 (t, J = 5.7 Hz, 2H), 3.83 (t, J = 5.4 Hz, 2H), 2.45 (s, 3H), 2.29-2.18 (m, 2H) ppm |
| 81 | 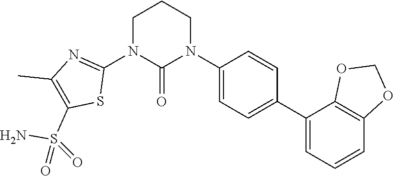 [SMILES: Cc1nc(N2CCCN(c3ccc(-c4cccc5c4OCO5)cc3)C2=O)sc1S(N)(=O)=O] | 473.0 | ¹H NMR (400 MHz, DMSO-d₆): δ 7.86-7.65 (m, J = 8.3 Hz, 2H), 7.56 (s, 2H), 7.52-7.41 (m, 2H), 7.14 (d, J = 6.8 Hz, 1H), 7.03-6.85 (m, 2H), 6.08 (s, 2H), 4.15 (t, J = 5.4 Hz, 2H), 3.83-3.78 (m, 2H), 2.43 (s, 3H), 2.27-2.16 (m, 2H) ppm |
| 82 | 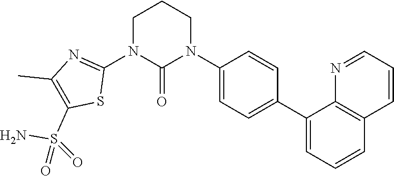 [SMILES: Cc1nc(N2CCCN(c3ccc(-c4cccc5cccnc45)cc3)C2=O)sc1S(N)(=O)=O] | 480.3 | ¹H NMR (400 MHz, DMSO-d₆): δ 8.93 (d, J = 2.4 Hz, 1H), 8.47 (d, J = 8.3 Hz, 1H), 8.03 (d, J = 7.3 Hz, 1H), 7.81 (d, J = 6.4 Hz, 1H), 7.75-7.66 (m, 3H), 7.63-7.54 (m, 3H), 7.50-7.46 (m, 2H), 4.19 (t, J = 5.2 Hz, 2H), 3.85 (t, J = 5.6 Hz, 2H), 2.45 (s, 3H), 2.31-2.22 (m, 2H) ppm |
| 83 | 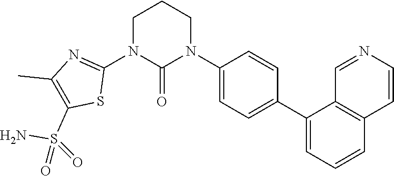 [SMILES: Cc1nc(N2CCCN(c3ccc(-c4cccc5ccncc45)cc3)C2=O)sc1S(N)(=O)=O] | 480.3 | ¹H NMR (400 MHz, DMSO-d₆): δ 9.21 (s, 1H), 8.56 (d, J = 5.6 Hz, 1H), 8.03 (d, J = 8.4 Hz, 1H), 7.93 (d, J = 5.3 Hz, 1H), 7.91-7.83 (m, 1H), 7.68-7.52 (m, 7H), 4.24- 4.14 (m, 2H), 3.97- 3.83 (m, 2H), 2.46 (s, 3H), 2.34-2.24 (m, 2H) ppm |
| 84 | 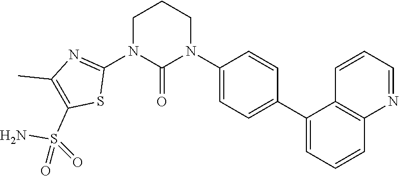 [SMILES: Cc1nc(N2CCCN(c3ccc(-c4cccc5ncccc45)cc3)C2=O)sc1S(N)(=O)=O] | 480.5 | ¹H NMR (400 MHz, DMSO-d₆): δ 8.98-8.93 (m, 1H), 8.25 (d, J = 8.3 Hz, 1H), 8.08 (d, J = 8.3 Hz, 1H), 7.85 (t, J = 7.8 Hz, 1H), 7.61-7.52 (m, 8H), 4.22-4.15 (m, 2H), 3.92-3.83 (m, 2H), 2.46 (s, 3H), 2.31-2.21 (m, 2H) ppm |
| 85 | 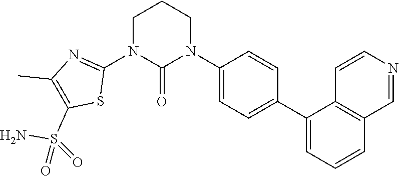 [SMILES: Cc1nc(N2CCCN(c3ccc(-c4cccc5cnccc45)cc3)C2=O)sc1S(N)(=O)=O] | 480.6 | ¹H NMR (400 MHz, DMSO-d₆): δ 9.41 (s, 1H), 8.52 (d, J = 6.0 Hz, 1H), 8.19 (dd, J = 2.7, 6.7 Hz, 1H), 7.81-7.75 (m, 2H), 7.75-7.68 (m, 1H), 7.57 (s, 6H), 4.19 (t, J = 6.0 Hz, 2H), 3.88 (t, J = 5.6 Hz, 2H), 2.45 (s, 3H), 2.33-2.21 (m, 2H) ppm |
| 86 | 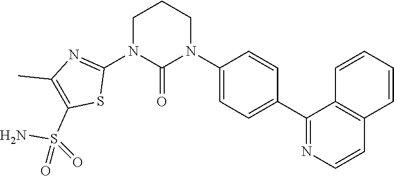 [SMILES: Cc1nc(N2CCCN(c3ccc(-c4nccc5ccccc45)cc3)C2=O)sc1S(N)(=O)=O] | 480.4 | ¹H NMR (400 MHz, DMSO-d₆): δ 8.68-8.52 (m, 1H), 8.08 (br s, 2H), 7.94-7.77 (m, 2H), 7.76- 7.65 (m, 3H), 7.63- 7.47 (m, 4H), 4.33-4.09 (m, 2H), 3.98-3.79 (m, 2H), 2.46 (s, 3H), 2.33- 2.23 (m, 2H) ppm |
| 87 | 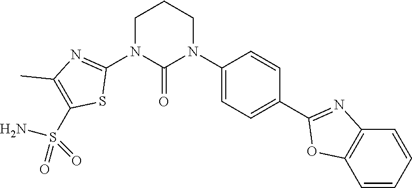 [SMILES: Cc1nc(N2CCCN(c3ccc(-c4nc5ccccc5o4)cc3)C2=O)sc1S(N)(=O)=O] | 470.2 | ¹H NMR (400 MHz, DMSO-d₆): δ 8.22 (d, J = 8.3 Hz, 2H), 7.84-7.75 (m, 2H), 7.68-7.60 (m, 2H), 7.56 (s, 2H), 7.43- 7.38 (m, 2H), 4.19-4.10 (m, 2H), 3.90-3.82 (m, 2H), 2.43 (s, 3H), 2.29- 2.17 (m, 2H) ppm |
| 88 | 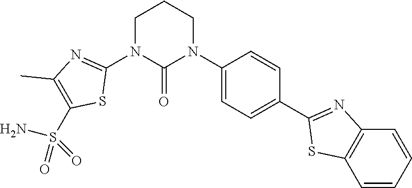 [SMILES: Cc1nc(N2CCCN(c3ccc(-c4nc5ccccc5s4)cc3)C2=O)sc1S(N)(=O)=O] | 486.1 | ¹H NMR (400 MHz, DMSO-d₆): δ 8.19-8.11 (m, 3H), 8.10-8.05 (m, 1H), 7.66-7.53 (m, 5H), 7.52-7.45 (m, 1H), 4.17 (t, J = 5.9 Hz, 2H), 3.87 (t, J = 5.5 Hz, 2H), 2.45 (s, 3H), 2.30-2.19 (m, 2H) ppm |
| 89 | 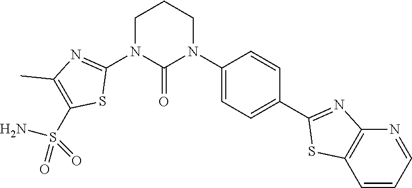 [SMILES: Cc1nc(N2CCCN(c3ccc(-c4nc5ncccc5s4)cc3)C2=O)sc1S(N)(=O)=O] | 487.1 | ¹H NMR (400 MHz, DMSO-d₆): δ 8.72 (dd, J = 1.6, 4.6 Hz, 1H), 8.69- 8.63 (m, 1H), 8.20 (d, J = 8.6 Hz, 2H), 7.65 (d, J = 8.6 Hz, 2H), 7.58 (s, 2H), 7.52-7.46 (m, 1H), 4.17 (t, J = 5.9 Hz, 2H), 3.88 (t, J = 5.4 Hz, 2H), 2.44 (s, 3H), 2.30-2.22 (m, 2H) ppm |
| 90 | 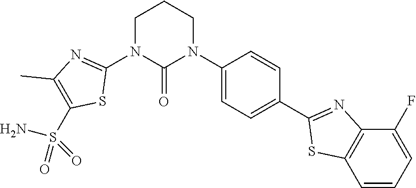 [SMILES: Cc1nc(N2CCCN(c3ccc(-c4nc5c(F)cccc5s4)cc3)C2=O)sc1S(N)(=O)=O] | 504.0 | ¹H NMR (400 MHz, DMSO-d₆): δ 8.15 (d, J = 8.8 Hz, 2H), 8.00 (dd, J = 0.8, 7.9 Hz, 1H), 7.63 (d, J = 8.8 Hz, 2H), 7.59- 7.55 (m, 2H), 7.53-7.46 (m, 1H), 7.45-7.34 (m, 1H), 4.17 (t, J = 6.0 Hz, 2H), 3.87 (t, J = 5.6 Hz, 2H), 2.45 (s, 3H), 2.32- 2.19 (m, 2H) ppm |
| 91 | 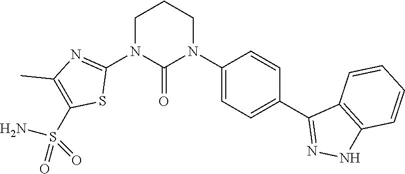 [SMILES: Cc1nc(N2CCCN(c3ccc(-c4n[nH]c5ccccc45)cc3)C2=O)sc1S(N)(=O)=O] | 469.1 | ¹H NMR (400 MHz, DMSO-d₆): δ 13.26 (s, 1H), 8.09 (d, J = 7.8 Hz, 1H), 8.06-8.00 (m, 2H), 7.66-7.49 (m, 6H), 7.45- 7.39 (m, 1H), 7.27- 7.19 (m, 1H), 4.24-4.13 (m, 2H), 3.90-3.78 (m, 2H), 2.45 (s, 3H), 2.30- 2.20 (m, 2H) ppm |
| 92 | 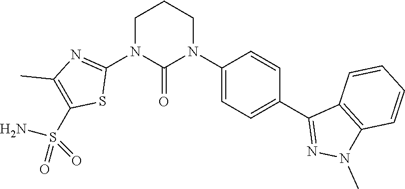 [SMILES: Cc1nc(N2CCCN(c3ccc(-c4nn(C)c5ccccc45)cc3)C2=O)sc1S(N)(=O)=O] | 483.3 | ¹H NMR (400 MHz, DMSO-d₆): δ 8.10 (d, J = 8.3 Hz, 1H), 8.01 (d, J = 8.6 Hz, 2H), 7.71 (d, J = 8.5 Hz, 1H), 7.59-7.45 (m, 5H), 7.28-7.23 (m, 1H), 4.18 (t, J = 5.9 Hz, 2H), 4.12 (s, 3H), 3.84 (t, J = 5.8 Hz, 2H), 2.45 (s, 3H), 2.28-2.21 (m, 2H) ppm |
| 93 | 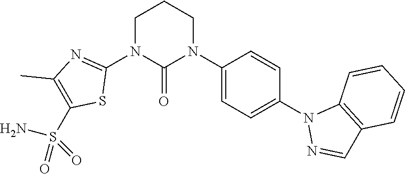 [SMILES: Cc1nc(N2CCCN(c3ccc(-n4ncc5ccccc54)cc3)C2=O)sc1S(N)(=O)=O] | 469.1 | ¹H NMR (400 MHz, DMSO-d₆): δ 8.39 (s, 1H), 7.94-7.77 (m, 4H), 7.62-7.45 (m, 5H), 7.32- 7.22 (m, 1H), 4.21- 4.12 (m, 2H), 3.89-3.78 (m, 2H), 2.43 (s, 3H), 2.31-2.17 (m, 2H) ppm |
| 94 | 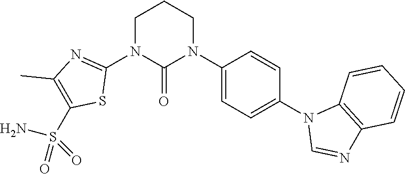 [SMILES: Cc1nc(N2CCCN(c3ccc(-n4cnc5ccccc54)cc3)C2=O)sc1S(N)(=O)=O] | 469.6 | ¹H NMR (400 MHz, DMSO-d₆): δ 8.61 (s, 1H), 7.81-7.78 (m, 1H), 7.77-7.71 (m, 2H), 7.69- 7.61 (m, 3H), 7.57 (s, 2H), 7.39-7.31 (m, 2H), 4.19 (t, J = 6.0 Hz, 2H), 3.86 (t, J = 5.6 Hz, 2H), 2.45 (s, 3H), 2.34-2.21 (m, 2H) ppm |
| 95 | 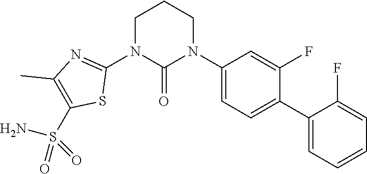 [SMILES: Cc1nc(N2CCCN(c3ccc(-c4ccccc4F)c(F)c3)C2=O)sc1S(N)(=O)=O] | 465.1 | ¹H NMR (400 MHz, DMSO-d₆): δ 7.58 (s, 2H), 7.54-7.45 (m, 4H), 7.40-7.29 (m, 3H), 4.19- 4.12 (m, 2H), 3.89- 3.80 (m, 2H), 2.45 (s, 3H), 2.28-2.18 (m, 2H) ppm |
| 96 | 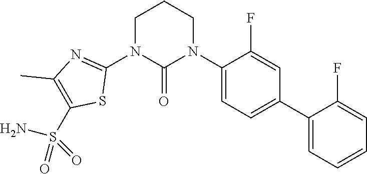 [SMILES: Cc1nc(N2CCCN(c3ccc(-c4ccccc4F)cc3F)C2=O)sc1S(N)(=O)=O] | 465.0 | ¹H NMR (400 MHz, DMSO-d₆): δ 7.63-7.54 (m, 5H), 7.50-7.43 (m, 2H), 7.42-7.28 (m, 2H), 4.23-4.14 (m, 2H), 3.80 (m, 2H), 2.45 (s, 3H), 2.29-2.18 (m, 2H) ppm |
| 97 | 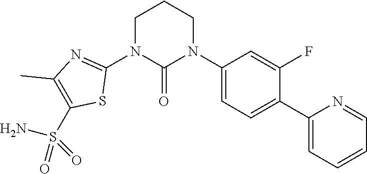 [SMILES: Cc1nc(N2CCCN(c3ccc(-c4ccccn4)c(F)c3)C2=O)sc1S(N)(=O)=O] | 447.9 | ¹H NMR (400 MHz, DMSO-d₆): δ 8.73 (d, J = 4.4 Hz, 1H), 8.00-7.90 (m, 2H), 7.81 (d, J = 7.8 Hz, 1H), 7.58 (s, 2H), 7.49-7.36 (m, 3H), 4.16 (t, J = 5.6 Hz, 2H), 3.85 (t, J = 5.6 Hz, 2H), 2.45 (s, 3H), 2.28-2.19 (m, 2H) ppm |
| 98 | 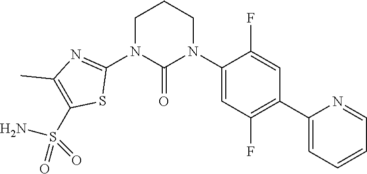 [SMILES: Cc1nc(N2CCCN(c3cc(F)c(-c4ccccn4)cc3F)C2=O)sc1S(N)(=O)=O] | 466.2 | ¹H NMR (400 MHz, DMSO-d₆): δ 8.77-8.73 (m, 1H), 7.99-7.93 (m, 1H), 7.89-7.83 (m, 2H), 7.64 (dd, J = 6.3, 11.4 Hz, 1H), 7.58 (s, 2H), 7.50-7.44 (m, 1H), 4.18 (t, J = 6.0 Hz, 2H), 3.78 (t, J = 5.6 Hz, 2H), 2.45 (s, 3H), 2.28-2.20 (m, 2H) ppm |
| 99 | 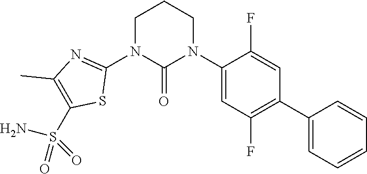 [SMILES: Cc1nc(N2CCCN(c3cc(F)c(-c4ccccc4)cc3F)C2=O)sc1S(N)(=O)=O] | 465.1 | ¹H NMR (400 MHz, DMSO-d₆): δ 7.64-7.43 (m, 9H), 4.18 (t, J = 5.9 Hz, 2H), 3.77 (t, J = 5.6 Hz, 2H), 2.45 (s, 3H), 2.32-2.17 (m, 2H) ppm |
| 100 | 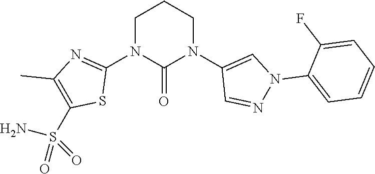 [SMILES: Cc1nc(N2CCCN(c3cnn(-c4ccccc4F)c3)C2=O)sc1S(N)(=O)=O] | 437.2 | ¹H NMR (400 MHz, DMSO-d₆): δ 8.42 (d, J = 2.5 Hz, 1H), 8.06 (s, 1H), 7.85-7.78 (m, 1H), 7.58 (s, 2H), 7.51-7.34 (m, 3H), 4.16 (t, J = 5.9 Hz, 2H), 3.85 (t, J = 5.8 Hz, 2H), 2.44 (s, 3H), 2.26- 2.17 (m, 2H) ppm |
| 101 | 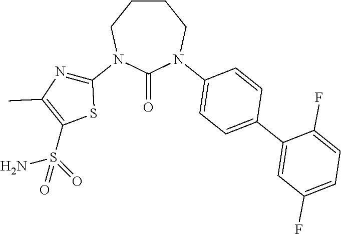 [SMILES: Cc1nc(N2CCCCN(c3ccc(-c4cc(F)ccc4F)cc3)C2=O)sc1S(N)(=O)=O] | 479.0 | ¹H NMR (400 MHz, DMSO-d₆): δ 7.62 (d, J = 7.8 Hz, 2H), 7.60-7.55 (m, 2H), 7.53-7.35 (m, 4H), 7.33-7.22 (m, 1H), 4.28-4.21 (m, 2H), 3.84- 3.78 (m, 2H), 2.45 (s, 3H), 2.02-1.93 (m, 2H), 1.89-1.81 (m, 2H) ppm |
| 102 | 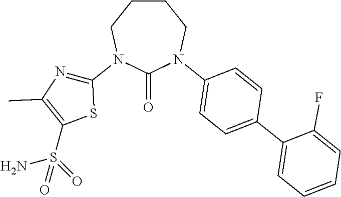 [SMILES: Cc1nc(N2CCCCN(c3ccc(-c4ccccc4F)cc3)C2=O)sc1S(N)(=O)=O] | 461.2 | ¹H NMR (400 MHz, DMSO-d₆): δ 7.62-7.53 (m, 5H), 7.52-7.38 (m, 3H), 7.38-7.27 (m, 2H), 4.29-4.21 (m, 2H), 3.87- 3.77 (m, 2H), 2.45 (s, 3H), 2.03-1.92 (m, 2H), 1.90-1.79 (m, 2H) ppm |
| 103 | 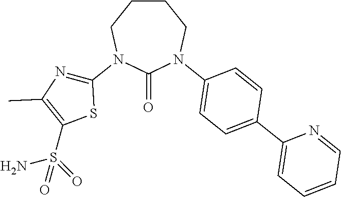 [SMILES: Cc1nc(N2CCCCN(c3ccc(-c4ccccn4)cc3)C2=O)sc1S(N)(=O)=O] | 444.1 | ¹H NMR (400 MHz, DMSO-d₆): δ 8.67 (d, J = 4.4 Hz, 1H), 8.12 (d, J = 8.3 Hz, 2H), 8.00-7.96 (m, 1H), 7.95-7.83 (m, 1H), 7.56 (br s, 2H), 7.53- 7.45 (m, 2H), 7.45- 7.27 (m, 1H), 4.32-4.17 (m, 2H), 3.86-3.75 (m, 2H), 2.45 (s, 3H), 2.04- 1.93 (m, 2H), 1.91-1.80 (m, 2H) ppm |
| 104 | 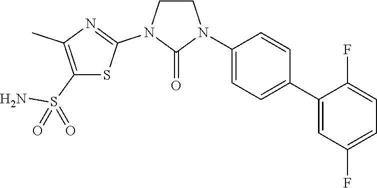 [SMILES: Cc1nc(N2CCN(c3ccc(-c4cc(F)ccc4F)cc3)C2=O)sc1S(N)(=O)=O] | 450.9 | ¹H NMR (400 MHz, DMSO-d₆): δ 7.77 (d, J = 8.9 Hz, 2H), 7.64 (dd, J = 1.4, 8.7 Hz, 2H), 7.47- 7.33 (m, 2H), 7.27-7.17 (m, 1H), 6.16 (br.s, 2H), 4.17-4.08 (m, 4H), 2.40 (s, 3H) ppm |
| 105 | 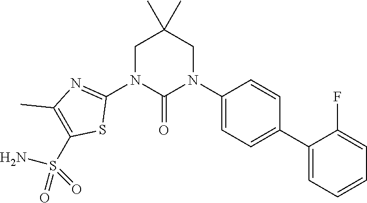 [SMILES: Cc1nc(N2CC(C)(C)CN(c3ccc(-c4ccccc4F)cc3)C2=O)sc1S(N)(=O)=O] | 475.4 | ¹H NMR (400 MHz, DMSO-d₆): δ 7.62-7.53 (m, 5H), 7.52-7.40 (m, 3H), 7.39-7.27 (m, 2H), 3.94 (s, 2H), 3.60 (s, 2H), 2.44 (s, 3H), 1.17 (s, 6H) ppm |
| 106 | 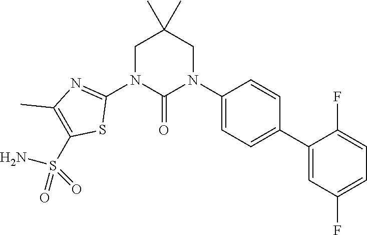 [SMILES: Cc1nc(N2CC(C)(C)CN(c3ccc(-c4cc(F)ccc4F)cc3)C2=O)sc1S(N)(=O)=O] | 493.1 | ¹H NMR (400 MHz, DMSO-d₆): δ 7.63 (d, J = 7.1 Hz, 2H), 7.55 (s, 2H), 7.52-7.36 (m, 4H), 7.32- 7.25 (m, 1H), 3.93 (s, 2H), 3.60 (s, 2H), 2.44 (s, 3H), 1.17 (s, 6H) ppm |
| 107 | 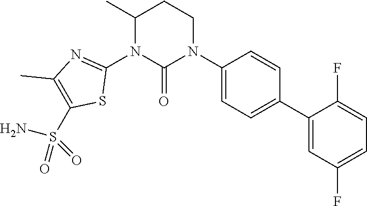 [SMILES: Cc1nc(N2C(=O)N(c3ccc(-c4cc(F)ccc4F)cc3)CCC2C)sc1S(N)(=O)=O] | 479.3 | ¹H NMR (400 MHz, DMSO-d₆): δ 7.64 (d, J = 7.8 Hz, 2H), 7.59-7.51 (m, 4H), 7.50-7.34 (m, 2H), 7.32-7.23 (m, 1H), 5.17-5.08 (m, 1H), 4.13- 4.02 (m, 1H), 3.74- 3.65 (m, 1H), 2.45 (s, 3H), 2.40-2.30 (m, 1H), 2.11-2.02 (m, 1H), 1.43 (d, J = 6.8 Hz, 3H) ppm |
| 108 | 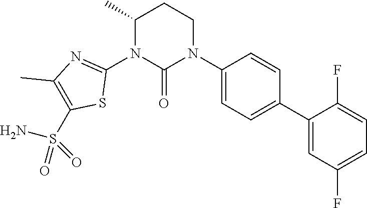 [SMILES: Cc1nc(N2C(=O)N(c3ccc(-c4cc(F)ccc4F)cc3)CC[C@H]2C)sc1S(N)(=O)=O] Single enantiomer, arbitrarily assigned stereochemistry | 479.3 | ¹H NMR (400 MHz, DMSO-d₆): δ 7.63 (m, 2H), 7.58-7.51 (m, 4H), 7.50-7.36 (m, 2H), 7.32- 7.25 (m, 1H), 5.16- 5.07 (m, 1H), 4.13-4.03 (m, 1H), 3.74-3.65 (m, 1H), 2.46 (s, 3H), 2.42- 2.30 (m, 1H), 2.10-2.01 (m, 1H), 1.43 (d, J = 6.4 Hz, 3H) ppm |
| 109 | 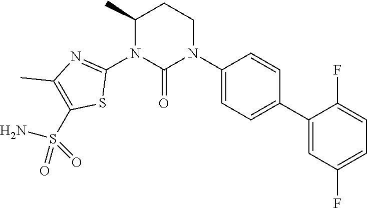 [SMILES: Cc1nc(N2C(=O)N(c3ccc(-c4cc(F)ccc4F)cc3)CC[C@@H]2C)sc1S(N)(=O)=O] Single enantiomer, arbitrarily assigned stereochemistry | 478.9 | ¹H NMR (400 MHz, DMSO-d₆): δ 7.66-7.61 (m, 2H), 7.58-7.50 (m, 4H), 7.49-7.42 (m, 1H), 7.41-7.34 (m, 1H), 7.32- 7.24 (m, 1H), 5.16- 5.06 (m, 1H), 4.14-4.01 (m, 1H), 3.73-3.64 (m, 1H), 2.45 (s, 3H), 2.39- 2.30 (m, 1H), 2.10-2.02 (m, 1H), 1.43 (d, J = 6.4 Hz, 3H) ppm |
| 110 | 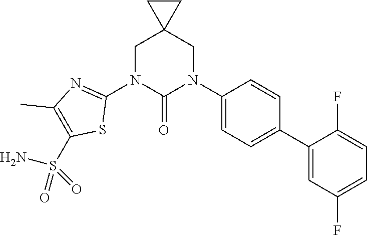 [SMILES: Cc1nc(N2CC3(CC3)CN(c3ccc(-c4cc(F)ccc4F)cc3)C2=O)sc1S(N)(=O)=O] | 490.1 | ¹H NMR (400 MHz, DMSO-d₆): δ 7.62 (d, J = 8.3 Hz, 2H), 7.57-7.53 (m, 2H), 7.49 (d, J = 8.3 Hz, 2H), 7.46-7.35 (m, 2H), 7.31-7.23 (m, 1H), 4.01 (s, 2H), 3.69 (s, 2H), 2.42 (s, 3H), 0.81 (d, J = 4.9 Hz, 4H) ppm |
| 111 | 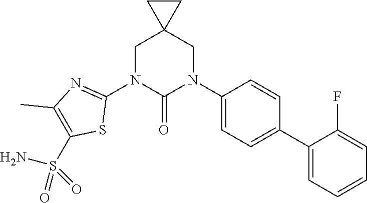 [SMILES: Cc1nc(N2CC3(CC3)CN(c3ccc(-c4ccccc4F)cc3)C2=O)sc1S(N)(=O)=O] | 473.2 | ¹H NMR (400 MHz, DMSO-d₆): δ 7.61-7.49 (m, 5H), 7.49-7.39 (m, 3H), 7.34-7.28 (m, 2H), 3.99 (s, 2H), 3.67 (s, 2H), 2.41 (s, 3H), 0.79 (d, J = 5.9 Hz, 4H) ppm. |
| 112 | 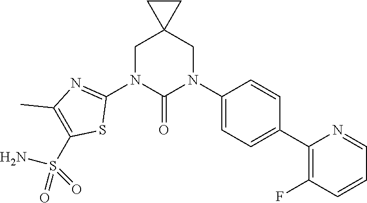 [SMILES: Cc1nc(N2CC3(CC3)CN(c3ccc(-c4ncccc4F)cc3)C2=O)sc1S(N)(=O)=O] | 473.9 | ¹H NMR (400 MHz, DMSO-d₆): δ 8.56 (br s, 1H), 7.96 (d, J = 8.3 Hz, 2H), 7.91-7.79 (m, 1H), 7.62-7.50 (m, 5H), 4.02 (s, 2H), 3.71 (s, 2H), 2.44 (s, 3H), 0.86-0.77 (m, 4H) ppm |
| 113 | 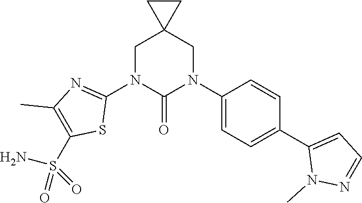 [SMILES: Cc1nc(N2CC3(CC3)CN(c3ccc(-c4ccnn4C)cc3)C2=O)sc1S(N)(=O)=O] | 459.3 | ¹H NMR (400 MHz, DMSO-d₆): δ 7.60-7.53 (m, 4H), 7.53-7.44 (m, 3H), 6.42 (d, J = 1.9 Hz, 1H), 4.01 (s, 2H), 3.88 (s, 3H), 3.69 (s, 2H), 2.43 (s, 3H), 0.87-0.75 (m, 4H) ppm |
| 114 | 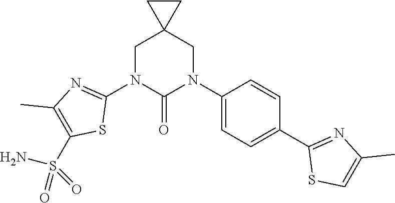 | 476.2 | ¹H NMR (400 MHz, DMSO-d₆): δ 7.93 (d, J = 8.3 Hz, 2H), 7.57 (s, 2H), 7.49 (d, J = 8.3 Hz, 2H), 7.33 (s, 1H), 4.00 (s, 2H), 3.68 (s, 2H), 2.45-2.42 (m, 6H), 0.80 (d, J = 5.4 Hz, 4H) ppm |
| 115 | 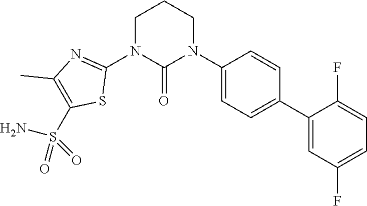 [SMILES: Cc1nc(N2CCCN(c3ccc(-c4cc(F)ccc4F)cc3)C2=O)sc1S(N)(=O)=O] | 452.1 | ¹H NMR (400 MHz, DMSO-d₆): δ 8.22 (br s, 2H), 7.67-7.61 (m, 2H), 7.55-7.36 (m, 4H), 7.32- 7.25 (m, 1H), 4.27 (t, J = 6.0 Hz, 2H), 3.86 (t, J = 5.6 Hz, 2H), 2.35-2.26 (m, 2H) ppm |
| 116 | 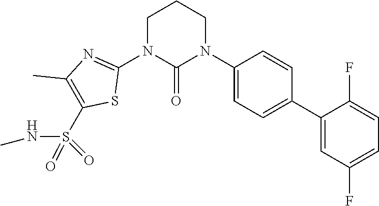 [SMILES: CNS(=O)(=O)c1sc(N2CCCN(c3ccc(-c4cc(F)ccc4F)cc3)C2=O)nc1C] | 479.1 | ¹H NMR (400 MHz, DMSO-d₆): δ 7.65-7.56 (m, 3H), 7.54-7.36 (m, 4H), 7.33-7.22 (m, 1H), 4.17 (t, J = 6.0 Hz, 2H), 3.82 (t, J = 5.6 Hz, 2H), 2.50-2.47 (m, 3H) merged in solvent peak, 2.45 (s, 3H), 2.54-2.21 (m, 2H) ppm |
| 117 | 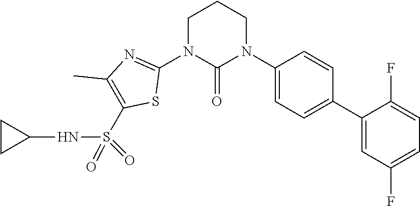 [SMILES: Cc1nc(N2CCCN(c3ccc(-c4cc(F)ccc4F)cc3)C2=O)sc1S(=O)(=O)NC1CC1] | 505.0 | ¹H NMR (400 MHz, DMSO-d₆): δ 8.03 (brs, 1H), 7.63 (d, J = 8.3 Hz, 2H), 7.51 (d, J = 8.3 Hz, 2H), 7.46-7.35 (m, 2H), 7.35-7.22 (m, 1H), 4.22- 4.15 (m, 2H), 3.87- 3.79 (m, 2H), 2.46 (s, 3H), 2.28-2.17 (m, 3H), 0.59-0.45 (m, 2H), 0.45- 0.40 (m, 2H) ppm |
| 118 | 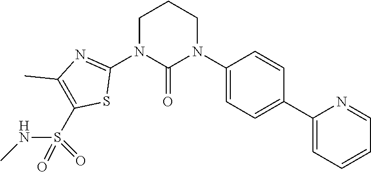 [SMILES: CNS(=O)(=O)c1sc(N2CCCN(c3ccc(-c4ccccn4)cc3)C2=O)nc1C] | 444.1 | ¹H NMR (400 MHz, DMSO-d₆): δ 8.70-8.65 (m, 1H), 8.13 (d, J = 8.3 Hz, 2H), 7.98 (d, J = 7.8 Hz, 1H), 7.95-7.84 (m, 1H), 7.58 (br s, 1H), 7.55- 7.47 (m, 2H), 7.46- 7.26 (m, 1H), 4.24-4.13 (m, 2H), 3.88-3.79 (m, 2H), 2.48-2.44 (m, 6H), 2.28-2.21 (m, 2H) ppm |
| 119 | 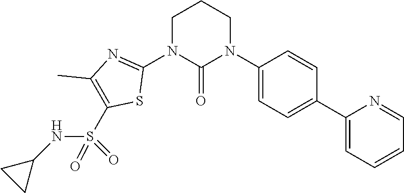 [SMILES: Cc1nc(N2CCCN(c3ccc(-c4ccccn4)cc3)C2=O)sc1S(=O)(=O)NC1CC1] | 470.0 | ¹H NMR (400 MHz, DMSO-d₆): δ 8.68 (d, J = 3.9 Hz, 1H), 8.13 (d, J = 8.3 Hz, 2H), 8.05-8.01 (m, 1H), 7.99 (d, J = 7.8 Hz, 1H), 7.96-7.83 (m, 1H), 7.51 (d, J = 8.8 Hz, 2H), 7.47-7.26 (m, 1H), 4.19 (t, J = 5.9 Hz, 2H), 3.83 (t, J = 5.4 Hz, 2H), 2.46 (s, 3H), 2.28-2.18 (m, 3H), 0.59-0.45 (m, 2H), 0.45-0.40 (m, 2H) ppm |
| 120 | 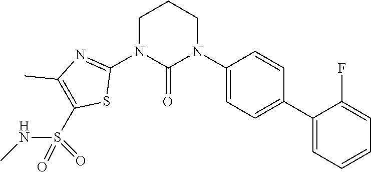 [SMILES: CNS(=O)(=O)c1sc(N2CCCN(c3ccc(-c4ccccc4F)cc3)C2=O)nc1C] | 461.2 | ¹H NMR (400 MHz, DMSO-d₆): δ 7.63-7.53 (m, 4H), 7.52-7.47 (m, 2H), 7.46-7.40 (m, 1H), 7.38-7.27 (m, 2H), 4.18 (t, J = 5.9 Hz, 2H), 3.82 (t, J = 5.5 Hz, 2H), 2.48- 2.47 (m, 3H), 2.45 (s, 3H), 2.29-2.20 (m, 2H) ppm |
| 121 | 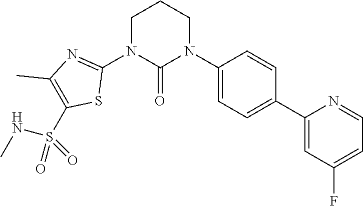 [SMILES: CNS(=O)(=O)c1sc(N2CCCN(c3ccc(-c4cc(F)ccn4)cc3)C2=O)nc1C] | 462.2 | ¹H NMR (400 MHz, DMSO-d₆): δ 8.70 (dd, J = 5.6, 9.1 Hz, 1H), 8.17 (d, J = 8.6 Hz, 2H), 7.95 (dd, J = 2.3, 11.1 Hz, 1H), 7.61-7.55 (m, 1H), 7.52 (d, J = 8.8 Hz, 2H), 7.34-7.27 (m, 1H), 4.18 (t, J = 6.0 Hz, 2H), 3.84 (t, J = 5.6 Hz, 2H), 2.49- 2.47 (m, 3H), 2.45 (s, 3H), 2.30-2.19 (m, 2H) ppm |
| 122 | 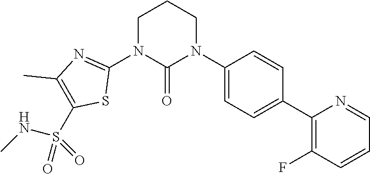 [SMILES: CNS(=O)(=O)c1sc(N2CCCN(c3ccc(-c4ncccc4F)cc3)C2=O)nc1C] | 462.2 | ¹H NMR (400 MHz, DMSO-d₆): δ 8.57 (s, 1H), 7.97 (d, J = 6.8 Hz, 2H), 7.91-7.80 (m, 1H), 7.63-7.46 (m, 4H), 4.23- 4.14 (m, 2H), 3.89- 3.80 (m, 2H), 2.50 (s, 3H), 2.46 (s, 3H), 2.30- 2.20 (m, 2H) ppm |
| 123 | 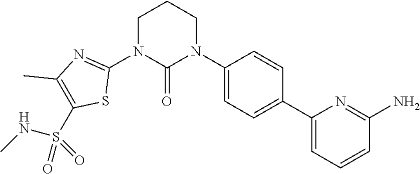 [SMILES: CNS(=O)(=O)c1sc(N2CCCN(c3ccc(-c4cccc(N)n4)cc3)C2=O)nc1C] | 459.1 | ¹H NMR (400 MHz, DMSO-d₆): δ 8.00 (d, J = 7.3 Hz, 2H), 7.58 (s, 1H), 7.43 (d, J = 7.3 Hz, 3H), 7.07 (d, J = 7.3 Hz, 1H), 6.42 (d, J = 7.3 Hz, 1H), 5.99 (s, 2H), 4.21-4.14 (m, 2H), 3.84-3.77 (m, 2H), 2.50 (s, 3H), 2.45 (s, 3H), 2.28-2.18 (m, 2H) ppm |
| 124 | 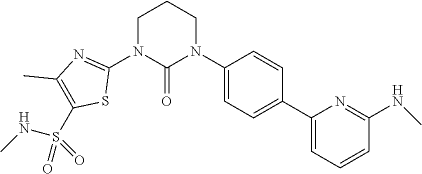 | 473.4 | ¹H NMR (400 MHz, DMSO-d₆): δ 8.31-8.17 (m, J = 8.0 Hz, 2H), 7.97- 7.88 (m, 1H), 7.87- 7.72 (m, 4H), 7.37 (d, J = 7.3 Hz, 1H), 6.90 (br s, 1H), 4.44 (t, J = 5.9 Hz, 2H), 3.82 (t, J = 5.2 Hz, 2H), 2.92 (s, 3H), 2.75- 2.70 (m, 6H), 2.25-2.22 (m, 2H) ppm |
| 125 | 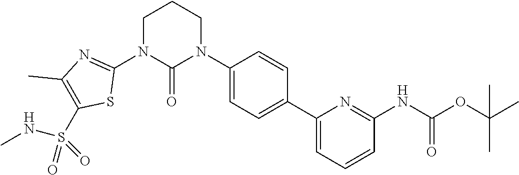 [SMILES: CNS(=O)(=O)c1sc(N2CCCN(c3ccc(-c4cccc(NC(=O)OC(C)(C)C)n4)cc3)C2=O)nc1C] | 559.1 | ¹H NMR (400 MHz, DMSO-d₆): δ 9.74 (s, 1H), 8.11 (d, J = 8.6 Hz, 2H), 7.85-7.79 (m, 1H), 7.75-7.71 (m, 1H), 7.64- 7.54 (m, 2H), 7.54- 7.44 (m, 2H), 4.18 (t, J = 5.9 Hz, 2H), 3.83 (t, J = 5.6 Hz, 2H), 2.49-2.48 (m, 3H), 2.45 (s, 3H), 2.28-2.19 (m, 2H), 1.50 (s, 9H) ppm |
| 126 | 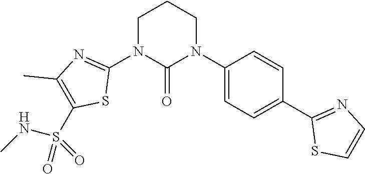 [SMILES: CNS(=O)(=O)c1sc(N2CCCN(c3ccc(-c4nccs4)cc3)C2=O)nc1C] | 450.2 | ¹H NMR (400 MHz, DMSO-d₆): δ 7.99 (d, J = 8.3 Hz, 2H), 7.96-7.91 (m, 1H), 7.80 (d, J = 3.4 Hz, 1H), 7.61-7.56 (m, 1H), 7.56-7.49 (m, 2H), 4.17 (t, J = 5.6 Hz, 2H), 3.83 (t, J = 5.4 Hz, 2H), 2.49-2.47 (m, 3H), merged in solvent peak, 2.45 (s, 3H), 2.27-2.20 (m, 2H) ppm |
| 127 | 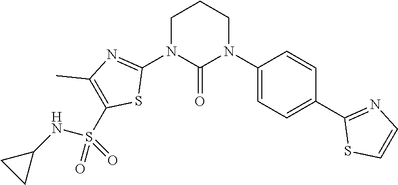 [SMILES: Cc1nc(N2CCCN(c3ccc(-c4nccs4)cc3)C2=O)sc1S(=O)(=O)NC1CC1] | 476.1 | ¹H NMR (400 MHz, DMSO-d₆): δ 8.04 (d, J = 2.6 Hz, 1H), 8.02-7.97 (m, 2H), 7.97-7.92 (m, 1H), 7.81 (d, J = 3.3 Hz, 1H), 7.58-7.50 (m, 2H), 4.19 (t, J = 6.0 Hz, 2H), 3.83 (t, J = 5.6 Hz, 2H), 2.47 (s, 3H), 2.28-2.18 (m, 3H), 0.57-0.50 (m, 2H), 0.46-0.39 (m, 2H) ppm |
| 128 | 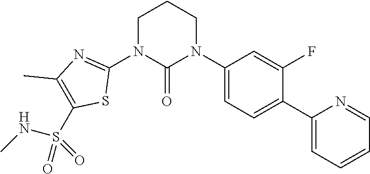 [SMILES: CNS(=O)(=O)c1sc(N2CCCN(c3ccc(-c4ccccn4)c(F)c3)C2=O)nc1C] | 462.1 | ¹H NMR (400 MHz, DMSO-d₆): δ 8.73 (d, J = 4.0 Hz, 1H), 8.00-7.89 (m, 2H), 7.81 (d, J = 7.9 Hz, 1H), 7.60 (d, J = 4.6 Hz, 1H), 7.49-7.35 (m, 3H), 4.17 (t, J = 6.0 Hz, 2H), 3.85 (t, J = 5.6 Hz, 2H), 2.49-2.47 (m, 3H), merged in solvent peak, 2.46 (s, 3H), 2.30-2.20 (m, 2H) ppm |
| 129 | 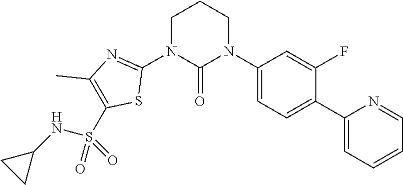 [SMILES: Cc1nc(N2CCCN(c3ccc(-c4ccccn4)c(F)c3)C2=O)sc1S(=O)(=O)NC1CC1] | 488.1 | ¹H NMR (400 MHz, DMSO-d₆): δ 8.76-8.72 (m, 1H), 8.07-8.03 (m, 1H), 8.03-7.90 (m, 2H), 7.84-7.79 (m, 1H), 7.49- 7.38 (m, 3H), 4.19 (t, J = 5.9 Hz, 2H), 3.86 (t, J = 5.6 Hz, 2H), 2.48 (s, 3H), 2.30-2.20 (m, 3H), 0.56- 0.50 (m, 2H), 0.46-0.40 (m, 2H) ppm |
| 130 | 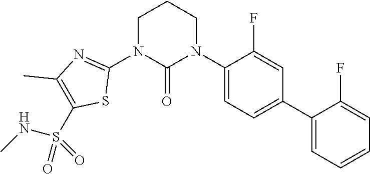 [SMILES: CNS(=O)(=O)c1sc(N2CCCN(c3ccc(-c4ccccc4F)cc3F)C2=O)nc1C] | 479.2 | ¹H NMR (400 MHz, DMSO-d₆): δ 7.63-7.57 (m, 3H), 7.56-7.51 (m, 1H), 7.50-7.44 (m, 2H), 7.40-7.29 (m, 2H), 4.21 (t, J = 6.0 Hz, 2H), 3.76 (t, J = 5.6 Hz, 2H), 2.48- 2.47 (m, 3H), 2.46 (s, 3H), 2.25 (t, J = 5.6 Hz, 2H) ppm |
| 131 | 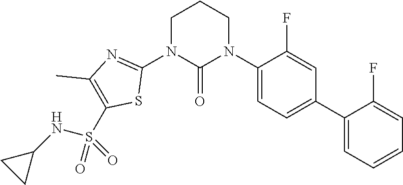 [SMILES: Cc1nc(N2CCCN(c3ccc(-c4ccccc4F)cc3F)C2=O)sc1S(=O)(=O)NC1CC1] | 505.0 | ¹H NMR (400 MHz, DMSO-d₆): δ 8.05 (d, J = 2.3 Hz, 1H), 7.55-7.41 (m, 4H), 7.41-7.31 (m, 3H), 4.18 (t, J = 6.0 Hz, 2H), 3.85 (t, J = 5.6 Hz, 2H), 2.47 (s, 3H), 2.28- 2.18 (m, 3H), 0.56-0.50 (m, 2H), 0.45-0.40 (m, 2H) ppm |
| 132 | 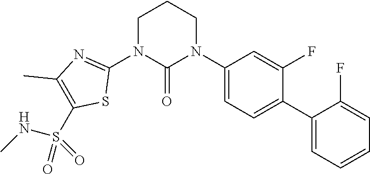 [SMILES: CNS(=O)(=O)c1sc(N2CCCN(c3ccc(-c4ccccc4F)c(F)c3)C2=O)nc1C] | 479.0 | ¹H NMR (400 MHz, DMSO-d₆): δ 7.63-7.54 (m, 1H), 7.54-7.42 (m, 4H), 7.42-7.29 (m, 3H), 4.17 (t, J = 5.9 Hz, 2H), 3.84 (t, J = 5.6 Hz, 2H), 2.49-2.47 (m, 3H), 2.45 (s, 3H), 2.27-2.19 (m, 2H) ppm |
| 133 | 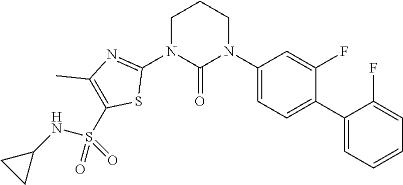 [SMILES: Cc1nc(N2CCCN(c3ccc(-c4ccccc4F)c(F)c3)C2=O)sc1S(=O)(=O)NC1CC1] | 505.2 | ¹H NMR (400 MHz, DMSO-d₆): δ 8.04 (s, 1H), 7.64-7.58 (m, 2H), 7.57-7.52 (m, 1H), 7.52- 7.44 (m, 2H), 7.40- 7.28 (m, 2H), 4.22 (t, J = 5.9 Hz, 2H), 3.77 (t, J = 5.6 Hz, 2H), 2.47 (s, 3H), 2.29-2.18 (m, 3H), 0.56- 0.49 (m, 2H), 0.46- 0.39 (m, 2H) ppm |
| 134 | 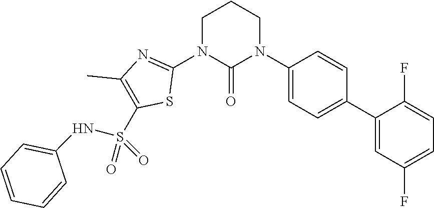 [SMILES: Cc1nc(N2CCCN(c3ccc(-c4cc(F)ccc4F)cc3)C2=O)sc1S(=O)(=O)Nc1ccccc1] | 541.1 | ¹H NMR (400 MHz, DMSO-d₆): δ 10.39 (brs, 1H), 7.62 (dd, J = 1.4, 8.5 Hz, 2H), 7.51-7.36 (m, 4H), 7.31-7.24 (m, 3H), 7.16-7.04 (m, 3H), 4.11 (t, J = 6.1 Hz, 2H), 3.78 (t, J = 5.6 Hz, 2H), 2.32 (s, 3H), 2.24-2.15 (m, 2H) ppm |
| 135 | 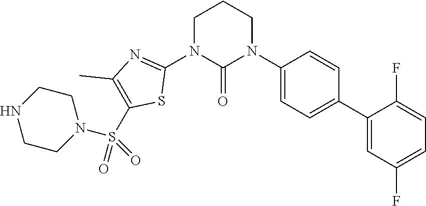 [SMILES: Cc1nc(N2CCCN(c3ccc(-c4cc(F)ccc4F)cc3)C2=O)sc1S(=O)(=O)N1CCNCC1] | 534.6 | ¹H NMR (400 MHz, DMSO-d₆): δ 7.63 (d, J = 7.8 Hz, 2H), 7.51 (d, J = 8.3 Hz, 2H), 7.46-7.36 (m, 2H), 7.35-7.20 (m, 1H), 4.24-4.15 (m, 2H), 3.88-3.78 (m, 2H), 2.92- 2.84 (m, 4H), 2.77- 2.69 (m, 4H), 2.50 (s, 3H), 2.31-2.21 (m, 3H) ppm |
| 136 | 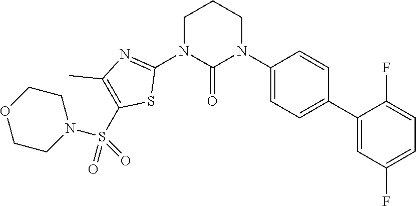 [SMILES: Cc1nc(N2CCCN(c3ccc(-c4cc(F)ccc4F)cc3)C2=O)sc1S(=O)(=O)N1CCOCC1] | 535.3 | ¹H NMR (400 MHz, DMSO-d₆): δ 7.63 (d, J = 7.8 Hz, 2H), 7.53-7.49 (m, 2H), 7.47-7.35 (m, 2H), 7.32-7.24 (m, 1H), 4.20 (t, J = 5.6 Hz, 2H), 3.87-3.78 (m, 2H), 3.69- 3.63 (m, 4H), 3.00- 2.94 (m, 4H), 2.50 (s, 3H), 2.29-2.21 (m, 2H) ppm |
| 137 | 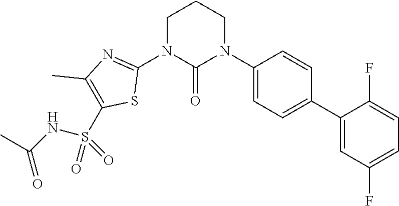 [SMILES: CC(=O)NS(=O)(=O)c1sc(N2CCCN(c3ccc(-c4cc(F)ccc4F)cc3)C2=O)nc1C] | 507.0 | ¹H NMR (400 MHz, DMSO-d₆): δ 12.57- 11.87 (m, 1H), 7.66- 7.58 (m, 2H), 7.54-7.48 (m, 2H), 7.48-7.35 (m, 2H), 7.31-7.23 (m, 1H), 4.16 (t, J = 6.0 Hz, 2H), 3.81 (t, J = 5.6 Hz, 2H), 2.46 (s, 3H), 2.26-2.19 (m, 2H), 1.85 (s, 3H) ppm |
| 138 | 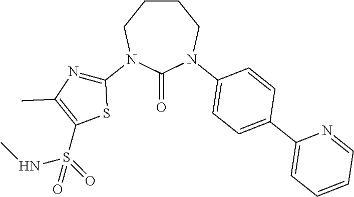 [SMILES: CNS(=O)(=O)c1sc(N2CCCCN(c3ccc(-c4ccccn4)cc3)C2=O)nc1C] | 458.2 | ¹H NMR (400 MHz, DMSO-d₆): δ 8.67 (d, J = 4.5 Hz, 1H), 8.12 (d, J = 8.6 Hz, 2H), 7.98 (d, J = 8.0 Hz, 1H), 7.94-7.84 (m, 1H), 7.60 (br s, 1H), 7.49 (d, J = 8.6 Hz, 2H), 7.36 (dd, J = 5.2, 6.8 Hz, 1H), 4.31-4.21 (m, 2H), 3.87-3.78 (m, 2H), 2.50- 2.49 (m, 3H, merged in solvent peak), 2.45 (s, 3H), 2.04-1.93 (m, 2H), 1.90-1.81 (m, 2H) ppm |
| 139 | 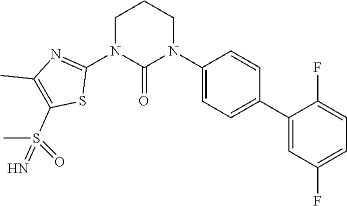 [SMILES: Cc1nc(N2CCCN(c3ccc(-c4cc(F)ccc4F)cc3)C2=O)sc1S(C)(=N)=O] | 463.1 | ¹H NMR (400 MHz, DMSO-d₆): δ 7.66-7.60 (m, 2H), 7.54-7.49 (m, 2H), 7.48-7.42 (m, 1H), 7.42-7.35 (m, 1H), 7.32- 7.24 (m, 1H), 5.32 (brs, 1H), 4.19 (t, J = 6.0 Hz, 2H), 3.83 (t, J = 5.6 Hz, 2H), 3.30 (s, 3H), 2.51 (s, 3H), 2.28-2.21 (m, 2H) ppm |
| 140 | 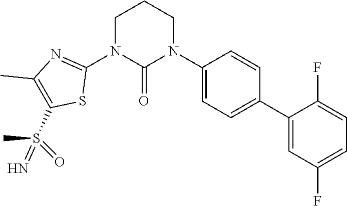 [SMILES: Cc1nc(N2CCCN(c3ccc(-c4cc(F)ccc4F)cc3)C2=O)sc1[S@@](C)(=N)=O] Single enantiomer, arbitrarily assigned stereochemistry | 463.1 | ¹H NMR (400 MHz, DMSO-d₆): δ 7.63 (d, J = 7.8 Hz, 2H), 7.54-7.34 (m, 4H), 7.28 (s, 1H), 4.59 (s, 1H), 4.22-4.11 (m, 2H), 3.89-3.76 (m, 2H), 3.12 (s, 3H), 2.50 (s, 3H), 2.29-2.18 (m, 2H) ppm |
| 141 | 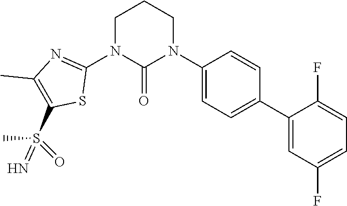 [SMILES: Cc1nc(N2CCCN(c3ccc(-c4cc(F)ccc4F)cc3)C2=O)sc1[S@](C)(=N)=O] Single enantiomer, arbitrarily assigned stereochemistry | 463.10 | ¹H NMR (400 MHz, DMSO-d₆): δ 7.66-7.59 (m, 2H), 7.54-7.34 (m, 4H), 7.33-7.22 (m, 1H), 4.59 (s, 1H), 4.20-4.12 (m, 2H), 3.88-3.76 (m, 2H), 3.12 (s, 3H), 2.50 (s, 3H), 2.29-2.18 (m, 2H) ppm |
| 142 | 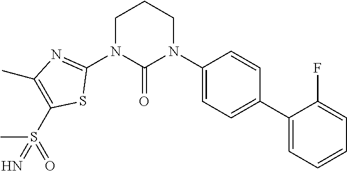 [SMILES: Cc1nc(N2CCCN(c3ccc(-c4ccccc4F)cc3)C2=O)sc1S(C)(=N)=O] | 445.7 | ¹H NMR (400 MHz, DMSO-d₆): δ 7.63-7.54 (m, 3H), 7.54-7.41 (m, 3H), 7.36-7.29 (m, 2H), 4.63-4.52 (m, 1H), 4.17 (t, J = 6.1 Hz, 2H), 3.83 (t, J = 5.7 Hz, 2H), 3.12 (d, J = 1.1 Hz, 3H), 2.27- 2.20 (m, 2H) ppm |
| 143 | 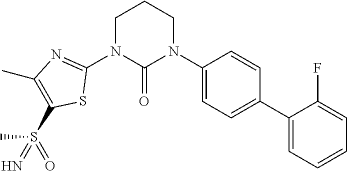 [SMILES: Cc1nc(N2CCCN(c3ccc(-c4ccccc4F)cc3)C2=O)sc1[S@](C)(=N)=O] Single enantiomer, arbitrarily assigned stereochemistry | 445.4 | ¹H NMR (400 MHz, DMSO-d₆): δ 7.60-7.54 (m, 3H), 7.49-7.42 (m, 3H), 7.36-7.29 (m, 2H), 4.57 (s, 1H), 4.21-4.13 (m, 2H), 3.86-3.78 (m, 2H), 3.11 (s, 3H), 2.50 (s, 3H), 2.28-2.17 (m, 2H) ppm |
| 144 | 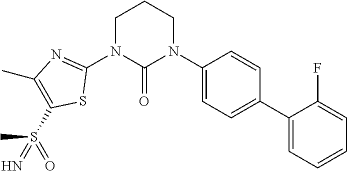 [SMILES: Cc1nc(N2CCCN(c3ccc(-c4ccccc4F)cc3)C2=O)sc1[S@@](C)(=N)=O] Single enantiomer, arbitrarily assigned stereochemistry | 445.3 | ¹H NMR (400 MHz, DMSO-d₆): δ 7.62-7.54 (m, 3H), 7.52-7.38 (m, 3H), 7.36-7.29 (m, 2H), 4.57 (s, 1H), 4.21-4.13 (m, 2H), 3.86-3.78 (m, 2H), 3.11 (s, 3H), 2.50 (s, 3H), 2.28-2.17 (m, 2H) ppm. |
| 145 | 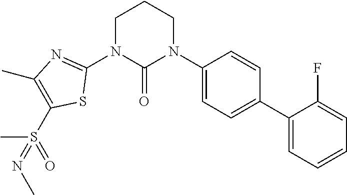 [SMILES: CN=S(C)(=O)c1sc(N2CCCN(c3ccc(-c4ccccc4F)cc3)C2=O)nc1C] | 459.3 | ¹H NMR (400 MHz, DMSO-d₆): δ 7.62-7.54 (m, 3H), 7.52-7.40 (m, 3H), 7.37-7.28 (m, 2H), 4.22-4.15 (m, 2H), 3.87- 3.79 (m, 2H), 3.23 (s, 3H), 2.57 (s, 3H), 2.45 (s, 3H), 2.29-2.18 (m, 2H) ppm |
| 146 | 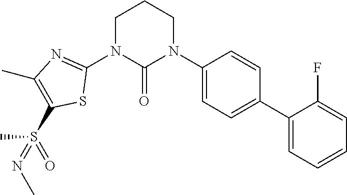 [SMILES: CN=[S@](C)(=O)c1sc(N2CCCN(c3ccc(-c4ccccc4F)cc3)C2=O)nc1C] Single enantiomer, arbitrarily assigned stereochemistry | 459.0 | ¹H NMR (400 MHz, DMSO-d₆): δ 7.63-7.53 (m, 3H), 7.52-7.40 (m, 3H), 7.38-7.28 (m, 2H), 4.22-4.14 (m, 2H), 3.86- 3.78 (m, 2H), 3.16 (s, 3H), 2.55 (s, 3H), 2.45 (s, 3H), 2.29-2.20 (m, 2H) ppm |
| 147 | 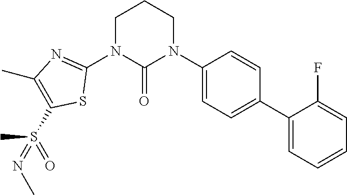 [SMILES: CN=[S@@](C)(=O)c1sc(N2CCCN(c3ccc(-c4ccccc4F)cc3)C2=O)nc1C] Single enantiomer, arbitrarily assigned stereochemistry | 459.1 | ¹H NMR (400 MHz, DMSO-d₆): δ 7.63-7.53 (m, 3H), 7.52-7.38 (m, 3H), 7.38-7.27 (m, 2H), 4.21-4.12 (m, 2H), 3.87- 3.75 (m, 2H), 3.16 (s, 3H), 2.55 (s, 3H), 2.45 (s, 3H), 2.29-2.17 (m, 2H) ppm |
| 148 | 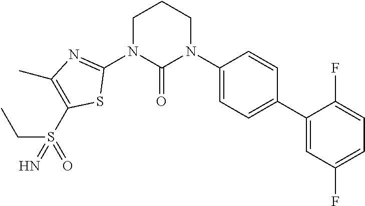 [SMILES: CCS(=N)(=O)c1sc(N2CCCN(c3ccc(-c4cc(F)ccc4F)cc3)C2=O)nc1C] | 477.6 | ¹H NMR (400 MHz, DMSO-d₆): δ 7.64-7.60 (m, 2H), 7.52-7.36 (m, 4H), 7.31-7.24 (m, 1H), 4.54 (s, 1H), 4.17 (t, J = 6.1 Hz, 2H), 3.82 (t, J = 5.6 Hz, 2H), 3.17 (q, J = 7.2 Hz, 2H), 2.48 (s, 3H), 2.27-2.20 (m, 2H), 1.15 (t, J = 7.3 Hz, 3H) ppm |
| 149 | 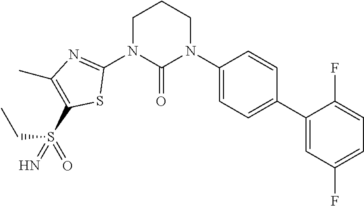 [SMILES: CC[S@@](=N)(=O)c1sc(N2CCCN(c3ccc(-c4cc(F)ccc4F)cc3)C2=O)nc1C] Single enantiomer, arbitrarily assigned stereochemistry | 477.3 | ¹H NMR (400 MHz, DMSO-d₆): δ 7.62 (d, J = 8.3 Hz, 2H), 7.53-7.35 (m, 4H), 7.32-7.23 (m, 1H), 4.56 (s, 1H), 4.20- 4.14 (m, 2H), 3.85-3.80 (m, 2H), 3.25-3.09 (m, 2H), 2,49 (s, 3H), 2.27- 2.20 (m, 2H), 1.15 (t, J = 7.3 Hz, 3H) ppm |
| 150 | 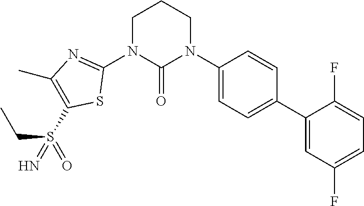 [SMILES: CC[S@](=N)(=O)c1sc(N2CCCN(c3ccc(-c4cc(F)ccc4F)cc3)C2=O)nc1C] Single enantiomer, arbitrarily assigned stereochemistry | 477.3 | ¹H NMR (400 MHz, DMSO-d₆): δ 7.62-7.58 (m, 2H), 7.49 (d, J = 8.3 Hz, 2H), 7.45-7.32 (m, 2H), 7.30-7.21 (m, 1H), 4.53 (s, 1H), 4.19-4.12 (m, 2H), 3.84-3.76 (m, 2H), 3.19-3.11 (m, 2H), 2.49 (s, 3H), 2.25-2.18 (m, 2H), 1.14 (t, J = 7.3 Hz, 3H) ppm |
| 151 | 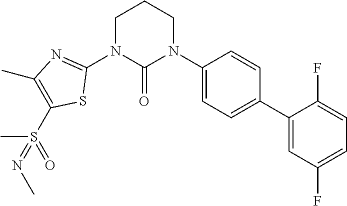 [SMILES: CN=S(C)(=O)c1sc(N2CCCN(c3ccc(-c4cc(F)ccc4F)cc3)C2=O)nc1C] | 477.2 | ¹H NMR (400 MHz, DMSO-d₆): δ 7.63 (dd, J = 1.5, 8.5 Hz, 2H), 7.54- 7.36 (m, 4H), 7.32-7.25 (m, 1H), 4.19 (t, J = 5.9 Hz, 2H), 3.83 (t, J = 5.6 Hz, 2H), 2.60 (s, 3H), 2.47 (s, 3H), 2.33-2.20 (m, 2H) ppm |
| 152 | 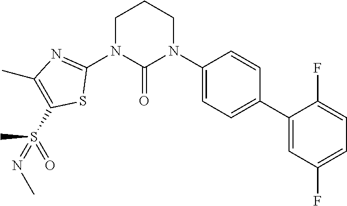 [SMILES: CN=[S@@](C)(=O)c1sc(N2CCCN(c3ccc(-c4cc(F)ccc4F)cc3)C2=O)nc1C] Single enantiomer, arbitrarily assigned stereochemistry | 477.6 | ¹H NMR (400 MHz, DMSO-d₆): δ 7.64 (d, J = 7.8 Hz, 2H), 7.52 (d, J = 8.3 Hz, 2H), 7.49-7.45 (m, 1H), 7.45-7.36 (m, 1H), 7.35-7.25 (m, 1H), 4.20 (t, J = 5.9 Hz, 2H), 3.84 (t, J = 5.1 Hz, 2H), 3.19 (s, 3H), 2.57 (s, 3H), 2.46 (s, 3H), 2.32-2.19 (m, 2H) ppm |
| 153 | 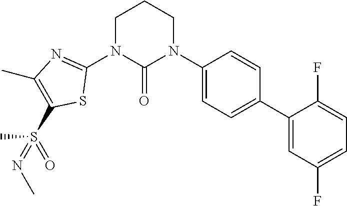 [SMILES: CN=[S@](C)(=O)c1sc(N2CCCN(c3ccc(-c4cc(F)ccc4F)cc3)C2=O)nc1C] Single enantiomer, arbitrarily assigned stereochemistry | 477.7 | ¹H NMR (400 MHz, DMSO-d₆): δ 7.63 (d, J = 8.3 Hz, 2H), 7.50 (d, J = 8.3 Hz, 2H), 7.47-7.42 (m, 1H), 7.41-7.34 (m, 1H), 7.30-7.23 (m, 1H), 4.22-4.14 (m, 2H), 3.86- 3.79 (m, 2H), 3.16 (s, 3H), 2.54 (s, 3H), 2.44 (s, 3H), 2.28-2.19 (m, 2H) ppm |
| 154 | 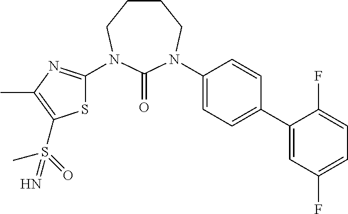 [SMILES: Cc1nc(N2CCCCN(c3ccc(-c4cc(F)ccc4F)cc3)C2=O)sc1S(C)(=N)=O] | 477.5 | ¹H NMR (400 MHz, DMSO-d₆): δ 7.62 (d, J = 8.3 Hz, 2H), 7.55-7.34 (m, 4H), 7.33-7.22 (m, 1H), 4.29-4.21 (m, 2H), 3.84-3.77 (m, 2H), 3.13 (s, 3H), 2.03-1.93 (m, 2H), 1.89-1.79 (m, 2H) ppm (Note: -NH and -CH₃ protons not observed) |
| 155 | 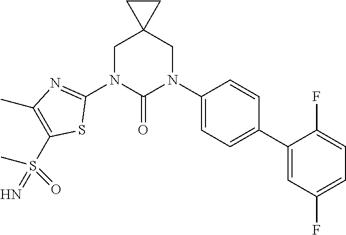 [SMILES: Cc1nc(N2CC3(CC3)CN(c3ccc(-c4cc(F)ccc4F)cc3)C2=O)sc1S(C)(=N)=O] | 489.2 | ¹H NMR (400 MHz, DMSO-d₆): δ 7.60 (d, J = 7.8 Hz, 2H), 7.47 (d, J = 8.3 Hz, 2H), 7.44-7.32 (m, 2H), 7.30-7.21 (m, 1H), 4.56 (s, 1H), 3.99 (s, 2H), 3.67 (s, 2H), 3.12- 3.11 (m, 3H), 2.45 (s, 3H), 0.79-0.78 (m, 4 H) ppm |
| 156 | 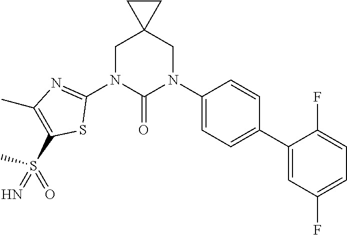 [SMILES: Cc1nc(N2CC3(CC3)CN(c3ccc(-c4cc(F)ccc4F)cc3)C2=O)sc1[S@](C)(=N)=O] Single enantiomer, arbitrarily assigned stereochemistry | 489.4 | ¹H NMR (400 MHz, DMSO-d₆): δ 7.65-7.59 (m, 2H), 7.51-7.36 (m, 4H), 7.33-7.22 (m, 1H), 4.57 (s, 1H), 4.01 (s, 2H), 3.69 (s, 2H), 3.15-3.09 (m, 3H), 2.47 (s, 3H), 0.81 (d, J = 4.4 Hz, 4H) ppm |
| 157 | 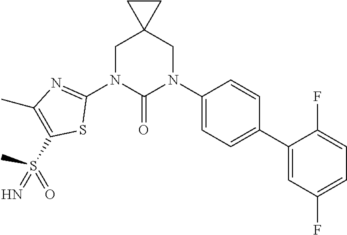 [SMILES: Cc1nc(N2CC3(CC3)CN(c3ccc(-c4cc(F)ccc4F)cc3)C2=O)sc1[S@@](C)(=N)=O] Single enantiomer, arbitrarily assigned stereochemistry | 489.5 | ¹H NMR (400 MHz, DMSO-d₆): δ 7.64-7.59 (m, 2H), 7.51-7.36 (m, 4H), 7.31-7.24 (m, 1H), 4.57 (s, 1H), 4.01 (s, 2H), 3.69 (s, 2H), 3.12 (d, J = 0.9 Hz, 3H), 2.47 (s, 3H), 0.81 (d, J = 4.5 Hz, 4H) ppm |
| 158 | 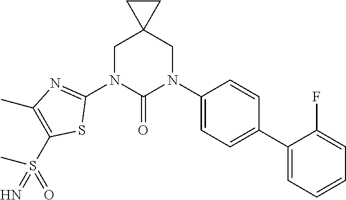 [SMILES: Cc1nc(N2CC3(CC3)CN(c3ccc(-c4ccccc4F)cc3)C2=O)sc1S(C)(=N)=O] | 471.3 | ¹H NMR (400 MHz, DMSO-d₆): δ 7.61-7.52 (m, 3H), 7.51-7.40 (m, 3H), 7.38-7.27 (m, 2H), 4.57 (s, 1H), 4.02 (s, 2H), 3.69 (s, 2H), 3.11 (m, 3H), 2.47 (s, 3H), 0.81 (d, J = 4.4 Hz, 4H) ppm |
| 159 | 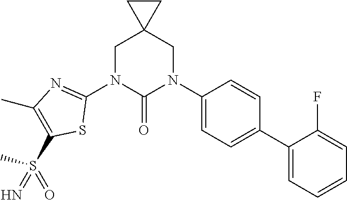 [SMILES: Cc1nc(N2CC3(CC3)CN(c3ccc(-c4ccccc4F)cc3)C2=O)sc1[S@](C)(=N)=O] Single enantiomer, arbitrarily assigned stereochemistry | 471.6 | ¹H NMR (400 MHz, DMSO-d₆): δ 7.61-7.53 (m, 3H), 7.51-7.40 (m, 3H), 7.38-7.27 (m, 2H), 4.57 (s, 1H), 4.01 (s, 2H), 3.69 (s, 2H), 3.12 (s, 3H), 2.47 (s, 3H), 0.81 (d, J = 4.1 Hz, 4H) ppm |
| 160 | 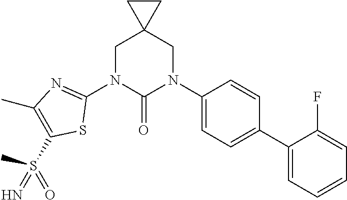 [SMILES: Cc1nc(N2CC3(CC3)CN(c3ccc(-c4ccccc4F)cc3)C2=O)sc1[S@@](C)(=N)=O] Single enantiomer, arbitrarily assigned stereochemistry | 471.4 | ¹H NMR (400 MHz, DMSO-d₆): δ 7.63-7.51 (m, 3H), 7.51-7.41 (m, 3H), 7.37-7.28 (m, 2H), 4.57 (s, 1H), 4.01 (s, 2H), 3.69 (s, 2H), 3.12 (s, 3H), 2.47 (s, 3H), 0.81 (d, J = 4.0 Hz, 4H) ppm |
| 161 | 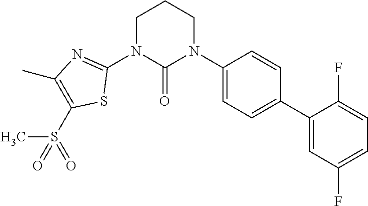 [SMILES: Cc1nc(N2CCCN(c3ccc(-c4cc(F)ccc4F)cc3)C2=O)sc1S(C)(=O)=O] | 463.1 | ¹H NMR (400 MHz, DMSO-d₆): δ 7.66-7.60 (m, 2H), 7.55-7.36 (m, 4H), 7.32-7.24 (m, 1H), 4.19 (t, J = 6.0 Hz, 2H), 3.83 (t, J = 5.6 Hz, 2H), 3.24 (s, 3H), 2.49 (s, 3H), 2.32-2.18 (m, 2H) ppm |
| 162 | 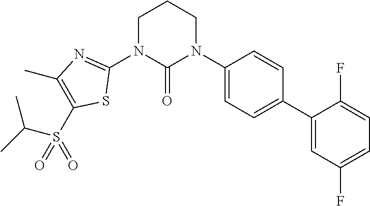 [SMILES: Cc1nc(N2CCCN(c3ccc(-c4cc(F)ccc4F)cc3)C2=O)sc1S(=O)(=O)C(C)C] | 492.2 | ¹H NMR (400 MHz, DMSO-d₆): δ 7.65-7.61 (m, 2H), 7.54-7.48 (m, 2H), 7.48-7.36 (m, 2H), 7.33-7.23 (m, 1H), 4.20 (t, J = 5.9 Hz, 2H), 3.83 (t, J = 5.4 Hz, 2H), 2.56- 2.54 (m, 1H), 2.50 (s, 3H), 2.28-2.22 (m, 2H), 1.23 (d, J = 6.8 Hz, 6H) ppm |
| 163 | 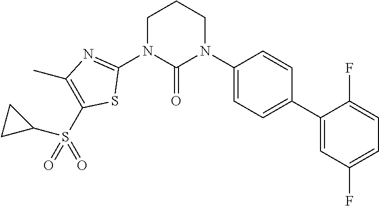 [SMILES: Cc1nc(N2CCCN(c3ccc(-c4cc(F)ccc4F)cc3)C2=O)sc1S(=O)(=O)C1CC1] | 490.1 | ¹H NMR (400 MHz, DMSO-d₆): δ 7.66-7.59 (m, 2H), 7.55-7.36 (m, 4H), 7.32-7.23 (m, 1H), 4.19 (t, J = 6.0 Hz, 2H), 3.83 (t, J = 5.6 Hz, 2H), 3.05-2.83 (m, 1H), 2.49 (s, 3H), 2.27-2.20 (m, 2H), 1.10-1.03 (m, 4H) ppm |
| 164 | 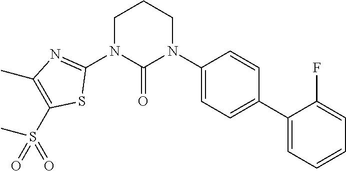 [SMILES: Cc1nc(N2CCCN(c3ccc(-c4ccccc4F)cc3)C2=O)sc1S(C)(=O)=O] | 446.2 | ¹H NMR (400 MHz, DMSO-d₆): δ 7.63-7.53 (m, 3H), 7.52-7.48 (m, 2H), 7.47-7.40 (m, 1H), 7.36-7.29 (m, 2H), 4.20 (t, J = 5.9 Hz, 2H), 3.83 (t, J = 5.6 Hz, 2H), 3.24 (s, 3H), 2.49 (s, 3H), 2.29- 2.20 (m, 2H) ppm |
| 165 | 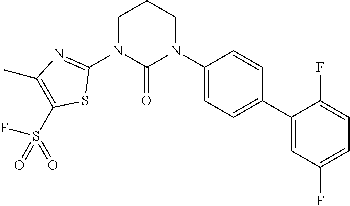 [SMILES: Cc1nc(N2CCCN(c3ccc(-c4cc(F)ccc4F)cc3)C2=O)sc1S(=O)(=O)F] | 468.2 | ¹H NMR (400 MHz, DMSO-d₆): δ 7.67-7.62 (m, 2H), 7.55-7.50 (m, 2H), 7.50-7.36 (m, 2H), 7.34-7.23 (m, 1H), 4.24 (t, J = 6.1 Hz, 2H), 3.85 (t, J = 5.6 Hz, 2H), 2.59 (s, 3H), 2.34-2.21 (m, 2H) ppm |
| 166 | 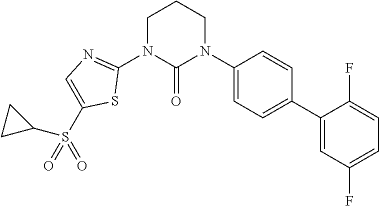 [SMILES: O=C1N(c2ccc(-c3cc(F)ccc3F)cc2)CCCN1c1ncc(S(=O)(=O)C2CC2)s1] | 476.0 | ¹H NMR (400 MHz, DMSO-d₆): δ 8.05 (s, 1H), 7.64 (d, J = 7.0 Hz, 2H), 7.52 (d, J = 8.6 Hz, 2H), 7.50-7.36 (m, 2H), 7.34-7.24 (m, 1H), 4.23 (t, J = 6.1 Hz, 2H), 3.84 (t, J = 5.8 Hz, 2H), 2.96- 2.88 (m, 1H), 2.31-2.25 (m, 2H), 1.14-1.04 (m, 4H) ppm |
| 167 | 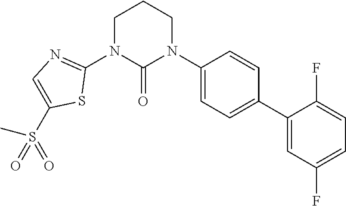 [SMILES: CS(=O)(=O)c1cnc(N2CCCN(c3ccc(-c4cc(F)ccc4F)cc3)C2=O)s1] | 450.2 | ¹H NMR (400 MHz, DMSO-d₆): δ 8.06 (s, 1H), 7.64 (d, J = 8.3 Hz, 2H), 7.52 (d, J = 8.3 Hz, 2H), 7.49-7.35 (m, 2H), 7.35-7.22 (m, 1H), 4.22 (t, J = 5.9 Hz, 2H), 3.87- 3.81 (m, 2H), 3.31 (s, 3H), 2.33-2.22 (m, 2H) ppm |
| 168 | 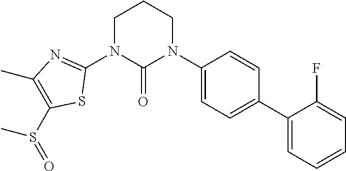 [SMILES: Cc1nc(N2CCCN(c3ccc(-c4ccccc4F)cc3)C2=O)sc1S(C)=O] | 430.2 | ¹H NMR (400 MHz, DMSO-d₆): δ 7.63-7.55 (m, 3H), 7.53-7.47 (m, 2H), 7.47-7.39 (m, 1H), 7.38-7.29 (m, 2H), 4.24- 4.17 (m, 2H), 3.88- 3.77 (m, 2H), 2.88 (s, 3H), 2.38 (s, 3H), 2.29- 2.20 (m, 2H) ppm |
| 169 | 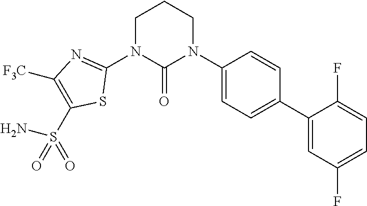 [SMILES: NS(=O)(=O)c1sc(N2CCCN(c3ccc(-c4cc(F)ccc4F)cc3)C2=O)nc1C(F)(F)F] | 519.1 | ¹H NMR (400 MHz, DMSO-d₆): δ 8.13 (br s, 2H), 7.68-7.59 (m, 2H), 7.56-7.49 (m, 2H), 7.48- 7.34 (m, 2H), 7.33- 7.21 (m, 1H), 4.22-4.11 (m, 2H), 3.89-3.78 (m, 2H), 2.31-2.19 (m, 2H) ppm |
| 170 | 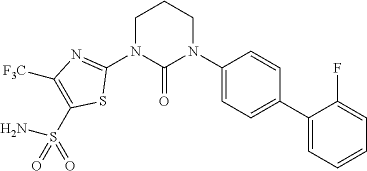 [SMILES: NS(=O)(=O)c1sc(N2CCCN(c3ccc(-c4ccccc4F)cc3)C2=O)nc1C(F)(F)F] | 501.1 | ¹H NMR (400 MHz, DMSO-d₆): δ 8.11 (s, 2H), 7.63-7.49 (m, 5H), 7.48-7.39 (m, 1H), 7.39- 7.26 (m, 2H), 4.16 (t, J = 6.0 Hz, 2H), 3.84 (t, J = 5.6 Hz, 2H), 2.32-2.21 (m, 2H) ppm |
| 171 | 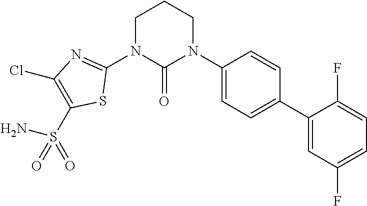 [SMILES: NS(=O)(=O)c1sc(N2CCCN(c3ccc(-c4cc(F)ccc4F)cc3)C2=O)nc1Cl] | 485.2 | ¹H NMR (400 MHz, DMSO-d₆): δ 7.94-7.81 (m, 2H), 7.64 (d, J = 7.3 Hz, 2H), 7.52 (d, J = 8.6 Hz, 2H), 7.48-7.34 (m, 2H), 7.32-7.25 (m, 1H), 4.14 (t, J = 6.0 Hz, 2H), 3.84 (t, J = 5.6 Hz, 2H), 2.31-2.20 (m, 2H) ppm |
| 172 | 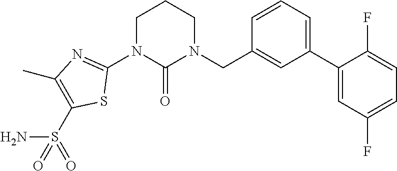 [SMILES: Cc1nc(N2CCCN(Cc3cccc(-c4cc(F)ccc4F)c3)C2=O)sc1S(N)(=O)=O] | 479.1 | ¹H NMR (400 MHz, DMSO-d₆): δ 7.51 (d, J = 7.3 Hz, 5H), 7.42-7.34 (m, 3H), 7.32-7.22 (m, 1H), 4.66 (s, 2H), 4.11- 4.03 (m, 2H), 3.44-3.36 (m, 2H), 2.42 (s, 3H), 2.10-1.99 (m, 2H) ppm |
| 173 | 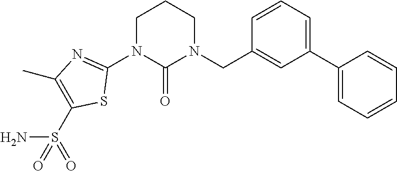 [SMILES: Cc1nc(N2CCCN(Cc3cccc(-c4ccccc4)c3)C2=O)sc1S(N)(=O)=O] | 443.2 | ¹H NMR (400 MHz, DMSO-d₆): δ 7.69-7.62 (m, 2H), 7.57 (br.s, 2H), 7.55-7.50 (m, 2H), 7.50- 7.42 (m, 3H), 7.41- 7.34 (m, 1H), 7.34-7.27 (m, 1H), 4.66 (s, 2H), 4.10-4.03 (m, 2H), 3.45- 3.36 (m, 2H), 2.42 (s, 3H), 2.09-1.99 (m, 2H) ppm |
| 174 | 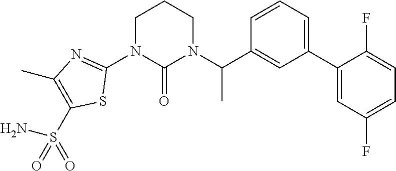 [SMILES: Cc1nc(N2CCCN(C(C)c3cccc(-c4cc(F)ccc4F)c3)C2=O)sc1S(N)(=O)=O] | 493.2 | ¹H NMR (400 MHz, DMSO-d₆): δ 7.54-7.31 (m, 8H), 7.31-7.19 (m, 1H), 5.80-5.69 (m, 1H), 4.15-4.02 (m, 1H), 3.98- 3.86 (m, 1H), 3.09- 2.93 (m, 2H), 2.41 (s, 3H), 2.08-1.85 (m, 2H), 1.58 (d, J = 6.8 Hz, 3H) ppm |
| 175 | 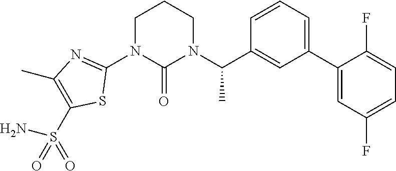 [SMILES: Cc1nc(N2CCCN([C@@H](C)c3cccc(-c4cc(F)ccc4F)c3)C2=O)sc1S(N)(=O)=O] Single enantiomer, arbitrarily assigned stereochemistry | 493.1 | ¹H NMR (400 MHz, DMSO-d₆): δ 7.62-7.31 (m, 8H), 7.33-7.18 (m, 1H), 5.82-5.67 (m, 1H), 4.17-4.00 (m, 1H), 4.01- 3.86 (m, 1H), 3.40-3.30 (m, 1H), 3.09-2.93 (m, 1H), 2.41 (s, 3H), 2.09- 1.83 (m, 2H), 1.57 (d, J = 6.8 Hz, 3H) ppm |
| 176 | 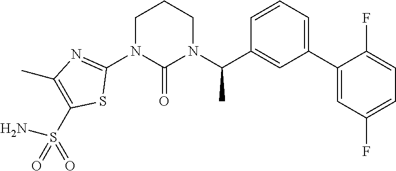 [SMILES: Cc1nc(N2CCCN([C@H](C)c3cccc(-c4cc(F)ccc4F)c3)C2=O)sc1S(N)(=O)=O] Single enantiomer, arbitrarily assigned stereochemistry | 493.2 | ¹H NMR (400 MHz, DMSO-d₆): δ 7.57-7.32 (m, 8H), 7.31-7.21 (m, 1H), 5.81-5.69 (m, 1H), 4.14-4.04 (m, 1H), 3.98- 3.87 (m, 1H), 3.40-3.30 (m, 1H), 3.05-2.94 (m, 1H), 2.41 (s, 3H), 2.07- 1.84 (m, 2H), 1.57 (d, J = 6.4 Hz, 3H) ppm |
| 177 | 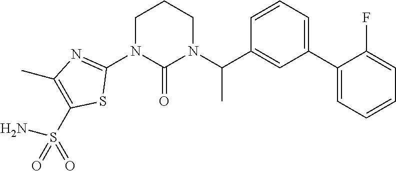 [SMILES: Cc1nc(N2CCCN(C(C)c3cccc(-c4ccccc4F)c3)C2=O)sc1S(N)(=O)=O] | 475.2 | ¹H NMR (400 MHz, DMSO-d₆): δ 7.57-7.37 (m, 8H), 7.35-7.27 (m, 2H), 5.76 (q, J = 7.0 Hz, 1H), 4.14-4.04 (m, 1H), 3.97-3.87 (m, 1H), 3.42- 3.33 (m, 1H), 3.09- 2.91 (m, 1H), 2.41 (s, 3H), 2.07-1.85 (m, 2H), 1.57 (d, J = 7.1 Hz, 3H) ppm |
| 178 | 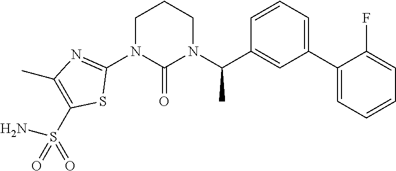 [SMILES: Cc1nc(N2CCCN([C@H](C)c3cccc(-c4ccccc4F)c3)C2=O)sc1S(N)(=O)=O] Single enantiomer, arbitrarily assigned stereochemistry | 474.9 | ¹H NMR (400 MHz, DMSO-d₆): δ 7.57-7.46 (m, 6H), 7.44-7.36 (m, 2H), 7.35-7.27 (m, 2H), 5.76 (q, J = 7.5 Hz, 1H), 4.14-4.05 (m, 1H), 3.96- 3.87 (m, 1H), 3.38- 3.33 (m, 1H), 3.08-2.92 (m, 1H), 2.41 (s, 3H), 2.05-1.97 (m, 1H), 1.94- 1.87 (m, 1H), 1.57 (d, J = 7.1 Hz, 3H) ppm |
| 179 | 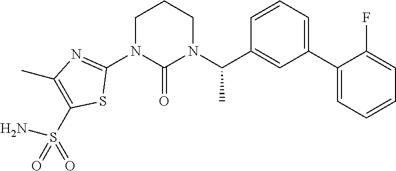 [SMILES: Cc1nc(N2CCCN([C@@H](C)c3cccc(-c4ccccc4F)c3)C2=O)sc1S(N)(=O)=O] Single enantiomer, arbitrarily assigned stereochemistry | 474.9 | ¹H NMR (400 MHz, DMSO-d₆): δ 7.57-7.37 (m, 8H), 7.34-7.28 (m, 2H), 5.76 (q, J = 7.5 Hz, 1H), 4.13-4.05 (m, 1H), 3.97-3.87 (m, 1H), 3.41- 3.32 (m, 1H), 3.09- 2.92 (m, 1H), 2.41 (s, 3H), 2.07-1.85 (m, 2H), 1.57 (d, J = 7.1 Hz, 3H) ppm |
| 180 | 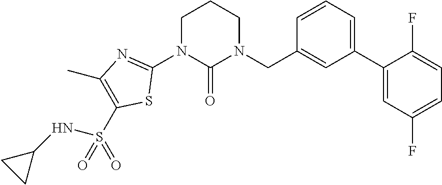 [SMILES: Cc1nc(N2CCCN(Cc3cccc(-c4cc(F)ccc4F)c3)C2=O)sc1S(=O)(=O)NC1CC1] | 519.0 | ¹H NMR (400 MHz, DMSO-d₆): δ 7.99 (s, 1H), 7.54-7.47 (m, 3H), 7.45-7.34 (m, 3H), 7.34- 7.20 (m, 1H), 4.66 (s, 2H), 4.09 (t, J = 5.9 Hz, 2H), 3.40 (t, J = 5.6 Hz, 2H), 2.44 (s, 3H), 2.40- 2.15 (m, 1H), 2.10-2.00 (m, 2H), 0.56-0.47 (m, 2H), 0.47-0.39 (m, 2H) ppm |
| 181 | 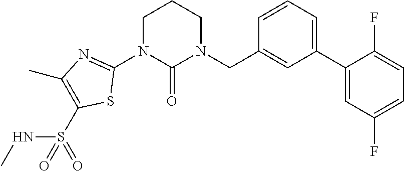 [SMILES: CNS(=O)(=O)c1sc(N2CCCN(Cc3cccc(-c4cc(F)ccc4F)c3)C2=O)nc1C] | 493.2 | ¹H NMR (400 MHz, DMSO-d₆): δ 7.57-7.46 (m, 4H), 7.46-7.32 (m, 3H), 7.30-7.21 (m, 1H), 4.66 (s, 2H), 4.08 (t, J = 6.0 Hz, 2H), 3.39 (t, J = 5.6 Hz, 2H), 2.48-2.46 (m, 3H), 2.42 (s, 3H), 2.06-2.03 (m, 2H) ppm |
| 182 | 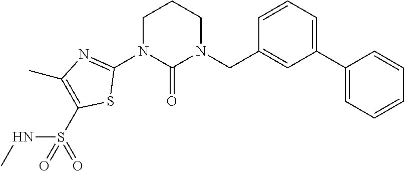 [SMILES: CNS(=O)(=O)c1sc(N2CCCN(Cc3cccc(-c4ccccc4)c3)C2=O)nc1C] | 457.1 | ¹H NMR (400 MHz, DMSO-d₆): δ 7.65 (d, J = 7.3 Hz, 2H), 7.60-7.52 (m, 3H), 7.50-7.42 (m, 3H), 7.41-7.34 (m, 1H), 7.34-7.27 (m, 1H), 4.66 (s, 2H), 4.08 (t, J = 6.0 Hz, 2H), 3.40 (t, J = 5.6 Hz, 2H), 2.49-2.47 (m, 3H), 2.43 (s, 3H), 2.10- 2.00 (m, 2H) ppm |
| 183 | 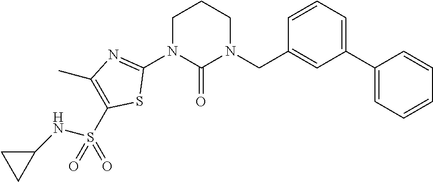 [SMILES: Cc1nc(N2CCCN(Cc3cccc(-c4ccccc4)c3)C2=O)sc1S(=O)(=O)NC1CC1] | 483.0 | ¹H NMR (400 MHz, DMSO-d₆): δ 7.99 (br s, 1H), 7.68-7.63 (m, 2H), 7.61-7.55 (m, 2H), 7.50- 7.42 (m, 3H), 7.40- 7.34 (m, 1H), 7.33-7.28 (m, 1H), 4.66 (s, 2H), 4.09 (t, J = 5.8 Hz, 2H), 3.43-3.38 (m, 2H), 2.43 (s, 3H), 2.29-2.13 (m, 1H), 2.09-2.01 (m, 2H), 0.59-0.49 (m, 2H), 0.45- 0.39 (m, 2H) ppm |
| 184 | 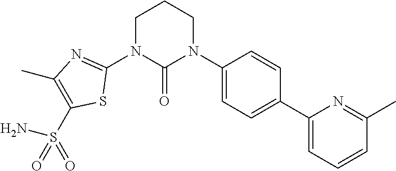 [SMILES: Cc1cccc(-c2ccc(N3CCCN(c4nc(C)c(S(N)(=O)=O)s4)C3=O)cc2)n1] | 444.4 | ¹H NMR (400 MHz, DMSO-d₆): δ 8.10 (d, J = 8.3 Hz, 2H), 7.80-7.75 (m, 2H), 7.56 (s, 2H), 7.52-7.46 (m, 2H), 7.25- 7.20 (m, 1H), 4.21- 4.13 (m, 2H), 3.86-3.80 (m, 2H), 2.54 (s, 3H), 2.45 (s, 3H), 2.27-2.19 (m, 2H). |
| 185 | 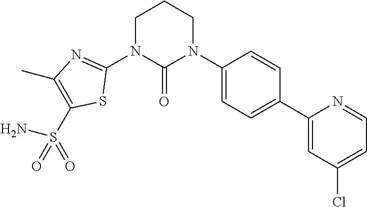 [SMILES: Cc1nc(N2CCCN(c3ccc(-c4cc(Cl)ccn4)cc3)C2=O)sc1S(N)(=O)=O] | 463.9 | ¹H NMR (400 MHz, DMSO-d₆): δ 8.65 (d, J = 5.4 Hz, 1H), 8.19-8.14 (m, 3H), 7.56 (s, 2H), 7.54-7.50 (m, 3H), 4.16 (t, J = 5.9 Hz, 2H), 3.84 (t, J = 5.6 Hz, 2H), 2.45 (s, 3H), 2.32-2.15 (m, 2H) |
| 186 | 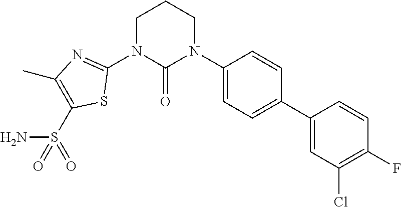 [SMILES: Cc1nc(N2CCCN(c3ccc(-c4ccc(F)c(Cl)c4)cc3)C2=O)sc1S(N)(=O)=O] | 481.1 | ¹H NMR (400 MHz, DMSO-d₆): δ 7.92 (dd, J = 2.3, 7.1 Hz, 1H), 7.79- 7.67 (m, 3H), 7.59-7.46 (m, 5H), 4.16 (t, J = 6.0 Hz, 2H), 3.81 (t, J = 5.6 Hz, 2H), 2.45 (s, 3H), 2.29-2.17 (m, 2H) |
| 187 | 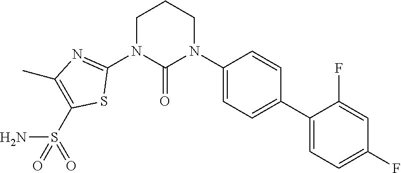 [SMILES: Cc1nc(N2CCCN(c3ccc(-c4ccc(F)cc4F)cc3)C2=O)sc1S(N)(=O)=O] | 465.3 | ¹H NMR (400 MHz, DMSO-d₆): δ 7.66-7.53 (m, 5H), 7.53-7.46 (m, 2H), 7.45-7.34 (m, 1H), 7.27-7.17 (m, 1H), 4.16 (br t, J = 5.6 Hz, 2H), 3.90-3.75 (m, 2H), 2.44 (s, 3H), 2.29-2.17 (m, 2H |
| 188 | 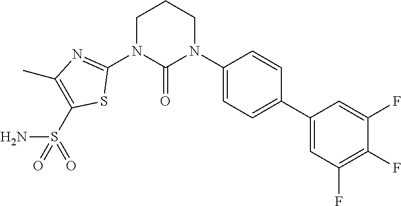 [SMILES: Cc1nc(N2CCCN(c3ccc(-c4cc(F)c(F)c(F)c4)cc3)C2=O)sc1S(N)(=O)=O] | 483.2 | ¹H NMR (400 MHz, DMSO-d₆): δ 7.88-7.64 (m, 4H), 7.56 (br s, 2H), 7.52-7.47 (m, 2H), 4.20- 4.10 (m, 2H), 3.87- 3.77 (m, 2H), 2.46 (m, 3H), 2.28-2.19 (m, 2H). |
| 189 | 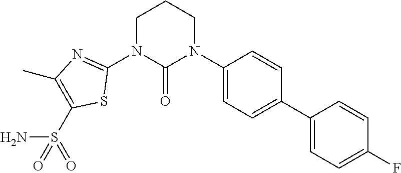 [SMILES: Cc1nc(N2CCCN(c3ccc(-c4ccc(F)cc4)cc3)C2=O)sc1S(N)(=O)=O] | 446.9 | ¹H NMR (400 MHz, DMSO-d₆): δ 7.76-7.66 (m, 4H), 7.57 (s, 2H), 7.46 (d, J = 8.6 Hz, 2H), 7.35-7.27 (m, 2H), 4.16 (t, J = 5.9 Hz, 2H), 3.81 (t, J = 5.6 Hz, 2H), 2.45 (s, 3H), 2.27-2.18 (m, 2H). |
| 190 | 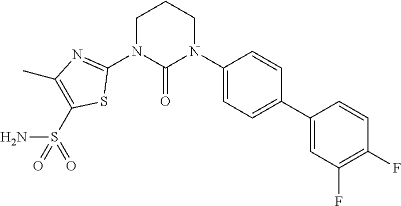 [SMILES: Cc1nc(N2CCCN(c3ccc(-c4ccc(F)c(F)c4)cc3)C2=O)sc1S(N)(=O)=O] | 465.2 | ¹H NMR (400 MHz, DMSO-d₆): δ 7.85-7.78 (m, 1H), 7.78-7.70 (m, 2H), 7.61-7.51 (m, 4H), 7.51-7.46 (m, 2H), 4.17 (t, J = 5.8 Hz, 2H), 3.85- 3.77 (m, 2H), 2.45 (s, 3H), 2.28-2.20 (m, 2H). |
| 191 | 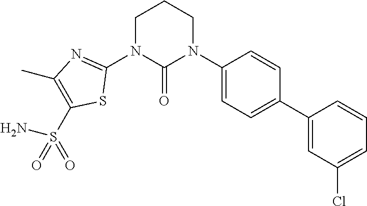 [SMILES: Cc1nc(N2CCCN(c3ccc(-c4cccc(Cl)c4)cc3)C2=O)sc1S(N)(=O)=O] | 463.3 | ¹H NMR (400 MHz, DMSO-d₆): δ 7.79-7.72 (m, 3H), 7.67 (d, J = 7.9 Hz, 1H), 7.57 (s, 2H), 7.54-7.42 (m, 4H), 4.16 (t, J = 5.9 Hz, 2H), 3.81 (t, J = 5.6 Hz, 2H), 2.44 (s, 3H), 2.28-2.19 (m, 2H). |
| 192 | 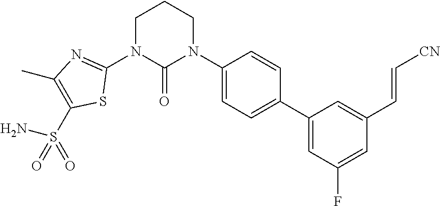 [SMILES: Cc1nc(N2CCCN(c3ccc(-c4cc(F)cc(/C=C/C#N)c4)cc3)C2=O)sc1S(N)(=O)=O] | 497.9 | ¹H NMR (400 MHz, DMSO-d₆): δ 7.87 (s, 1H), 7.83-7.75 (m, 2H), 7.75- 7.63 (m, 2H), 7.60- 7.49 (m, 5H), 6.70 (d, J = 16.8 Hz, 1H), 4.17 (t, J = 5.7 Hz, 2H), 3.86-3.79 (m, 2H), 2.45 (s, 3H), 2.27-2.19 (m, 2H). |
| 193 | 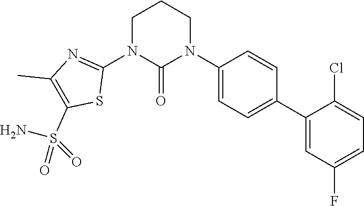 [SMILES: Cc1nc(N2CCCN(c3ccc(-c4cc(F)ccc4Cl)cc3)C2=O)sc1S(N)(=O)=O] | 481.2 | ¹H NMR (400 MHz, DMSO-d₆): δ 7.66-7.60 (m, 1H), 7.56 (s, 2H), 7.53-7.47 (m, 4H), 7.38- 7.26 (m, 2H), 4.20- 4.13 (m, 2H), 3.87-3.80 (m, 2H), 2.45 (s, 3H), 2.28-2.20 (m, 2H). |
| 194 | 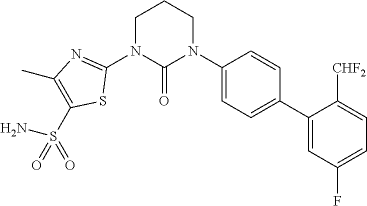 [SMILES: Cc1nc(N2CCCN(c3ccc(-c4cc(F)ccc4C(F)F)cc3)C2=O)sc1S(N)(=O)=O] | 497.4 | ¹H NMR (400 MHz, DMSO-d₆): δ 7.86-7.78 (m, 1H), 7.59-7.48 (m, 4H), 7.48-7.39 (m, 3H), 7.39-7.21 (m, 1H), 6.81 (t, J = 54.4 Hz, 1H), 4.24- 4.12 (m, 2H), 3.89- 3.79 (m, 2H), 2.45 (s, 3H), 2.28-2.21 (m, 2H). |
| 195 | 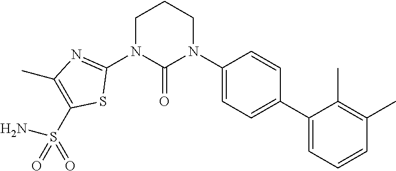 [SMILES: Cc1cccc(-c2ccc(N3CCCN(c4nc(C)c(S(N)(=O)=O)s4)C3=O)cc2)c1C] | 457.4 | ¹H NMR (400 MHz, DMSO-d₆): δ 7.57 (s, 2H), 7.44 (d, J = 7.8 Hz, 2H), 7.40-7.27 (m, 2H), 7.22- 7.13 (m, 2H), 7.06-7.04 (m, 1H), 4.22-4.15 (m, 2H), 3.87-3.79 (m, 2H), 2.46 (s, 3H), 2.31 (s, 3H), 2.28-2.20 (m, 2H), 2.14 (s, 3H). |
| 196 | 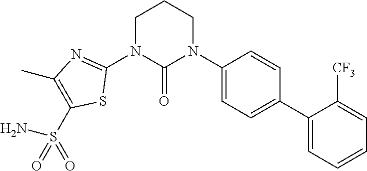 [SMILES: Cc1nc(N2CCCN(c3ccc(-c4ccccc4C(F)(F)F)cc3)C2=O)sc1S(N)(=O)=O] | 497.2 | ¹H NMR (400 MHz, DMSO-d₆): δ 7.84 (d, J = 7.9 Hz, 1H), 7.77-7.69 (m, 1H), 7.66-7.57 (m, 1H), 7.54 (s, 2H), 7.49- 7.39 (m, 3H), 7.39-7.29 (m, 2H), 4.19-4.12 (m, 2H), 3.85-3.79 (m, 2H), 2.43 (s, 3H), 2.27-2.18 (m, 2H). |
| 197 | 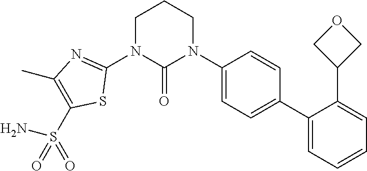 [SMILES: Cc1nc(N2CCCN(c3ccc(-c4ccccc4C4COC4)cc3)C2=O)sc1S(N)(=O)=O] | 485.3 | ¹H NMR (400 MHz, DMSO-d₆): δ 7.64 (d, J = 7.5 Hz, 1H), 7.56 (s, 2H), 7.51-7.39 (m, 3H), 7.39- 7.32 (m, 1H), 7.30- 7.22 (m, 3H), 4.70-4.59 (m, 2H), 4.57-4.51 (m, 2H), 4.50-4.43 (m, 1H), 4.17 (t, J = 5.7 Hz, 2H), 3.83 (t, J = 5.0 Hz, 2H), 2.45 (s, 3H), 2.29-2.19 (m, 2H). |
| 198 | 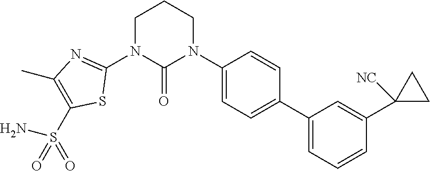 [SMILES: Cc1nc(N2CCCN(c3ccc(-c4cccc(C5(C#N)CC5)c4)cc3)C2=O)sc1S(N)(=O)=O] | 494.6 | ¹H NMR (400 MHz, DMSO-d₆): δ 7.73 (d, J = 8.3 Hz, 2H), 7.62 (d, J = 7.5 Hz, 1H), 7.56 (s, 2H), 7.54-7.51 (m, 1H), 7.51- 7.46 (m, 3H), 7.45- 7.32 (m, 1H), 4.20-4.13 (m, 2H), 3.84-3.79 (m, 2H), 2.45 (s, 3H), 2.28- 2.17 (m, 2H), 1.82-1.76 (m, 2H), 1.67-1.61 (m, 2H). |
| 199 | 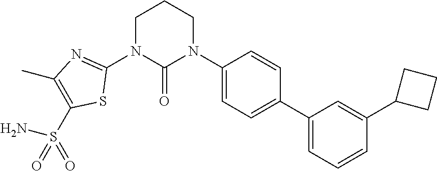 [SMILES: Cc1nc(N2CCCN(c3ccc(-c4cccc(C5CCC5)c4)cc3)C2=O)sc1S(N)(=O)=O] | 483.2 | ¹H NMR (400 MHz, DMSO-d₆): δ 7.73-7.66 (m, 2H), 7.59-7.54 (m, 2H), 7.51-7.44 (m, 4H), 7.42-7.36 (m, 1H), 7.28- 7.20 (m, 1H), 4.21- 4.11 (m, 2H), 3.86-3.77 (m, 2H), 3.67-3.55 (m, 1H), 2.45 (s, 3H), 2.39- 2.28 (m, 2H), 2.28-2.11 (m, 4H), 2.07-1.95 (m, 1H), 1.91-1.78 (m, 1H). |
| 200 | 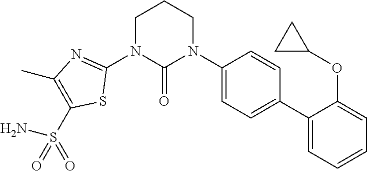 [SMILES: Cc1nc(N2CCCN(c3ccc(-c4ccccc4OC4CC4)cc3)C2=O)sc1S(N)(=O)=O] | 483.2 | ¹H NMR (400 MHz, DMSO-d₆): δ 7.56 (s, 2H), 7.51-7.45 (m, 2H), 7.45- 7.28 (m, 5H), 7.10- 7.03 (m, 1H), 4.20-4.13 (m, 2H), 3.90-3.83 (m, 1H), 3.83-3.77 (m, 2H), 2.44 (s, 3H), 2.27-2.17 (m, 2H), 0.83-0.75 (m, 2H), 0.69-0.62 (m, 2H). |
| 201 | 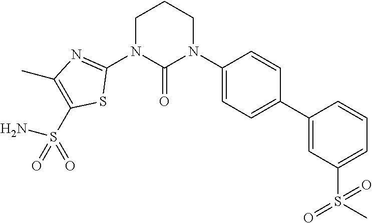 [SMILES: Cc1nc(N2CCCN(c3ccc(-c4cccc(S(C)(=O)=O)c4)cc3)C2=O)sc1S(N)(=O)=O] | 507.4 | ¹H NMR (400 MHz, DMSO-d₆): δ 8.18 (s, 1H), 8.06 (d, J = 7.8 Hz, 1H), 7.92 (d, J = 7.8 Hz, 1H), 7.82 (d, J = 8.3 Hz, 2H), 7.79-7.73 (m, 1H), 7.59- 7.51 (m, 4H), 4.17 (t, J = 5.9 Hz, 2H), 3.83 (t, J = 5.4 Hz, 2H), 2.45 (s, 3H), 2.28-2.20 (m, 2H). [Note: -3H merged in solvent peak] |
| 202 | 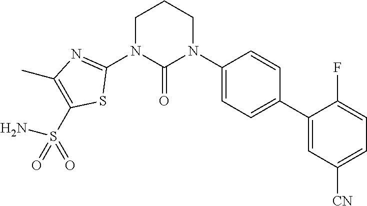 [SMILES: Cc1nc(N2CCCN(c3ccc(-c4cc(C#N)ccc4F)cc3)C2=O)sc1S(N)(=O)=O] | 472.3 | ¹H NMR (400 MHz, DMSO-d₆): δ 8.13-8.08 (m, 1H), 7.96-7.90 (m, 1H), 7.67-7.61 (m, 2H), 7.60-7.48 (m, 5H), 4.19- 4.12 (m, 2H), 3.84- 3.78 (m, 2H), 2.43 (s, 3H), 2.25-2.18 (m, 2H). |
| 203 | 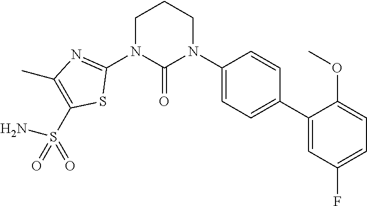 [SMILES: COc1ccc(F)cc1-c1ccc(N2CCCN(c3nc(C)c(S(N)(=O)=O)s3)C2=O)cc1] | 477.4 | ¹H NMR (400 MHz, DMSO-d₆): δ 7.57-7.53 (m, 4H), 7.42 (d, J = 8.3 Hz, 2H), 7.23-7.09 (m, 3H), 4.16 (t, J = 5.9 Hz, 2H), 3.81 (t, J = 5.6 Hz, 2H), 3.77 (s, 3H), 2.45 (s, 3H), 2.27-2.18 (m, 2H). |
| 204 | 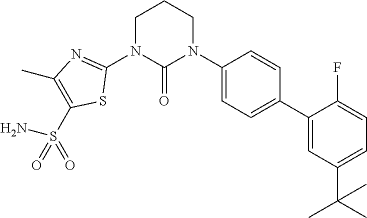 [SMILES: Cc1nc(N2CCCN(c3ccc(-c4cc(C(C)(C)C)ccc4F)cc3)C2=O)sc1S(N)(=O)=O] | 503.3 | ¹H NMR (400 MHz, DMSO-d₆): δ 7.62-7.53 (m, 4H), 7.51-7.46 (m, 4H), 7.28-7.17 (m, 1H), 4.20-4.15 (m, 2H), 3.85- 3.79 (m, 2H), 2.45 (s, 3H), 2.28-2.20 (m, 2H), 1.32 (s, 9H). |
| 205 | 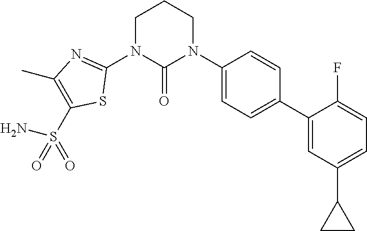 [SMILES: Cc1nc(N2CCCN(c3ccc(-c4cc(C5CC5)ccc4F)cc3)C2=O)sc1S(N)(=O)=O] | 487.3 | ¹H NMR (400 MHz, DMSO-d₆): δ 7.62-7.51 (m, 3H), 7.51-7.44 (m, 2H), 7.25-7.06 (m, 4H), 4.22-4.12 (m, 2H), 3.87- 3.77 (m, 2H), 2.45 (s, 3H), 2.28-2.19 (m, 2H), 2.04-1.94 (m, 1H), 0.96 (d, J = 6.8 Hz, 2H), 0.75- 0.68 (m, 2H). |
| 206 | 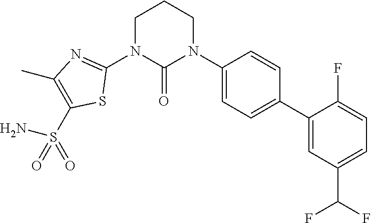 [SMILES: Cc1nc(N2CCCN(c3ccc(-c4cc(C(F)F)ccc4F)cc3)C2=O)sc1S(N)(=O)=O] | 497.5 | ¹H NMR (400 MHz, DMSO-d₆): δ 7.77 (d, J = 7.3 Hz, 1H), 7.70-7.60 (m, 3H), 7.59-7.47 (m, 5H), 7.09 (t, J = 56 Hz, 1H), 4.17 (t, J = 5.6 Hz, 2H), 3.83 (t, J = 5.4 Hz, 2H), 2.45 (s, 3H), 2.28- 2.19 (m, 2H). |
| 207 | 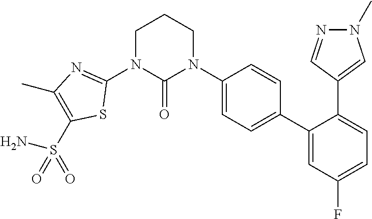 [SMILES: Cc1nc(N2CCCN(c3ccc(-c4cc(F)ccc4-c4cnn(C)c4)cc3)C2=O)sc1S(N)(=O)=O] | 527.5 | ¹H NMR (400 MHz, DMSO-d₆): δ 7.56 (s, 2H), 7.53-7.47 (m, 1H), 7.45- 7.44 (m, 1H), 7.41- 7.36 (m, 2H), 7.28-7.21 (m, 3H), 7.17-7.12 (m, 1H), 6.92 (d, J = 0.7 Hz, 1H), 4.16 (t, J = 6.1 Hz, 2H), 3.81 (t, J = 5.7 Hz, 2H), 3.74 (s, 3H), 2.44 (s, 3H), 2.27-2.19 (m, 2H). |
| 208 | 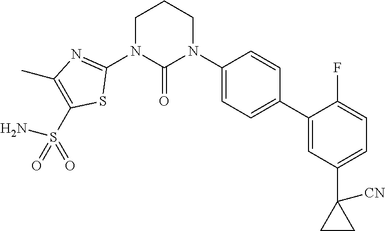 [SMILES: Cc1nc(N2CCCN(c3ccc(-c4cc(C5(C#N)CC5)ccc4F)cc3)C2=O)sc1S(N)(=O)=O] | 512.4 | ¹H NMR (400 MHz, DMSO-d₆): δ 7.62 (d, J = 7.9 Hz, 2H), 7.55 (s, 2H), 7.53-7.47 (m, 2H), 7.44- 7.40 (m, 2H), 7.40- 7.33 (m, 1H), 4.22-4.12 (m, 2H), 3.87-3.76 (m, 2H), 2.45 (s, 3H), 2.28- 2.19 (m, 2H), 1.81-1.70 (m, 2H), 1.66-1.57 (m, 2H). |
| 209 | 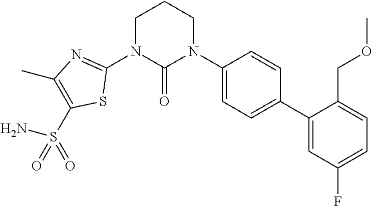 [SMILES: COCc1ccc(F)cc1-c1ccc(N2CCCN(c3nc(C)c(S(N)(=O)=O)s3)C2=O)cc1] | 491.1 | ¹H NMR (400 MHz, DMSO-d₆): δ 7.60-7.51 (m, 3H), 7.51-7.42 (m, 4H), 7.25-7.23 (m, 1H), 7.15 (dd, J = 2.8, 9.9 Hz, 1H), 4.29 (s, 2H), 4.17 (t, J = 5.9 Hz, 2H), 3.83 (t, J = 5.6 Hz, 2H), 3.26 (s, 3H), 2.45 (s, 3H), 2.42- 2.19 (m, 2H). |
| 210 | 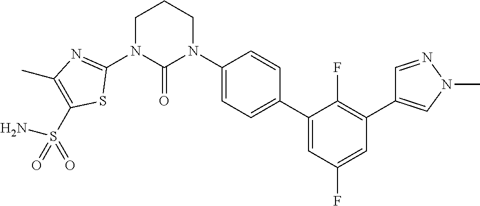 [SMILES: Cc1nc(N2CCCN(c3ccc(-c4cc(F)cc(-c5cnn(C)c5)c4F)cc3)C2=O)sc1S(N)(=O)=O] | 545.4 | ¹H NMR (400 MHz, DMSO-d₆): δ 8.23 (s, 1H), 7.99 (s, 1H), 7.69-7.60 (m, 3H), 7.58-7.48 (m, 4H), 7.28-7.20 (m, 1H), 4.22-4.12 (m, 2H), 3.91 (s, 3H), 3.87-3.80 (m, 2H), 2.45 (s, 3H), 2.29- 2.20 (m, 2H). |
| 211 | 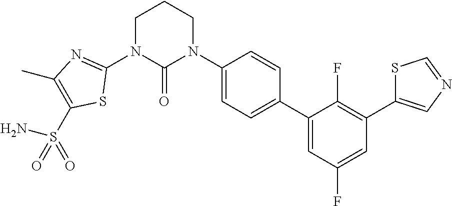 [SMILES: Cc1nc(N2CCCN(c3ccc(-c4cc(F)cc(-c5cncs5)c4F)cc3)C2=O)sc1S(N)(=O)=O] | 548.4 | ¹H NMR (400 MHz, DMSO-d₆): δ 9.27 (s, 1H), 8.54 (s, 1H), 7.88-7.82 (m, 1H), 7.70-7.65 (m, 2H), 7.60-7.52 (m, 4H), 7.51-7.46 (m, 1H), 4.17 (t, J = 5.8 Hz, 2H), 3.84 (t, J = 5.4 Hz, 2H), 2.45 (s, 3H), 2.29-2.20 (m, 2H). |
| 212 | 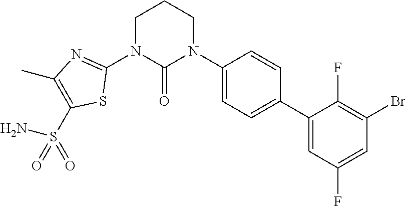 [SMILES: Cc1nc(N2CCCN(c3ccc(-c4cc(F)cc(Br)c4F)cc3)C2=O)sc1S(N)(=O)=O] | 542.7 | ¹H NMR (400 MHz, DMSO-d₆): δ 7.78-7.73 (m, 1H), 7.66-7.60 (m, 2H), 7.57 (s, 2H), 7.55- 7.48 (m, 3H), 4.16 (t, J = 5.9 Hz, 2H), 3.83 (t, J = 5.6 Hz, 2H), 2.44 (s, 3H), 2.27-2.19 (m, 2H). |
| 213 | 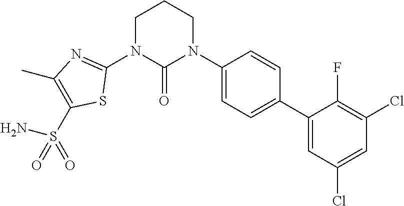 [SMILES: Cc1nc(N2CCCN(c3ccc(-c4cc(Cl)cc(Cl)c4F)cc3)C2=O)sc1S(N)(=O)=O] | 515.3 | ¹H NMR (400 MHz, DMSO-d₆): δ 7.82 (s, 1H), 7.64 (d, J = 7.3 Hz, 3H), 7.58-7.49 (m, 4H), 4.20- 4.13 (m, 2H), 3.86- 3.79 (m, 2H), 2.45 (s, 3H), 2.28-2.19 (m, 2H). |
| 214 | 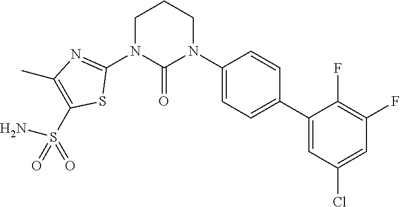 [SMILES: Cc1nc(N2CCCN(c3ccc(-c4cc(Cl)cc(F)c4F)cc3)C2=O)sc1S(N)(=O)=O] | 499.3 | ¹H NMR (400 MHz, DMSO-d₆): δ 7.81-7.69 (m, 1H), 7.68-7.59 (m, 2H), 7.59-7.47 (m, 5H), 4.21-4.11 (m, 2H), 3.88- 3.78 (m, 2H), 2.45 (s, 3H), 2.27-2.18 (m, 2H). |
| 215 | 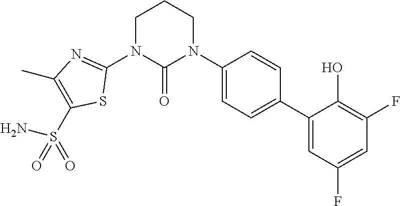 [SMILES: Cc1nc(N2CCCN(c3ccc(-c4cc(F)cc(F)c4O)cc3)C2=O)sc1S(N)(=O)=O] | 481.0 | ¹H NMR (400 MHz, DMSO-d₆): δ 9.63 (brs, 1H), 7.65-7.58 (m, 2H), 7.56 (s, 2H), 7.48-7.40 (m, 2H), 7.28-7.19 (m, 1H), 7.08-7.01 (m, 1H), 4.16 (t, J = 5.9 Hz, 2H), 3.81 (t, J = 5.6 Hz, 2H), 2.44 (s, 3H), 2.27-2.18 (m, 2H). |
| 216 | 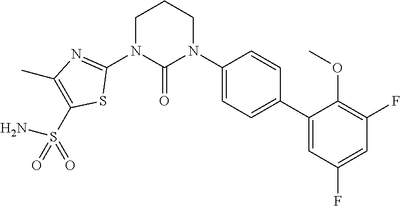 [SMILES: COc1c(F)cc(F)cc1-c1ccc(N2CCCN(c3nc(C)c(S(N)(=O)=O)s3)C2=O)cc1] | 495.5 | ¹H NMR (400 MHz, DMSO-d₆): δ 7.60-7.54 (m, 4H), 7.53-7.43 (m, 2H), 7.41-7.32 (m, 1H), 7.14 (d, J = 9.3 Hz, 1H), 4.16 (t, J = 5.6 Hz, 2H), 3.85-3.78 (m, 2H), 3.68 (s, 3H), 2.45 (s, 3H), 2.28- 2.19 (m, 2H) |
| 217 | 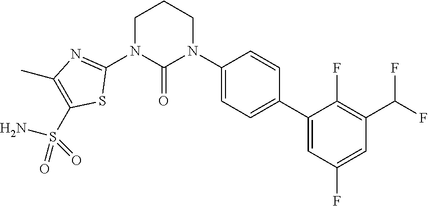 [SMILES: Cc1nc(N2CCCN(c3ccc(-c4cc(F)cc(C(F)F)c4F)cc3)C2=O)sc1S(N)(=O)=O] | 515.4 | ¹H NMR (400 MHz, DMSO-d₆): δ 7.74-7.62 (m, 3H), 7.59-7.51 (m, 5H), 7.28 (t, J = 54 Hz, 1H), 4.17 (t, J = 6.0 Hz, 2H), 3.83 (t, J = 5.6 Hz, 2H), 2.45 (s, 3H), 2.28- 2.20 (m, 2H). |
| 218 | 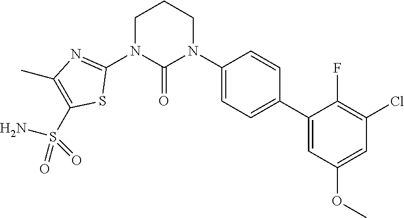 [SMILES: COc1cc(Cl)c(F)c(-c2ccc(N3CCCN(c4nc(C)c(S(N)(=O)=O)s4)C3=O)cc2)c1] | 511.4 | ¹H NMR (400 MHz, DMSO-d₆): δ 7.61 (d, J = 7.8 Hz, 2H), 7.56 (s, 2H), 7.51 (d, J = 8.3 Hz, 2H), 7.23-7.18 (m, 1H), 7.07- 7.03 (m, 1H), 4.17 (t, J = 5.9 Hz, 2H), 3.82 (s, 3H), 3.81-3.76 (m, 2H), 2.45 (s, 3H), 2.28-2.18 (m, 2H). |
| 219 | 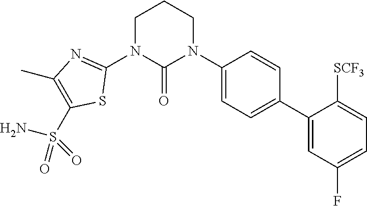 | 547.4 | ¹H NMR (400 MHz, CD₃OD-d₄): δ 8.50 (s, 1H), 7.92-7.86 (m, 1H), 7.50-7.46 (m, 2H), 7.43- 7.38 (m, 3H), 7.33- 7.23 (m, 2H), 4.28 (t, J = 6.1 Hz, 2H), 3.90 (t, J = 5.7 Hz, 2H), 2.52 (s, 3H), 2.36-2.28 (m, 2H). |
| 220 | 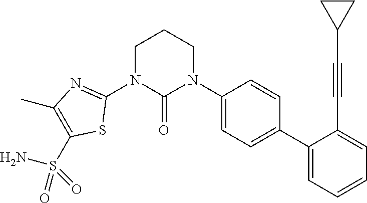 [SMILES: Cc1nc(N2CCCN(c3ccc(-c4ccccc4C#CC4CC4)cc3)C2=O)sc1S(N)(=O)=O] | 492.9 | ¹H NMR (400 MHz, DMSO-d₆): δ 7.60-7.50 (m, 4H), 7.48-7.36 (m, 5H), 7.34-7.27 (m, 1H), 4.18-4.12 (m, 2H), 3.84- 3.77 (m, 2H), 2.43 (s, 3H), 2.27-2.18 (m, 2H), 0.84-0.76 (m, 2H), 0.63- 0.56 (m, 2H). |
| 221 | 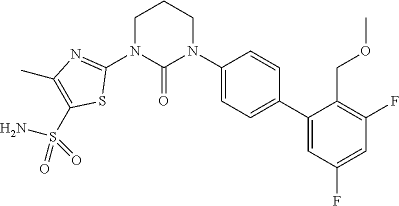 [SMILES: COCc1c(F)cc(F)cc1-c1ccc(N2CCCN(c3nc(C)c(S(N)(=O)=O)s3)C2=O)cc1] | 509.2 | ¹H NMR (400 MHz, DMSO-d₆): δ 7.58-7.47 (m, 6H), 7.41-7.23 (m, 1H), 7.12 (d, J = 8.3 Hz, 1H), 4.27-4.13 (m, 4H), 3.89-3.80 (m, 2H), 3.26 (s, 3H), 2.45 (s, 3H), 2.29- 2.18 (m, 2H). |
| 222 | 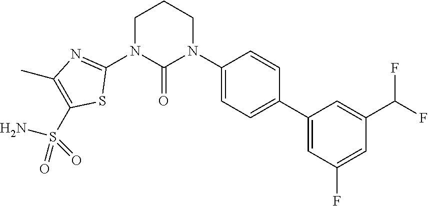 [SMILES: Cc1nc(N2CCCN(c3ccc(-c4cc(F)cc(C(F)F)c4)cc3)C2=O)sc1S(N)(=O)=O] | 497.4 | ¹H NMR (400 MHz, DMSO-d₆): δ 7.84-7.75 (m, 4H), 7.56 (s, 2H), 7.54-7.49 (m, 2H), 7.49- 7.40 (m, 1H), 7.11 (t, J = 55.6 Hz, 1H), 4.16 (t, J = 5.9 Hz, 2H), 3.82 (t, J = 5.5 Hz, 2H), 2.45 (s, 3H), 2.28-2.18 (m, 2H) |
| 223 | 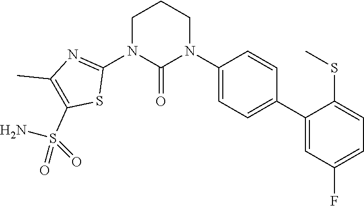 [SMILES: CSc1ccc(F)cc1-c1ccc(N2CCCN(c3nc(C)c(S(N)(=O)=O)s3)C2=O)cc1] | 493.1 | ¹H NMR (400 MHz, DMSO-d₆): δ 7.57 (s, 2H), 7.51-7.36 (m, 5H), 7.30- 7.22 (m, 1H), 7.13 (dd, J = 2.8, 9.4 Hz, 1H), 4.16 (t, J = 5.9 Hz, 2H), 3.82 (t, J = 5.4 Hz, 2H), 2.45 (s, 3H), 2.38 (s, 3H), 2.27- 2.19 (m, 2H) |
| 224 | 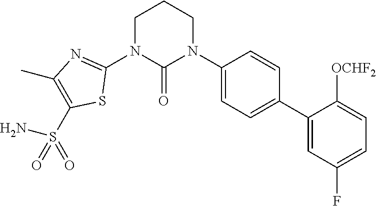 [SMILES: Cc1nc(N2CCCN(c3ccc(-c4cc(F)ccc4OC(F)F)cc3)C2=O)sc1S(N)(=O)=O] | 531.1 | ¹H NMR (400 MHz, DMSO-d₆): δ 7.60-7.49 (m, 7H), 7.49-7.45 (m, 1H), 7.43-7.34 (m, 1H), 4.16 (t, J = 5.9 Hz, 2H), 3.83 (t, J = 5.6 Hz, 2H), 2.44 (s, 3H), 2.28-2.19 (m, 2H) |
| 225 | 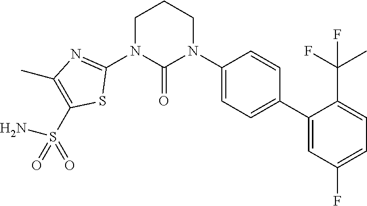 [SMILES: Cc1nc(N2CCCN(c3ccc(-c4cc(F)ccc4C(C)(F)F)cc3)C2=O)sc1S(N)(=O)=O] | 511.4 | ¹H NMR (400 MHz, DMSO-d₆): δ 7.73 (dd, J = 5.8, 8.9 Hz, 1H), 7.57 (s, 2H), 7.47-7.41 (m, 2H), 7.40-7.32 (m, 3H), 7.13 (dd, J = 2.6, 9.4 Hz, 1H), 4.16 (t, J = 6.0 Hz, 2H), 3.83 (t, J = 5.6 Hz, 2H), 2.44 (s, 3H), 2.29-2.18 (m, 2H), 1.72 (t, J = 19.0 Hz, 3H) |
| 226 | 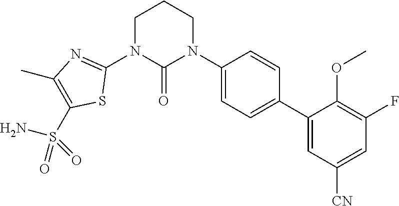 [SMILES: COc1c(F)cc(C#N)cc1-c1ccc(N2CCCN(c3nc(C)c(S(N)(=O)=O)s3)C2=O)cc1] | 502.2 | ¹H NMR (400 MHz, DMSO-d₆): δ 7.97-7.90 (m, 1H), 7.72 (s, 1H), 7.59-7.51 (m, 4H), 7.51- 7.43 (m, 2H), 4.18- 4.10 (m, 2H), 3.89-3.76 (m, 5H), 2.43 (s, 3H), 2.27-2.16 (m, 2H). |
| 227 | 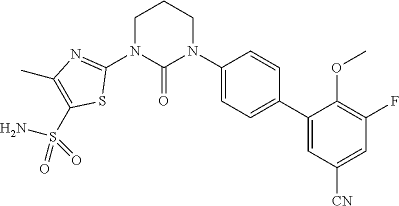 [SMILES: COc1c(F)cc(C#N)cc1-c1ccc(N2CCCN(c3nc(C)c(S(N)(=O)=O)s3)C2=O)cc1] | 545.4 | ¹H NMR (400 MHz, DMSO-d₆): δ 7.81 (dd, J = 1.9, 11.6 Hz, 1H), 7.62- 7.54 (m, 5H), 7.52-7.48 (m, 2H), 4.17 (t, J = 6.0 Hz, 2H), 3.85 (s, 3H), 3.82-3.79 (m, 2H), 2.45 (s, 3H), 2.32-2.17 (m, 2H). |
| 228 | 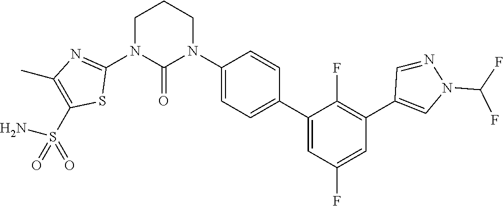 [SMILES: Cc1nc(N2CCCN(c3ccc(-c4cc(F)cc(-c5cnn(C(F)F)c5)c4F)cc3)C2=O)sc1S(N)(=O)=O] | 581.1 | 1H NMR (400 MHz, DMSO-d6): δ 8.76 (s, 1H), 8.38 (s, 1H), 7.98 (s, 2H), 7.79 (d, J = 3.20 Hz, 2H), 7.54 (d, J = 8.80 Hz, 4H), 7.39 (t, J = 2.80 Hz, 1H), 4.18 (t, J = 5.60 Hz, 2H), 3.85 (t, J = 5.60 Hz, 2H), 2.46 (s, 3H), 8.38 (s, 2H), |
| 229 | 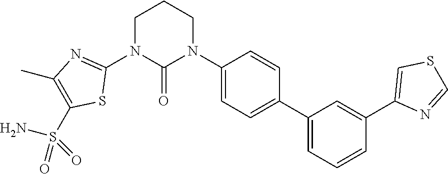 [SMILES: Cc1nc(N2CCCN(c3ccc(-c4cccc(-c5cscn5)c4)cc3)C2=O)sc1S(N)(=O)=O] | 512.4 | ¹H NMR (400 MHz, DMSO-d₆): δ 9.24 (s, 1H), 8.35-8.28 (m, 2H), 8.01 (d, J = 7.3 Hz, 1H), 7.79 (d, J = 7.8 Hz, 2H), 7.68 (d, J = 6.8 Hz, 1H), 7.60- 7.47 (m, 5H), 4.21-4.13 (m, 2H), 3.90-3.79 (m, 2H), 2.45 (s, 3H), 2.30- 2.19 (m, 2H). |
| 230 | 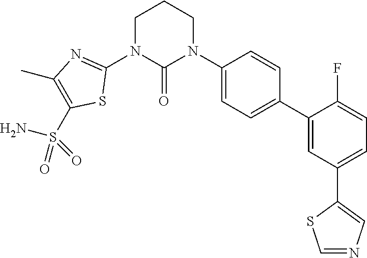 [SMILES: Cc1nc(N2CCCN(c3ccc(-c4cc(-c5cncs5)ccc4F)cc3)C2=O)sc1S(N)(=O)=O] | 530.2 | ¹H NMR (400 MHz, DMSO-d₆): δ 9.00 (s, 1H), 7.77 (s, 1H), 7.72-7.60 (m, 2H), 7.55 (s, 2H), 7.42-7.34 (m, 2H), 7.33- 7.22 (m, 3H), 4.14 (t, J = 5.5 Hz, 2H), 3.82-3.76 (m, 2H), 2.45 (s, 3H), 2.26-2.17 (m, 2H). |
| 231 | 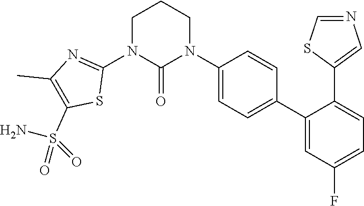 [SMILES: Cc1nc(N2CCCN(c3ccc(-c4cc(F)ccc4-c4cncs4)cc3)C2=O)sc1S(N)(=O)=O] | 530.1 | ¹H NMR (400 MHz, DMSO-d₆): δ 8.99 (s, 1H), 7.77 (s, 1H), 7.69-7.64 (m, 1H), 7.56 (br s, 2H), 7.43-7.22 (m, 6H), 4.19- 4.10 (m, 2H), 3.83- 3.75 (m, 2H), 2.44 (s, 3H), 2.26-2.13 (m, 2H). |
| 232 | 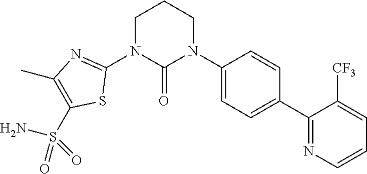 [SMILES: Cc1nc(N2CCCN(c3ccc(-c4ncccc4C(F)(F)F)cc3)C2=O)sc1S(N)(=O)=O] | 498.0 | ¹H NMR (400 MHz, DMSO-d₆): δ 8.92 (d, J = 3.9 Hz, 1H), 8.33 (d, J = 7.5 Hz, 1H), 7.73-7.61 (m, 1H), 7.57 (s, 2H), 7.54-7.48 (m, 4H), 4.17 (t, J = 5.7 Hz, 2H), 3.88- 3.81 (m, 2H), 2.45 (s, 3H), 2.31-2.19 (m, 2H). |
| 233 | 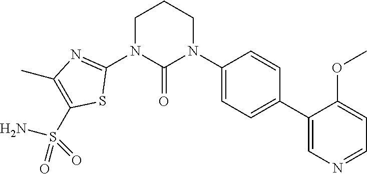 [SMILES: COc1ccncc1-c1ccc(N2CCCN(c3nc(C)c(S(N)(=O)=O)s3)C2=O)cc1] | 460.2 | ¹H NMR (400 MHz, DMSO-d₆): δ 8.46 (d, J = 5.8 Hz, 1H), 8.39 (s, 1H), 7.60-7.51 (m, 4H), 7.50- 7.39 (m, 2H), 7.18 (d, J = 5.8 Hz, 1H), 4.17 (t, J = 5.9 Hz, 2H), 3.88 (s, 3H), 3.81 (t, J = 5.6 Hz, 2H), 2.44 (s, 3H), 2.29-2.19 (m, 2H). |
| 234 | 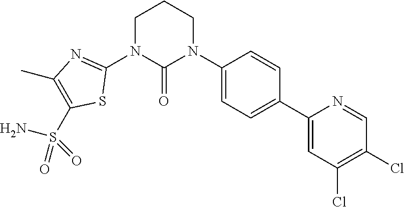 [SMILES: Cc1nc(N2CCCN(c3ccc(-c4cc(Cl)c(Cl)cn4)cc3)C2=O)sc1S(N)(=O)=O] | 498.3 | ¹H NMR (400 MHz, DMSO-d₆): δ 8.83 (s, 1H), 8.41-8.36 (s, 1H), 8.17 (d, J = 7.6 Hz, 2H), 7.63- 7.44 (m, 4H), 4.24-4.10 (m, 2H), 3.88-3.79 (m, 2H), 2.45 (s, 3H), 2.30- 2.16 (m, 2H). |
| 235 | 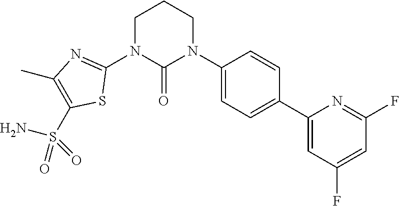 [SMILES: Cc1nc(N2CCCN(c3ccc(-c4cc(F)cc(F)n4)cc3)C2=O)sc1S(N)(=O)=O] | 466.4 | ¹H NMR (400 MHz, DMSO-d₆): δ 8.14 (d, J = 8.6 Hz, 2H), 7.99 (d, J = 9.5 Hz, 1H), 7.58-7.52 (m, 4H), 7.23 (d, J = 8.6 Hz, 1H), 4.16 (t, J = 5.8 Hz, 2H), 3.84 (t, J = 5.5 Hz, 2H), 2.45 (s, 3H), 2.32-2.16 (m, 2H). |
| 236 | 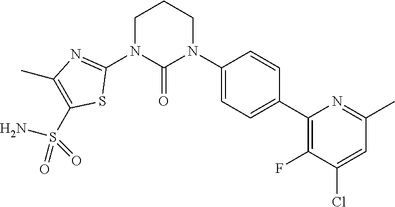 [SMILES: Cc1cc(Cl)c(F)c(-c2ccc(N3CCCN(c4nc(C)c(S(N)(=O)=O)s4)C3=O)cc2)n1] | 496.4 | ¹H NMR (400 MHz, DMSO-d₆): δ 7.95-7.89 (m, 2H), 7.63-7.59 (m, 1H), 7.58-7.52 (m, 4H), 4.17 (t, J = 5.9 Hz, 2H), 3.84 (t, J = 5.6 Hz, 2H), 2.45 (s, 3H), 2.28-2.19 (m, 2H) [Note: -CH₃ merged in solvent peak]. |
| 237 | 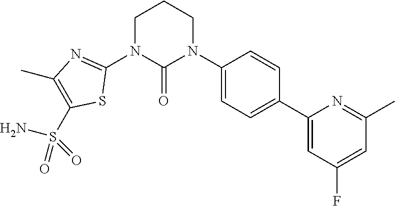 [SMILES: Cc1cc(F)cc(-c2ccc(N3CCCN(c4nc(C)c(S(N)(=O)=O)s4)C3=O)cc2)n1] | 462.6 | ¹H NMR (400 MHz, DMSO-d₆): δ 8.14 (d, J = 8.8 Hz, 2H), 7.73 (d, J = 10.8 Hz, 1H), 7.55 (s, 2H), 7.53-7.48 (m, 2H), 7.19-7.14 (m, 1H), 4.17 (t, J = 5.9 Hz, 2H), 3.83 (t, J = 5.4 Hz, 2H), 2.56 (s, 3H), 2.45 (s, 3H), 2.29- 2.18 (m, 2H) |
| 238 | 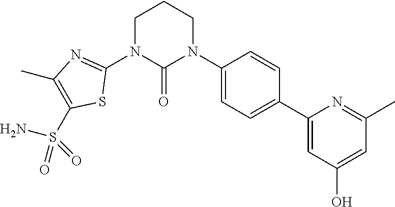 [SMILES: Cc1cc(O)cc(-c2ccc(N3CCCN(c4nc(C)c(S(N)(=O)=O)s4)C3=O)cc2)n1] | 460.4 | ¹H NMR (400 MHz, DMSO-d₆): δ 8.00-7.85 (m, 2H), 7.70-7.37 (m, 4H), 7.13-6.79 (m, 1H), 6.69-6.29 (m, 1H), 4.16 (t, J = 5.9 Hz, 2H), 3.81 (t, J = 5.5 Hz, 2H), 2.44 (s, 3H), 2.37 (s, 3H), 2.27- 2.19 (m, 2H). [Note: -OH proton not observed] |
| 239 | 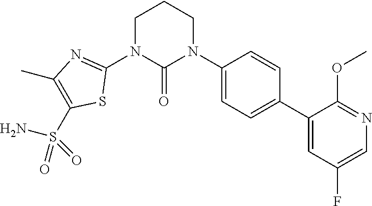 [SMILES: COc1ncc(F)cc1-c1ccc(N2CCCN(c3nc(C)c(S(N)(=O)=O)s3)C2=O)cc1] | 478.4 | ¹H NMR (400 MHz, DMSO-d₆): δ 8.18 (d, J = 3.0 Hz, 1H), 7.82 (dd, J = 2.9, 8.8 Hz, 1H), 7.64 (d, J = 8.5 Hz, 2H), 7.56 (s, 2H), 7.50-7.42 (m, 2H), 4.17 (t, J = 5.8 Hz, 2H), 3.89 (s, 3H), 3.81 (t, J = 5.2 Hz, 2H), 2.44 (s, 3H), 2.32-2.14 (m, 2H). |
| 240 | 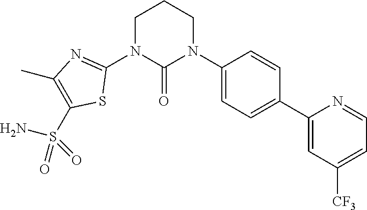 [SMILES: Cc1nc(N2CCCN(c3ccc(-c4cc(C(F)(F)F)ccn4)cc3)C2=O)sc1S(N)(=O)=O] | 498.4 | ¹H NMR (400 MHz, DMSO-d₆): δ 8.95 (d, J = 4.9 Hz, 1H), 8.32 (s, 1H), 8.28-8.22 (m, 2H), 7.78- 7.71 (m, 1H), 7.61- 7.50 (m, 4H), 4.20-4.12 (m, 2H), 3.90-3.81 (m, 2H), 2.45 (s, 3H), 2.30- 2.19 (m, 2H) |
| 241 | 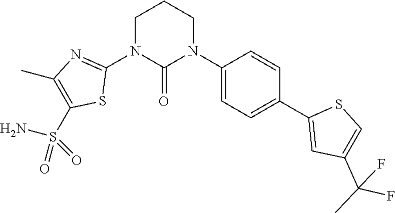 [SMILES: Cc1nc(N2CCCN(c3ccc(-c4cc(C(C)(F)F)cs4)cc3)C2=O)sc1S(N)(=O)=O] | 499.2 | ¹H NMR (400 MHz, DMSO-d₆): δ 7.84 (s, 1H), 7.75-7.63 (m, 3H), 7.53 (s, 2H), 7.43 (d, J = 7.8 Hz, 2H), 4.18-4.08 (m, 2H), 3.83-3.72 (m, 2H), 2.42 (s, 3H), 2.26-2.15 (m, 2H), 2.01 (t, J = 18.6 Hz, 3H). |
| 242 | 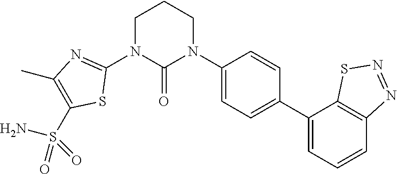 [SMILES: Cc1nc(N2CCCN(c3ccc(-c4cccc5nnsc45)cc3)C2=O)sc1S(N)(=O)=O] | 484.4 | ¹H NMR (400 MHz, DMSO-d₆): δ 8.75 (d, J = 8.3 Hz, 1H), 8.01 (d, J = 6.8 Hz, 1H), 7.95-7.87 (m, 1H), 7.80 (d, J = 8.3 Hz, 2H), 7.60 (d, J = 8.3 Hz, 2H), 7.56-7.52 (m, 2H), 4.20-4.12 (m, 2H), 3.89-3.80 (m, 2H), 2.43 (s, 3H), 2.27-2.19 (m, 2H). |
| 243 | 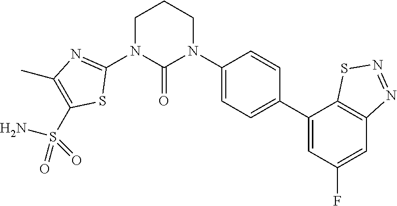 [SMILES: Cc1nc(N2CCCN(c3ccc(-c4cc(F)cc5nnsc45)cc3)C2=O)sc1S(N)(=O)=O] | 505.1 | ¹H NMR (400 MHz, DMSO-d₆): δ 8.68 (dd, J = 2.3, 8.5 Hz, 1H), 8.05 (dd, J = 2.3, 9.6 Hz, 1H), 7.86 (d, J = 8.6 Hz, 2H), 7.66-7.60 (m, 2H), 7.58 (s, 2H), 4.18 (t, J = 5.9 Hz, 2H), 3.87 (t, J = 5.6 Hz, 2H), 2.45 (s, 3H), 2.30-2.21 (m, 2H). |
| 244 | 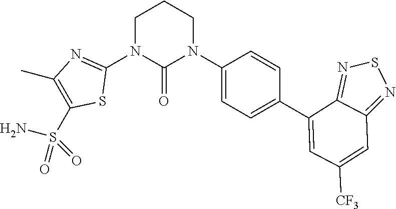 [SMILES: Cc1nc(N2CCCN(c3ccc(-c4cc(C(F)(F)F)cc5nsnc45)cc3)C2=O)sc1S(N)(=O)=O] | 555.4 | ¹H NMR (400 MHz, DMSO-d₆): δ 8.61 (s, 1H), 8.13-8.00 (m, 3H), 7.68- 7.45 (m, 4H), 4.27- 4.11 (m, 2H), 3.95-3.76 (m, 2H), 2.44 (s, 3H), 2.29-2.17 (m, 2H). |
| 245 | 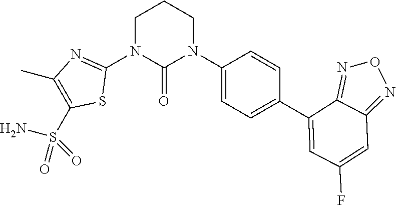 [SMILES: Cc1nc(N2CCCN(c3ccc(-c4cc(F)cc5nonc45)cc3)C2=O)sc1S(N)(=O)=O] | 489.0 | 1H-NMR (400 MHz, DMSO-d₆) 8.17 (d, J = 8.80 Hz, 2H), 7.97-7.94 (m, 2H), 7.63 (d, J = 8.40 Hz, 2H),7.59 (s, 2H), 4.18 (t, J = 6.00 Hz, 2H), 3.87 (t, J = 6.00 Hz, 2H), 2.46 (s, 3H) , 2.26 (t, J = 5.60 Hz, 2H). |
| 246 | 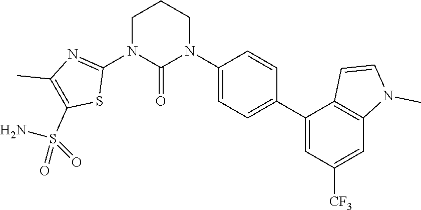 | 550.2 | (400 MHz, DMSO-d6 ) 7.92 (s, 1H), 7.76 (d, J = 2.00 Hz, 2H), 7.69 (d, J = 2.80 Hz, 1H), 7.55 (t, J = 1.60 Hz, 4H), 7.38 (s, 1H), 6.69 (d, J = 2.40 Hz, 1H), 4.19 (t, J = 6.00 Hz, 2H), 3.95 (s, 3H), 3.87 (t, J = 5.60 Hz, 2H), 2.46 (s, 3H), 2.08 (t, 2H) |
| 247 | 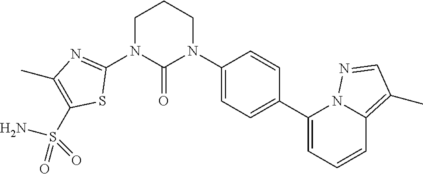 [SMILES: Cc1nc(N2CCCN(c3ccc(-c4cccc5c(C)cnn45)cc3)C2=O)sc1S(N)(=O)=O] | 483.4 | ¹H NMR (400 MHz, DMSO-d₆): δ 8.02-7.95 (m, 2H), 7.86 (s, 1H), 7.66 (d, J = 9.0 Hz, 1H), 7.54 (d, J = 8.5 Hz, 4H), 7.31-7.23 (m, 1H), 7.00 (d, J = 6.9 Hz, 1H), 4.18 (t, J = 5.9 Hz, 2H), 3.86 (t, J = 5.3 Hz, 2H), 2.45 (s, 3H), 2.32 (s, 3H), 2.29- 2.21 (m, 2H). |
| 248 | 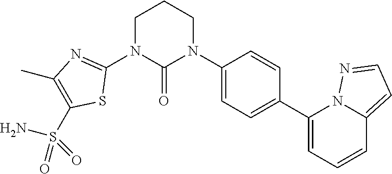 [SMILES: Cc1nc(N2CCCN(c3ccc(-c4cccc5ccnn45)cc3)C2=O)sc1S(N)(=O)=O] | 469.1 | ¹H NMR (400 MHz, DMSO-d₆): δ 8.05-7.97 (m, 3H), 7.74 (d, J = 8.8 Hz, 1H), 7.59-7.52 (m, 4H), 7.36-7.28 (m, 1H), 7.05 (d, J = 6.8 Hz, 1H), 6.77-6.73 (m, 1H), 4.18 (t, J = 5.6 Hz, 2H), 3.86 (t, J = 5.4 Hz, 2H), 2.46 (s, 3H), 2.30-2.21 (m, 2H). |
| 249 | 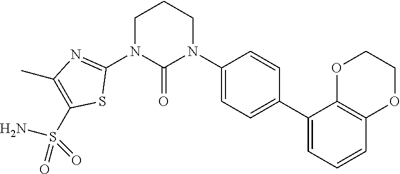 [SMILES: Cc1nc(N2CCCN(c3ccc(-c4cccc5c4OCCO5)cc3)C2=O)sc1S(N)(=O)=O] | 487.0 | ¹H NMR (400 MHz, DMSO-d₆): δ 7.56-7.47 (m, 4H), 7.42-7.34 (m, 2H), 6.90-6.81 (m, 3H), 4.32-4.19 (m, 4H), 4.17- 4.11 (m, 2H), 3.82- 3.75 (m, 2H), 2.43 (s, 3H), 2.26-2.15 (m, 2H). |
| 250 | 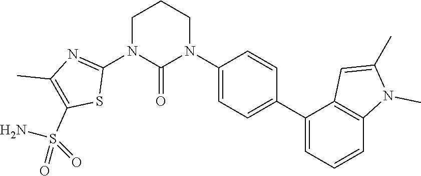 | 496.4 | ¹H NMR (400 MHz, DMSO-d₆): δ 7.68 (d, J = 7.8 Hz, 2H), 7.56 (s, 2H), 7.48 (d, J = 7.8 Hz, 2H), 7.42-7.36 (m, 1H), 7.24- 7.14 (m, 1H), 7.12- 7.06 (m, 1H), 6.38 (s, 1H), 4.23-4.13 (m, 2H), 3.89-3.79 (m, 2H), 3.71 (s, 3H), 2.45 (s, 3H), 2.43 (s, 3H), 2.29-2.21 (m, 2H). |
| 251 | 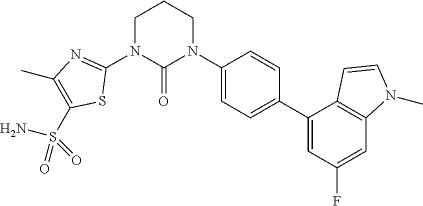 [SMILES: Cc1nc(N2CCCN(c3ccc(-c4cc(F)cc5c4ccn5C)cc3)C2=O)sc1S(N)(=O)=O] | 500.5 | ¹H NMR (400 MHz, DMSO-d₆): δ 7.72 (d, J = 8.4 Hz, 2H), 7.57 (s, 2H), 7.55-7.49 (m, 2H), 7.45- 7.40 (m, 1H), 7.40- 7.31 (m, 1H), 7.02 (dd, J = 2.0, 10.6 Hz, 1H), 6.56 (d, J = 3.1 Hz, 1H), 4.18 (t, J = 5.8 Hz, 2H), 3.84 (t, J = 5.5 Hz, 2H), 3.80 (s, 3H), 2.45 (s, 3H), 2.29- 2.20 (m, 2H). |
| 252 | 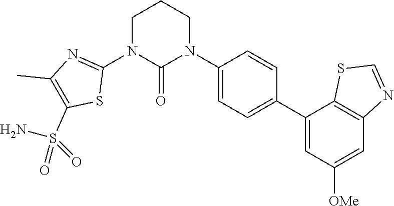 [SMILES: COc1cc(-c2ccc(N3CCCN(c4nc(C)c(S(N)(=O)=O)s4)C3=O)cc2)c2scnc2c1] | 516.0 | ¹H NMR (400 MHz, DMSO-d₆): δ 9.42 (s, 1H), 7.76 (d, J = 8.3 Hz, 2H), 7.66 (s, 1H), 7.62-7.49 (m, 4H), 7.23 (s, 1H), 4.22-4.12 (m, 2H), 3.91 (s, 3H), 3.87-3.80 (m, 2H), 2.44 (s, 3H), 2.30- 2.17 (m, 2H). |
| 253 | 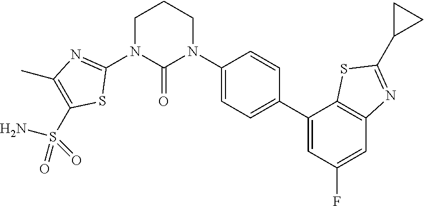 [SMILES: Cc1nc(N2CCCN(c3ccc(-c4cc(F)cc5nc(C6CC6)sc45)cc3)C2=O)sc1S(N)(=O)=O] | 544.1 | ¹H NMR (400 MHz, DMSO-d₆): δ 7.79-7.70 (m, 3H), 7.63-7.54 (m, 4H), 7.44 (dd, J = 2.2, 10.1 Hz, 1H), 4.17 (t, J = 5.7 Hz, 2H), 3.85 (t, J = 5.4 Hz, 2H), 2.45 (s, 3H), 2.29-2.21 (m, 2H), 1.30- 1.24 (m, 2H), 1.19- 1.13 (m, 2H). [Note: -One -CH of cyclopropyl not observed, merged in solvent peak] |
| 254 | 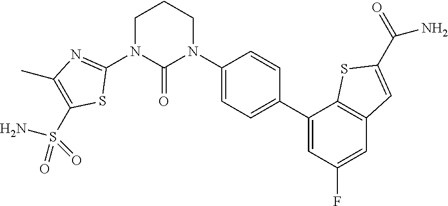 [SMILES: Cc1nc(N2CCCN(c3ccc(-c4cc(F)cc5cc(C(N)=O)sc45)cc3)C2=O)sc1S(N)(=O)=O] | 546.4 | ¹H NMR (400 MHz, DMSO-d₆): δ 8.29 (s, 1H), 8.13 (s, 1H), 7.86-7.78 (m, 3H), 7.69-7.64 (m, 1H), 7.62-7.53 (m, 4H), 7.51-7.45 (m, 1H), 4.22- 4.15 (m, 2H), 3.91- 3.83 (m, 2H), 2.46 (s, 3H), 2.31-2.21 (m, 2H). |
| 255 | 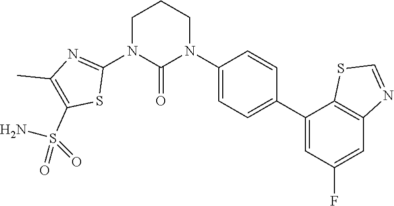 [SMILES: Cc1nc(N2CCCN(c3ccc(-c4cc(F)cc5ncsc45)cc3)C2=O)sc1S(N)(=O)=O] | 504.2 | ¹H NMR (400 MHz, DMSO-d₆): δ 9.56 (s, 1H), 7.98 (d, J = 9.3 Hz, 1H), 7.85-7.78 (m, 2H), 7.63- 7.52 (m, 5H), 4.22- 4.14 (m, 2H), 3.90-3.83 (m, 2H), 2.46 (s, 3H), 2.30-2.20 (m, 2H). |
| 256 | 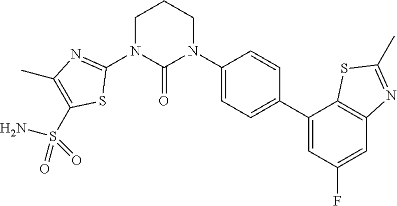 [SMILES: Cc1nc2cc(F)cc(-c3ccc(N4CCCN(c5nc(C)c(S(N)(=O)=O)s5)C4=O)cc3)c2s1] | 518.4 | ¹H NMR (400 MHz, DMSO-d₆): δ 7.84-7.74 (m, 3H), 7.61-7.54 (m, 4H), 7.48 (dd, J = 2.2, 10.0 Hz, 1H), 4.18 (t, J = 5.9 Hz, 2H), 3.85 (t, J = 5.4 Hz, 2H), 2.83 (s, 3H), 2.45 (s, 3H), 2.29-2.20 (m, 2H). |
| 257 | 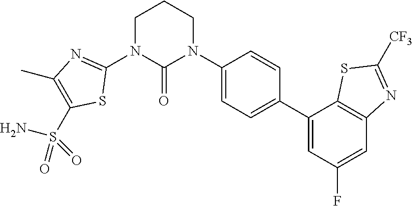 [SMILES: Cc1nc(N2CCCN(c3ccc(-c4cc(F)cc5nc(C(F)(F)F)sc45)cc3)C2=O)sc1S(N)(=O)=O] | 572.1 | ¹H NMR (400 MHz, DMSO-d₆): δ 8.24 (dd, J = 2.4, 9.0 Hz, 1H), 7.89- 7.81 (m, 3H), 7.65-7.60 (m, 2H), 7.58 (s, 2H), 4.18 (t, J = 6.0 Hz, 2H), 3.86 (t, J = 5.6 Hz, 2H), 2.45 (s, 3H), 2.30-2.21 (m, 2H) |
| 258 | 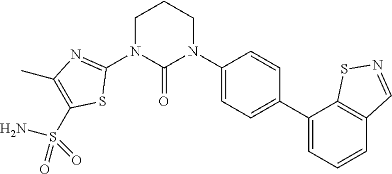 [SMILES: Cc1nc(N2CCCN(c3ccc(-c4cccc5cnsc45)cc3)C2=O)sc1S(N)(=O)=O] | 486.4 | ¹H NMR (400 MHz, DMSO-d₆): δ 9.26 (s, 1H), 8.27 (d, J = 7.3 Hz, 1H), 7.88-7.75 (m, 3H), 7.72- 7.66 (m, 1H), 7.64- 7.55 (m, 4H), 4.23-4.14 (m, 2H), 3.92-3.82 (m, 2H), 2.45 (s, 3H), 2.30- 2.22 (m, 2H). |
| 259 | 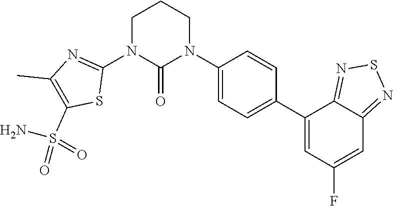 [SMILES: Cc1nc(N2CCCN(c3ccc(-c4cc(F)cc5nsnc45)cc3)C2=O)sc1S(N)(=O)=O] | 505.6 | 1H NMR (400 MHz, DMSO-d6): δ 8.09 (d, J = 2.00 Hz, 2H), 7.93-7.98 (m, 2H), 7.61-7.58 (m, 4H), 4.21-4.18 (m, 2H), 3.89-3.86 (m, 2H), 2.53 (s, 3H), 2.34-2.33 (m, 2H), |
| 260 | 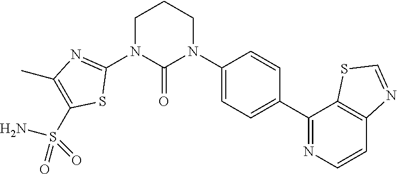 [SMILES: Cc1nc(N2CCCN(c3ccc(-c4nccc5ncsc45)cc3)C2=O)sc1S(N)(=O)=O] | 487.3 | ¹H NMR (400 MHz, DMSO-d₆): δ 9.75 (s, 1H), 8.80-8.77 (d, J = 5.2 Hz, 1H), 8.08 (d, J = 6.8 Hz, 3H), 7.61 (d, J = 8.3 Hz, 2H), 7.54 (s, 2H), 4.21- 4.12 (m, 2H), 3.90-3.81 (m, 2H), 2.43 (s, 3H), 2.27-2.16 (m, 2H). |
| 261 | 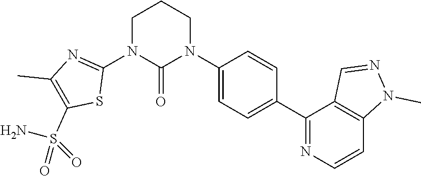 | 484.3 | ¹H NMR (400 MHz, DMSO-d₆): δ 8.51 (s, 1H), 8.49-8.44 (m, 1H), 8.15 (d, J = 8.3 Hz, 2H), 7.65 (d, J = 5.4 Hz, 1H), 7.60- 7.50 (m, 4H), 4.19-4.14 (m, 2H), 4.10 (s, 3H), 3.89-3.79 (m, 2H), 2.43 (s, 3H), 2.28-2.16 (m, 2H). |
| 262 | 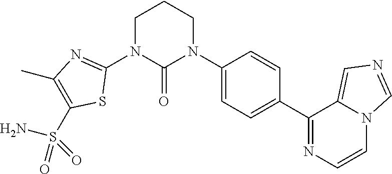 [SMILES: Cc1nc(N2CCCN(c3ccc(-c4nccn5cncc45)cc3)C2=O)sc1S(N)(=O)=O] | 470.3 | ¹H NMR (400 MHz, CD₃OD-d₄): δ 8.57 (s, 1H), 8.26-8.20 (m, 1H), 8.13-8.06 (m, 2H), 8.01- 7.97 (m, 1H), 7.69- 7.59 (m, 3H), 4.33-4.25 (m, 2H), 3.96-3.90 (m, 2H), 2.52(s, 3H), 2.38- 2.30 (m, 2H). [Note: -NH₂ not observed]. |
| 263 | 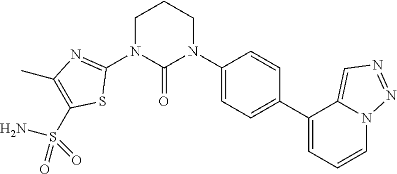 [SMILES: Cc1nc(N2CCCN(c3ccc(-c4cccn5nncc45)cc3)C2=O)sc1S(N)(=O)=O] | 468.2 | ¹H NMR (400 MHz, DMSO-d₆): δ 9.11 (d, J = 7.0 Hz, 1H), 8.38 (s, 1H), 7.85 (d, J = 7.9 Hz, 2H), 7.64-7.52 (m, 4H), 7.34- 7.25 (m, 2H), 4.19 (t, J = 5.7 Hz, 2H), 3.91-3.82 (m, 2H), 2.46 (s, 3H), 2.30-2.21 (m, 2H). |
| 264 | 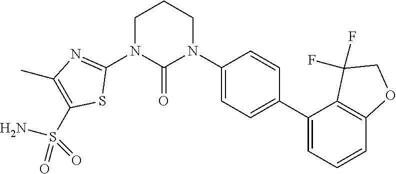 [SMILES: Cc1nc(N2CCCN(c3ccc(-c4cccc5c4C(F)(F)CO5)cc3)C2=O)sc1S(N)(=O)=O] | 507.1 | ¹H NMR (400 MHz, DMSO-d₆): δ 7.67-7.42 (m, 7H), 7.11 (d, J = 7.5 Hz, 2H), 4.82 (t, J = 16.9 Hz, 2H), 4.16 (t, J = 4.8 Hz, 2H), 3.83 (t, J = 4.8 Hz, 2H), 2.45 (s, 3H), 2.28-2.18 (m, 2H) |
| 265 | 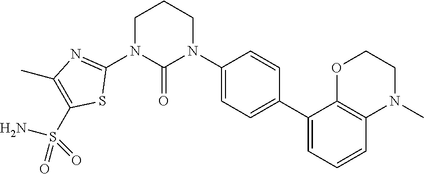 [SMILES: Cc1nc(N2CCCN(c3ccc(-c4cccc5c4OCCN5C)cc3)C2=O)sc1S(N)(=O)=O] | 518.2 | ¹H NMR (400 MHz, DMSO-d₆): δ 7.56 (s, 2H), 7.49 (d, J = 8.5 Hz, 2H), 7.38 (d, J = 8.5 Hz, 2H), 6.55 (dd, J = 2.9, 11.4 Hz, 1H), 6.38 (dd, J = 2.9, 9.3 Hz, 1H), 4.24- 4.07 (m, 4H), 3.79 (t, J = 5.4 Hz, 2H), 2.89 (s, 3H), 2.44 (s, 3H), 2.27-2.17 (m, 2H). [Note: -Two protons merged in solvent peak] |
| 266 | 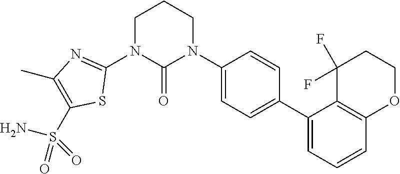 [SMILES: Cc1nc(N2CCCN(c3ccc(-c4cccc5c4C(F)(F)CCO5)cc3)C2=O)sc1S(N)(=O)=O] | 521.5 | ¹H NMR (400 MHz, DMSO-d₆): δ 7.57 (s, 2H), 7.47-7.35 (m, 5H), 6.99 (d, J = 8.1 Hz, 1H), 6.84 (d, J = 7.5 Hz, 1H), 4.35 (t, J = 5.2 Hz, 2H), 4.16 (t, J = 5.9 Hz, 2H), 3.82 (t, J = 5.6 Hz, 2H), 2.44 (s, 3H), 2.43-2.38 (m, 2H), 2.32-2.16 (m, 2H) |
| 267 | 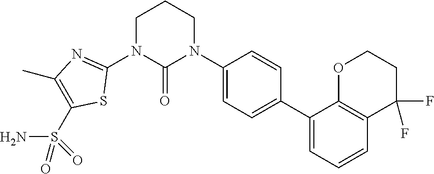 [SMILES: Cc1nc(N2CCCN(c3ccc(-c4cccc5c4OCCC5(F)F)cc3)C2=O)sc1S(N)(=O)=O] | 521.3 | ¹H NMR (400 MHz, DMSO-d₆): δ 7.61-7.52 (m, 5H), 7.48 (d, J = 7.5 Hz, 1H), 7.45-7.41 (m, 2H), 7.23-7.11 (m, 1H), 4.38-4.32 (m, 2H), 4.17 (t, J = 5.9 Hz, 2H), 3.81 (t, J = 5.6 Hz, 2H), 2.63- 2.54 (m, 2H), 2.44 (s, 3H), 2.28-2.17 (m, 2H). |
| 268 | 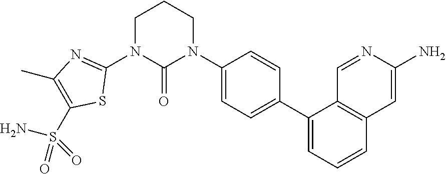 [SMILES: Cc1nc(N2CCCN(c3ccc(-c4cccc5cc(N)ncc45)cc3)C2=O)sc1S(N)(=O)=O] | 495.2 | ¹H NMR (400 MHz, DMSO-d₆): δ 8.70 (s, 1H), 7.62-7.47 (m, 8H), 7.06 (dd, J = 1.1, 6.6 Hz, 1H), 6.69 (s, 1H), 6.01 (s, 2H), 4.18 (t, J = 6.1 Hz, 2H), 3.87 (t, J = 5.4 Hz, 2H), 2.45 (s, 3H), 2.29-2.22 (m, 2H). |
| 269 | 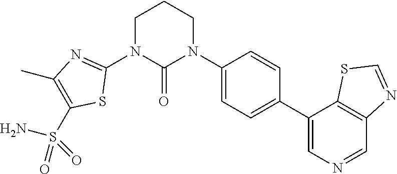 [SMILES: Cc1nc(N2CCCN(c3ccc(-c4cncc5ncsc45)cc3)C2=O)sc1S(N)(=O)=O] | 487.3 | ¹H NMR (400 MHz, DMSO-d₆): δ 9.60 (s, 1H), 9.38-9.35 (m, 1H), 8.71 (s, 1H), 7.83 (d, J = 8.3 Hz, 2H), 7.60 (d, J = 8.3 Hz, 2H), 7.54 (s, 2H), 4.16 (t, J = 5.6 Hz, 2H), 3.88-3.82 (m, 2H), 2.44 (s, 3H), 2.27-2.20 (m, 2H). |
| 270 | 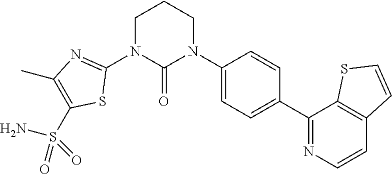 [SMILES: Cc1nc(N2CCCN(c3ccc(-c4nccc5ccsc45)cc3)C2=O)sc1S(N)(=O)=O] | 484.0 | ¹H NMR (400 MHz, DMSO-d₆): δ 8.62 (d, J = 4.8 Hz, 1H), 8.20 (d, J = 5.3 Hz, 1H), 8.15-8.06 (m, 2H), 7.99-7.80 (m, 1H), 7.73-7.49 (m, 5H), 4.24-4.14 (m, 2H), 3.94- 3.84 (m, 2H), 2.54 (s, 3H), 2.35-2.20 (m, 2H). |
| 271 | 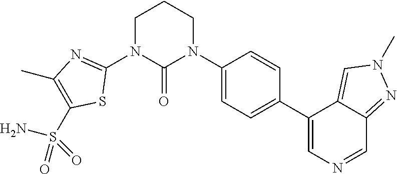 [SMILES: Cc1nc(N2CCCN(c3ccc(-c4cncc5nn(C)cc45)cc3)C2=O)sc1S(N)(=O)=O] | 484.1 | (400 MHz, DMSO-d6): δ 9.14 (s, 1H), 8.77 (s, 1H), 8.29 (s, 1H), 7.84 (d, J = −8.00 Hz, 2H), 7.55-7.58 (m, 4H), 4.30 (s, 3H), 4.09-4.19 (m, 2H), 3.84- 3.87 (m, 2H), 2.50 (s, 3H), 2.20-2.27 (m, 2H), |
| 272 | 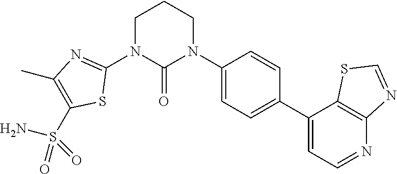 [SMILES: Cc1nc(N2CCCN(c3ccc(-c4ccnc5ncsc45)cc3)C2=O)sc1S(N)(=O)=O] | 487.0 | ¹H NMR (400 MHz, DMSO-d₆): δ 9.81 (s, 1H), 8.84 (d, J = 4.9 Hz, 1H), 7.88 (d, J = 8.8 Hz, 2H), 7.70 (d, J = 5.0 Hz, 1H), 7.65 (d, J = 8.6 Hz, 2H), 6.05 (br s, 2H), 4.18 (t, J = 5.9 Hz, 2H), 3.87 (t, J = 5.5 Hz, 2H), 2.45 (s, 3H), 2.28-2.21 (m, 2H) |
| 273 | 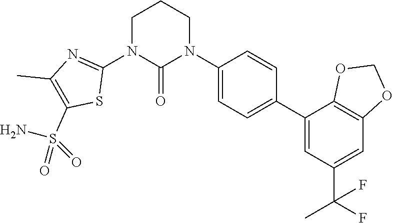 [SMILES: Cc1nc(N2CCCN(c3ccc(-c4cc(C(C)(F)F)cc5c4OCO5)cc3)C2=O)sc1S(N)(=O)=O] | 537.4 | ¹H NMR (400 MHz, DMSO-d₆): δ 7.80 (d, J = 8.6 Hz, 2H), 7.56 (s, 2H), 7.54-7.46 (m, 2H), 7.34- 7.27 (m, 1H), 7.13 (d, J = 1.6 Hz, 1H), 6.18 (s, 2H), 4.16 (t, J = 5.9 Hz, 2H), 3.81 (t, J = 5.6 Hz, 2H), 2.44 (s, 3H), 2.27- 2.20 (m, 2H), 2.01 (t, J = 18.8 Hz, 3H) |
| 274 | 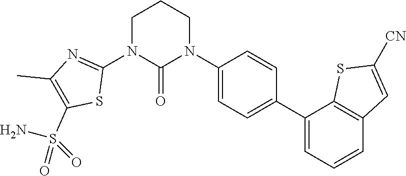 [SMILES: Cc1nc(N2CCCN(c3ccc(-c4cccc5cc(C#N)sc45)cc3)C2=O)sc1S(N)(=O)=O] | 510.4 | ¹H NMR (400 MHz, DMSO-d₆): δ 8.52-8.48 (m, 1H), 8.10-8.04 (m, 1H), 7.79-7.73 (m, 2H), 7.71-7.65 (m, 2H), 7.60- 7.51 (m, 4H), 4.20- 4.12 (m, 2H), 3.88-3.80 (m, 2H), 2.43 (s, 3H), 2.29-2.18 (m, 2H). |
| 275 | 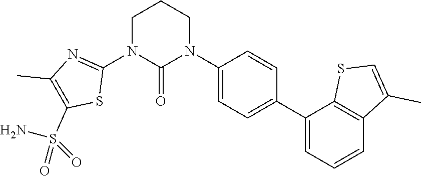 | 499.1 | ¹H NMR (400 MHz, DMSO-d₆): δ 7.83-7.72 (m, 3H), 7.61-7.52 (m, 5H), 7.49-7.43 (m, 2H), 4.21-4.14 (m, 2H), 3.91- 3.82 (m, 2H), 2.48-2.43 (m, 6H), 2.32-2.18 (m, 2H). |
| 276 | 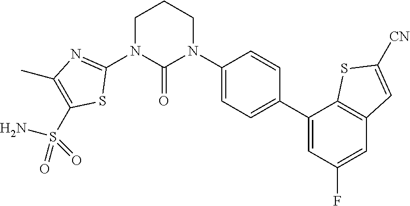 [SMILES: Cc1nc(N2CCCN(c3ccc(-c4cc(F)cc5cc(C#N)sc45)cc3)C2=O)sc1S(N)(=O)=O] | 528.4 | ¹H NMR (400 MHz, DMSO-d₆): δ 8.48 (s, 1H), 7.94 (d, J = 8.8 Hz, 1H), 7.81 (d, J = 8.8 Hz, 2H), 7.68 (d, J = 9.8 Hz, 1H), 7.62 (d, J = 8.3 Hz, 2H), 7.59-7.55 (m, 2H), 4.18 (t, J = 5.9 Hz, 2H), 3.89- 3.84 (m, 2H), 2.46 (s, 3H), 2.30-2.22 (m, 2H). |
| 277 | 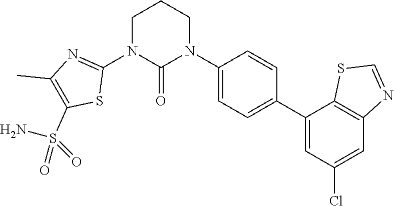 [SMILES: Cc1nc(N2CCCN(c3ccc(-c4cc(Cl)cc5ncsc45)cc3)C2=O)sc1S(N)(=O)=O] | 520.2 | ¹H NMR (400 MHz, DMSO-d₆): δ 9.56 (s, 1H), 8.23 (d, J = 2.0 Hz, 1H), 7.81 (d, J = 8.5 Hz, 2H), 7.68 (d, J = 1.8 Hz, 1H), 7.62-7.60 (m, 1H), 7.59- 7.55 (m, 3H), 4.17 (t, J = 5.6 Hz, 2H), 3.86 (t, J = 5.6 Hz, 2H), 2.45 (s, 3H), 2.29-2.21 (m, 2H). |
| 278 | 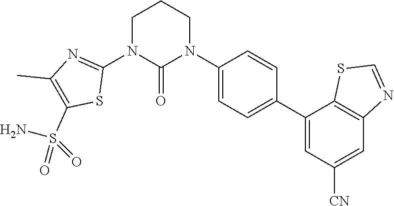 [SMILES: Cc1nc(N2CCCN(c3ccc(-c4cc(C#N)cc5ncsc45)cc3)C2=O)sc1S(N)(=O)=O] | 511.2 | ¹H NMR (400 MHz, DMSO-d₆): δ 9.69 (s, 1H), 8.70 (d, J = 1.3 Hz, 1H), 8.06 (d, J = 1.2 Hz, 1H), 7.87-7.83 (m, 2H), 7.67- 7.62 (m, 2H), 7.60 (brs, 2H), 4.20 (t, J = 6.0 Hz, 2H), 3.89 (t, J = 5.5 Hz, 2H), 2.47 (s, 3H), 2.34- 2.21 (m, 2H) |
| 279 | 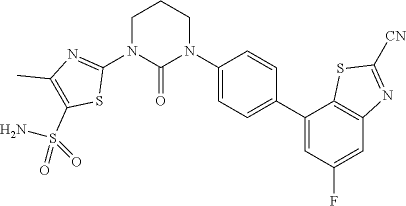 [SMILES: Cc1nc(N2CCCN(c3ccc(-c4cc(F)cc5nc(C#N)sc45)cc3)C2=O)sc1S(N)(=O)=O] | 529.4 | ¹H NMR (400 MHz, DMSO-d₆): δ 8.23 (dd, J = 2.3, 8.9 Hz, 1H), 7.89- 7.79 (m, 3H), 7.67-7.59 (m, 2H), 7.58 (s, 2H), 4.18 (t, J = 5.8 Hz, 2H), 3.87 (t, J = 5.4 Hz, 2H), 2.44 (s, 3H), 2.31-2.19 (m, 2H). |
| 280 | 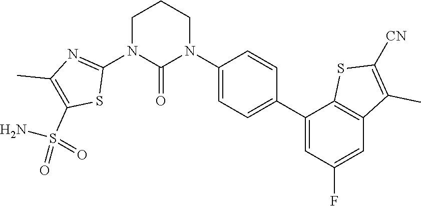 | 542.0 | ¹H NMR (400 MHz, DMSO-d₆): δ 8.38 (d, J = 7.2 Hz, 1H), 8.04 (d, J = 11.2 Hz, 1H), 7.69 (dd, J = 1.6, 8.8 Hz, 2H), 7.58- 7.54 (m, 4H), 4.18 (t, J = 6.0 Hz, 2H), 3.85 (t, J = 6.0 Hz, 2H), 2.67 (s, 3H), 2.50 (s, 3H), 2.34-2.26 (m, 2H). |
| 281 | 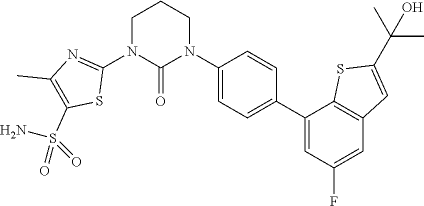 [SMILES: Cc1nc(N2CCCN(c3ccc(-c4cc(F)cc5cc(C(C)(C)O)sc45)cc3)C2=O)sc1S(N)(=O)=O] | 561.0 | ¹H NMR (400 MHz, DMSO-d₆): δ 7.79-7.77 (m, 2H), 7.63-7.57 (m, 5H), 7.37-7.31 (m, 1H), 7.30-7.27 (m, 1H), 5.70 (s, 1H), 4.19 (t, J = 6.0 Hz, 2H), 3.86 (t, J = 5.6 Hz, 2H), 2.46 (s, 3H), 2.33-2.27 (m, 2H), 1.57 (s, 6H). |
| 282 | 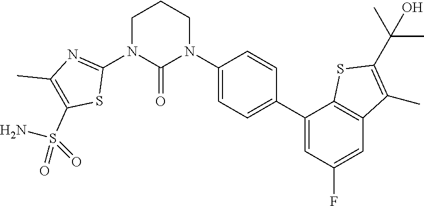 [SMILES: Cc1nc(N2CCCN(c3ccc(-c4cc(F)cc5c(C)c(C(C)(C)O)sc45)cc3)C2=O)sc1S(N)(=O)=O] | 575.1 | ¹H NMR (400 MHz, DMSO-d₆): δ 7.78-7.75 (m, 2H), 7.58-7.51 (m, 5H), 7.28 (dd, J = 2.4, 9.6 Hz, 1H), 5.74 (s, 1H), 4.19 (t, J = 6.0 Hz, 2H), 3.86 (t, J = 5.6 Hz, 2H), 2.53 (s, 6H), 2.47 (t, J = 8.4 Hz, 2H), 1.59 (s, 6H). |
| 283 | 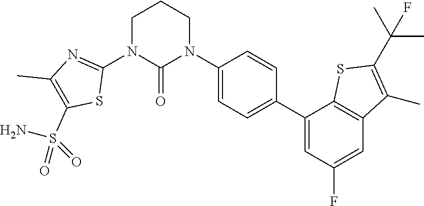 [SMILES: Cc1nc(N2CCCN(c3ccc(-c4cc(F)cc5c(C)c(C(C)(C)F)sc45)cc3)C2=O)sc1S(N)(=O)=O] | 577.1 | ¹H-NMR (400 MHz, DMSO-d6) 7.77 (d, J = 2.00 Hz, 2H), 7.58 (d, J = 2.00 Hz, 4H), 4.19 (t, J = 6.00 Hz, 2H), 3.86 (t, J = 6.00 Hz, 2H), 2.46 (s, 6H), 2.26 (s, 2H), 1.84 (s, 3H), 1.79 (s, 3H). |
| 284 | 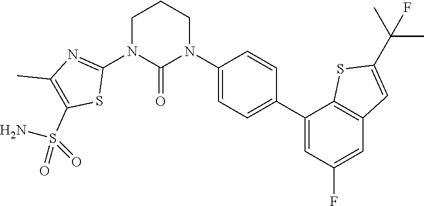 [SMILES: Cc1nc(N2CCCN(c3ccc(-c4cc(F)cc5cc(C(C)(C)F)sc45)cc3)C2=O)sc1S(N)(=O)=O] | 563.1 | ¹H NMR (400 MHz, DMSO-d₆): δ 7.81-7.78 (m, 2H), 7.71 (dd, J = 2.4, 9.2 Hz, 1H), 7.60- 7.54 (m, 5H), 7.39 (dd, J = 2.4, 10.0 Hz, 1H), 4.19 (t, J = 6.0 Hz, 2H), 3.86 (t, J = 5.6 Hz, 2H), 2.46 (s, 3H), 2.26 (t, J = 5.6 Hz, 2H), 1.85 (s, 3H), 1.80 (s, 3H). |
| 285 | 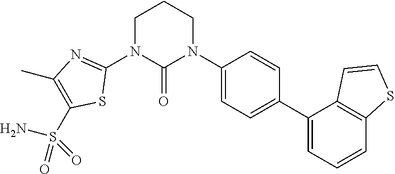 [SMILES: Cc1nc(N2CCCN(c3ccc(-c4cccc5sccc45)cc3)C2=O)sc1S(N)(=O)=O] | 485.1 | 1H NMR (400 MHz, DMSO-d6): δ 8.06 (d, J = 8.00 Hz, 1H), 7.84 (d, J = 5.60 Hz, 1H), 7.64 (d, J = 8.80 Hz, 2H), 7.48-7.56 (m, 4H), 7.43-7.42 (m, 2H), 7.43 (d, J = 1.20 Hz, 1H), 4.20-4.17 (m, 2H), 3.88-3.85 (m, 2H), 2.50 (s, 3H), 2.31-2.26 (m, 2H) |
| 286 | 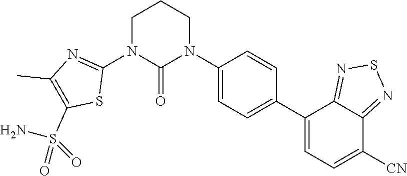 [SMILES: Cc1nc(N2CCCN(c3ccc(-c4ccc(C#N)c5nsnc45)cc3)C2=O)sc1S(N)(=O)=O] | 512.1 | 1H NMR (400 MHz, DMSO-d6): δ 8.01 (d, J = 7.60 Hz, 1H), 7.51-7.42 (m, 4H), 7.36-7.30 (m, 3H), 4.22-4.19 (m, 2H), 3.80-3.77 (m, 2H), 2.54 (s, 3H), 2.27-2.24 (m, 2H) |
| 287 | 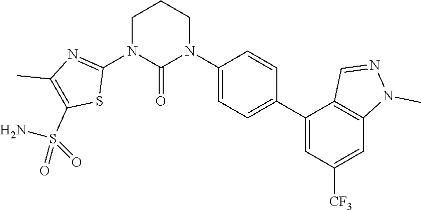 | 551.1( | ¹H NMR (400 MHz, DMSO-d₆): δ 8.34 (s, 1H), 8.22 (s, 1H), 7.85 (d, J = 1.60 Hz, 2H), 7.59 (d, J = 1.60 Hz, 4H), 7.52 (s, 1H), 4.21 (s, 5H), 3.87 (t, J = 5.60 Hz, 2H), 2.33 (s, 3H), 2.28-2.25 (m, 2H) |
| 288 | 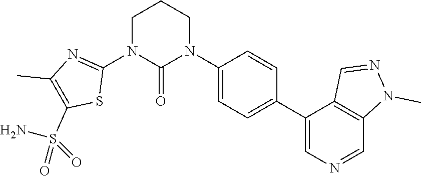 | 484.1 | 1H NMR (400 MHz, DMSO-d6): δ 9.20 (s, 1H), 8.45 (s, 1H), 8.37 (s, 1H), 7.88 (d, J = 8.80 Hz, 2H), 7.60-7.58 (m, 4H), 4.25 (s, 3H), 4.20-4.19 (m, 3H), 3.88-3.85 (m, 2H), 2.51 (s, 3H), 2.33- 2.28 (m, 2H) |
| 289 | 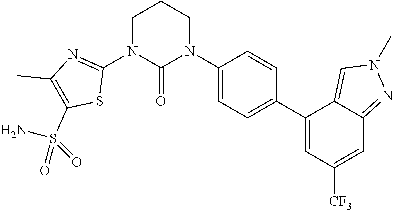 | 551.4 | ¹H NMR (400 MHz, DMSO-d₆): δ 8.76 (s, 1H), 8.07 (s, 1H), 7.84 (d, J = 8.40 Hz, 2H), 7.57 (d, J = 8.80 Hz, 4H), 7.37 (d, J = 1.20 Hz, 1H), 4.27 (s, 3H), 4.21-4.18 (m, 2H), 3.86 (t, J = 6.00 Hz, 2H), 2.46 (s, 3H), 2.26 (t, J = 6.00 Hz, 2H) |
| 290 | 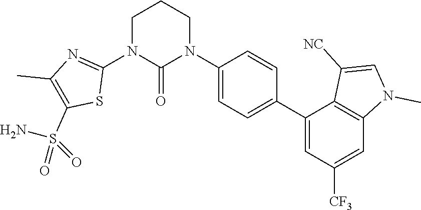 | 575.1 | 1H NMR (400 MHz, DMSO-d6): δ 8.58 (s, 1H), 8.15 (s, 1H), 7.62 (d, J = 2.00 Hz, 2H), 7.58- 7.53 (m, 4H), 7.45 (d, J = 1.20 Hz, 1H), 4.20-4.02 (m, 2H), 3.98 (s, 3H), 3.86-3.39 (m, 2H), 2.50 (s, 3H), 2.34-2.33 (m, 2H) |
| 291 | 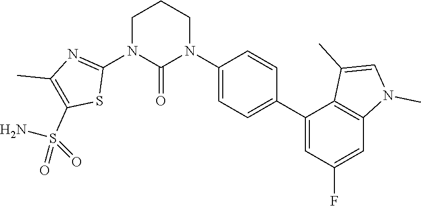 | 514.2 | 1H NMR (400 MHz, DMSO-d6): δ 7.49 (d, J = 2.00 Hz, 2H), 7.44-7.42 (m, 4H), 7.31 (d, J = 2.00 Hz, 1H), 7.28 (s, 2H), 6.73 (d, J = 2.40 Hz, 1H), 6.74-6.71 (m, 2H), 3.86- 3.83 (m, 2H), 3.73 (s, 3H), 2.51 (s, 3H), 2.34- 2.25 (m, 2H), |
| 292 | 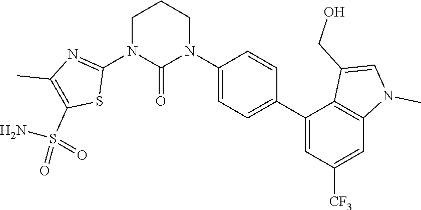 | 580.0 | 1H NMR (400 MHz, DMSO-d6): δ 7.88 (s, 1H), 7.49-7.54 (m, 7H), 7.10 (s, 1H), 4.75-4.72 (m, 1H), 4.20-4.18 (m, 4H), 3.91 (s, 3H), 3.88- 3.85 (m, 2H), 2.56 (s, 3H), 2.57-2.52 (m, 2H), |
| 293 | 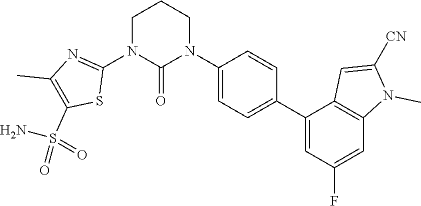 | 525.0 | ¹H-NMR (400 MHz, DMSO-d₆) 7.75 (d, J = 8.80 Hz, 2H), 7.61- 7.54 (m, 7H), 4.19 (t, J = 6.00 Hz, 2H), 3.93 (s, 3H), 3.85 (t, J = 4.80 Hz, 2H), 2.46 (s, 3H), 2.26 (d, J = 5.20 Hz, 2H). |
| 294 | 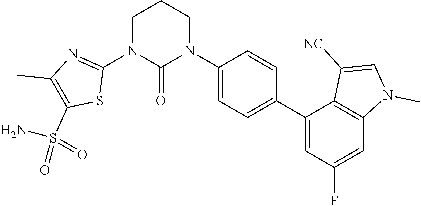 | 525.1 | 1H NMR (400 MHz, DMSO-d6): δ 8.36 (s, 1H), 7.63-7.51 (m, 7H), 7.12 (d, J = 1.60 Hz, 1H), 4.20-4.17 (m, 2H), 3.89 (s, 3H), 3.86-3.83 (m, 2H), 2.51 (s, 3H), 2.05- 2.08 (m, 2H), |
| 295 | 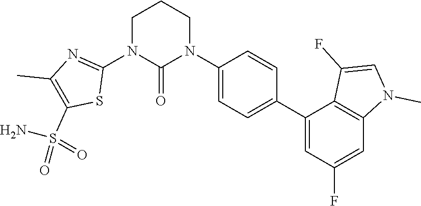 | 518.1 | 1H NMR (400 MHz, DMSO-d6): δ 7.62-7.58 (m, 4H), 7.50-7.46 (m, 3H), 7.41 (d, J = 2.40 Hz, 1H), 6.98 (d, J = 2.40 Hz, 1H), 4.19-4.16 (m, 2H), 3.86-3.83 (m, 2H), 3.75 (s, 3H), 2.46 (s, 3H), 2.34-2.25 (m, 2H), |
| 296 | 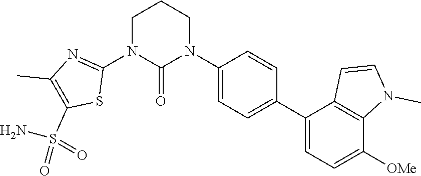 | 512.1 | ¹H NMR (400 MHz, DMSO-d₆) δ 7.62 (d, J = 8.3 Hz, 2H), 7.58-7.51 (m, 2H), 7.45 (d, J = 8.3 Hz, 2H), 7.27 (d, J = 2.9 Hz, 1H), 7.02 (d, J = 7.8 Hz, 1H), 6.76 (d, J = 8.3 Hz, 1H), 6.49 (d, J = 2.9 Hz, 1H), 4.18 (t, J = 5.9 Hz, 2H), 4.04 (s, 3H), 3.93 (s, 3H), 3.86-3.79 (m, 2H), 2.45 (s, 3H), 2.29-2.19 (m, 2H). |
| 297 | 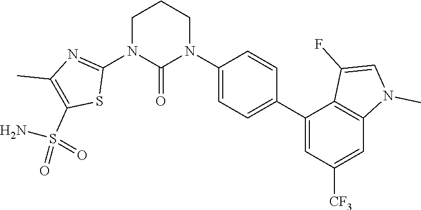 | 568.1 | 1H NMR (400 MHz, DMSO-d6): δ 7.97 (d, J = 2.00 Hz, 1H), 7.74 (d, J = 2.00 Hz, 1H), 7.63 (d, J = 3.20 Hz, 2H), 7.65 (d, J = 2.80 Hz, 2H), 7.55 (d, J = 23.20 Hz, 2H), 7.30 (d, J = 0.80 Hz, 1H), 4.20- 4.17 (m, 2H), 3.89 (s, 3H), 3.87-3.84 (m, 2H), 2.52 (s, 3H), 2.34-2.27 (m, 2H), |
| 298 | 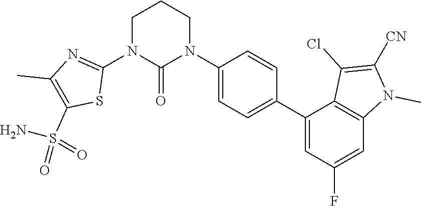 | 560.1 | 1H NMR (400 MHz, DMSO-d6): δ 7.74 (d, J = 2.00 Hz, 1H), 7.72-7.71 (m, 2H), 7.58-7.51 (m, 4H), 7.10 (d, J = 2.00 Hz, 1H), 4.19-4.16 (m, 2H), 3.93 (s, 3H), 3.87-3.85 (m, 2H), 2.51 (s, 3H), 2.51 (m, 2H), |
| 299 | 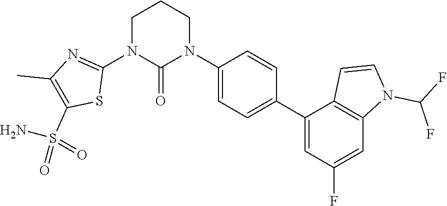 [SMILES: Cc1nc(N2CCCN(c3ccc(-c4cc(F)cc5c4ccn5C(F)F)cc3)C2=O)sc1S(N)(=O)=O] | 536.1 | ¹H NMR (400 MHz, DMSO-d₆): δ 8.05 (t, J = 59.6 Hz, 1H), 7.77-7.68 (m, 3H), 7.59-7.53 (m, 5H), 7.23 (dd, J = 1.9, 10.4 Hz, 1H), 6.80 (d, J = 3.5 Hz, 1H), 4.18 (t, J = 5.6 Hz, 2H), 3.85 (t, J = 5.2 Hz, 2H), 2.45 (s, 3H), 2.31-2.19 (m, 2H) |
| 300 | 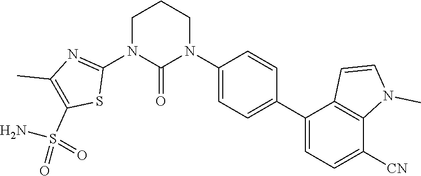 | 507.0 | ¹H NMR (400 MHz, DMSO-d₆): δ 7.77-7.68 (m, 2H), 7.58-7.54 (m, 4H), 7.28 (d, J = 7.8 Hz, 1H), 6.70 (d, J = 3.0 Hz, 1H), 4.17-4.16 (m, 2H), 4.13 (s, 3H), 3.88-3.82 (m, 2H), 2.45 (s, 3H), 2.29-2.18 (m, 2H). [NH₂ not observed] |
| 301 | 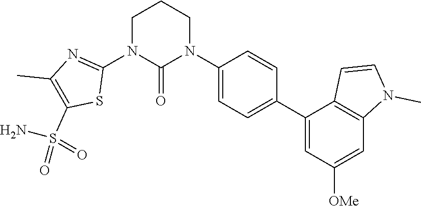 | 512.1 | ¹H NMR (400 MHz, DMSO-d₆): δ 7.75-7.66 (m, 2H), 7.57 (s, 2H), 7.49 (d, J = 8.5 Hz, 2H), 7.27 (d, J = 3.3 Hz, 1H), 7.01 (d, J = 1.6 Hz, 1H), 6.79 (d, J = 2.1 Hz, 1H), 6.46 (dd, J = 0.6, 3.3 Hz, 1H), 4.17 (t, J = 5.9 Hz, 2H), 3.86 (s, 3H), 3.85- 3.81 (m, 2H), 3.78 (s, 3H), 2.45 (s, 3H), 2.31- 2.17 (m, 2H) |
| 302 | 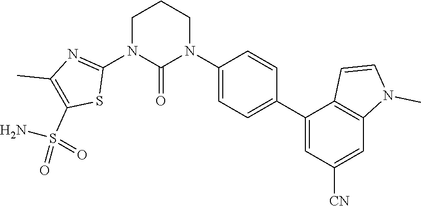 | 507.1 | ¹H NMR (400 MHz, DMSO-d₆): δ 8.15-8.08 (m, 1H), 7.80-7.69 (m, 3H), 7.60-7.42 (m, 5H), 6.73-6.66 (m, 1H), 4.24- 4.12 (m, 2H), 3.92 (s, 3H), 3.87-3.81 (m, 2H), 2.46 (s, 3H), 2.30-2.20 (m, 2H). |
| 303 | 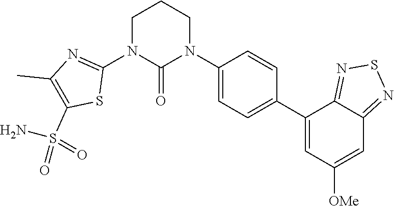 [SMILES: COc1cc(-c2ccc(N3CCCN(c4nc(C)c(S(N)(=O)=O)s4)C3=O)cc2)c2nsnc2c1] | 516.9 | ¹H NMR (400 MHz, DMSO-d₆): δ 8.07-7.97 (m, 2H), 7.62-7.44 (m, 6H), 4.24-4.13 (m, 2H), 3.97 (s, 3H), 3.91-3.80 (m, 2H), 2.46 (s, 3H), 2.32-2.21 (m, 2H) |
| 304 | 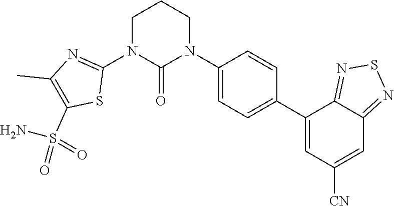 [SMILES: Cc1nc(N2CCCN(c3ccc(-c4cc(C#N)cc5nsnc45)cc3)C2=O)sc1S(N)(=O)=O] | 512.2 | ¹H NMR (400 MHz, DMSO-d₆): δ 8.87 (d, J = 1.3 Hz, 1H), 8.19 (d, J = 1.3 Hz, 1H), 8.09 (d, J = 8.7 Hz, 2H), 7.62-7.55 (m, 4H), 4.18 (t, J = 6.1 Hz, 2H), 3.87 (t, J = 5.3 Hz, 2H), 2.45 (s, 3H), 2.30-2.21 (m, 2H) |
| 305 | 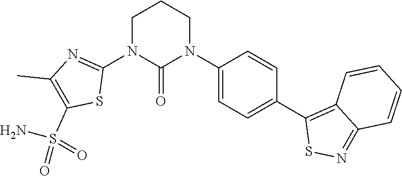 [SMILES: Cc1nc(N2CCCN(c3ccc(-c4snc5ccccc45)cc3)C2=O)sc1S(N)(=O)=O] | 486.0 | ¹H NMR (400 MHz, DMSO-d₆): δ 8.02-7.97 (m, 1H), 7.88-7.80 (m, 3H), 7.69-7.63 (m, 2H), 7.59-7.53 (m, 3H), 7.42- 7.35 (m, 1H), 4.23- 4.15 (m, 2H), 3.91-3.84 (m, 2H), 2.45 (s, 3H), 2.31-2.22 (m, 2H). |
| 306 | 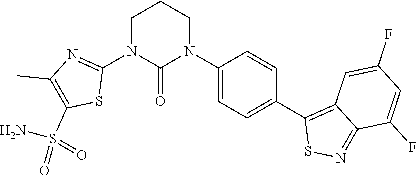 [SMILES: Cc1nc(N2CCCN(c3ccc(-c4snc5c(F)cc(F)cc45)cc3)C2=O)sc1S(N)(=O)=O] | 522.3 | ¹H NMR (400 MHz, DMSO-d₆): δ 7.86 (d, J = 8.5 Hz, 2H), 7.68-7.57 (m, 6H), 4.18 (t, J = 5.9 Hz, 2H), 3.87 (t, J = 5.5 Hz, 2H), 2.46 (s, 3H), 2.29-2.22 (m, 2H). |
| 307 | 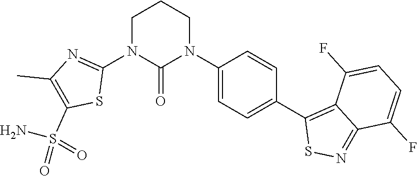 [SMILES: Cc1nc(N2CCCN(c3ccc(-c4snc5c(F)ccc(F)c45)cc3)C2=O)sc1S(N)(=O)=O] | 522.4 | ¹H NMR (400 MHz, DMSO-d₆): δ 7.82-7.70 (m, 2H), 7.63-7.58 (m, 2H), 7.56 (s, 2H), 7.45- 7.27 (m, 1H), 7.13-7.00 (m, 1H), 4.16 (t, J = 5.7 Hz, 2H), 3.86 (t, J = 4.8 Hz, 2H), 2.44 (s, 3H), 2.29-2.18 (m, 2H). |
| 308 | 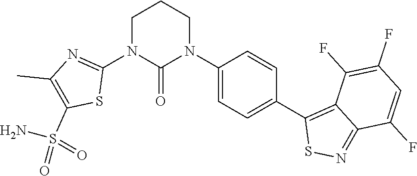 [SMILES: Cc1nc(N2CCCN(c3ccc(-c4snc5c(F)cc(F)c(F)c45)cc3)C2=O)sc1S(N)(=O)=O] | 539.8 | ¹H NMR (400 MHz, DMSO-d₆): δ 7.94-7.71 (m, 3H), 7.67-7.51 (m, 4H), 4.23-4.08 (m, 2H), 3.91-3.78 (m, 2H), 2.28- 2.17 (m, 2H). [Note: -Methyl proton merged in solvent peak] |
| 309 | 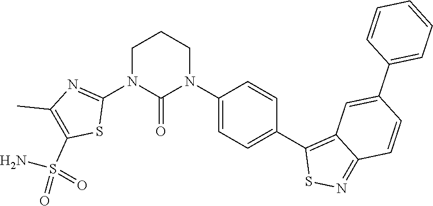 [SMILES: Cc1nc(N2CCCN(c3ccc(-c4snc5ccc(-c6ccccc6)cc45)cc3)C2=O)sc1S(N)(=O)=O] | 562.4 | ¹H NMR (400 MHz, DMSO-d₆): δ 8.11 (s, 1H), 7.97-7.88 (m, 4H), 7.87- 7.74 (m, 2H), 7.66 (d, J = 8.8 Hz, 2H), 7.57 (s, 2H), 7.55-7.47 (m, 2H), 7.47-7.38 (m, 1H), 4.22- 4.14 (m, 2H), 3.92- 3.83 (m, 2H), 2.47 (s, 3H), 2.30-2.21 (m, 2H). |
| 310 | 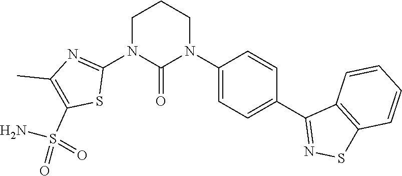 [SMILES: Cc1nc(N2CCCN(c3ccc(-c4nsc5ccccc45)cc3)C2=O)sc1S(N)(=O)=O] | 486.3 | ¹H NMR (400 MHz, DMSO-d₆): δ 8.29 (dd, J = 8.3, 17.1 Hz, 2H), 7.95 (d, J = 7.9 Hz, 2H), 7.73- 7.51 (m, 6H), 4.24-4.12 (m, 2H), 3.94-3.80 (m, 2H), 2.45 (s, 3H), 2.30- 2.22 (m, 2H). |
| 311 | 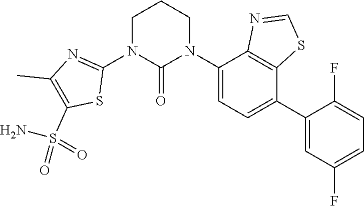 [SMILES: Cc1nc(N2CCCN(c3ccc(-c4cc(F)ccc4F)c4scnc34)C2=O)sc1S(N)(=O)=O] | 522.4 | ¹H NMR (400 MHz, DMSO-d₆): δ 9.47 (s, 1H), 7.73-7.61 (m, 2H), 7.61- 7.37 (m, 5H), 4.32- 4.20 (m, 2H), 3.96-3.84 (m, 2H), 2.37-2.24 (m, 2H), [Note: -CH₃ merged with solvent peak, not observed] |
| 312 | 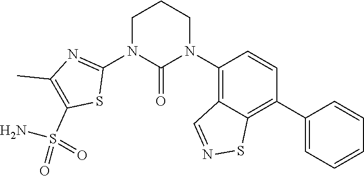 [SMILES: Cc1nc(N2CCCN(c3ccc(-c4ccccc4)c4sncc34)C2=O)sc1S(N)(=O)=O] | 486.3 | ¹H NMR (400 MHz, DMSO-d₆): δ 9.19 (s, 1H), 7.84 (d, J = 7.6 Hz, 1H), 7.81-7.76 (m, 2H), 7.69- 7.48 (m, 6H), 4.31- 4.22 (m, 2H), 3.97-3.88 (m, 2H), 2.46 (s, 3H), 2.34-2.30 (m, 2H) |
| 313 | 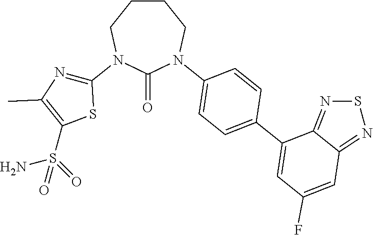 [SMILES: Cc1nc(N2CCCCN(c3ccc(-c4cc(F)cc5nsnc45)cc3)C2=O)sc1S(N)(=O)=O] | 519.2 | ¹H NMR (400 MHz, DMSO-d₆): δ 8.08 (d, J = 7.8 Hz, 2H), 7.99-7.89 (m, 2H), 7.62-7.50 (m, 4H), 4.31-4.23 (m, 2H), 3.90-3.80 (m, 2H), 2.46 (s, 3H), 2.06-1.96 (m, 2H), 1.91-1.81 (m, 2H). |
| 314 | 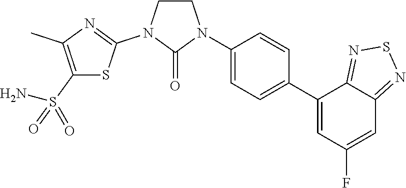 [SMILES: Cc1nc(N2CCN(c3ccc(-c4cc(F)cc5nsnc45)cc3)C2=O)sc1S(N)(=O)=O] | 491.3 | ¹H NMR (400 MHz, DMSO-d₆): δ 8.15 (d, J = 8.9 Hz, 2H), 7.95-7.89 (m, 2H), 7.85 (d, J = 8.8 Hz, 2H), 7.67 (s, 2H), 4.18 (s, 4H), 2.47 (s, 3H). |
| 315 | 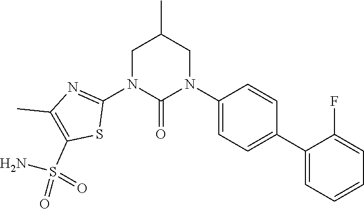 [SMILES: Cc1nc(N2CC(C)CN(c3ccc(-c4ccccc4F)cc3)C2=O)sc1S(N)(=O)=O] | 461.3 | ¹H NMR (400 MHz, DMSO-d₆): δ 7.63-7.53 (m, 5H), 7.52-7.46 (m, 2H), 7.46-7.40 (m, 1H), 7.39-7.27 (m, 2H), 4.43- 4.34 (m, 1H), 3.81- 3.73 (m, 1H), 3.71-3.57 (m, 2H), 2.45 (s, 3H), 1.12 (d, J = 6.6 Hz, 3H). [Note: one proton not observed] |
| 316 | 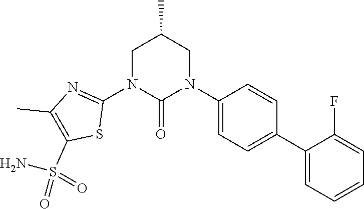 [SMILES: Cc1nc(N2C[C@H](C)CN(c3ccc(-c4ccccc4F)cc3)C2=O)sc1S(N)(=O)=O] | 460.1 | ¹H NMR (400 MHz, CD₃OD-d₄): δ 7.66-7.56 (m, 2H), 7.53-7.42 (m, 3H), 7.42-7.33 (m, 1H), 7.29-7.15 (m, 2H), 4.56- 4.48 (m, 1H), 3.84- 3.58 (m, 3H), 2.59-2.49 (m, 4H), 1.21 (d, J = 6.4 Hz, 3H). [Note: -NH₂ protons not observed]. |
| 317 | 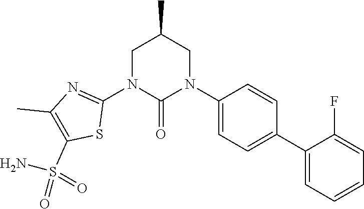 [SMILES: Cc1nc(N2C[C@@H](C)CN(c3ccc(-c4ccccc4F)cc3)C2=O)sc1S(N)(=O)=O] Single enantiomer, arbitrarily assigned stereochemistry | 460.1 | ¹H NMR (400 MHz, CD₃OD-d₄): δ 7.61 (d, J = 7.8 Hz, 2H), 7.53-7.42 (m, 3H), 7.41-7.33 (m, 1H), 7.30-7.14 (m, 2H), 4.56-4.48 (m, 1H), 3.84- 3.58 (m, 3H), 2.59- 2.49 (m, 4H), 1.21 (d, J = 6.4 Hz, 3H). [Note: -NH₂ protons not observed]. |
| 318 | 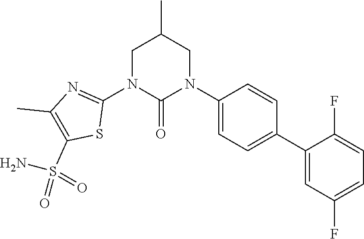 [SMILES: Cc1nc(N2CC(C)CN(c3ccc(-c4cc(F)ccc4F)cc3)C2=O)sc1S(N)(=O)=O] | 479.4 | ¹H NMR (400 MHz, DMSO-d₆): δ 7.64-7.57 (m, 2H), 7.57-7.45 (m, 4H), 7.44-7.32 (m, 2H), 7.30-7.20 (m, 1H), 4.40- 4.32 (m, 1H), 3.78- 3.70 (m, 1H), 3.68-3.55 (m, 2H), 2.42 (s, 3H), 1.09 (d, J = 5.9 Hz, 3H). [Note: -One proton merged in solvent peak] |
| 319 | 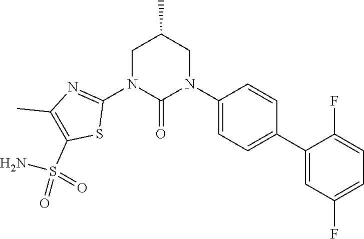 [SMILES: Cc1nc(N2C[C@H](C)CN(c3ccc(-c4cc(F)ccc4F)cc3)C2=O)sc1S(N)(=O)=O] Single enantiomer, arbitrarily assigned stereochemistry | 479.4 | ¹H NMR (400 MHz, DMSO-d₆): δ 7.63 (d, J = 8.3 Hz, 2H), 7.56 (brs, 2H), 7.53-7.48 (m, 2H), 7.47-7.35 (m, 2H), 7.33- 7.23 (m, 1H), 4.43- 4.35 (m, 1H), 3.80-3.73 (m, 1H), 3.70-3.57 (m, 2H), 2.45 (s, 3H), 1.12 (d, J = 6.4 Hz, 3H). [Note: -one proton merged in solvent peak]. |
| 320 | 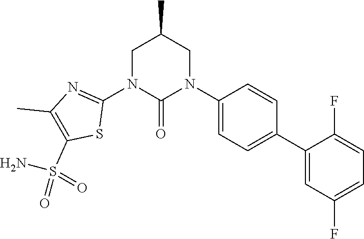 [SMILES: Cc1nc(N2C[C@@H](C)CN(c3ccc(-c4cc(F)ccc4F)cc3)C2=O)sc1S(N)(=O)=O] Single enantiomer, arbitrarily assigned stereochemistry | 479.4 | ¹H NMR (400 MHz, DMSO-d₆): δ 7.62 (d, J = 6.8 Hz, 2H), 7.56 (brs, 2H), 7.53-7.47 (m, 2H), 7.46-7.34 (m, 2H), 7.31- 7.22 (m, 1H), 4.44- 4.33 (m, 1H), 3.83-3.73 (m, 1H), 3.69-3.55 (m, 2H), 2.45 (s, 3H), 1.11 (d, J = 5.2 Hz, 3H). [Note: -one proton merged in solvent peak]. |
| 321 | 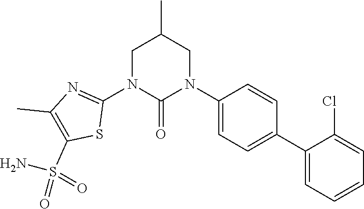 [SMILES: Cc1nc(N2CC(C)CN(c3ccc(-c4ccccc4Cl)cc3)C2=O)sc1S(N)(=O)=O] | 477.4 | ¹H NMR (400 MHz, DMSO-d₆): δ 7.60-7.53 (m, 3H), 7.49-7.37 (m, 7H), 4.42-4.32 (m, 1H), 3.81-3.72 (m, 1H), 3.69- 3.56 (m, 2H), 2.43 (s, 3H), 1.10 (d, J = 6.5 Hz, 3H). [Note: -One proton merged in solvent peak]. |
| 322 | 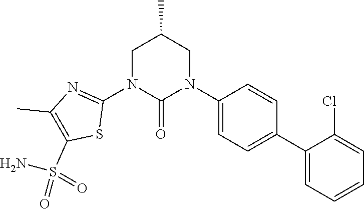 [SMILES: Cc1nc(N2C[C@H](C)CN(c3ccc(-c4ccccc4Cl)cc3)C2=O)sc1S(N)(=O)=O] Single enantiomer, arbitrarily assigned stereochemistry | 477.4 | ¹H NMR (400 MHz, DMSO-d₆): δ 7.61-7.55 (m, 1H), 7.51-7.38 (m, 9H), 4.38 (dd, J = 3.3, 12.6 Hz, 1H), 3.77 (dd, J = 2.7, 11.6 Hz, 1H), 3.70- 3.58 (m, 2H), 2.44 (s, 3H), 1.12 (d, J = 6.6 Hz, 3H). [Note: One proton merged in solvent peak] |
| 323 | 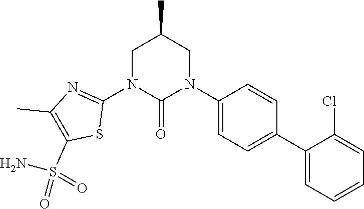 [SMILES: Cc1nc(N2C[C@@H](C)CN(c3ccc(-c4ccccc4Cl)cc3)C2=O)sc1S(N)(=O)=O] Single enantiomer, arbitrarily assigned stereochemistry | 477.4 | ¹H NMR (400 MHz, DMSO-d₆): δ 7.60-7.56 (m, 1H), 7.51-7.38 (m, 9H), 4.38 (dd, J = 4.9, 12.0 Hz, 1H), 3.80-3.73 (m, 1H), 3.69-3.57 (m, 2H), 2.44 (s, 3H), 1.12 (d, J = 6.8 Hz, 3H). [Note: One proton merged in solvent peak]. |
| 324 | 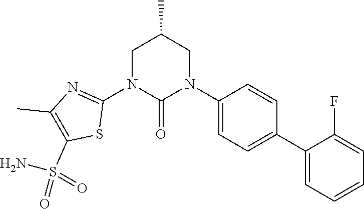 [SMILES: Cc1nc(N2C[C@H](C)CN(c3ccc(-c4ccccc4F)cc3)C2=O)sc1S(N)(=O)=O] Single enantiomer, arbitrarily assigned stereochemistry | 459.2 | ¹H NMR (400 MHz, CD₃OD-d₄): δ 7.66-7.56 (m, 2H), 7.53-7.42 (m, 3H), 7.42-7.33 (m, 1H), 7.29-7.15 (m, 2H), 4.56- 4.48 (m, 1H), 3.84- 3.58 (m, 3H), 2.59-2.49 (m, 4H), 1.21 (d, J = 6.4 Hz, 3H). [Note: -NH₂ protons not observed]. |
| 325 | 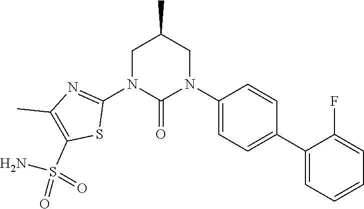 [SMILES: Cc1nc(N2C[C@@H](C)CN(c3ccc(-c4ccccc4F)cc3)C2=O)sc1S(N)(=O)=O] Single enantiomer, arbitrarily assigned stereochemistry | 459.2 | ¹H NMR (400 MHz, CD₃OD-d₄): δ 7.61 (d, J = 7.8 Hz, 2H), 7.53-7.42 (m, 3H), 7.41-7.33 (m, 1H), 7.30-7.14 (m, 2H), 4.56-4.48 (m, 1H), 3.84- 3.58 (m, 3H), 2.59- 2.49 (m, 4H), 1.21 (d, J = 6.4 Hz, 3H). [Note: -NH₂ protons not observed]. |
| 326 | 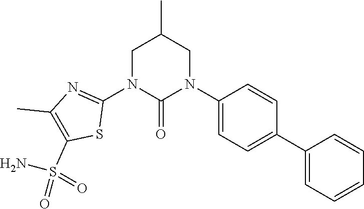 [SMILES: Cc1nc(N2CC(C)CN(c3ccc(-c4ccccc4)cc3)C2=O)sc1S(N)(=O)=O] | 443.4 | ¹H NMR (400 MHz, DMSO-d₆): δ 7.81-7.71 (m, 4H), 7.64 (s, 2H), 7.59-7.50 (m, 4H), 7.47- 7.42 (m, 1H), 4.49- 4.41 (m, 1H), 3.85-3.78 (m, 1H), 3.76-3.62 (m, 2H), 2.51 (s, 3H), 1.18 (d, J = 6.6 Hz, 3H). [Note: -One proton merged in solvent peak] |
| 327 | 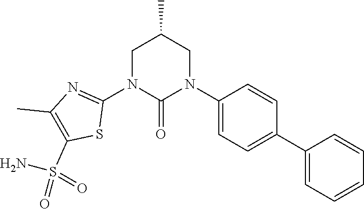 [SMILES: Cc1nc(N2C[C@H](C)CN(c3ccc(-c4ccccc4)cc3)C2=O)sc1S(N)(=O)=O] Single enantiomer, arbitrarily assigned stereochemistry | 443.4 | ¹H NMR (400 MHz, DMSO-d₆): δ 7.73-7.66 (m, 4H), 7.54-7.44 (m, 6H), 7.41-7.35 (m, 1H), 4.42-4.34 (m, 1H), 3.79- 3.72 (m, 1H), 3.69- 3.56 (m, 2H), 2.44 (s, 3H), 1.11 (d, J = 6.6 Hz, 3H). [Note: -One proton merged in solvent peak]. |
| 328 | 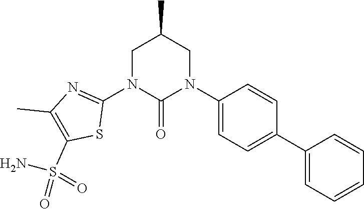 [SMILES: Cc1nc(N2C[C@@H](C)CN(c3ccc(-c4ccccc4)cc3)C2=O)sc1S(N)(=O)=O] Single enantiomer, arbitrarily assigned stereochemistry | 443.5 | ¹H NMR (400 MHz, DMSO-d₆): δ 7.73-7.64 (m, 4H), 7.56-7.44 (m, 6H), 7.41-7.33 (m, 1H), 4.42-4.34 (m, 1H), 3.80- 3.72 (m, 1H), 3.70- 3.55 (m, 2H), 2.44 (s, 3H), 1.11 (d, J = 6.6 Hz, 3H). [Note: -One proton merged in solvent peak] |
| 329 | 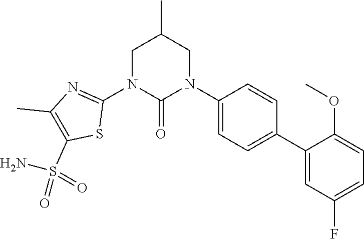 [SMILES: COc1ccc(F)cc1-c1ccc(N2CC(C)CN(c3nc(C)c(S(N)(=O)=O)s3)C2=O)cc1] | 491.5 | ¹H NMR (400 MHz, DMSO-d₆): δ 7.54 (d, J = 7.9 Hz, 2H), 7.47-7.35 (m, 2H), 7.24-7.08 (m, 5H), 4.42-4.32 (m, 1H), 3.77 (s, 3H), 3.75-3.71 (m, 1H), 3.69-3.53 (m, 2H), 2.43 (s, 3H), 1.11 (d, J = 6.5 Hz, 3H). [Note: -One proton merged in solvent peak]. |
| 330 | 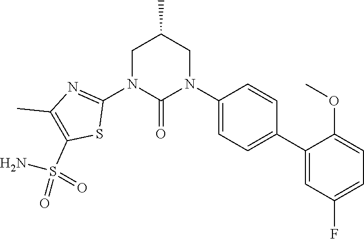 [SMILES: COc1ccc(F)cc1-c1ccc(N2C[C@@H](C)CN(c3nc(C)c(S(N)(=O)=O)s3)C2=O)cc1] Single enantiomer, arbitrarily assigned stereochemistry | 491.5 | ¹H NMR (400 MHz, DMSO-d₆): δ 7.55-7.51 (m, 2H), 7.41 (d, J = 8.6 Hz, 2H), 7.21-7.07 (m, 5H), 4.41-4.32 (m, 1H), 3.77 (s, 3H), 3.76-3.71 (m, 1H), 3.68-3.55 (m, 2H), 2.42 (s, 3H), 1.11 (d, J = 6.6 Hz, 3H). [Note: -One proton merged in solvent peak]. |
| 331 | 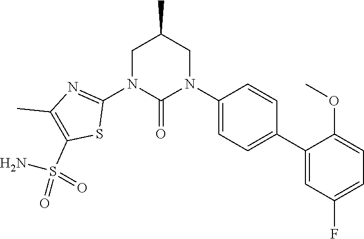 [SMILES: COc1ccc(F)cc1-c1ccc(N2C[C@H](C)CN(c3nc(C)c(S(N)(=O)=O)s3)C2=O)cc1] Single enantiomer, arbitrarily assigned stereochemistry | 491.4 | ¹H NMR (400 MHz, DMSO-d₆): δ 7.54 (d, J = 8.3 Hz, 2H), 7.41 (d, J = 8.3 Hz, 2H), 7.21-7.06 (m, 5H), 4.41-4.31 (m, 1H), 3.77 (s, 3H), 3.74- 3.71 (m, 1H), 3.67-3.55 (m, 2H), 2.42 (s, 3H), 1.11 (d, J = 6.4 Hz, 3H). [Note: -One proton merged in solvent peak]. |
| 332 | 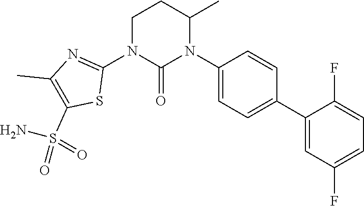 [SMILES: Cc1nc(N2CCC(C)N(c3ccc(-c4cc(F)ccc4F)cc3)C2=O)sc1S(N)(=O)=O] | 478.1 | ¹H NMR (400 MHz, DMSO-d₆): δ 7.65 (d, J = 7.8 Hz, 2H), 7.54 (s, 2H), 7.50-7.35 (m, 4H), 7.33- 7.24 (m, 1H), 4.38- 4.27 (m, 1H), 4.20-4.04 (m, 2H), 2.45 (s, 3H), 2.10-2.02 (m, 2H), 1.16 (d, J = 6.4 Hz, 3H). |
| 333 | 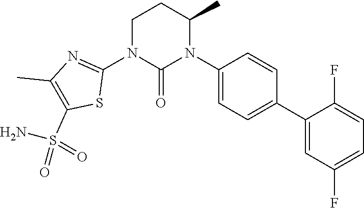 [SMILES: Cc1nc(N2CC[C@@H](C)N(c3ccc(-c4cc(F)ccc4F)cc3)C2=O)sc1S(N)(=O)=O] Single enantiomer, arbitrarily assigned stereochemistry | 479.2 | ¹H NMR (400 MHz, DMSO-d₆): δ 7.65 (d, J = 7.4 Hz, 2H), 7.55 (s, 2H), 7.49-7.35 (m, 4H), 7.33- 7.24 (m, 1H), 4.37- 4.26 (m, 1H), 4.19-4.04 (m, 2H), 2.45 (s, 3H), 2.40-2.37 (m, 1H), 2.11- 2.01 (m, 1H), 1.16 (d, J = 6.5 Hz, 3H). |
| 334 | 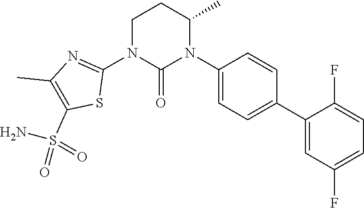 [SMILES: Cc1nc(N2CC[C@H](C)N(c3ccc(-c4cc(F)ccc4F)cc3)C2=O)sc1S(N)(=O)=O] Single enantiomer, arbitrarily assigned stereochemistry | 479.2 | ¹H NMR (400 MHz, DMSO-d₆): δ 7.67-7.62 (m, 2H), 7.55 (s, 2H), 7.50-7.36 (m, 4H), 7.34- 7.21 (m, 1H), 4.38- 4.27 (m, 1H), 4.20-4.03 (m, 2H), 2.45 (s, 3H), 2.42-2.34 (m, 1H), 2.10- 2.00 (m, 1H), 1.16 (d, J = 6.5 Hz, 3H). |
| 335 | 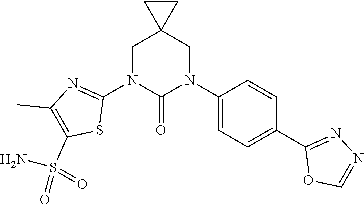 [SMILES: Cc1nc(N2CC3(CC3)CN(c3ccc(-c4nnco4)cc3)C2=O)sc1S(N)(=O)=O] | 447.0 | ¹H NMR (400 MHz, DMSO-d₆): δ 9.35 (s, 1H), 8.05 (d, J = 8.8 Hz, 2H), 7.63 (d, J = 8.6 Hz, 2H), 7.61-7.56 (s, 2H), 4.01 (s, 2H), 3.72 (s, 2H), 2.43 (s, 3H), 0.87-0.74 (m, 4H) |
| 336 | 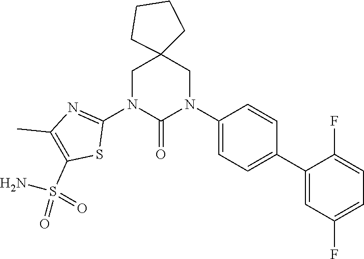 [SMILES: Cc1nc(N2CC3(CCCC3)CN(c3ccc(-c4cc(F)ccc4F)cc3)C2=O)sc1S(N)(=O)=O] | 519.3 | ¹H NMR (400 MHz, DMSO-d₆): δ 7.63-7.57 (m, 2H), 7.56-7.50 (m, 2H), 7.49-7.32 (m, 4H), 7.30-7.21 (m, 1H), 3.99 (s, 2H), 3.64 (s, 2H), 2.43 (s, 3H), 1.74-1.54 (m, 8H). |
| 337 | 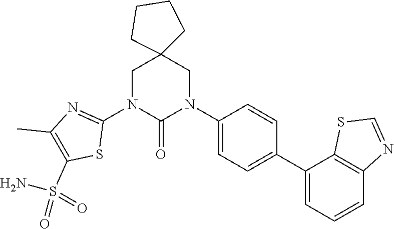 [SMILES: Cc1nc(N2CC3(CCCC3)CN(c3ccc(-c4cccc5ncsc45)cc3)C2=O)sc1S(N)(=O)=O] | 540.3 | ¹H NMR (400 MHz, DMSO-d₆): δ 9.47 (s, 1H), 8.13 (d, J = 7.8 Hz, 1H), 7.78 (d, J = 7.8 Hz, 2H), 7.73-7.66 (m, 1H), 7.65- 7.52 (m, 5H), 4.03 (s, 2H), 3.70 (s, 2H), 2.45 (s, 3H), 1.76-1.57 (m, 8H). |
| 338 | 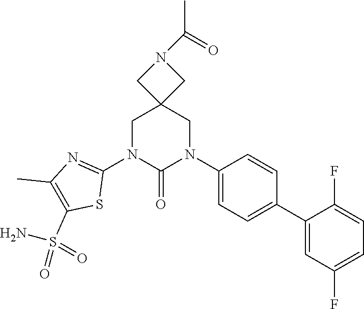 [SMILES: CC(=O)N1CC2(C1)CN(c1ccc(-c3cc(F)ccc3F)cc1)C(=O)N(c1nc(C)c(S(N)(=O)=O)s1)C2] | 548.1 | ¹H NMR (400 MHz, DMSO-d₆): δ 7.67-7.62 (m, 4H), 7.53 (dd, J = 2.00, 6.80 Hz, 2H), 7.49- 7.46 (m, 1H), 7.45-7.38 (m, 1H), 7.32-7.27 (m, 1H), 4.41 (d, J = 12.40 Hz, 1H), 4.30 (d, J= 12.40 Hz, 1H), 4.15-4.06 (m, 2H), 4.03-3.98 (m, 2H), 3.82 (s, 2H), 2.47 (s, 3H), 1.78 (s, 3H), |
| 339 | 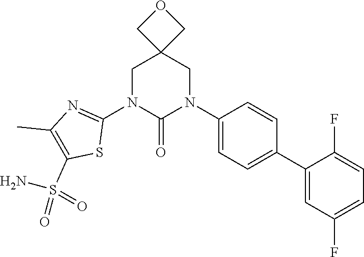 [SMILES: Cc1nc(N2CC3(COC3)CN(c3ccc(-c4cc(F)ccc4F)cc3)C2=O)sc1S(N)(=O)=O] | 507.1 | ¹H NMR (400 MHz, DMSO-d₆): δ 7.65 (d, J = 8.0 Hz, 2H), 7.60 (br s, 2H), 7.55-7.49 (m, 2H), 7.49-7.44 (m, 1H), 7.44- 7.34 (m, 1H), 7.34- 7.25 (m, 1H), 4.55 (s, 4H), 4.47-4.40 (s, 2H), 4.08 (s, 2H). [Note: -Methyl merged in solvent peak] |
| 340 | 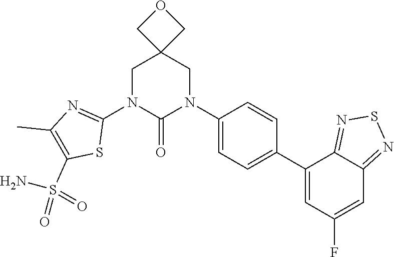 [SMILES: Cc1nc(N2CC3(COC3)CN(c3ccc(-c4cc(F)cc5nsnc45)cc3)C2=O)sc1S(N)(=O)=O] | 546.8 | ¹H NMR (400 MHz, DMSO-d₆): δ 8.10 (d, J = 8.6 Hz, 2H), 7.99-7.92 (m, 2H), 7.60 (d, J = 8.7 Hz, 4H), 4.57 (s, 4H), 4.46 (s, 2H), 4.12 (s, 2H). [Note: -CH₃ merged in solvent peak] |
| 341 | 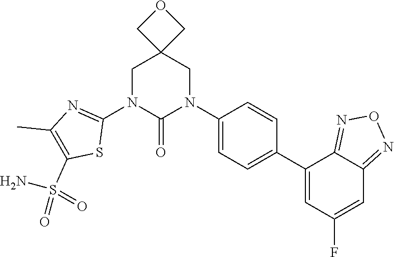 [SMILES: Cc1nc(N2CC3(COC3)CN(c3ccc(-c4cc(F)cc5nonc45)cc3)C2=O)sc1S(N)(=O)=O] | 531.0 | 1H NMR (400 MHz, DMSO-d6): δ 8.19 (d, J = 8.80 Hz, 2H), 7.99-7.95 (m, 2H), 7.65-7.62 (m, 4H), 4.57 (s, 4H), 4.46 (s, 2H), 4.13 (s, 2H), 2.51 (s, 3H) |
| 342 | 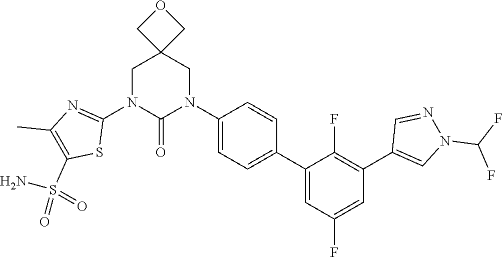 [SMILES: Cc1nc(N2CC3(COC3)CN(c3ccc(-c4cc(F)cc(-c5cnn(C(F)F)c5)c4F)cc3)C2=O)sc1S(N)(=O)=O] | 623.0 | 1H NMR (400 MHz, DMSO-d6): δ 8.76 (d, J = 1.60 Hz, 1H), 8.47 (s, 1H), 8.39 (s, 1H), 8.06- 7.76 (m, 2H), 7.70 (d, J = 6.80 Hz, 2H), 7.61-7.54 (m, 4H), 7.41-7.38 (m, 1H), 4.57 (s, 4H), 4.45 (s, 2H), 4.10 (s, 2H), 2.51 (s, 3H). |
| 343 | 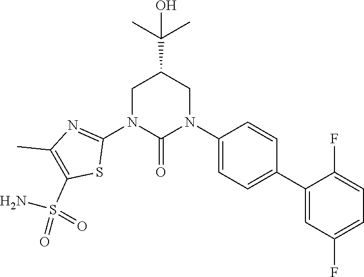 [SMILES: Cc1nc(N2C[C@H](C(C)(C)O)CN(c3ccc(-c4cc(F)ccc4F)cc3)C2=O)sc1S(N)(=O)=O] Single enantiomer, arbitrarily assigned stereochemistry | 523.4 | ¹H NMR (400 MHz, DMSO-d₆): δ 7.63 (d, J = 7.8 Hz, 2H), 7.59-7.34 (m, 6H), 7.33-7.24 (m, 1H), 4.78 (s, 1H), 4.62- 4.52 (m, 1H), 3.90-3.80 (m, 1H), 3.79-3.65 (m, 2H), 2.46 (s, 3H), 2.39- 2.28 (m, 1H), 1.20 (d, J = 10.4 Hz, 6H). |
| 344 | 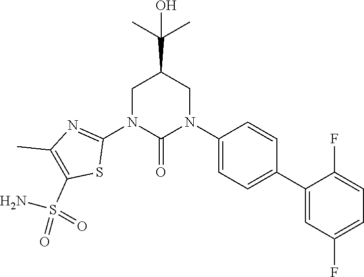 [SMILES: Cc1nc(N2C[C@@H](C(C)(C)O)CN(c3ccc(-c4cc(F)ccc4F)cc3)C2=O)sc1S(N)(=O)=O] Single enantiomer, arbitrarily assigned stereochemistry | 523.4 | ¹H NMR (400 MHz, DMSO-d₆): δ 7.65-7.61 (m, 2H), 7.60-7.49 (m, 4H), 7.48-7.36 (m, 2H), 7.33-7.23 (m, 1H), 4.78 (s, 1H), 4.60-4.53 (m, 1H), 3.89-3.81 (m, 1H), 3.79-3.66 (m, 2H), 2.46 (s, 3H), 2.39-2.29 (m, 1H), 1.20 (d, J = 10.4 Hz, 6H). |
| 345 | 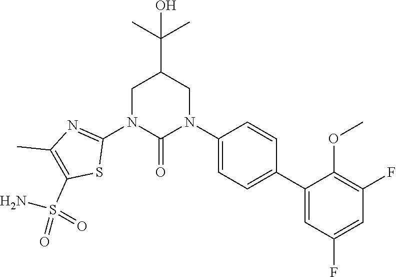 [SMILES: COc1c(F)cc(F)cc1-c1ccc(N2CC(C(C)(C)O)CN(c3nc(C)c(S(N)(=O)=O)s3)C2=O)cc1] | 553.2 | ¹H NMR (400 MHz, CD₃OD-d₄): δ 7.63-7.57 (m, 2H), 7.51-7.43 (m, 2H), 7.06-6.94 (m, 2H), 4.77-4.65 (m, 1H), 4.58 (s, 1H), 3.95-3.75 (m, 3H), 3.66 (s, 3H), 2.53 (s, 3H), 2.50-2.36 (m, 1H), 1.33 (s, 6H). |
| 346 | 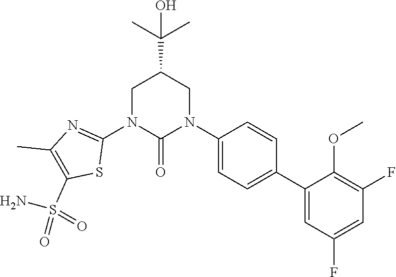 [SMILES: COc1c(F)cc(F)cc1-c1ccc(N2C[C@@H](C(C)(C)O)CN(c3nc(C)c(S(N)(=O)=O)s3)C2=O)cc1] Single enantiomer, arbitrarily assigned stereochemistry | 553.2 | ¹H NMR (400 MHz, DMSO-d₆): δ 7.62-7.54 (m, 4H), 7.53-7.47 (m, 2H), 7.41-7.33 (m, 1H), 7.17-7.11 (m, 1H), 4.77 (s, 1H), 4.61-4.53 (m, 1H), 3.89-3.82 (m, 2H), 3.78-3.70 (m, 2H), 3.67 (s, 3H), 2.49 (s, 3H), 1.23- 1.17 (m, 6H). |
| 347 | 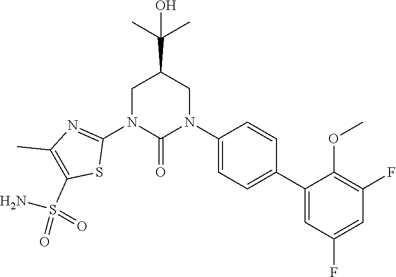 [SMILES: COc1c(F)cc(F)cc1-c1ccc(N2C[C@H](C(C)(C)O)CN(c3nc(C)c(S(N)(=O)=O)s3)C2=O)cc1] Single enantiomer, arbitrarily assigned stereochemistry | 553.2 | ¹H NMR (400 MHz, DMSO-d₆): δ 7.62-7.54 (m, 4H), 7.53-7.47 (m, 2H), 7.41-7.33 (m, 1H), 7.17-7.11 (m, 1H), 4.77 (s, 1H), 4.61-4.53 (m, 1H), 3.89-3.82 (m, 2H), 3.78-3.70 (m, 2H), 3.67 (s, 3H), 2.49 (s, 3H), 1.23- 1.17 (m, 6H). |
| 348 | 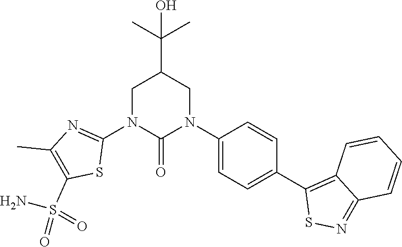 [SMILES: Cc1nc(N2CC(C(C)(C)O)CN(c3ccc(-c4snc5ccccc45)cc3)C2=O)sc1S(N)(=O)=O] | 544.2 | ¹H NMR (400 MHz, DMSO-d₆): δ 8.05-7.76 (m, 4H), 7.71-7.47 (m, 5H), 7.43-7.32 (m, 1H), 4.84-4.79 (m, 1H), 4.57 (d, J = 9.3 Hz, 1H), 3.90 (t, J = 10.5 Hz, 1H), 3.82- 3.70 (m, 2H), 1.22 (d, J = 8.8 Hz, 6H). [Note: Methyl merged in solvent peak] |
| 349 | 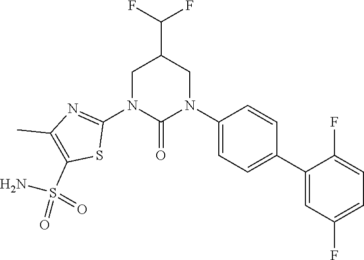 [SMILES: Cc1nc(N2CC(C(F)F)CN(c3ccc(-c4cc(F)ccc4F)cc3)C2=O)sc1S(N)(=O)=O] | 515.3 | ¹H NMR (400 MHz, DMSO-d₆): δ 7.67-7.62 (m, 2H), 7.60 (br s, 2H), 7.56-7.37 (m, 4H), 7.32- 7.25 (m, 1H), 6.64- 6.27 (m, 1H), 4.42-4.34 (m, 1H), 4.23-4.12 (m, 1H), 4.06-3.95 (m, 1H), 3.91-3.82 (m, 1H), 3.13- 3.00 (m, 1H), 2.46 (s, 3H) |
| 350 | 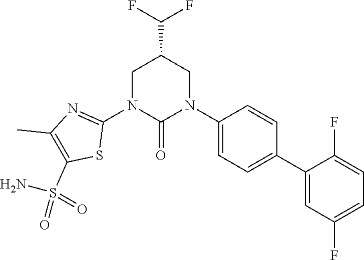 [SMILES: Cc1nc(N2C[C@H](C(F)F)CN(c3ccc(-c4cc(F)ccc4F)cc3)C2=O)sc1S(N)(=O)=O] Single enantiomer, arbitrarily assigned stereochemistry | 515.4 | ¹H NMR (400 MHz, DMSO-d₆): δ 7.66-7.62 (m, 2H), 7.60 (s, 2H), 7.56-7.37 (m, 4H), 7.34- 7.24 (m, 1H), 6.62- 6.26 (m, 1H), 4.42-4.34 (m, 1H), 4.23-4.12 (m, 1H), 4.04-3.97 (m, 1H), 3.91-3.82 (m, 1H), 3.15- 2.97 (m, 1H), 2.46 (s, 3H) |
| 351 | 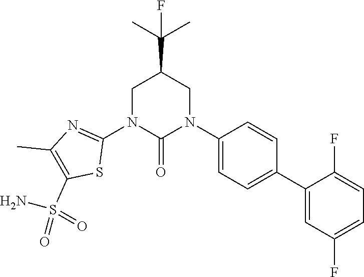 [SMILES: Cc1nc(N2C[C@@H](C(C)(C)F)CN(c3ccc(-c4cc(F)ccc4F)cc3)C2=O)sc1S(N)(=O)=O] Single enantiomer, arbitrarily assigned stereochemistry | 525.4 | ¹H NMR (400 MHz, DMSO-d₆): δ 7.66-7.61 (m, 2H), 7.59 (s, 2H), 7.56-7.51 (m, 2H), 7.49- 7.36 (m, 2H), 7.34- 7.23 (m, 1H), 4.59-4.52 (m, 1H), 3.95-3.81 (m, 2H), 3.81-3.70 (m, 1H), 2.78-2.68 (m, 1H), 2.46 (s, 3H), 1.47 (d, J = 9.6 Hz, 3H), 1.42 (d, J = 9.6 Hz, 3H). |
| 352 | 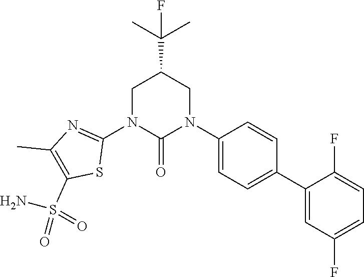 [SMILES: Cc1nc(N2C[C@H](C(C)(C)F)CN(c3ccc(-c4cc(F)ccc4F)cc3)C2=O)sc1S(N)(=O)=O] Single enantiomer, arbitrarily assigned stereochemistry | 525.3 | ¹H NMR (400 MHz, DMSO-d₆): δ 7.68-7.63 (m, 2H), 7.62 (s, 2H), 7.54-7.37 (m, 4H), 7.32- 7.26 (m, 1H), 4.47- 4.41 (m, 1H), 4.38-4.32 (m, 1H), 4.15-4.09 (m, 1H), 4.02-3.96 (m, 1H), 3.76-3.64 (m, 1H), 2.47 (s, 3H). |
| 353 | 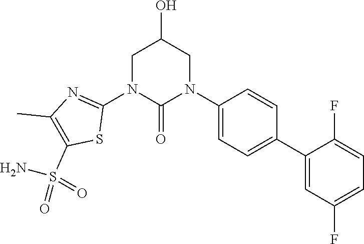 [SMILES: Cc1nc(N2CC(O)CN(c3ccc(-c4cc(F)ccc4F)cc3)C2=O)sc1S(N)(=O)=O] | 481.4 | ¹H NMR (400 MHz, DMSO-d₆): δ 7.65-7.61 (m, 2H), 7.57 (s, 2H), 7.54-7.36 (m, 4H), 7.31- 7.25 (m, 1H), 6.63 (s, 1H), 5.61 (d, J = 2.9 Hz, 1H), 4.37-4.31 (m, 2H), 4.06-3.99 (m, 2H), 2.45 (s, 3H). |
| 354 | 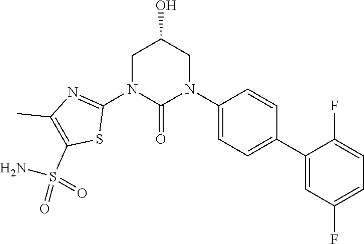 [SMILES: Cc1nc(N2C[C@H](O)CN(c3ccc(-c4cc(F)ccc4F)cc3)C2=O)sc1S(N)(=O)=O] Single enantiomer, arbitrarily assigned stereochemistry | 481.5 | ¹H NMR (400 MHz, DMSO-d₆): δ 7.65-7.61 (m, 2H), 7.57 (s, 2H), 7.52-7.34 (m, 4H), 7.32- 7.25 (m, 1H), 5.62- 5.59 (m, 1H), 4.37-4.31 (m, 2H), 4.08-3.98 (m, 2H), 3.66-3.59 (m, 1H), 2.45 (s, 3H). |
| 355 | 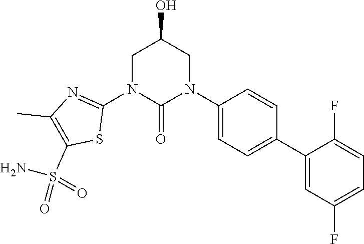 [SMILES: Cc1nc(N2C[C@@H](O)CN(c3ccc(-c4cc(F)ccc4F)cc3)C2=O)sc1S(N)(=O)=O] Single enantiomer, arbitrarily assigned stereochemistry | 481.4 | ¹H NMR (400 MHz, DMSO-d₆): δ 7.65-7.61 (m, 2H), 7.57 (s, 2H), 7.52-7.34 (m, 4H), 7.32- 7.22 (m, 1H), 5.62- 5.59 (m, 1H), 4.38-4.30 (m, 2H), 4.09-3.97 (m, 2H), 3.67-3.59 (m, 1H), 2.45 (s, 3H). |
| 356 | 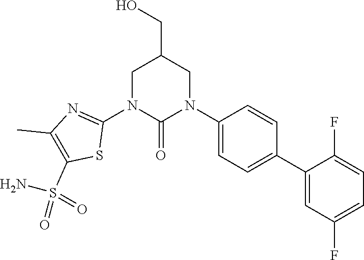 [SMILES: Cc1nc(N2CC(CO)CN(c3ccc(-c4cc(F)ccc4F)cc3)C2=O)sc1S(N)(=O)=O] | 495.4 | ¹H NMR (400 MHz, DMSO-d₆): δ 7.63 (d, J = 8.3 Hz, 2H), 7.57 (s, 2H), 7.53-7.35 (m, 4H), 7.35- 7.21 (m, 1H), 5.01- 4.94 (m, 1H), 4.42-4.32 (m, 1H), 3.90-3.77 (m, 2H), 3.77-3.66 (m, 1H), 3.60-3.52 (m, 2H), 2.45 (s, 3H). [Note: -One proton merged in solvent peak]. |
| 357 | 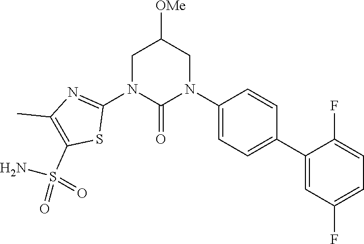 [SMILES: COC1CN(c2ccc(-c3cc(F)ccc3F)cc2)C(=O)N(c2nc(C)c(S(N)(=O)=O)s2)C1] | 495.3 | ¹H NMR (400 MHz, DMSO-d₆): δ 7.72-7.58 (m, 4H), 7.47-7.16 (m, 5H), 5.09-4.99 (m, 1H), 4.24 (t, J = 8.8 Hz, 1H), 3.92-3.87 (m, 3H), 3.12 (s, 3H), 2.33 (s, 3H). |
| 358 | 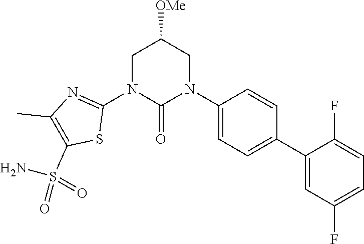 [SMILES: CO[C@@H]1CN(c2ccc(-c3cc(F)ccc3F)cc2)C(=O)N(c2nc(C)c(S(N)(=O)=O)s2)C1] Single enantiomer, arbitrarily assigned stereochemistry | 495.3 | ¹H NMR (400 MHz, DMSO-d₆): δ 7.73-7.57 (m, 4H), 7.48-7.31 (m, 4H), 7.29-7.18 (m, 1H), 5.09-4.98 (m, 1H), 4.30- 4.17 (m, 1H), 3.94-3.80 (m, 3H), 3.12 (s, 3H), 2.33 (s, 3H). |
| 359 | 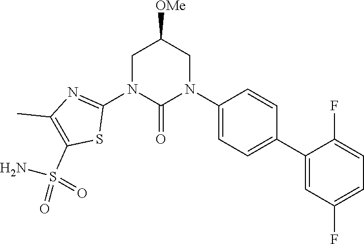 [SMILES: CO[C@H]1CN(c2ccc(-c3cc(F)ccc3F)cc2)C(=O)N(c2nc(C)c(S(N)(=O)=O)s2)C1] Single enantiomer, arbitrarily assigned stereochemistry | 495.4 | ¹H NMR (400 MHz, DMSO-d₆): δ 7.72-7.57 (m, 4H), 7.48-7.32 (m, 4H), 7.30-7.19 (m, 1H), 5.09-4.99 (m, 1H), 4.28- 4.19 (m, 1H), 3.93- 3.82 (m, 3H), 3.12 (s, 3H), 2.33 (s, 3H). |
| 360 | 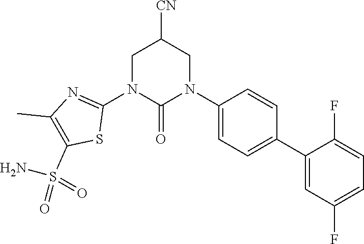 [SMILES: Cc1nc(N2CC(C#N)CN(c3ccc(-c4cc(F)ccc4F)cc3)C2=O)sc1S(N)(=O)=O] | 489.9 | ¹H NMR (400 MHz, DMSO-d₆): δ 7.67 (d, J = 7.8 Hz, 2H), 7.61 (s, 2H), 7.51-7.36 (m, 4H), 7.33- 7.24 (m, 1H), 4.71-4.62 (m, 1H), 4.27-4.16 (m, 2H), 4.06-3.96 (m, 2H), 2.47 (s, 3H). |
| 361 | 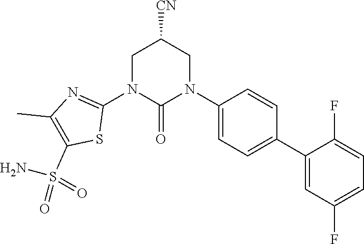 [SMILES: Cc1nc(N2C[C@H](C#N)CN(c3ccc(-c4cc(F)ccc4F)cc3)C2=O)sc1S(N)(=O)=O] Single enantiomer, arbitrarily assigned stereochemistry | 490.3 | ¹H NMR (400 MHz, DMSO-d₆): δ 7.70-7.64 (m, 2H), 7.59 (brs, 2H), 7.52-7.35 (m, 4H), 7.33- 7.23 (m, 1H), 4.70- 4.61 (m, 1H), 4.26-4.16 (m, 2H), 4.05-3.98 (m, 2H), 2.47 (s, 3H). |
| 362 | 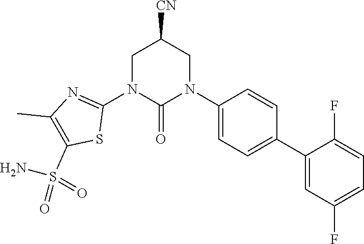 [SMILES: Cc1nc(N2C[C@@H](C#N)CN(c3ccc(-c4cc(F)ccc4F)cc3)C2=O)sc1S(N)(=O)=O] Single enantiomer, arbitrarily assigned stereochemistry | 490.4 | ¹H NMR (400 MHz, DMSO-d₆): δ 7.71-7.63 (m, 2H), 7.59 (s, 2H), 7.52-7.35 (m, 4H), 7.34- 7.24 (m, 1H), 4.71- 4.61 (m, 1H), 4.27-4.14 (m, 2H), 4.05-3.95 (m, 2H), 2.47 (s, 3H). |
| 363 | 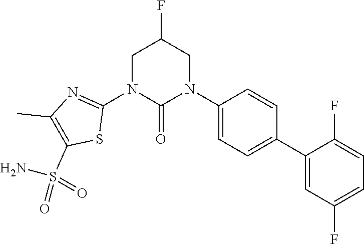 [SMILES: Cc1nc(N2CC(F)CN(c3ccc(-c4cc(F)ccc4F)cc3)C2=O)sc1S(N)(=O)=O] | 483.4 | ¹H NMR (400 MHz, DMSO-d₆): δ 7.69-7.56 (m, 4H), 7.54-7.33 (m, 4H), 7.33-7.24 (m, 1H), 5.63-5.38 (m, 1H), 4.78- 4.64 (m, 1H), 4.36-3.90 (m, 3H), 2.46 (s, 3H). |
| 364 | 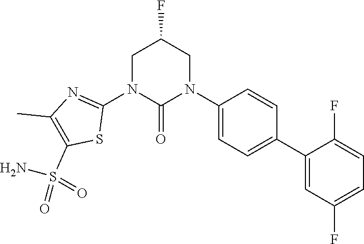 [SMILES: Cc1nc(N2C[C@H](F)CN(c3ccc(-c4cc(F)ccc4F)cc3)C2=O)sc1S(N)(=O)=O] Single enantiomer, arbitrarily assigned stereochemistry | 483.4 | ¹H NMR (400 MHz, DMSO-d₆): δ 7.69-7.64 (m, 2H), 7.60 (s, 2H), 7.55-7.36 (m, 4H), 7.32- 7.24 (m, 1H), 5.51 (d, J = 45.6 Hz, 1H), 4.80- 4.63 (m, 1H), 4.36-4.13 (m, 2H), 4.12-3.91 (m, 1H). [Note: -CH₃ merged in solvent peak]. |
| 365 | 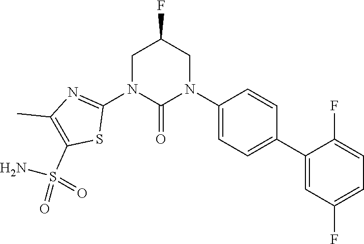 [SMILES: Cc1nc(N2C[C@@H](F)CN(c3ccc(-c4cc(F)ccc4F)cc3)C2=O)sc1S(N)(=O)=O] Single enantiomer, arbitrarily assigned stereochemistry | 483.4 | ¹H NMR (400 MHz, DMSO-d₆): δ 7.68-7.63 (m, 2H), 7.61 (s, 2H), 7.52-7.37 (m, 4H), 7.32- 7.23 (m, 1H), 5.51 (d, J = 46.8 Hz, 1H), 4.78- 4.64 (m, 1H), 4.36-4.13 (m, 2H), 4.12-3.91 (m, 1H), 2.46 (s, 3H). |
| 366 | 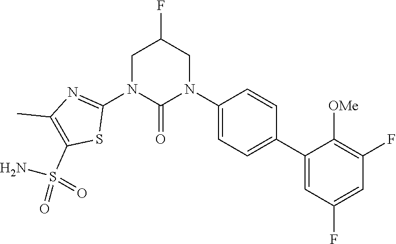 [SMILES: COc1c(F)cc(F)cc1-c1ccc(N2CC(F)CN(c3nc(C)c(S(N)(=O)=O)s3)C2=O)cc1] | 511.3 | ¹H NMR (400 MHz, DMSO-d₆): δ 7.64-7.58 (m, 4H), 7.50-7.44 (m, 2H), 7.41-7.34 (m, 1H), 7.15 (d, J = 8.1 Hz, 1H), 5.60-5.42 (m, 1H), 4.71 (t, J = 11.2 Hz, 1H), 4.37- 4.03 (m, 2H), 4.02- 3.90 (m, 1H), 3.68 (s, 3H), 2.46 (s, 3H). |
| 367 | 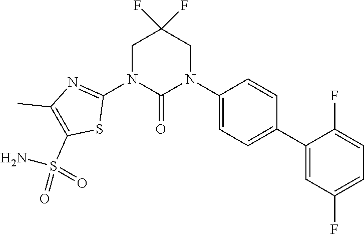 [SMILES: Cc1nc(N2CC(F)(F)CN(c3ccc(-c4cc(F)ccc4F)cc3)C2=O)sc1S(N)(=O)=O] | 501.4 | ¹H NMR (400 MHz, DMSO-d₆): δ 7.70-7.64 (m, 4H), 7.51 (d, J = 8.3 Hz, 2H), 7.48-7.35 (m, 2H), 7.33-7.24 (m, 1H), 4.60 (t, J = 12.5 Hz, 2H), 4.36 (t, J = 11.7 Hz, 2H), 2.47 (s, 3H). |
| 368 | 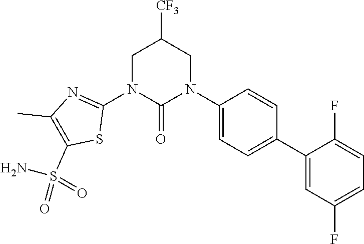 [SMILES: Cc1nc(N2CC(C(F)(F)F)CN(c3ccc(-c4cc(F)ccc4F)cc3)C2=O)sc1S(N)(=O)=O] | 533.4 | ¹H NMR (400 MHz, DMSO-d₆): δ 7.68-7.61 (m, 4H), 7.52-7.37 (m, 4H), 7.32-7.25 (m, 1H), 4.44 (dd, J = 4.9, 13.1 Hz, 1H), 4.40-4.30 (m, 1H), 4.15-4.08 (m, 1H), 4.03-3.96 (m, 1H), 3.76- 3.65 (m, 1H), 2.46 (s, 3H) |
| 369 | 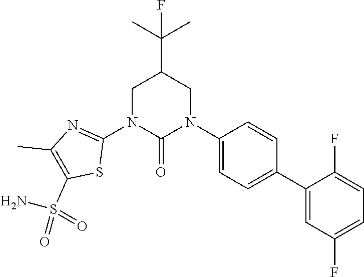 [SMILES: Cc1nc(N2CC(C(C)(C)F)CN(c3ccc(-c4cc(F)ccc4F)cc3)C2=O)sc1S(N)(=O)=O] | 525.4 | ¹H NMR (400 MHz, DMSO-d₆): δ 7.66-7.60 (m, 2H), 7.58 (s, 2H), 7.56-7.51 (m, 2H), 7.49- 7.36 (m, 2H), 7.33- 7.23 (m, 1H), 4.55 (dd, J = 3.1, 12.6 Hz, 1H), 3.93- 3.82 (m, 2H), 3.78- 3.71 (m, 1H), 2.79-2.68 (m, 1H), 2.46 (s, 3H), 1.47 (d, J = 9.9 Hz, 3H), 1.42 (d, J = 9.9 Hz, 3H) |
| 370 | 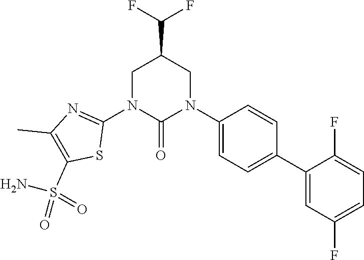 [SMILES: Cc1nc(N2C[C@@H](C(F)F)CN(c3ccc(-c4cc(F)ccc4F)cc3)C2=O)sc1S(N)(=O)=O] Single enantiomer, arbitrarily assigned stereochemistry | 515.2 | ¹H NMR (400 MHz, DMSO-d₆): δ 7.67-7.62 (m, 2H), 7.60 (s, 2H), 7.54-7.48 (m, 2H), 7.48- 7.36 (m, 2H), 7.34- 7.24 (m, 1H), 6.60-6.25 (m, 1H), 4.42-4.33 (m, 1H), 4.22-4.14 (m, 1H), 4.03-3.96 (m, 1H), 3.91- 3.84 (m, 1H), 3.13- 3.00 (m, 1H), 2.46 (s, 3H). |
| 371 | 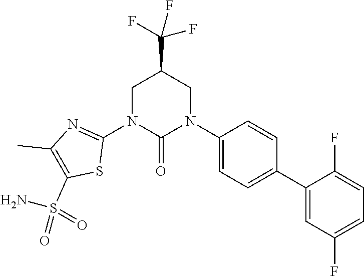 [SMILES: Cc1nc(N2C[C@@H](C(F)(F)F)CN(c3ccc(-c4cc(F)ccc4F)cc3)C2=O)sc1S(N)(=O)=O] Single enantiomer, arbitrarily assigned stereochemistry | 533.2 | ¹H NMR (400 MHz, DMSO-d₆): δ 7.68-7.63 (m, 2H), 7.62 (s, 2H), 7.54-7.37 (m, 4H), 7.32- 7.26 (m, 1H), 4.47- 4.41 (m, 1H), 4.38-4.32 (m, 1H), 4.15-4.09 (m, 1H), 3.99 (dd, J = 6.8, 12.2 Hz, 1H), 3.75-3.67 (m, 1H), 2.47 (s, 3H). |
| 372 | 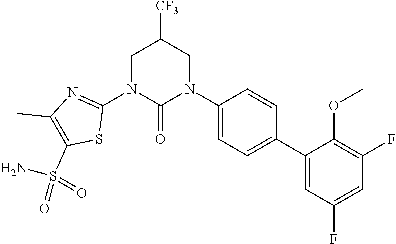 [SMILES: COc1c(F)cc(F)cc1-c1ccc(N2CC(C(F)(F)F)CN(c3nc(C)c(S(N)(=O)=O)s3)C2=O)cc1] | 563.1 | ¹H NMR (400 MHz, DMSO-d₆): δ 7.61 (d, J = 8.4 Hz, 4H), 7.47 (d, J = 8.5 Hz, 2H), 7.43-7.35 (m, 1H), 7.20-7.11 (m, 1H), 4.44 (dd, J = 4.9, 12.9 Hz, 1H), 4.39-4.31 (m, 1H), 4.15-4.08 (m, 1H), 4.03-3.96 (m, 1H), 3.68 (s, 3H), 2.46 (s, 3H). [Note: -One proton merged in solvent peak] |
| 373 | 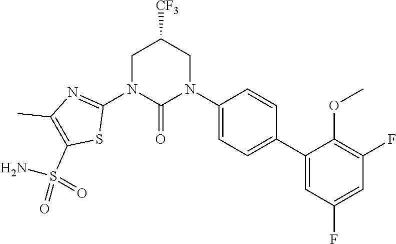 [SMILES: COc1c(F)cc(F)cc1-c1ccc(N2C[C@@H](C(F)(F)F)CN(c3nc(C)c(S(N)(=O)=O)s3)C2=O)cc1] Single enantiomer, arbitrarily assigned stereochemistry | 563.2 | ¹H NMR (400 MHz, DMSO-d₆): δ 7.64-7.59 (m, 4H), 7.47 (d, J = 8.6 Hz, 2H), 7.41-7.34 (m, 1H), 7.15 (dd, J = 1.9, 9.2 Hz, 1H), 4.45 (dd, J = 5.1, 13.2 Hz, 1H), 4.39- 4.32 (m, 1H), 4.12 (dd, J = 4.6, 12.2 Hz, 1H), 3.99 (dd, J = 6.5, 12.0 Hz, 1H), 3.77-3.69 (m, 1H), 3.68 (s, 3H), 2.47 (s, 3H). |
| 374 | 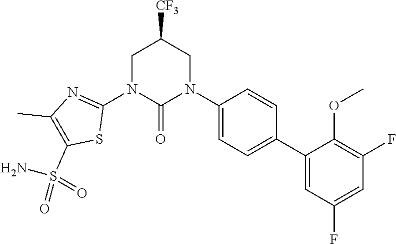 [SMILES: COc1c(F)cc(F)cc1-c1ccc(N2C[C@H](C(F)(F)F)CN(c3nc(C)c(S(N)(=O)=O)s3)C2=O)cc1] Single enantiomer, arbitrarily assigned stereochemistry | 563.2 | ¹H NMR (400 MHz, DMSO-d₆): δ 7.65-7.57 (m, 4H), 7.47 (d, J = 8.6 Hz, 2H), 7.42-7.34 (m, 1H), 7.17-7.12 (m, 1H), 4.44 (dd, J = 4.9, 13.1 Hz, 1H), 4.39-4.32 (m, 1H), 4.15-4.08 (m, 1H), 4.03-3.96 (m, 1H), 3.78- 3.69 (m, 1H), 3.68 (s, 3H), 2.47 (s, 3H). |
| 375 | 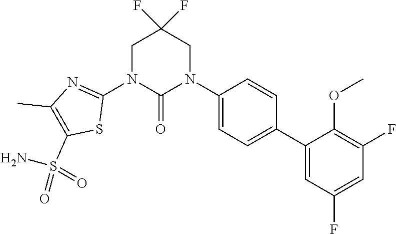 [SMILES: COc1c(F)cc(F)cc1-c1ccc(N2CC(F)(F)CN(c3nc(C)c(S(N)(=O)=O)s3)C2=O)cc1] | 531.4 | ¹H NMR (400 MHz, DMSO-d₆): δ 7.68 (s, 2H), 7.65-7.59 (m, 2H), 7.49 (d, J = 8.5 Hz, 2H), 7.46- 7.32 (m, 1H), 7.18-7.11 (m, 1H), 4.60 (t, J = 12.7 Hz, 2H), 4.36 (t, J = 11.8 Hz, 2H), 3.68 (s, 3H), 2.47 (s, 3H) |
| 376 | 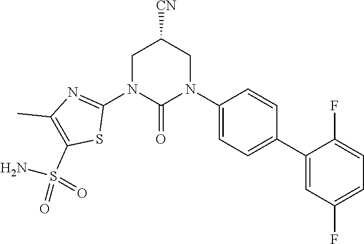 [SMILES: Cc1nc(N2C[C@H](C#N)CN(c3ccc(-c4cc(F)ccc4F)cc3)C2=O)sc1S(N)(=O)=O] Single enantiomer, arbitrarily assigned stereochemistry | 490.3 | ¹H NMR (400 MHz, DMSO-d₆): δ 7.70-7.64 (m, 2H), 7.59 (brs, 2H), 7.52-7.35 (m, 4H), 7.33- 7.23 (m, 1H), 4.70- 4.61 (m, 1H), 4.26-4.16 (m, 2H), 4.05-3.98 (m, 2H), 2.47 (s, 3H). |
| 377 | 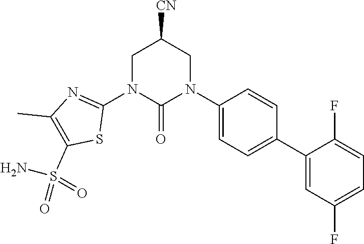 [SMILES: Cc1nc(N2C[C@@H](C#N)CN(c3ccc(-c4cc(F)ccc4F)cc3)C2=O)sc1S(N)(=O)=O] Single enantiomer, arbitrarily assigned stereochemistry | 490.4 | ¹H NMR (400 MHz, DMSO-d₆): δ 7.71-7.63 (m, 2H), 7.59 (s, 2H), 7.52-7.35 (m, 4H), 7.34- 7.24 (m, 1H), 4.71- 4.61 (m, 1H), 4.27-4.14 (m, 2H), 4.05-3.95 (m, 2H), 2.47 (s, 3H). |
| 378 | 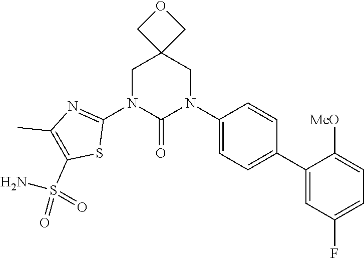 [SMILES: COc1ccc(F)cc1-c1ccc(N2CC3(COC3)CN(c3nc(C)c(S(N)(=O)=O)s3)C2=O)cc1] | 519.1 | 1H NMR (400 MHz, DMSO-d6): δ 7.60-7.56 (m, 3H), 7.44 (dd, J = 2.00, 6.80 Hz, 2H), 7.22- 7.20 (m, 2H), 7.19-7.13 (m, 2H), 4.56 (s, 4H), 4.44 (s, 2H), 4.07 (s, 2H), 3.79 (s, 3H), 2.50 (s, 3H) |
| 379 | 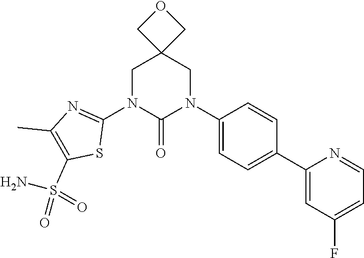 [SMILES: Cc1nc(N2CC3(COC3)CN(c3ccc(-c4cc(F)ccn4)cc3)C2=O)sc1S(N)(=O)=O] | 490.2 | ¹H NMR (400 MHz, DMSO-d₆): δ 8.71 (dd, J = 5.7, 9.2 Hz, 1H), 8.19 (d, J = 8.6 Hz, 2H), 7.97 (dd, J = 2.3, 11.1 Hz, 1H), 7.59 (s, 2H), 7.57-7.50 (m, 2H), 7.35-7.24 (m, 1H), 4.56 (s, 4H), 4.44 (s, 2H), 4.09 (s, 2H), 2.48 (s, 3H). |
| 380 | 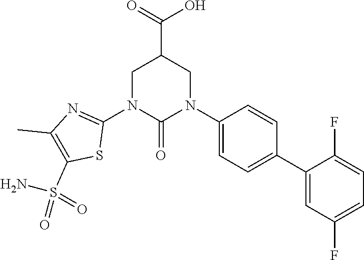 [SMILES: Cc1nc(N2CC(C(=O)O)CN(c3ccc(-c4cc(F)ccc4F)cc3)C2=O)sc1S(N)(=O)=O] | 509.3 | ¹H NMR (400 MHz, DMSO-d₆): δ 13.13 (brs, 1H), 7.63 (d, J = 7.4 Hz, 2H), 7.60-7.54 (m, 2H), 7.52-7.36 (m, 4H), 7.32- 7.24 (m, 1H), 4.52- 4.42 (m, 1H), 4.28-4.20 (m, 1H), 4.06-3.93 (m, 2H), 3.47-3.38 (m, 1H), 2.46 (s, 3H). |
| 381 | 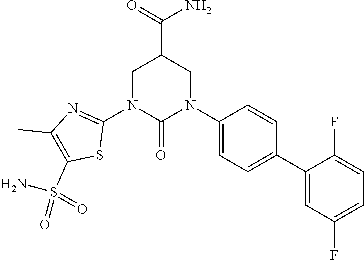 [SMILES: Cc1nc(N2CC(C(N)=O)CN(c3ccc(-c4cc(F)ccc4F)cc3)C2=O)sc1S(N)(=O)=O] | 508.4 | ¹H NMR (400 MHz, DMSO-d₆): δ 7.71 (brs, 1H), 7.66-7.62 (m, 2H), 7.58 (s, 2H), 7.52-7.37 (m, 4H), 7.32-7.24 (m, 2H), 4.37-4.21 (m, 2H), 4.00 (d, J = 5.9 Hz, 2H), 3.27-3.20 (m, 1H), 2.46 (s, 3H). |
| 382 | 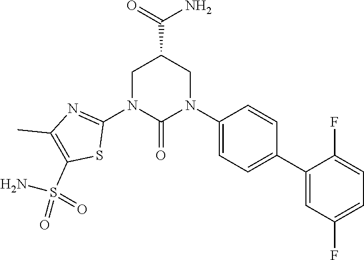 [SMILES: Cc1nc(N2C[C@H](C(N)=O)CN(c3ccc(-c4cc(F)ccc4F)cc3)C2=O)sc1S(N)(=O)=O] Single enantiomer, arbitrarily assigned stereochemistry | 508.4 | ¹H NMR (400 MHz, DMSO-d₆): δ 7.70 (s, 1H), 7.63 (d, J = 7.8 Hz, 2H), 7.57 (s, 2H). 7.52-7.36 (m, 4H), 7.33-7.20 (m, 2H), 4.37-4.29 (m, 1H), 4.27-4.19 (m, 1H), 3.99 (d, J = 5.4 Hz, 2H), 3.26- 3.19 (m, 1H), 2.46 (s, 3H). |
| 383 | 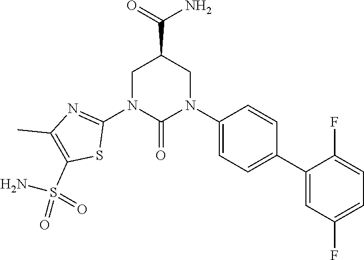 [SMILES: Cc1nc(N2C[C@@H](C(N)=O)CN(c3ccc(-c4cc(F)ccc4F)cc3)C2=O)sc1S(N)(=O)=O] Single enantiomer, arbitrarily assigned stereochemistry | 508.0 | ¹H NMR (400 MHz, DMSO-d₆): δ 7.70 (s, 1H), 7.63 (d, J = 8.3 Hz, 2H), 7.59-7.53 (m, 1H), 7.52- 7.35 (m, 5H), 7.32- 7.20 (m, 2H), 4.38-4.29 (m, 1H), 4.29-4.19 (m, 1H), 3.99 (d, J = 4.9 Hz, 2H), 3.26-3.19 (m, 1H), 2.46 (s, 3H). |
| 384 | 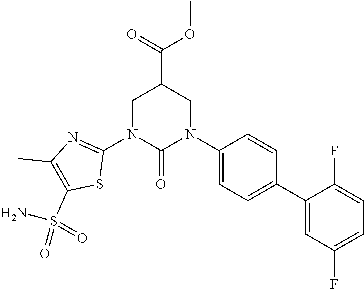 [SMILES: COC(=O)C1CN(c2ccc(-c3cc(F)ccc3F)cc2)C(=O)N(c2nc(C)c(S(N)(=O)=O)s2)C1] | 523.2 | ¹H NMR (400 MHz, DMSO-d₆): δ 7.64 (d, J = 7.3 Hz, 2H), 7.58 (s, 2H), 7.49-7.36 (m, 4H), 7.33- 7.24 (m, 1H), 4.52- 4.43 (m, 1H), 4.34-4.26 (m, 1H), 4.13-4.06 (m, 1H), 4.03-3.96 (m, 1H), 3.71 (s, 3H), 3.66-3.58 (m, 1H), 2.47 (s, 3H) |
| 385 | 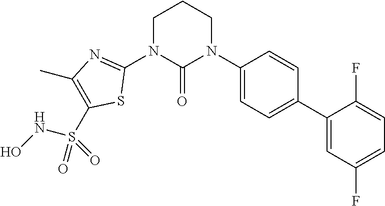 [SMILES: Cc1nc(N2CCCN(c3ccc(-c4cc(F)ccc4F)cc3)C2=O)sc1S(=O)(=O)NO] | 481.1 | ¹H NMR (400 MHz, DMSO-d₆): δ 9.62 (s, 1H), 9.51 (s, 1H), 7.63 (d, J = 7.8 Hz, 2H), 7.54-7.49 (m, 2H), 7.48-7.35 (m, 2H), 7.32-7.23 (m, 1H), 4.19 (t, J = 5.6 Hz, 2H), 3.83 (t, J = 5.1 Hz, 2H), 2.29-2.20 (m, 2H). [Note: -CH₃ merged in solvent peak]. |
| 386 | 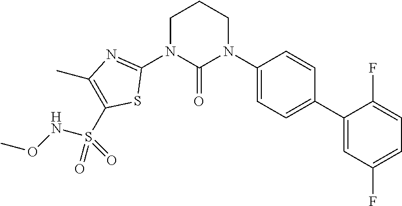 [SMILES: CONS(=O)(=O)c1sc(N2CCCN(c3ccc(-c4cc(F)ccc4F)cc3)C2=O)nc1C] | 494.9 | ¹H NMR (400 MHz, DMSO-d₆): δ 10.46 (s, 1H), 7.63 (d, J = 7.3 Hz, 2H), 7.55-7.49 (m, 2H), 7.48-7.36 (m, 2H), 7.33- 7.25 (m, 1H), 4.19 (t, J = 5.9 Hz, 2H), 3.83 (t, J = 5.5 Hz, 2H), 3.67 (s, 3H), 2.28-2.21 (m, 2H). [Note: -CH₃ merged in solvent peak]. |
| 387 | 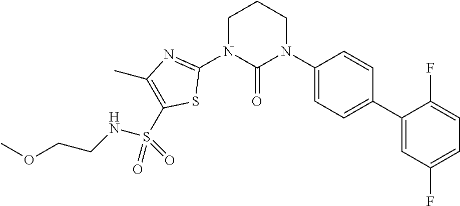 [SMILES: COCCNS(=O)(=O)c1sc(N2CCCN(c3ccc(-c4cc(F)ccc4F)cc3)C2=O)nc1C] | 523.2 | ¹H NMR (400 MHz, DMSO-d₆): δ 7.91 (s, 1H), 7.65-7.60 (m, 2H), 7.53- 7.48 (m, 2H), 7.48- 7.42 (m, 1H), 7.42-7.36 (m, 1H), 7.31-7.24 (m, 1H), 4.17 (t, J = 5.6 Hz, 2H), 3.82 (t, J = 5.6 Hz, 2H), 3.19 (s, 3H), 2.96 (t, J = 5.8 Hz, 2H), 2.45 (s, 3H), 2.27-2.19 (m, 2H). [Note: -Two protons merged in solvent peak]. |
| 388 | 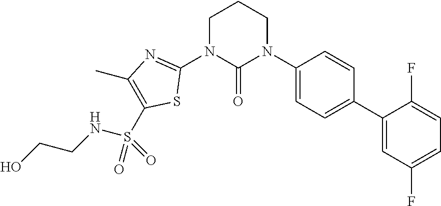 [SMILES: Cc1nc(N2CCCN(c3ccc(-c4cc(F)ccc4F)cc3)C2=O)sc1S(=O)(=O)NCCO] | 509.2 | ¹H NMR (400 MHz, DMSO-d₆): δ 7.80-7.73 (m, 1H), 7.65-7.59 (m, 2H), 7.53-7.49 (m, 2H), 7.48-7.35 (m, 2H), 7.32- 7.24 (m, 1H), 4.68 (t, J = 5.6 Hz, 1H), 4.17 (t, J = 6 Hz, 2H), 3.82 (t, J = 5.6 Hz, 2H), 3.39 (q, J = 6.4 Hz, 2H), 2.90-2.83 (m, 2H), 2.45 (s, 3H), 2.28- 2.20 (m, 2H). |
| 389 | 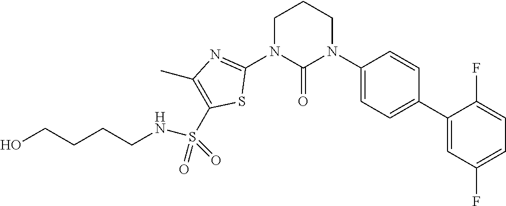 [SMILES: Cc1nc(N2CCCN(c3ccc(-c4cc(F)ccc4F)cc3)C2=O)sc1S(=O)(=O)NCCCCO] | 537.2 | ¹H NMR (400 MHz, DMSO-d₆): δ 7.77-7.69 (m, 1H), 7.62 (d, J = 7.3 Hz, 2H), 7.54-7.48 (m, 2H), 7.47-7.35 (m, 2H), 7.33-7.23 (m, 1H), 4.37 (t, J = 5.1 Hz, 1H), 4.17 (t, J = 5.6 Hz, 2H), 3.88- 3.74 (m, 2H), 3.41-3.33 (m, 2H), 2.88-2.73 (m, 2H), 2.45 (s, 3H), 2.28- 2.19 (m, 2H), 1.48-1.33 (m, 4H) |
| 390 | 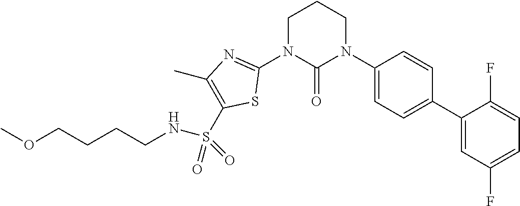 [SMILES: COCCCCNS(=O)(=O)c1sc(N2CCCN(c3ccc(-c4cc(F)ccc4F)cc3)C2=O)nc1C] | 551.0 | ¹H NMR (400 MHz, DMSO-d₆): δ 7.78-7.70 (m, 1H), 7.63 (d, J = 7.1 Hz, 2H), 7.56-7.36 (m, 4H), 7.32-7.24 (m, 1H), 4.17 (t, J = 5.6 Hz, 2H), 3.82 (t, J = 5.4 Hz, 2H), 3.28-3.23 (m, 2H), 3.18 (s, 3H), 2.85-2.78 (m, 2H), 2.45 (s, 3H), 2.30- 2.17 (m, 2H), 1.55-1.35 (m, 4H). |
| 391 | 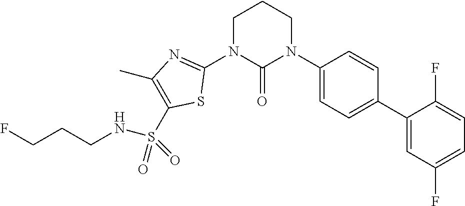 [SMILES: Cc1nc(N2CCCN(c3ccc(-c4cc(F)ccc4F)cc3)C2=O)sc1S(=O)(=O)NCCCF] | 525.5 | ¹H NMR (400 MHz, DMSO-d₆): δ 7.84 (s, 1H), 7.63 (d, J = 7.1 Hz, 2H), 7.54-7.36 (m, 4H), 7.31- 7.24 (m, 1H), 4.51 (t, J = 5.7 Hz, 1H), 4.39 (t, J = 5.8 Hz, 1H), 4.18 (t, J = 6.0 Hz, 2H), 3.82 (t, J = 5.4 Hz, 2H), 2.92 (t, J = 6.7 Hz, 2H), 2.46 (s, 3H), 2.31-2.17 (m, 2H), 1.86- 1.71 (m, 2H). |
| 392 | 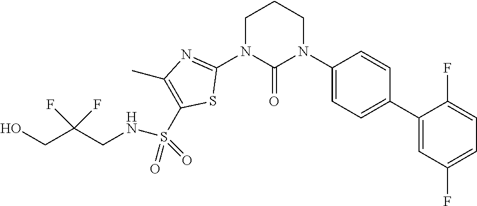 [SMILES: Cc1nc(N2CCCN(c3ccc(-c4cc(F)ccc4F)cc3)C2=O)sc1S(=O)(=O)NCC(F)(F)CO] | 559.4 | ¹H NMR (400 MHz, DMSO-d₆): δ 8.34 (brs, 1H), 7.63 (d, J = 7.1 Hz, 2H), 7.56-7.36 (m, 4H), 7.33-7.24 (m, 1H), 5.48 (t, J = 6.2 Hz, 1H), 4.17 (t, J = 5.7 Hz, 2H), 3.82 (t, J = 5.5 Hz, 2H), 3.65- 3.53 (m, 2H), 2.46 (s, 3H), 2.32-2.16 (m, 2H). [Note: -2H merged in solvent peak] |
| 393 | 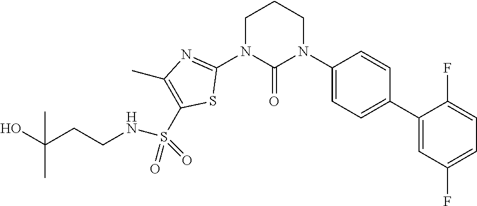 [SMILES: Cc1nc(N2CCCN(c3ccc(-c4cc(F)ccc4F)cc3)C2=O)sc1S(=O)(=O)NCCC(C)(C)O] | 551.3 | ¹H NMR (400 MHz, DMSO-d₆): δ 7.67-7.57 (m, 3H), 7.54-7.36 (m, 4H), 7.31-7.25 (m, 1H), 4.31 (s, 1H), 4.18 (t, J = 5.9 Hz, 2H), 3.82 (t, J = 5.6 Hz, 2H), 2.93-2.85 (m, 2H), 2.45 (s, 3H), 2.32-2.17 (m, 2H), 1.57- 1.48 (m, 2H), 1.04 (s, 6H). |
| 394 | 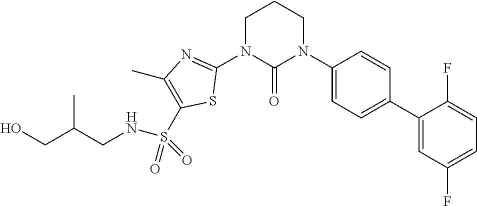 [SMILES: Cc1nc(N2CCCN(c3ccc(-c4cc(F)ccc4F)cc3)C2=O)sc1S(=O)(=O)NCC(C)CO] | 537.3 | ¹H NMR (400 MHz, DMSO-d₆): δ 7.72-7.65 (m, 1H), 7.65-7.60 (m, 2H), 7.57-7.36 (m, 4H), 7.36-7.20 (m, 1H), 4.48 (t, J = 5.2 Hz, 1H), 4.23- 4.11 (m, 2H), 3.82 (t, J = 5.6 Hz, 2H), 3.28-3.18 (m, 2H), 2.93-2.84 (m, 1H), 2.61-2.54 (m, 1H), 2.45 (s, 3H), 2.32-2.17 (m, 2H), 1.72-1.57 (m, 1H), 0.80 (d, J = 6.8 Hz, 3H). |
| 395 | 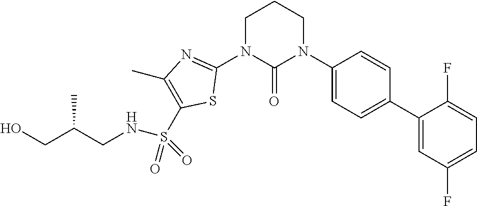 [SMILES: Cc1nc(N2CCCN(c3ccc(-c4cc(F)ccc4F)cc3)C2=O)sc1S(=O)(=O)NC[C@@H](C)CO] | 537.3 | ¹H NMR (400 MHz, DMSO-d₆): δ 7.72-7.66 (m, 1H), 7.66-7.59 (m, 2H), 7.56-7.36 (m, 4H), 7.36-7.20 (m, 1H), 4.48 (t, J = 5.2 Hz, 1H), 4.23- 4.11 (m, 2H), 3.82 (t, J = 5.6 Hz, 2H), 3.28-3.18 (m, 2H), 2.93-2.84 (m, 1H), 2.61-2.54 (m, 1H), 2.45 (s, 3H), 2.32-2.17 (m, 2H), 1.69-1.60 (m, 1H), 0.80 (d, J = 6.8 Hz, 3H). |
| 396 | 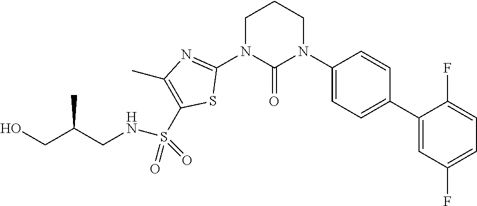 [SMILES: Cc1nc(N2CCCN(c3ccc(-c4cc(F)ccc4F)cc3)C2=O)sc1S(=O)(=O)NC[C@H](C)CO] | 537.3 | ¹H NMR (400 MHz, DMSO-d₆): δ 7.71-7.66 (m, 1H), 7.65-7.59 (m, 2H), 7.56-7.36 (m, 4H), 7.33-7.23 (m, 1H), 4.48 (t, J = 5.2 Hz, 1H), 4.17 (t, J = 5.9 Hz, 2H), 3.82 (t, J = 5.6 Hz, 2H), 3.28- 3.18 (m, 2H), 2.92-2.85 (m, 1H), 2.61-2.54 (m, 1H), 2.45 (s, 3H), 2.29- 2.20 (m, 2H), 1.71-1.59 (m, 1H), 0.80 (d, J = 6.8 Hz, 3H). |
| 397 | 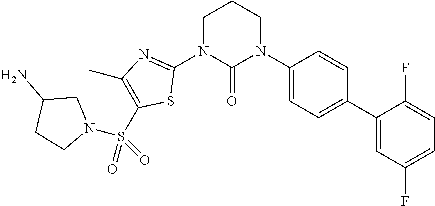 [SMILES: Cc1nc(N2CCCN(c3ccc(-c4cc(F)ccc4F)cc3)C2=O)sc1S(=O)(=O)N1CCC(N)C1] | 534.3 | ¹H NMR (400 MHz, DMSO-d₆): δ 7.63 (d, J = 7.2 Hz, 2H), 7.55-7.36 (m, 4H), 7.32-7.25 (m, 1H), 4.19 (t, J = 5.9 Hz, 2H), 3.83 (t, J = 5.4 Hz, 2H), 3.38-3.35 (m, 2H), 3.24-3.15 (m, 1H), 2.88- 2.73 (m, 1H), 2.28- 2.21 (m, 2H), 1.93-1.82 (m, 2H), 1.54-1.45 (m, 1H) [Note: -CH₃ and 2H merged in solvent peak] |
| 398 | 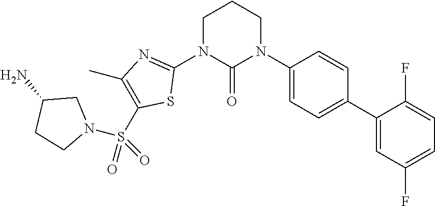 [SMILES: Cc1nc(N2CCCN(c3ccc(-c4cc(F)ccc4F)cc3)C2=O)sc1S(=O)(=O)N1CC[C@H](N)C1] | 534.5 | ¹H NMR (400 MHz, DMSO-d₆): δ 7.63 (d, J = 7.8 Hz, 2H), 7.51 (d, J = 8.3 Hz, 2H), 7.48-7.35 (m, 2H), 7.33-7.24 (m, 1H), 4.25-4.16 (m, 2H), 3.88-3.79 (m, 2H), 3.43- 3.34 (m, 2H), 3.24- 3.15 (m, 1H), 2.88-2.74 (m, 1H), 2.31-2.18 (m, 2H), 2.00-1.80 (m, 2H), 1.59-1.45 (m, 1H). [Note: -CH₃ and 2H merged in solvent peak] |
| 399 | 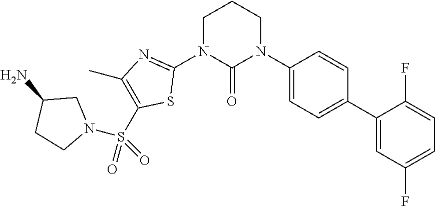 [SMILES: Cc1nc(N2CCCN(c3ccc(-c4cc(F)ccc4F)cc3)C2=O)sc1S(=O)(=O)N1CC[C@@H](N)C1] | 534.5 | ¹H NMR (400 MHz, DMSO-d₆): δ 7.67-7.59 (m, 2H), 7.55-7.34 (m, 4H), 7.33-7.23 (m, 1H), 4.24-4.15 (m, 2H), 3.88- 3.78 (m, 2H), 2.98- 2.87 (m, 1H), 2.30-2.21 (m, 2H), 2.03-1.89 (m, 1H), 1.66-1.53 (m, 1H). [Note: -NH₂ and-CH₃ merged in solvent peak] |
| 400 | 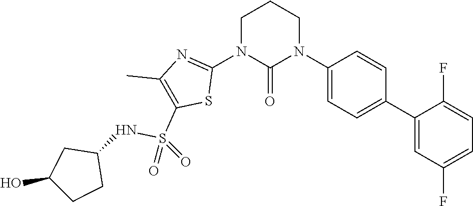 [SMILES: Cc1nc(N2CCCN(c3ccc(-c4cc(F)ccc4F)cc3)C2=O)sc1S(=O)(=O)N[C@@H]1CC[C@@H](O)C1] | 549.2 | ¹H NMR (400 MHz, DMSO-d₆): δ 7.81 (d, J = 7.7 Hz, 1H), 7.63 (d, J = 7.1 Hz, 2H), 7.53-7.49 (m, 2H), 7.48-7.35 (m, 2H), 7.31-7.25 (m, 1H), 4.47 (d, J = 3.7 Hz, 1H), 4.18 (t, J = 5.7 Hz, 2H), 4.11-4.04 (m, 1H), 3.82 (t, J = 5.6 Hz, 2H), 3.70- 3.64 (m, 1H), 2.45 (s, 3H), 2.29-2.19 (m, 2H), 1.88-1.75 (m, 2H), 1.67- 1.59 (m, 1H), 1.50- 1.42 (m, 1H), 1.38-1.26 (m, 2H). |
| 401 | 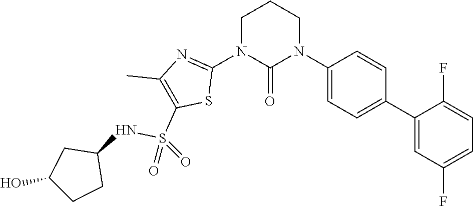 [SMILES: Cc1nc(N2CCCN(c3ccc(-c4cc(F)ccc4F)cc3)C2=O)sc1S(=O)(=O)N[C@H]1CC[C@H](O)C1] | 549.5 | ¹H NMR (400 MHz, DMSO-d₆): δ 7.83 (d, J = 7.8 Hz, 1H), 7.63 (d, J = 7.1 Hz, 2H), 7.54-7.48 (m, 2H), 7.48-7.35 (m, 2H), 7.31-7.25 (m, 1H), 4.58 (d, J = 4.2 Hz, 1H), 4.17 (t, J = 5.6 Hz, 2H), 3.97-3.90 (m, 1H), 3.82 (t, J = 5.5 Hz, 2H), 3.50- 3.37 (m, 2H), 2.44 (s, 3H), 2.27-2.21 (m, 2H), 2.01-1.91 (m, 1H), 1.69- 1.40 (m, 3H), 1.30- 1.22 (m, 1H). |
| 402 | 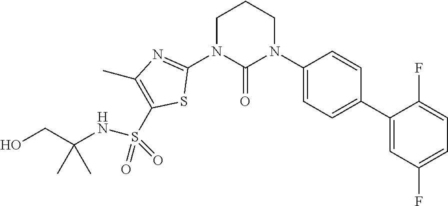 [SMILES: Cc1nc(N2CCCN(c3ccc(-c4cc(F)ccc4F)cc3)C2=O)sc1S(=O)(=O)NC(C)(C)CO] | 537.2 | ¹H NMR (400 MHz, DMSO-d₆): δ 7.62 (d, J = 7.1 Hz, 2H), 7.55-7.36 (m, 5H), 7.32-7.24 (m, 1H), 4.77 (t, J = 5.8 Hz, 1H), 4.17 (t, J = 5.8 Hz, 2H), 3.82 (t, J = 5.6 Hz, 2H), 3.21 (d, J = 6.0 Hz, 2H), 2.44 (s, 3H), 2.29- 2.18 (m, 2H), 1.08 (s, 6H). |
| 403 | 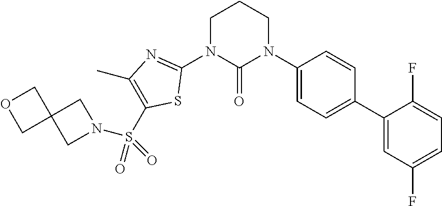 [SMILES: Cc1nc(N2CCCN(c3ccc(-c4cc(F)ccc4F)cc3)C2=O)sc1S(=O)(=O)N1CC2(COC2)C1] | 547.3 | ¹H NMR (400 MHz, DMSO-d₆): δ 7.65-7.62 (m, 2H), 7.53-7.37 (m, 4H), 7.31-7.25 (m, 1H), 4.57 (s, 4H), 4.21 (t, J = 5.9 Hz, 2H), 3.92 (s, 4H), 3.87-3.81 (m, 2H), 2.29- 2.21 (m, 2H). [Note: -Methyl merged in solvent peak] |
| 404 | 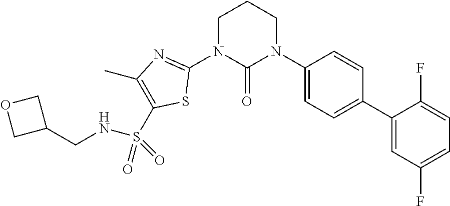 [SMILES: Cc1nc(N2CCCN(c3ccc(-c4cc(F)ccc4F)cc3)C2=O)sc1S(=O)(=O)NCC1COC1] | 535.3 | ¹H NMR (400 MHz, DMSO-d₆): δ 7.98-7.91 (m, 1H), 7.64-7.61 (m, 2H), 7.52-7.36 (m, 4H), 7.31-7.25 (m, 1H), 4.56 (dd, J = 6.2, 7.6 Hz, 2H), 4.21-4.16 (m, 4H), 3.82 (t, J = 5.5 Hz, 2H), 3.13- 3.06 (m, 2H), 3.06-2.92 (m, 1H), 2.46 (s, 3H), 2.28-2.19 (m, 2H) |
| 405 | 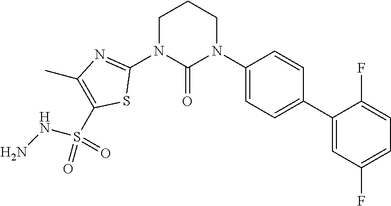 [SMILES: Cc1nc(N2CCCN(c3ccc(-c4cc(F)ccc4F)cc3)C2=O)sc1S(=O)(=O)NN] | 480.1 | ¹H-NMR (400 MHz, DMSO-d6) 8.42 (s, 1H), 7.64 (d, J = 6.80 Hz, 2H), 7.51 (d, J = 8.40 Hz, 2H), 7.40- 7.46 (m, 2H), 7.30-7.29 (m, 1H), 4.19 (t, J = 5.60 Hz, 4H), 3.83 (t, J = 5.60 Hz, 2H) , 2.48 (s, 3H), 2.08 (s, 2H). |
| 406 | 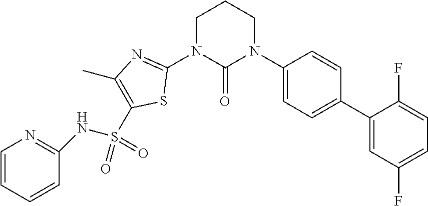 [SMILES: Cc1nc(N2CCCN(c3ccc(-c4cc(F)ccc4F)cc3)C2=O)sc1S(=O)(=O)Nc1ccccn1] | 542.1 | ¹H NMR (400 MHz, DMSO-d₆): δ 8.06-7.94 (m, 1H), 7.82-7.68 (m, 1H), 7.62 (d, J = 7.6 Hz, 2H), 7.54-7.35 (m, 4H), 7.33-7.25 (m, 1H), 7.17- 7.08 (m, 1H), 6.93- 6.77 (m, 1H), 4.12 (t, J = 5.7 Hz, 2H), 3.84-3.71 (m, 2H), 2.46 (s, 3H), 2.24-2.17 (m, 2H). [Note: -NH proton not observed] |
| 407 | 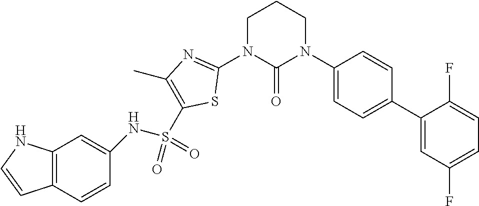 [SMILES: Cc1nc(N2CCCN(c3ccc(-c4cc(F)ccc4F)cc3)C2=O)sc1S(=O)(=O)Nc1ccc2cc[nH]c2c1] | 580.2 | ¹H NMR (400 MHz, DMSO-d₆): δ 11.04 (s, 1H), 10.10 (s, 1H), 7.60 (d, J = 7.1 Hz, 2H), 7.50- 7.35 (m, 5H), 7.31-7.21 (m, 3H), 6.79 (dd, J = 1.8, 8.4 Hz, 1H), 6.36- 6.30 (m, 1H), 4.07 (t, J = 5.9 Hz, 2H), 3.76 (t, J = 5.5 Hz, 2H), 2.28 (s, 3H), 2.23-2.12 (m, 2H) |
| 408 | 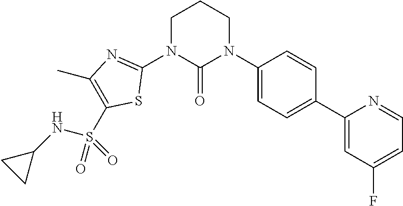 [SMILES: Cc1nc(N2CCCN(c3ccc(-c4cc(F)ccn4)cc3)C2=O)sc1S(=O)(=O)NC1CC1] | 488.6 | ¹H NMR (400 MHz, DMSO-d₆): δ 8.70 (dd, J = 5.7, 8.8 Hz, 1H), 8.17 (d, J = 8.3 Hz, 2H), 8.02 (s, 1H), 7.99-7.91 (m, 1H), 7.53 (d, J = 8.3 Hz, 2H), 7.40-7.19 (m, 1H), 4.24- 4.14 (m, 2H), 3.89- 3.79 (m, 2H), 2.47 (s, 3H), 2.36-2.12 (m, 3H), 0.56-0.49 (m, 2H), 0.45- 0.40 (m, 2H). |
| 409 | 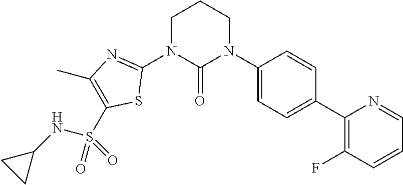 [SMILES: Cc1nc(N2CCCN(c3ccc(-c4ncccc4F)cc3)C2=O)sc1S(=O)(=O)NC1CC1] | 488.2 | ¹H NMR (400 MHz, DMSO-d₆): δ 8.56 (s, 1H), 8.03 (s, 1H), 7.97 (d, J = 7.8 Hz, 2H), 7.89-7.79 (m, 1H), 7.58-7.52 (m, 2H), 7.52-7.46 (m, 1H), 4.19 (t, J = 5.6 Hz, 2H), 3.89-3.79 (m, 2H), 2.47 (s, 3H), 2.29-2.17 (m, 3H), 0.56-0.49 (m, 2H), 0.46-0.37 (m, 2H). |
| 410 | 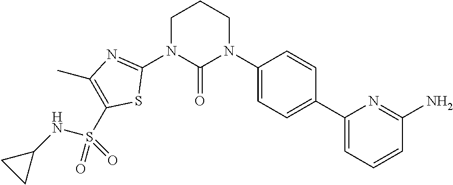 [SMILES: Cc1nc(N2CCCN(c3ccc(-c4cccc(N)n4)cc3)C2=O)sc1S(=O)(=O)NC1CC1] | 485.2 | ¹H NMR (400 MHz, DMSO-d₆): δ 8.00 (d, J = 8.3 Hz, 3H), 7.55-7.34 (m, 3H), 7.07 (d, J = 7.0 Hz, 1H), 6.42 (d, J = 8.3 Hz, 1H), 5.97 (s, 2H), 4.26-4.11 (m, 2H), 3.88- 3.74 (m, 2H), 2.46 (s, 3H), 2.28-2.17 (m, 3H), 0.56-0.49 (m, 2H), 0.46- 0.38 (m, 2H). |
| 411 | 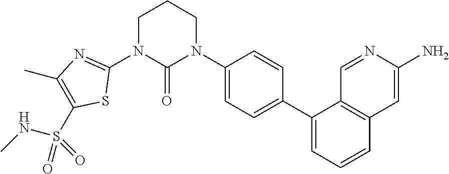 [SMILES: CNS(=O)(=O)c1sc(N2CCCN(c3ccc(-c4cccc5cc(N)ncc45)cc3)C2=O)nc1C] | 508.9 | ¹H NMR (400 MHz, DMSO-d₆): δ 8.70 (s, 1H), 7.63-7.46 (m, 7H), 7.06 (d, J = 6.4 Hz, 1H), 6.71- 6.68 (m, 1H), 5.98 (s, 2H), 4.20 (t, J = 5.4 Hz, 2H), 3.87 (t, J = 4.4 Hz, 2H), 2.46 (s, 3H), 2.30- 2.23 (m, 2H). [Note: One -CH₃ merged in solvent peak] |
| 412 | 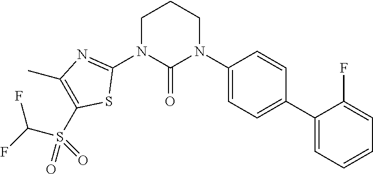 [SMILES: Cc1nc(N2CCCN(c3ccc(-c4ccccc4F)cc3)C2=O)sc1S(=O)(=O)C(F)F] | 482.0 | 1H NMR (400 MHz, DMSO-d6): δ 7.63-7.58 (m, 3H), 7.57-7.51 (m, 2H), 7.37-7.29 (m, 4H), 4.24 (t, J = 6.40 Hz, 2H), 3.85 (t, J = 6.00 Hz, 2H), 2.55 (s, 3H), 2.50-2.27 (m, 2H), |
| 413 | 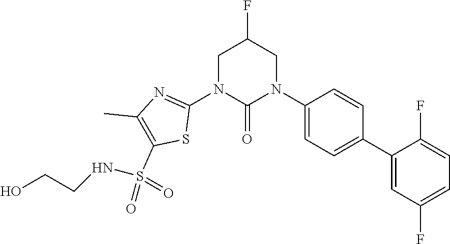 [SMILES: Cc1nc(N2CC(F)CN(c3ccc(-c4cc(F)ccc4F)cc3)C2=O)sc1S(=O)(=O)NCCO] | 527.4 | ¹H NMR (400 MHz, DMSO-d₆): δ 7.81 (s, 1H), 7.65 (d, J = 7.1 Hz, 2H), 7.53-7.42 (m, 3H), 7.41- 7.36 (m, 1H), 7.32- 7.25 (m, 1H), 5.61-5.42 (m, 1H), 4.76-4.65 (m, 2H), 4.35-4.05 (m, 2H), 4.01-3.91 (m, 1H), 3.43- 3.35 (m, 2H), 2.88 (t, J = 6.4 Hz, 2H), 2.46 (s, 3H). |
| 414 | 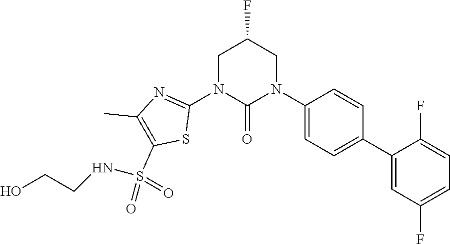 [SMILES: Cc1nc(N2C[C@H](F)CN(c3ccc(-c4cc(F)ccc4F)cc3)C2=O)sc1S(=O)(=O)NCCO] Single enantiomer, arbitrarily assigned stereochemistry | 527.4 | ¹H NMR (400 MHz, DMSO-d₆): δ 7.85-7.78 (m, 1H), 7.65 (dd, J = 1.5, 8.4 Hz, 2H), 7.53- 7.37 (m, 4H), 7.32-7.25 (m, 1H), 5.61-5.44 (m, 1H), 4.76-4.66 (m, 2H), 4.35-4.05 (m, 2H), 4.01- 3.90 (m, 1H), 3.43- 3.34 (m, 2H), 2.88 (q, J = 6.3 Hz, 2H), 2.46 (s, 3H). |
| 415 | 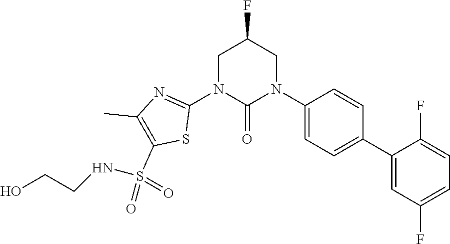 [SMILES: Cc1nc(N2C[C@@H](F)CN(c3ccc(-c4cc(F)ccc4F)cc3)C2=O)sc1S(=O)(=O)NCCO] Single enantiomer, arbitrarily assigned stereochemistry | 527.4 | ¹H NMR (400 MHz, DMSO-d₆): δ 7.86-7.78 (m, 1H), 7.65 (dd, J = 1.5, 8.6 Hz, 2H), 7.51- 7.37 (m, 4H), 7.37-7.21 (m, 1H), 5.65-5.42 (m, 1H), 4.76-4.66 (m, 2H), 4.42-4.03 (m, 2H), 4.01- 3.90 (m, 1H), 3.43- 3.35 (m, 2H), 2.88 (q, J = 6.4 Hz, 2H), 2.46 (s, 3H). |
| 416 | 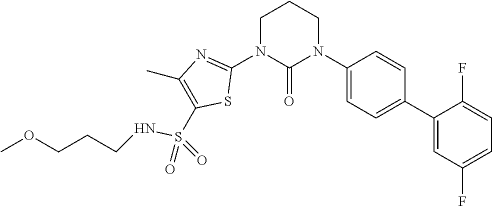 [SMILES: COCCCNS(=O)(=O)c1sc(N2CCCN(c3ccc(-c4cc(F)ccc4F)cc3)C2=O)nc1C] | 537.2 | ¹H NMR (400 MHz, DMSO-d₆): δ 7.78-7.71 (m, 1H), 7.63 (dd, J = 1.4, 8.5 Hz, 2H), 7.51 (d, J = 8.6 Hz, 2H), 7.48- 7.42 (m, 1H), 7.42-7.35 (m, 1H), 7.32-7.25 (m, 1H), 4.17 (t, J = 6.0 Hz, 2H), 3.82 (t, J = 5.6 Hz, 2H), 3.30-3.28 (m, 2H), 3.17 (s, 3H), 2.92-2.80 (m, 2H), 2.45 (s, 3H), 2.30-2.18 (m, 2H), 1.62 (quin, J = 6.6 Hz, 2H). |
| 417 | 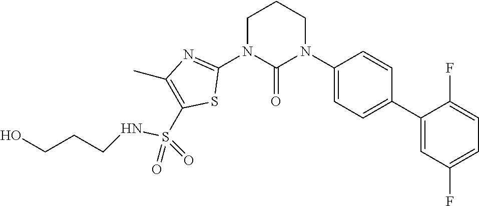 [SMILES: Cc1nc(N2CCCN(c3ccc(-c4cc(F)ccc4F)cc3)C2=O)sc1S(=O)(=O)NCCCO] | 523.1 | ¹H NMR (400 MHz, DMSO-d₆): δ 7.70-7.58 (m, 3H), 7.56-7.35 (m, 4H), 7.35-7.21 (m, 1H), 4.43 (t, J = 4.9 Hz, 1H), 4.17 (t, J = 5.9 Hz, 2H), 3.82 (t, J = 5.4 Hz, 2H), 3.41-3.35 (m, 2H), 2.98- 2.76 (m, 2H), 2.45 (s, 3H), 2.29-2.20 (m, 2H), 1.55 (quin, J = 6.7 Hz, 2H |
| 418 | 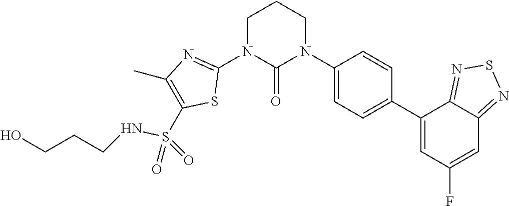 [SMILES: Cc1nc(N2CCCN(c3ccc(-c4cc(F)cc5nsnc45)cc3)C2=O)sc1S(=O)(=O)NCCCO] | 563.5 | ¹H NMR (400 MHz, DMSO-d₆): δ 8.07 (d, J = 6.8 Hz, 2H), 7.97-7.87 (m, 2H), 7.71-7.63 (m, 1H), 7.57 (d, J = 6.8 Hz, 2H), 4.45-4.36 (m, 1H), 4.22-4.13 (m, 2H), 3.89- 3.80 (m, 2H), 3.40- 3.34 (m, 2H), 2.90-2.81 (m, 2H), 2.44 (s, 3H), 2.28-2.21 (m, 2H), 1.62- 1.44 (m, 2H). |
| 419 | 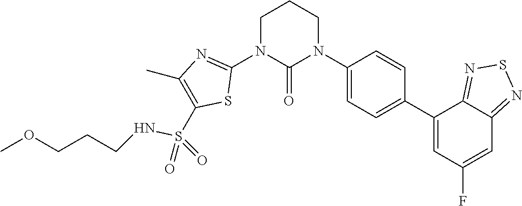 [SMILES: COCCCNS(=O)(=O)c1sc(N2CCCN(c3ccc(-c4cc(F)cc5nsnc45)cc3)C2=O)nc1C] | 577.3 | ¹H NMR (400 MHz, DMSO-d₆): δ 8.09 (d, J = 6.4 Hz, 2H), 7.96-7.91 (m, 2H), 7.77-7.68 (m, 1H), 7.59 (d, J = 6.8 Hz, 2H), 4.26-4.13 (m, 2H), 3.91-3.82 (m, 2H), 3.18 (s, 3H), 2.93-2.82 (m, 2H), 2.37-2.29 (m, 5H), 2.28-2.20 (m, 2H), 1.67- 1.57 (m, 2H). |
| 420 | 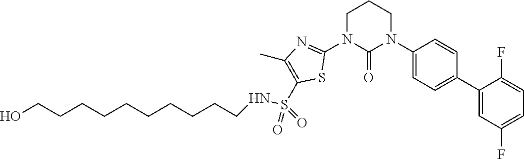 [SMILES: Cc1nc(N2CCCN(c3ccc(-c4cc(F)ccc4F)cc3)C2=O)sc1S(=O)(=O)NCCCCCCCCCCO] | 621.6 | ¹H NMR (400 MHz, DMSO-d₆): δ 7.75-7.70 (m, 1H), 7.65-7.60 (m, 2H), 7.50 (d, J = 8.7 Hz, 2H), 7.47-7.36 (m, 2H), 7.32-7.24 (m, 1H), 4.29 (t, J = 5.2 Hz, 1H), 4.17 (t, J = 6.0 Hz, 2H), 3.82 (t, J = 5.6 Hz, 2H), 3.39- 3.33 (m, 2H), 2.80 (q, J = 6.8 Hz, 2H), 2.45 (s, 3H), 2.27-2.20 (m, 2H), 1.43- 1.33 (m, 4H), 1.27- 1.16 (m, 12H). |
| 421 | 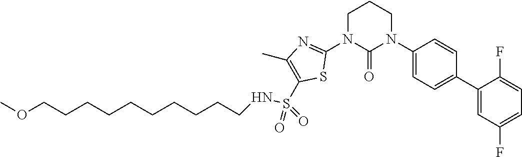 [SMILES: COCCCCCCCCCCNS(=O)(=O)c1sc(N2CCCN(c3ccc(-c4cc(F)ccc4F)cc3)C2=O)nc1C] | 635.2 | ¹H NMR (400 MHz, DMSO-d₆): δ 7.74-7.66 (m, 1H), 7.65-7.57 (m, 2H), 7.54-7.31 (m, 4H), 7.30-7.21 (m, 1H), 4.20- 4.10 (m, 2H), 3.85- 3.76 (m, 2H), 3.17 (s, 3H), 2.83-2.73 (m, 2H), 2.43 (s, 3H), 2.26-2.16 (m, 2H), 1.50-1.40 (m, 2H), 1.38-1.29 (m, 2H), 1.28-1.10 (m, 12H). [Note: -two protons are merged in solvent peak] |
| 422 | 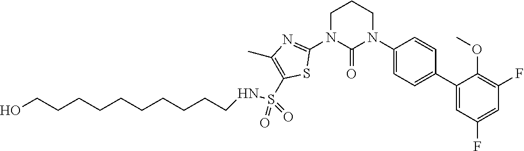 [SMILES: COc1c(F)cc(F)cc1-c1ccc(N2CCCN(c3nc(C)c(S(=O)(=O)NCCCCCCCCCCO)s3)C2=O)cc1] | 651.5 | ¹H NMR (400 MHz, DMSO-d₆): δ 7.77-7.66 (m, 1H), 7.61-7.56 (m, 2H), 7.51-7.45 (m, 2H), 7.45-7.30 (m, 1H), 7.19- 7.08 (m, 1H), 4.30 (t, J = 5.1 Hz, 1H), 4.17 (t, J = 5.8 Hz, 2H), 3.82 (t, J = 5.4 Hz, 2H), 3.67 (s, 3H), 3.41-3.34 (m, 2H), 2.84- 2.76 (m, 2H), 2.45 (s, 3H), 2.30-2.17 (m, 2H), 1.43-1.33 (m, 4H), 1.29- 1.12 (m, 12H). |
| 423 | 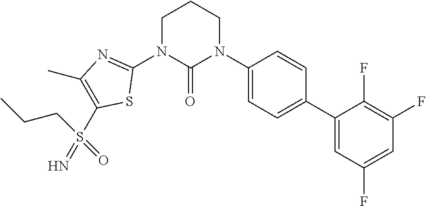 [SMILES: CCCS(=N)(=O)c1sc(N2CCCN(c3ccc(-c4cc(F)cc(F)c4F)cc3)C2=O)nc1C] | 509.2 | ¹H NMR (400 MHz, DMSO-d₆): δ 7.65 (d, J = 7.3 Hz, 2H), 7.60-7.50 (m, 3H), 7.39-7.32 (m, 1H), 4.54 (s, 1H), 4.17 (t, J = 5.6 Hz, 2H), 3.83 (t, J = 5.1 Hz, 2H), 3.19-3.11 (m, 2H), 2.28-2.19 (m, 2H), 1.70-1.55 (m, 2H), 0.92 (t, J = 7.6 Hz, 3H). [Note: -CH₃ merged in solvent peak] |
| 424 | 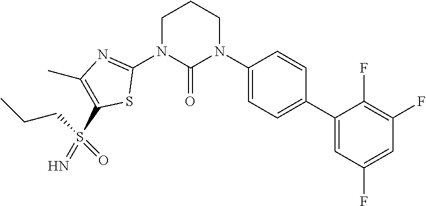 [SMILES: CCC[S@@](=N)(=O)c1sc(N2CCCN(c3ccc(-c4cc(F)cc(F)c4F)cc3)C2=O)nc1C] Single enantiomer, arbitrarily assigned stereochemistry | 509.2 | ¹H NMR (400 MHz, DMSO-d₆): δ 7.65 (dd, J = 1.5, 8.5 Hz, 2H), 7.61- 7.51 (m, 3H), 7.38-7.33 (m, 1H), 4.54 (s, 1H), 4.17 (t, J = 5.9 Hz, 2H), 3.83 (t, J = 5.6 Hz, 2H), 3.14 (dd, J = 6.4, 9.4 Hz, 2H), 2.32-2.16 (m, 2H), 1.68-1.57 (m, 2H), 0.92 (t, J = 7.4 Hz, 3H). [Note: -CH₃ merged in solvent peak] |
| 425 | 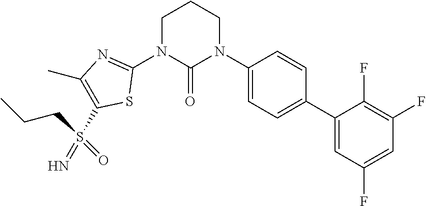 [SMILES: CCC[S@](=N)(=O)c1sc(N2CCCN(c3ccc(-c4cc(F)cc(F)c4F)cc3)C2=O)nc1C] Single enantiomer, arbitrarily assigned stereochemistry | 509.2 | ¹H NMR (400 MHz, DMSO-d₆): δ 7.68-7.62 (m, 2H), 7.60-7.51 (m, 3H), 7.38-7.32 (m, 1H), 4.54 (s, 1H), 4.17 (t, J = 6.0 Hz, 2H), 3.83-3.83 (m, 2H), 3.18-3.10 (m, 2H), 2.32-2.15 (m, 2H), 1.69-1.55 (m, 2H), 0.92 (t, J = 7.4 Hz, 3H). [Note: -CH₃ merged in solvent peak] |
| 426 | 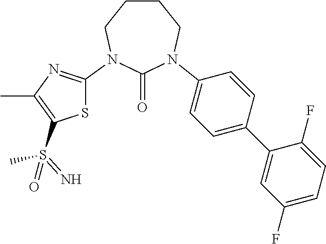 [SMILES: Cc1nc(N2CCCCN(c3ccc(-c4cc(F)ccc4F)cc3)C2=O)sc1[S@@](C)(=N)=O] Single enantiomer, arbitrarily assigned stereochemistry | 477.4 | ¹H NMR (400 MHz, DMSO-d₆): δ 7.62 (d, J = 7.8 Hz, 2H), 7.52-7.35 (m, 4H), 7.31-7.24 (m, 1H), 4.58 (s, 1H), 4.32- 4.20 (m, 2H), 3.88-3.76 (m, 2H), 3.13 (s, 3H), 2.02-1.93 (m, 2H), 1.91- 1.81 (m, 2H). [Note: -Methyl protons merged in solvent peak]. |
| 427 | 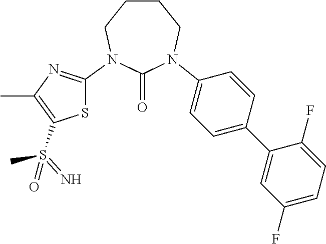 [SMILES: Cc1nc(N2CCCCN(c3ccc(-c4cc(F)ccc4F)cc3)C2=O)sc1[S@](C)(=N)=O] Single enantiomer, arbitrarily assigned stereochemistry | 477.4 | ¹H NMR (400 MHz, DMSO-d₆): δ 7.62 (d, J = 7.1 Hz, 2H), 7.52-7.35 (m, 4H), 7.31-7.23 (m, 1H), 4.60 (s, 1H), 4.28- 4.21 (m, 2H), 3.84-3.78 (m, 2H), 3.12 (s, 3H), 2.01-1.93 (m, 2H), 1.90- 1.81 (m, 2H). [Note: -Methyl protons merged in solvent peak]. |
| 428 | 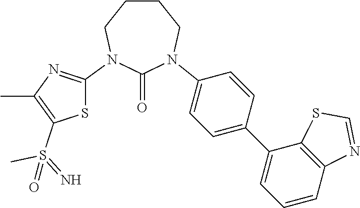 [SMILES: Cc1nc(N2CCCCN(c3ccc(-c4cccc5ncsc45)cc3)C2=O)sc1S(C)(=N)=O] | 498.4 | ¹H NMR (400 MHz, DMSO-d₆): δ 9.47 (s, 1H), 8.13 (d, J = 7.8 Hz, 1H), 7.78 (d, J = 7.8 Hz, 2H), 7.73-7.66 (m, 1H), 7.65- 7.61 (m, 1H), 7.59- 7.53 (m, 2H), 4.58 (s, 1H), 4.30-4.23 (m, 2H), 3.89-3.80 (m, 2H), 3.13 (s, 3H), 2.05-1.94 (m, 2H), 1.93-1.84 (m, 2H). [Note: -Methyl proton merged in solvent peak]. |
| 429 | 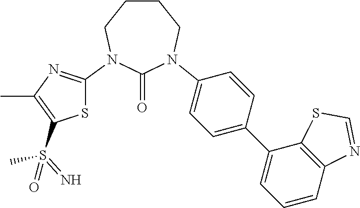 [SMILES: Cc1nc(N2CCCCN(c3ccc(-c4cccc5ncsc45)cc3)C2=O)sc1[S@@](C)(=N)=O] Single enantiomer, arbitrarily assigned stereochemistry | 498.4 | ¹H NMR (400 MHz, DMSO-d₆): δ 9.48 (s, 1H), 8.13 (d, J = 7.9 Hz, 1H), 7.78 (d, J = 8.8 Hz, 2H), 7.73-7.65 (m, 1H), 7.65- 7.61 (m, 1H), 7.60- 7.53 (m, 2H), 4.59 (s, 1H), 4.31-4.21 (m, 2H), 3.89-3.79 (m, 2H), 3.13 (s, 3H), 2.05-1.94 (m, 2H), 1.93-1.79 (m, 2H). [Note: -Methyl protons merged in solvent peak]. |
| 430 | 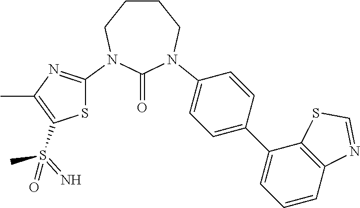 [SMILES: Cc1nc(N2CCCCN(c3ccc(-c4cccc5ncsc45)cc3)C2=O)sc1[S@](C)(=N)=O] Single enantiomer, arbitrarily assigned stereochemistry | 498.4 | ¹H NMR (400 MHz, DMSO-d₆): δ 9.45 (s, 1H), 8.10 (d, J = 7.8 Hz, 1H), 7.79-7.72 (m, 2H), 7.70- 7.63 (m, 1H), 7.63- 7.57 (m, 1H), 7.57-7.51 (m, 2H), 4.56 (s, 1H), 4.27-4.21 (m, 2H), 3.86- 3.79 (m, 2H), 3.11 (s, 3H), 2.02-1.91 (m, 2H), 1.91-1.81 (m, 2H). [Note: -Methyl protons merged in solvent peak]. |
| 431 | 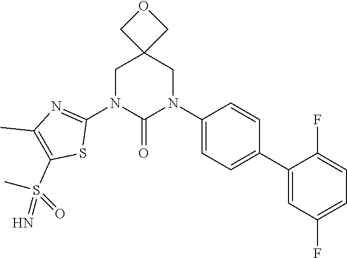 [SMILES: Cc1nc(N2CC3(COC3)CN(c3ccc(-c4cc(F)ccc4F)cc3)C2=O)sc1S(C)(=N)=O] | 505.2 | ¹H NMR (400 MHz, DMSO-d₆): δ 7.68-7.63 (m, 2H), 7.56-7.50 (m, 2H), 7.48-7.35 (m, 2H), 7.32-7.25 (m, 1H), 4.60 (s, 1H), 4.55 (s, 4H), 4.44 (s, 2H), 4.08 (s, 2H), 3.13 (s, 3H), 2.52 (s, 3H). |
| 432 | 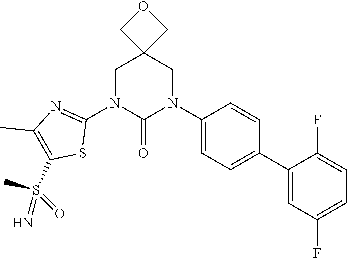 [SMILES: Cc1nc(N2CC3(COC3)CN(c3ccc(-c4cc(F)ccc4F)cc3)C2=O)sc1[S@@](C)(=N)=O] Single enantiomer, arbitrarily assigned stereochemistry | 505.2 | ¹H NMR (400 MHz, DMSO-d₆): δ 7.67-7.62 (m, 2H), 7.54-7.37 (m, 4H), 7.32-7.26 (m, 1H), 4.55 (s, 4H), 4.44 (s, 2H), 4.09 (s, 2H), 3.13 (s, 3H), 2.52 (s, 3H) |
| 433 | 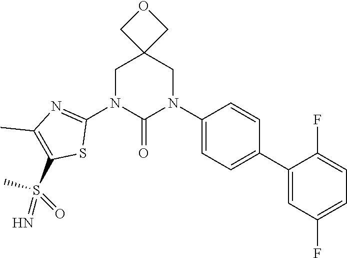 [SMILES: Cc1nc(N2CC3(COC3)CN(c3ccc(-c4cc(F)ccc4F)cc3)C2=O)sc1[S@](C)(=N)=O] Single enantiomer, arbitrarily assigned stereochemistry | 505.2 | ¹H NMR (400 MHz, DMSO-d₆): δ 7.65 (d, J = 7.2 Hz, 2H), 7.54-7.37 (m, 4H), 7.32-7.26 (m, 1H), 4.55 (s, 4H), 4.44 (s, 2H), 4.08 (s, 2H), 3.14 (s, 3H), 2.53 (s, 3H). |
| 434 | 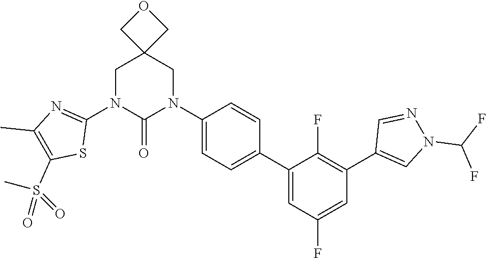 [SMILES: Cc1nc(N2CC3(COC3)CN(c3ccc(-c4cc(F)cc(-c5cnn(C(F)F)c5)c4F)cc3)C2=O)sc1S(C)(=O)=O] | 622.0 | 1H NMR (400 MHz, DMSO-d6): δ 8.76 (d, J = 1.20 Hz, 1H), 8.39 (s, 1H), 7.91 (s, 1H), 7.82- 7.77 (m, 1H), 7.71 (d, J = 7.20 Hz, 2H), 7.55 (d, J = 8.40 Hz, 2H), 7.42-7.38 (m, 1H), 4.58 (s, 4H), 4.48 (s, 2H), 4.11 (s, 2H), 3.28 (s, 3H), 2.55 (s, 3H), |
| 435 | 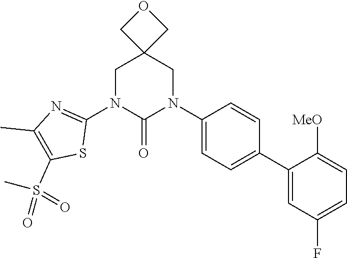 [SMILES: COc1ccc(F)cc1-c1ccc(N2CC3(COC3)CN(c3nc(C)c(S(C)(=O)=O)s3)C2=O)cc1] | 518.1 | 1H NMR (400 MHz, DMSO-d6): δ 7.59-7.56 (m, 2H), 7.45-7.43 (m, 2H), 7.22-7.20 (m, 1H), 7.19-7.13 (m, 2H), 4.57 (s, 4H), 4.47 (s, 2H), 4.08 (s, 2H), 3.79 (s, 3H), 3.27 (s, 3H), 2.54 (s, 3H) |
| 436 | 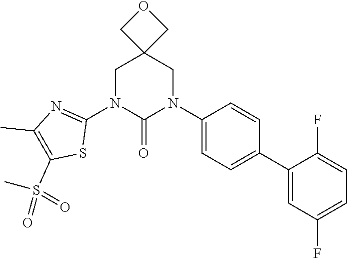 [SMILES: Cc1nc(N2CC3(COC3)CN(c3ccc(-c4cc(F)ccc4F)cc3)C2=O)sc1S(C)(=O)=O] | 506.1 | 1H NMR (400 MHz, DMSO-d6): δ 7.67-7.65 (m, 2H), 7.54-7.48 (m, 2H), 7.47-7.42 (m, 1H), 7.41-7.38 (m, 1H), 7.32- 7.30 (m, 1H), 4.57 (s, 4H), 4.47 (s, 2H), 4.10 (s, 2H), 3.27 (s, 3H), 2.55 (s, 3H) |
| 437 | 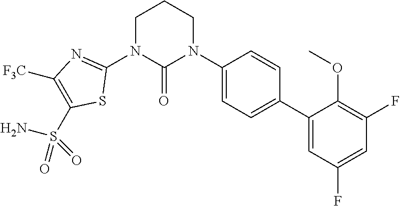 [SMILES: COc1c(F)cc(F)cc1-c1ccc(N2CCCN(c3nc(C(F)(F)F)c(S(N)(=O)=O)s3)C2=O)cc1] | 549.4 | ¹H NMR (400 MHz, DMSO-d₆): δ 8.13 (s, 2H), 7.63-7.56 (m, 2H), 7.54- 7.47 (m, 2H), 7.41- 7.32 (m, 1H), 7.18-7.11 (m, 1H), 4.17 (t, J = 6.0 Hz, 2H), 3.84 (t, J = 5.6 Hz, 2H), 3.67 (s, 3H), 2.31-2.21 (m, 2H). |
| 438 | 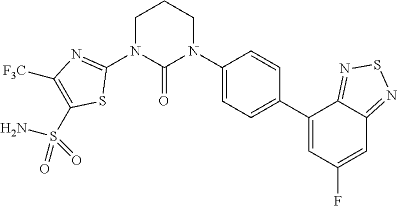 [SMILES: NS(=O)(=O)c1sc(N2CCCN(c3ccc(-c4cc(F)cc5nsnc45)cc3)C2=O)nc1C(F)(F)F] | 559.3 | ¹H NMR (400 MHz, CD₃OD-d₄): δ 8.02 (d, J = 8.5 Hz, 2H), 7.63-7.47 (m, 2H), 7.44-7.36 (m, 1H), 7.25 (d, J = 7.9 Hz, 1H), 4.25 (t, J = 6.1 Hz, 2H), 3.82 (t, J = 5.6 Hz, 2H), 2.33-2.21 (m, 2H) |
| 439 | 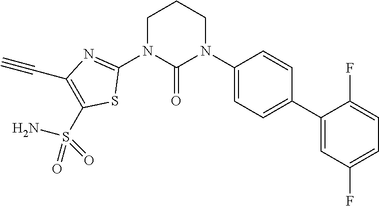 [SMILES: C#Cc1nc(N2CCCN(c3ccc(-c4cc(F)ccc4F)cc3)C2=O)sc1S(N)(=O)=O] | 475.1 | ¹H NMR (400 MHz, DMSO-d₆): δ 7.80 (s, 2H), 7.63 (d, J = 7.1 Hz, 2H), 7.55-7.49 (m, 2H), 7.48- 7.36 (m, 2H), 7.32- 7.26 (m, 1H), 4.61 (s, 1H), 4.15 (t, J = 5.9 Hz, 2H), 3.83 (t, J = 5.5 Hz, 2H), 2.28-2.20 (m, 2H) |
| 440 | 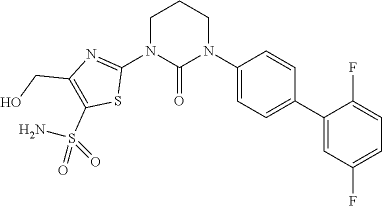 [SMILES: NS(=O)(=O)c1sc(N2CCCN(c3ccc(-c4cc(F)ccc4F)cc3)C2=O)nc1CO] | 481.2 | ¹H NMR (400 MHz, DMSO-d₆): δ 7.63 (d, J = 8.3 Hz, 2H), 7.55-7.49 (m, 4H), 7.49-7.36 (m, 2H), 7.33-7.23 (m, 1H), 5.06 (br.s, 1H), 4.64 (br.s, 2H), 4.21 (t, J = 5.9 Hz, 2H), 3.84 (t, J = 5.6 Hz, 2H), 2.32-2.20 (m, 2H). |
| 441 | 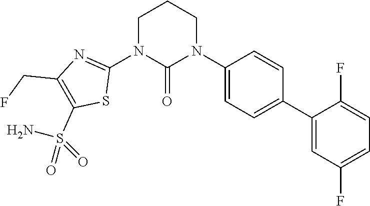 [SMILES: NS(=O)(=O)c1sc(N2CCCN(c3ccc(-c4cc(F)ccc4F)cc3)C2=O)nc1CF] | 483.2 | ¹H NMR (400 MHz, DMSO-d₆): δ 7.83 (s, 2H), 7.63 (d, J = 7.1 Hz, 2H), 7.52 (d, J = 8.6 Hz, 2H), 7.50-7.36 (m, 2H), 7.32- 7.25 (m, 1H), 5.54 (d, J = 47.2 Hz, 2H), 4.20 (t, J = 5.9 Hz, 2H), 3.84 (t, J = 5.7 Hz, 2H), 2.30-2.22 (m, 2H) |

### Biological Assay Data

### Cell Culture

Vero cells were cultured in Dulbecco's Modified Eagle Medium (DMEM) supplemented with 10% fetal bovine serum and 100 units/mL penicillin and streptomycin. The cells were passaged 2-3 times per week to maintain sub-confluent densities.

### Assays

### HSV-1 Antiviral Assay

Vero cells were seeded into 96-well plates at a density of 2.5×10³ cells per well and allowed to attach overnight. Following attachment, the media was replaced with 50 μL of infection medium (DMEM supplemented with 2% fetal bovine serum and 100 units/mL penicillin and streptomycin). A Tecan D300e digital dispenser was then used to add compounds to the culture using an 8-point 3-fold serial dilution format. The DMSO concentration was normalized to 0.5% for all treatments. Following compound addition, 50 μL of infection medium containing 80 TCID₅₀ HSV-1 was added to the cells and incubated at 37° C. for 4 days. After the incubation, the plates were equilibrated to room temperature, the media was removed, and 60 of a 1:1 dilution of Cell titer glow and phosphate buffered saline was added to the cells. Following a 5-minute incubation, cell viability was quantified by measuring luminance using a Tecan Infinite M1000 Pro plate reader.

### HSV-2 Antiviral Assay

Vero cells were seeded into 96-well plates at a density of 1.0×10⁴ cells per well and allowed to attach overnight. Following attachment, the media was replaced with 50 uL of infection medium (DMEM supplemented with 2% fetal bovine serum and 100 units/mL penicillin and streptomycin). A Tecan D300c digital dispenser was then used to add compounds to the culture using an 8-point 3-fold serial dilution format. The DMSO concentration was normalized to 0.5% for all treatments. Following compound addition, 50 μL of infection medium containing 160 TCID₅₀ HSV-2 G strain was added to the cells and incubated at 37° C. for 5 days. After the incubation. 10 μL/well of WST-8 chromogenic reagent was added and the plates incubated at 37° C. for 3 hours. Following the incubation, cell viability was quantified by measuring the absorbance at 460 nm and 620 nm using a Tecan Infinite M1000 Pro plate reader.

Table 2 provides assay data for exemplified compounds of the invention grouped in the following ranges: A indicates EC₅₀<100 nM; B indicates EC₅₀ of >100 to <1,000 nM; C indicates EC₅₀ of >1,000 to <5,000 nM; NA indicates not available.

TABLE 2
Assay data for exemplified compounds of the invention.

| Compound Number | HSV-1 | HSV-2 |
|---|---|---|
| 1 | A | A |
| 2 | B | B |
| 3 | B | B |
| 4 | B | B |
| 5 | A | A |
| 6 | A | A |
| 7 | A | A |
| 8 | A | A |
| 9 | A | A |
| 10 | A | A |
| 11 | NA | A |
| 12 | A | A |
| 13 | A | A |
| 14 | A | A |
| 15 | B | NA |
| 16 | B | B |
| 17 | A | A |
| 18 | A | A |
| 19 | NA | C |
| 20 | NA | B |
| 21 | NA | B |
| 22 | A | A |
| 23 | NA | C |
| 24 | A | A |
| 25 | A | A |
| 26 | A | A |
| 27 | NA | A |
| 28 | C | C |
| 29 | C | NA |
| 30 | NA | A |
| 31 | NA | A |
| 32 | NA | B |
| 33 | B | NA |
| 34 | B | NA |
| 35 | A | A |
| 36 | A | A |
| 37 | NA | B |
| 38 | NA | C |
| 39 | B | NA |
| 40 | C | C |
| 41 | B | B |
| 42 | A | A |
| 43 | A | A |
| 44 | C | NA |
| 45 | C | C |
| 46 | B | NA |
| 47 | NA | B |
| 48 | NA | A |
| 49 | NA | B |
| 50 | NA | B |
| 51 | C | NA |
| 52 | C | C |
| 53 | B | NA |
| 54 | B | B |
| 55 | A | A |
| 56 | B | B |
| 57 | NA | C |
| 58 | NA | A |
| 59 | C | C |
| 60 | C | C |
| 61 | B | NA |
| 62 | C | NA |
| 63 | A | NA |
| 64 | NA | B |
| 65 | NA | B |
| 66 | A | NA |
| 67 | A | A |
| 68 | A | A |
| 69 | NA | A |
| 70 | NA | A |
| 71 | A | NA |
| 72 | NA | A |
| 73 | A | A |
| 74 | B | B |
| 75 | C | C |
| 76 | C | NA |
| 77 | B | B |
| 78 | B | B |
| 79 | A | A |
| 80 | A | A |
| 81 | NA | B |
| 82 | B | NA |
| 83 | A | NA |
| 84 | B | A |
| 85 | A | A |
| 86 | B | B |
| 87 | C | NA |
| 88 | B | C |
| 89 | C | C |
| 90 | B | B |
| 91 | B | NA |
| 92 | NA | A |
| 93 | A | NA |
| 94 | NA | C |
| 95 | B | B |
| 96 | A | A |
| 97 | A | A |
| 98 | A | A |
| 99 | A | A |
| 100 | C | NA |
| 101 | A | A |
| 102 | A | A |
| 103 | A | A |
| 104 | C | NA |
| 105 | A | A |
| 106 | A | NA |
| 107 | NA | A |
| 108 | NA | A |
| 109 | NA | A |
| 110 | A | A |
| 111 | A | NA |
| 112 | NA | A |
| 113 | C | C |
| 114 | NA | A |
| 115 | C | NA |
| 116 | A | A |
| 117 | A | A |
| 118 | A | A |
| 119 | A | A |
| 120 | A | A |
| 121 | A | A |
| 122 | A | A |
| 123 | A | A |
| 124 | A | A |
| 125 | B | NA |
| 126 | A | A |
| 127 | A | A |
| 128 | A | A |
| 129 | B | NA |
| 130 | A | A |
| 131 | B | B |
| 132 | B | A |
| 133 | A | A |
| 134 | B | NA |
| 135 | B | NA |
| 136 | B | NA |
| 137 | C | NA |
| 138 | A | A |
| 139 | B | NA |
| 140 | B | B |
| 141 | A | A |
| 142 | NA | B |
| 143 | NA | A |
| 144 | NA | B |
| 145 | NA | B |
| 146 | NA | B |
| 147 | NA | A |
| 148 | A | NA |
| 149 | B | NA |
| 150 | A | NA |
| 151 | NA | B |
| 152 | NA | C |
| 153 | NA | B |
| 154 | NA | A |
| 155 | A | A |
| 156 | A | A |
| 157 | B | A |
| 158 | B | NA |
| 159 | B | NA |
| 160 | B | NA |
| 161 | A | A |
| 162 | B | NA |
| 163 | B | B |
| 164 | A | A |
| 165 | C | NA |
| 166 | B | B |
| 167 | B | NA |
| 168 | B | NA |
| 169 | A | NA |
| 170 | A | NA |
| 171 | A | A |
| 172 | A | A |
| 173 | A | A |
| 174 | C | C |
| 175 | C | C |
| 176 | C | C |
| 177 | C | NA |
| 178 | C | C |
| 179 | C | NA |
| 180 | B | NA |
| 181 | A | B |
| 182 | A | A |
| 183 | B | NA |
| 184 | NA | A |
| 185 | NA | A |
| 186 | NA | B |
| 187 | NA | B |
| 188 | NA | B |
| 189 | NA | B |
| 190 | NA | B |
| 191 | NA | A |
| 192 | NA | A |
| 193 | A | A |
| 194 | A | A |
| 195 | NA | A |
| 196 | NA | A |
| 197 | NA | C |
| 198 | NA | A |
| 199 | NA | B |
| 200 | NA | B |
| 201 | NA | C |
| 202 | NA | A |
| 203 | NA | A |
| 204 | NA | C |
| 205 | NA | B |
| 206 | NA | A |
| 207 | NA | B |
| 208 | NA | A |
| 209 | A | A |
| 210 | A | A |
| 211 | A | A |
| 212 | A | A |
| 213 | NA | A |
| 214 | NA | A |
| 215 | NA | A |
| 216 | A | A |
| 217 | NA | A |
| 218 | NA | A |
| 219 | NA | B |
| 220 | NA | A |
| 221 | NA | A |
| 222 | NA | A |
| 223 | NA | A |
| 224 | NA | A |
| 225 | NA | A |
| 226 | NA | B |
| 227 | NA | B |
| 228 | A | A |
| 229 | NA | A |
| 230 | NA | B |
| 231 | NA | B |
| 232 | NA | B |
| 233 | NA | B |
| 234 | NA | A |
| 235 | A | A |
| 236 | NA | A |
| 237 | NA | A |
| 238 | NA | C |
| 239 | NA | A |
| 240 | NA | B |
| 241 | NA | C |
| 242 | A | A |
| 243 | A | A |
| 244 | NA | A |
| 245 | A | A |
| 246 | NA | A |
| 247 | NA | A |
| 248 | NA | A |
| 249 | NA | A |
| 250 | NA | A |
| 251 | A | A |
| 252 | NA | A |
| 253 | NA | A |
| 254 | NA | B |
| 255 | A | A |
| 256 | NA | A |
| 257 | NA | A |
| 258 | NA | A |
| 259 | A | A |
| 260 | NA | A |
| 261 | NA | B |
| 262 | NA | C |
| 263 | NA | B |
| 264 | NA | A |
| 265 | NA | A |
| 266 | NA | B |
| 267 | NA | B |
| 268 | NA | A |
| 269 | NA | B |
| 270 | NA | A |
| 271 | NA | C |
| 272 | NA | C |
| 273 | NA | B |
| 274 | A | A |
| 275 | NA | A |
| 276 | NA | A |
| 277 | A | A |
| 278 | NA | B |
| 279 | NA | B |
| 280 | NA | C |
| 281 | NA | B |
| 282 | NA | B |
| 283 | NA | B |
| 284 | NA | B |
| 285 | NA | A |
| 286 | NA | C |
| 287 | NA | A |
| 288 | NA | B |
| 289 | NA | C |
| 290 | NA | C |
| 291 | NA | A |
| 292 | NA | C |
| 293 | A | A |
| 294 | NA | B |
| 295 | NA | A |
| 296 | NA | B |
| 297 | NA | B |
| 298 | NA | A |
| 299 | NA | A |
| 300 | NA | B |
| 301 | NA | A |
| 302 | NA | B |
| 303 | NA | A |
| 304 | NA | A |
| 305 | A | A |
| 306 | NA | A |
| 307 | NA | B |
| 308 | NA | B |
| 309 | NA | A |
| 310 | A | A |
| 311 | NA | B |
| 312 | NA | C |
| 313 | NA | A |
| 314 | NA | A |
| 315 | NA | A |
| 316 | A | A |
| 317 | NA | a |
| 318 | A | A |
| 319 | NA | A |
| 320 | NA | A |
| 321 | NA | A |
| 322 | A | A |
| 323 | A | A |
| 324 | A | A |
| 325 | NA | A |
| 326 | NA | A |
| 327 | NA | A |
| 328 | NA | A |
| 329 | NA | A |
| 330 | NA | A |
| 331 | NA | A |
| 332 | NA | A |
| 333 | NA | A |
| 334 | NA | A |
| 335 | NA | C |
| 336 | NA | A |
| 337 | NA | A |
| 338 | B | B |
| 339 | A | A |
| 340 | NA | A |
| 341 | NA | A |
| 342 | A | A |
| 343 | A | A |
| 344 | NA | A |
| 345 | NA | B |
| 346 | NA | B |
| 347 | NA | B |
| 348 | NA | B |
| 349 | NA | A |
| 350 | NA | A |
| 351 | NA | A |
| 352 | NA | A |
| 353 | NA | A |
| 354 | NA | A |
| 355 | NA | A |
| 356 | NA | A |
| 357 | NA | C |
| 358 | NA | C |
| 359 | NA | C |
| 360 | NA | A |
| 361 | A | A |
| 362 | NA | A |
| 363 | A | A |
| 364 | NA | A |
| 365 | NA | A |
| 366 | NA | A |
| 367 | NA | A |
| 368 | NA | A |
| 369 | A | A |
| 370 | NA | A |
| 371 | NA | A |
| 372 | NA | A |
| 373 | NA | A |
| 374 | NA | A |
| 375 | NA | A |
| 376 | A | A |
| 377 | NA | A |
| 378 | NA | A |
| 379 | NA | A |
| 380 | NA | C |
| 381 | NA | B |
| 382 | NA | B |
| 383 | NA | B |
| 384 | NA | A |
| 385 | NA | B |
| 386 | NA | A |
| 387 | NA | A |
| 388 | NA | A |
| 389 | NA | A |
| 390 | NA | A |
| 391 | NA | A |
| 392 | NA | A |
| 393 | NA | A |
| 394 | NA | A |
| 395 | NA | A |
| 396 | NA | A |
| 397 | NA | B |
| 398 | NA | B |
| 399 | NA | B |
| 400 | NA | A |
| 401 | NA | A |
| 402 | NA | B |
| 403 | NA | A |
| 404 | NA | A |
| 405 | NA | B |
| 406 | NA | A |
| 407 | NA | B |
| 408 | NA | A |
| 409 | NA | A |
| 410 | NA | A |
| 411 | NA | A |
| 412 | NA | B |
| 413 | NA | A |
| 414 | NA | A |
| 415 | NA | A |
| 416 | NA | A |
| 417 | A | A |
| 418 | NA | A |
| 419 | NA | A |
| 420 | NA | A |
| 421 | NA | A |
| 422 | NA | A |
| 423 | NA | A |
| 424 | NA | A |
| 425 | NA | A |
| 426 | NA | A |
| 427 | NA | A |
| 428 | NA | A |
| 429 | NA | A |
| 430 | NA | A |
| 431 | NA | A |
| 432 | NA | A |
| 433 | NA | A |
| 434 | A | A |
| 435 | NA | A |
| 436 | A | A |
| 437 | NA | A |
| 438 | NA | C |
| 439 | NA | A |
| 440 | NA | A |
| 441 | A | A |

It will be understood that various details of the presently disclosed subject matter may be changed without departing from the scope of the presently disclosed subject matter. Furthermore, the foregoing description is for the purpose of illustration only, and not for the purpose of limitation.

All publications, patents, and patent applications cited in this specification are incorporated herein by reference for the teaching to which such citation is used.

Test compounds for the experiments described herein were employed in free or salt form.

The specific responses observed may vary according to and depending on the particular active compound selected or whether there are present carriers, as well as the type of formulation and mode of administration employed, and such expected variations or differences in the results are contemplated in accordance with practice of the present disclosure.

Although specific embodiments of the present disclosure are herein illustrated and described in detail, the disclosure is not limited thereto. The above detailed descriptions are provided as exemplary of the present disclosure and should not be construed as constituting any limitation of the disclosure. Modifications will be obvious to those skilled in the art, and all modifications that do not depart from the spirit of the disclosure are intended to be included with the scope of the appended claims.

1. A compound of Formula II: 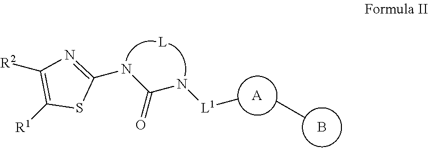
or a pharmaceutically acceptable salt thereof, wherein: 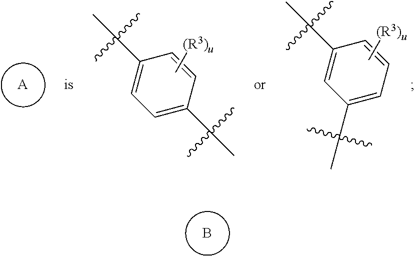
is selected from the group consisting of: 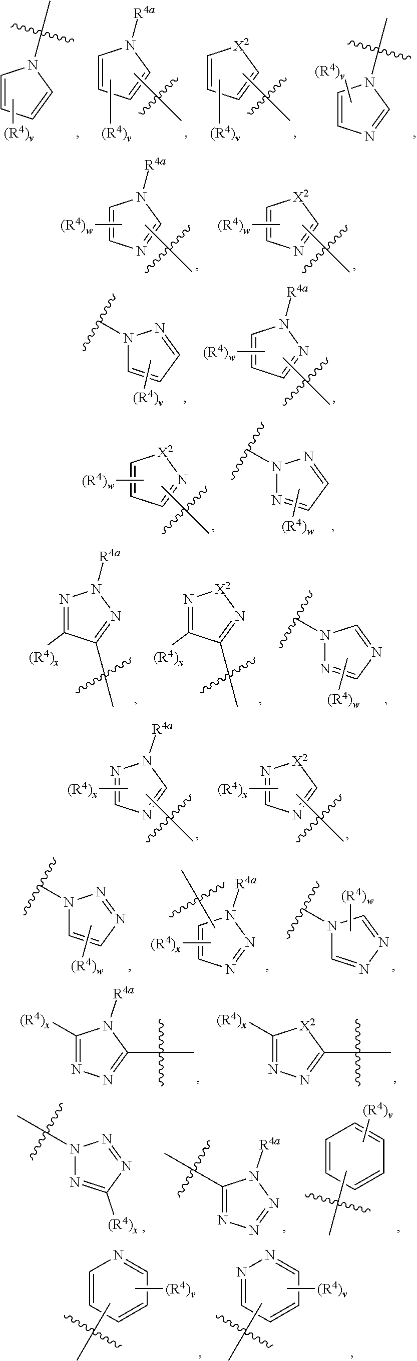 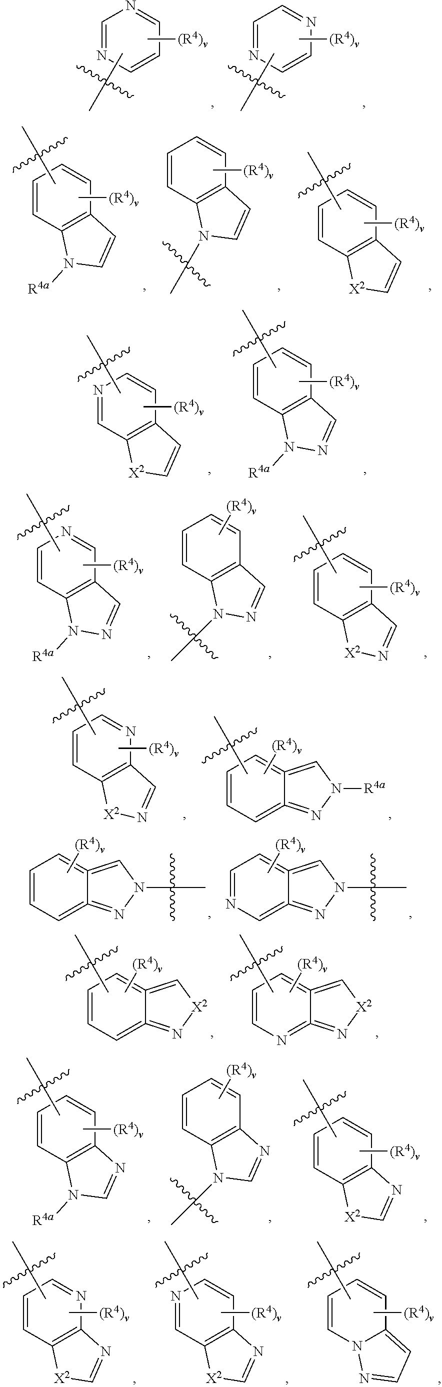 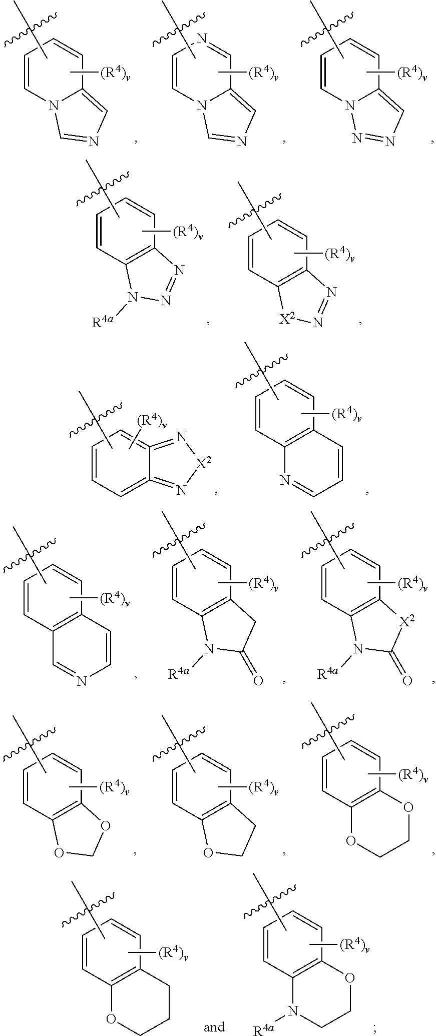
X² and X⁴ are independently selected from the group consisting of O and S;
X⁵ is OH₂, OF₂, O, S or NR^{y},
L is 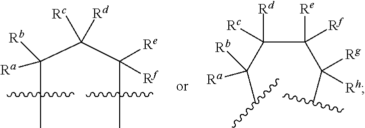
L¹ is a bond; or L¹ is —CH₂— when 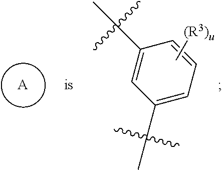
R^{a}, R^{b}, R^{c}, R^{d}, R^{e}, R^{f}, R^{g} and R^{h} are independently selected from the group consisting of hydrogen, halo, CN, OH, NRⁿR^{m}, —C(O)OH, —C(O)OC₁₋₄alkyl, —C(O)NRⁿR^{m}, —SO₂NRⁿR^{m}, C₁₋₄alkyl, C₂₋₄alkenyl, C₂₋₄alkynyl, haloC₁₋₄alkyl, hydroxyC₁₋₄alkyl, and C₁₋₄alkoxy; or two R-groups together with the carbon atom to which they are attached form a C₃₋₆monocycloalkyl, 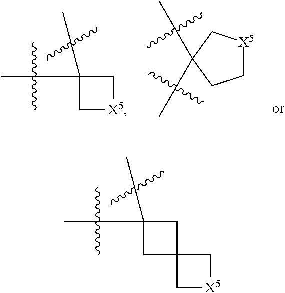
group;
Rⁿ and R^{m} are independently selected for each occurrence from the group consisting or hydrogen and C₁₋₄alkyl;
R^{y} is hydrogen, C₁₋₄alkyl or acetyl;
R¹ is 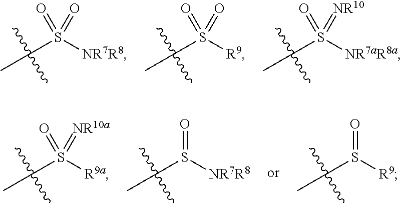
R² is hydrogen, halo, CN, OH, C₁₋₄alkyl, C₂₋₄alkenyl, C₂₋₄alkynyl, haloC₁₋₄alkyl, C₁₋₄alkoxy, hydroxyC₁₋₄alkyl or haloC₁₋₄alkoxy;
R⁴ is independently selected for each occurrence from the group consisting of halo, CN, OH, NRⁿR^{m}, C₁₋₄alkyl, haloC₁₋₄alkyl, C₂₋₄alkenyl, C₂₋₄alkynyl optionally substituted with hydroxyC₁₋₃alkyl, cyclopropyl optionally substituted with halo or cyano, and R^{4b}, provided that only one R⁴ group can be R^{4b};
R^{4a} is hydrogen, C₁₋₄alkyl, halo C₁₋₄alkyl and hydroxyC₁₋₄alkyl;
R^{4b} is selected from the group consisting of: 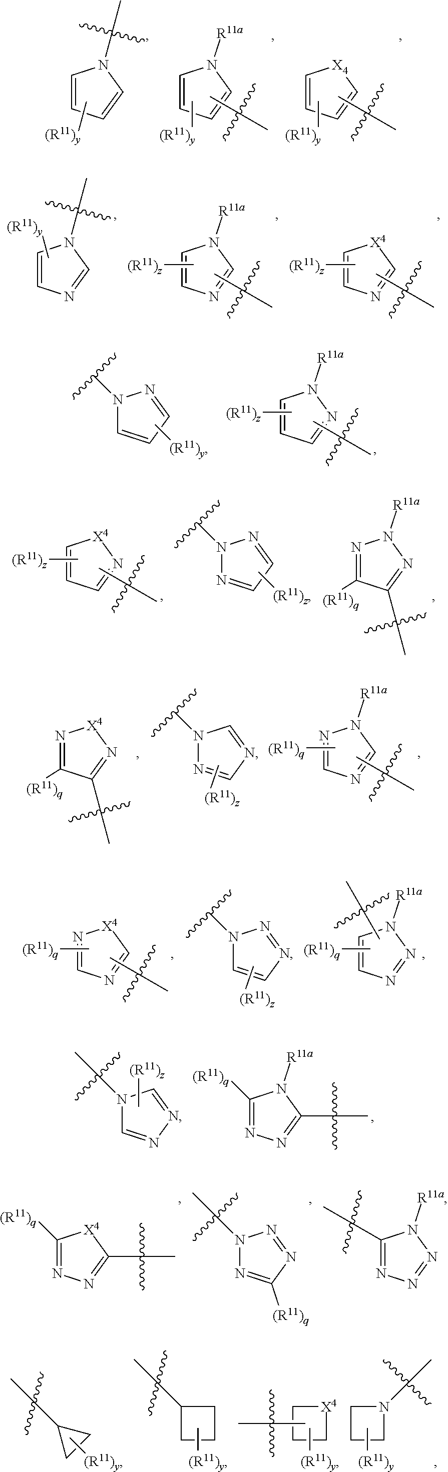 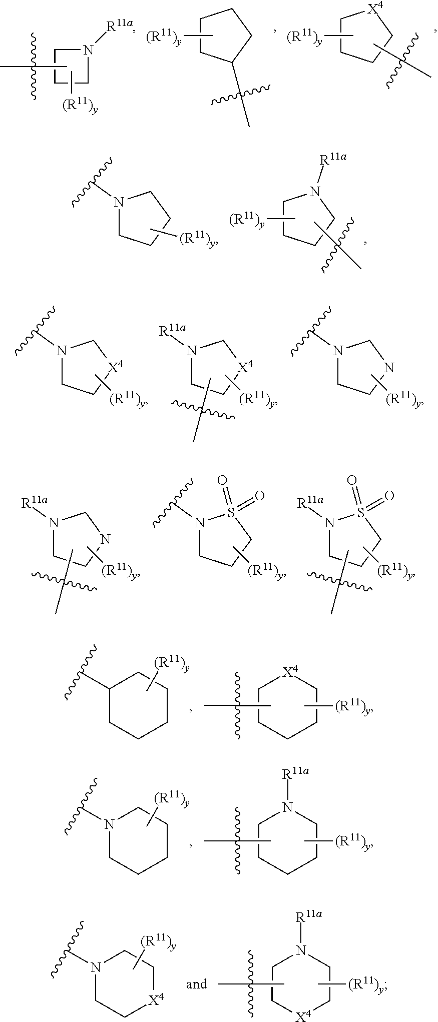
R⁷ and R⁸ are independently selected from the group consisting of hydrogen, OH, acetyl, C₁₋₁₀alkyl, haloC₁₋₁₀alkyl, hydroxyC₁₋₁₀alkyl, C₁₋₄alkoxyC₁₋₁₀alkyl, C₃₋₆monocycloalkyl, phenyl, pyridyl or indolyl; or R⁷ and R³ together with the N-atom to which they are attached form an arizidinyl, azetidinyl, pyrrolidinly, piperidinyl, morpholinyl or thiomorpholinyl group, wherein the arizidinyl, azetidinyl, pyrrolidinly or piperidinyl group is optionally substituted with halo, CN or OH;
R^{7a} and R^{8a} are independently selected from the group consisting of hydrogen, C₁₋₄alkyl and C₃₋₆monocycloalkyl, or R⁷ and R⁸ together with the N atom to which they are attached form an arizidinyl, azetidinyl, pyrrolidinyl, or piperidinyl morpholinyl or thiomorpholinyl group;
R⁹ and R^{9a} are independently selected from the group consisting or C₁₋₄alkyl and haloC₁₋₄alkyl;
R¹⁰ and R^{10a} are independently selected from the group consisting of hydrogen and C₁₋₄alkyl;
R¹¹ is independently selected from the group consisting of halo, CN, OH, NRⁿR^{m}, C₁₋₄alkyl, haloC₁₋₄alkyl, C₂₋₄alkenyl, C₂₋₄alkynyl, and C₃₋₆monocycloalkyl;
R^{11a} is hydrogen, C₁₋₄alkyl, halo C₁₋₄alkyl and hydroxyC₁₋₄alkyl;
q and x are independently selected from the group consisting of 0 and 1;
w and z are independently selected from the group consisting of 0, 1 and 2; and
v and y are independently selected from the group consisting of 0, 1, 2 and 3.

2. The compound of claim 1, or a pharmaceutically acceptable salt thereof, wherein: 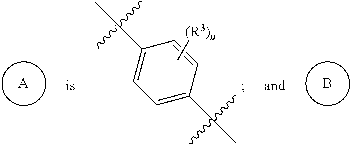
is selected from the group consisting of:

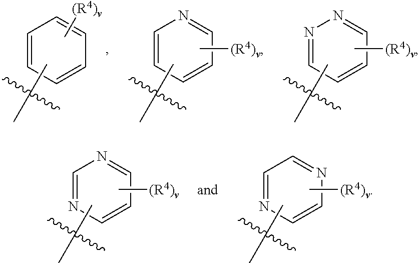

3. The compound of claim 2, or a pharmaceutically acceptable salt thereof, wherein:

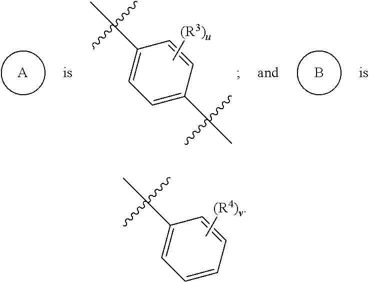

4. The compound of claim 1, or a pharmaceutically acceptable salt thereof, wherein: 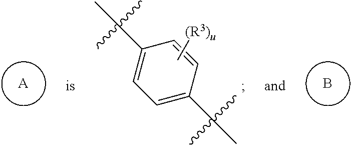
is selected from the group consisting of:

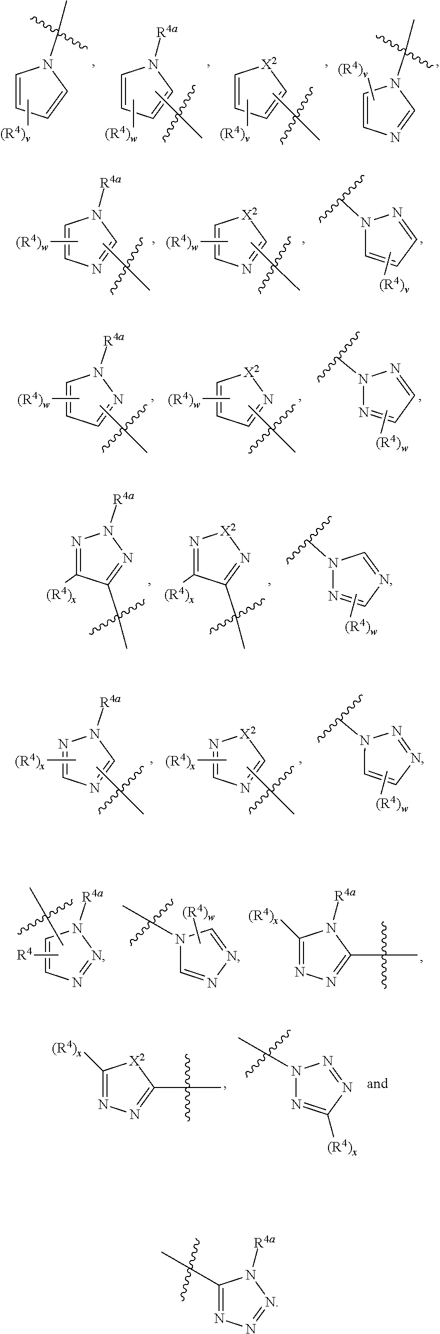

5. The compound of claim 1, or a pharmaceutically acceptable salt thereof, wherein: 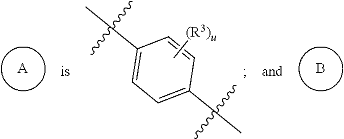
is selected from the group consisting of:

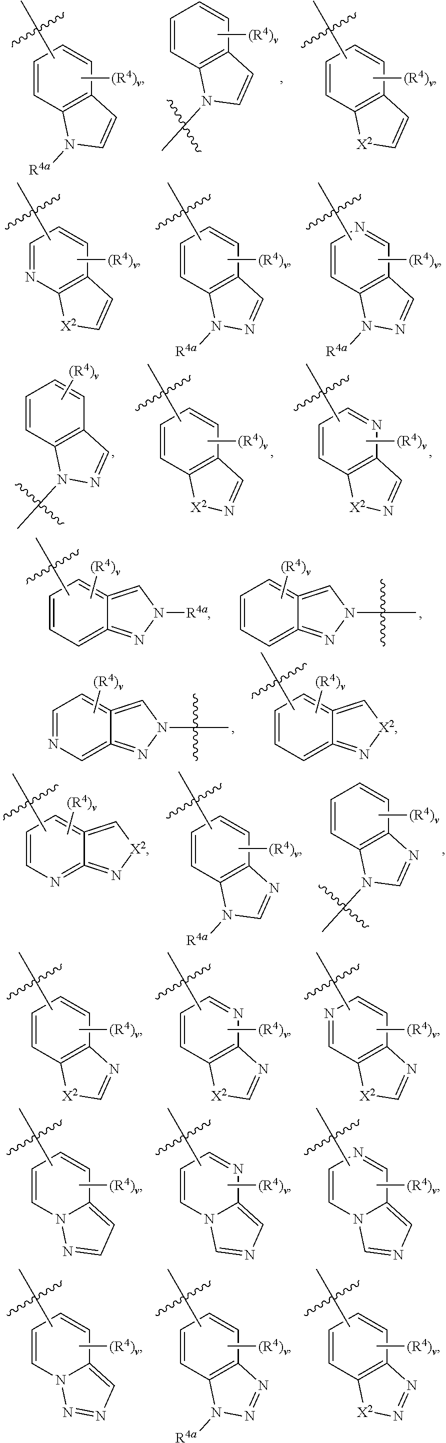

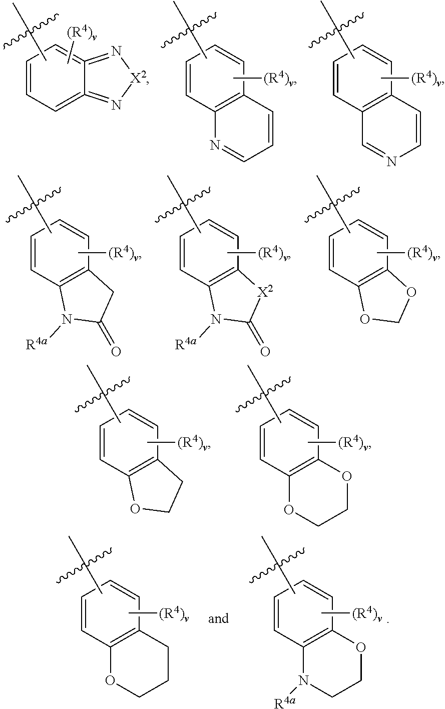

6. The compound of claim 1, or a pharmaceutically acceptable salt thereof, wherein

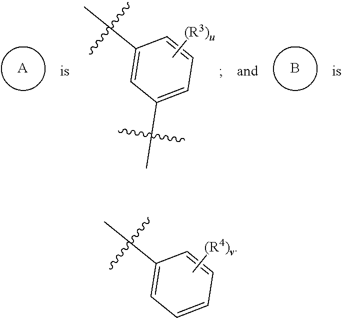

7. The compound of claim 1, or a pharmaceutically acceptable salt thereof, wherein: L is

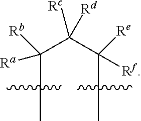

8. The compound of claim 7, or a pharmaceutically acceptable salt thereof, wherein: L is

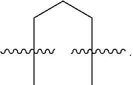 [SMILES: *CCC*]

9. The compound of claim 1, or a pharmaceutically acceptable salt thereof, wherein: L is

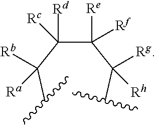

10. The compound of claim 9, or a pharmaceutically acceptable salt thereof, wherein: L is

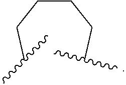 [SMILES: *CCCC*]

11. The compound of claim 1, or a pharmaceutically acceptable salt thereof, wherein: R² is H, C₁, F, CH₃ or CF₃.

12. The compound of claim 11, or a pharmaceutically acceptable salt thereof, wherein: R² is CH₃.

13. The compound of claim 1, or a pharmaceutically acceptable salt thereof, wherein: R¹ is

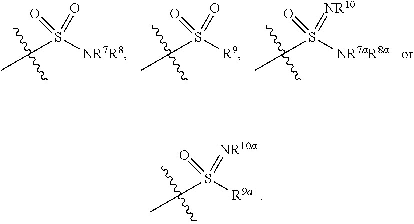

14. The compound of claim 13, or a pharmaceutically acceptable salt thereof, wherein: R¹ is

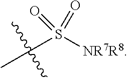

15. The compound of claim 14, or a pharmaceutically acceptable salt thereof, wherein: R¹ is R¹ is

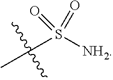 [SMILES: *S(N)(=O)=O]

16. The compound of claim 13, or a pharmaceutically acceptable salt thereof, wherein: R¹ is

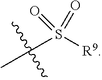

17. The compound of claim 16, or a pharmaceutically acceptable salt thereof, wherein: R¹ is

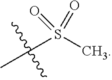 [SMILES: *S(C)(=O)=O]

18. The compound of claim 13, or a pharmaceutically acceptable salt thereof, wherein: R¹ is

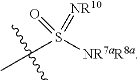

19. The compound of claim 18, or a pharmaceutically acceptable salt thereof, wherein: R¹ is

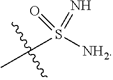 [SMILES: *S(=N)(N)=O]

20. The compound of claim 13, or a pharmaceutically acceptable salt thereof, wherein: R¹ is

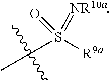

21. The compound of claim 20, or a pharmaceutically acceptable salt thereof, wherein: R¹ is

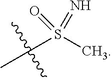 [SMILES: *S(C)(=N)=O]

22. The compound of claim 1, or a pharmaceutically acceptable salt thereof, wherein: R³ is halo for each occurrence and u is 0, 1, 2 or 3.

23. The compound of claim 22, or a pharmaceutically acceptable salt thereof, wherein: u is 0.

24. The compound of claim 1, or a pharmaceutically acceptable salt thereof, wherein: R⁴ is independently selected for each occurrence from the group consisting of halo, CN, methyl, CHF₂, CF₃, acetylenyl, and cyclopropyl.

25. The compound of claim 1, or a pharmaceutically acceptable salt thereof, wherein: R⁴ is independently selected from halo for all occurrences.

26. A pharmaceutical composition comprising a compound of claim 1, or a pharmaceutically acceptable salt thereof, and a pharmaceutically acceptable excipient.

27. A method for the treatment or prophylaxis of an HSV infection in a subject in need thereof, the method comprising: administering to the subject a therapeutically effective amount of a compound of claim 1, or a pharmaceutically acceptable salt thereof.

28. A method for the treatment or prophylaxis of an HSV infection in a subject in need thereof, the method comprising: administering to the subject a therapeutically effective amount of a pharmaceutical composition of claim 26.

29. The method of claim 27, wherein infection is an HSV-1 infection.

30. The method of claim 27, wherein infection is an HSV-2 infection.

31. (canceled)
